(19) United States
(12) Patent Application Publication
(10) Pub. No.: US 2003/0170309 A1
(43) Pub. Date: Sep. 11, 2003

(54) Pharmaceutical compositions containing polymer and drug assemblies

(76) Inventors: Walter C. Babcock, Bend, OR (US); Marshall D. Crew, Bend, OR (US); Dwayne T. Friesen, Bend, OR (US); Mark D. Rabenstein, Bend, OR (US); Ravi M. Shanker, Groton, CT (US); Daniel T. Smithey, Bend, OR (US)

Correspondence Address: PFIZER INC., PATENT DEPARTMENT, MS8260-1611, EASTERN POINT ROAD, GROTON, CT, 06340, US

(21) Appl. No.: 10/173,945

(22) Filed: Jun. 17, 2002

Related U.S. Application Data
(60) Provisional application No. 60/300,259, filed on Jun. 22, 2001.

(51) Int. Cl. A61K 9/14

(52) U.S. Cl. 424/486

(57) ABSTRACT

Solutions containing polymer/drug assemblies of a low-solubility drug and polymer are disclosed. In addition, solid aggregated polymer/drug assemblies are disclosed comprising a low-solubility drug and polymer.

This application claims the benefit of priority of provisional Patent Application Serial No. 60/300,259 filed Jun. 22, 2001.

### BACKGROUND OF THE INVENTION

The present invention relates to pharmaceutical compositions containing drug and polymer assemblies, and in particular to compositions of low-solubility drugs which provide improved drug concentrations.

Several methods have been described to utilize polymers to increase the concentration of low-solubility drugs. One conventional method attempts to mix a polymer with the drug to enhance the dissolution rate of the drug. For example, Martin et al., U.S. Pat. No. 4,344,934 mixed poorly-soluble drugs with polymers such as hydroxypropyl methyl cellulose (HPMC) and added an aqueous surfactant solution to the drug-polymer mixture. While this results in improved dissolution, there is only slight enhancement of drug concentration relative to the equilibrium concentration. Piergiorgio et al., U.S. Pat. No. 4,880,623 used solvent processing to co-precipitate nifedipine with PEG and adsorbed this onto polymers such as HPMC, or onto other excipients. While increased drug bioavailability was observed, no comparison was made between different drug forms. Uedo et al., U.S. Pat. No. 5,093,372 mixed the sparingly-soluble drug exifone with polymers such as HPMC to increase bioavailability. However, this did not result in any enhanced drug concentration of the drug/polymer mixture relative to the bulk crystalline form of the drug.

Utilizing solubility-improved forms of drugs such as more soluble salt forms, more soluble polymorphs, or amorphous drug forms may result in a temporary improvement in the concentration of the drug in the solution, where the dissolution rate exceeds the crystallization or precipitation rate. However, such improvements are often only short lived. Eventually, the low-solubility drug returns to a lowest energy crystalline or amorphous state and crystallizes or otherwise precipitates from solution. When this occurs rapidly, increases in bioavailability via this approach are often limited.

EP 0 499 299 A2 discloses another method for improving the concentration of drug in aqueous solution by using a polymer along with a milling process to reduce the drug particle size to improve dissolution. EP 0 499 299 A2 discloses dispersible particles consisting essentially of a crystalline drug substance having a surface modifier adsorbed on the surface thereof in an amount sufficient to maintain a particle size of about 400 nm. The surface modifier may be selected from a wide range of excipients, including polymers.

Usui, et al., Inhibitory Effects of Water-soluble Polymers on Precipitation of RS-8359, Int'l J. of Pharmaceutics 154 (1997) 59-66, disclose the use of three polymers, namely hydroxy propyl methyl cellulose, hydroxy propyl cellulose, and polyvinylpyrrolidone to inhibit precipitation of the low-solubility drug RS-8359. The drug and polymer were dissolved in a mixture of 0.5 N HCl and methanol, and then added to a phosphate buffer solution. Usui et al. observed that the particular polymers inhibited crystallization of the drug.

It is also known that formulating a drug in a solid amorphous dispersion of the drug and a polymer may enhance the maximum concentration of the drug in an aqueous solution, and likewise may also enhance bioavailability of the drug. For example, Curatolo et al. EP 0 901 786 A2 disclose spray-dried amorphous dispersions of low-solubility drugs and the polymer hydroxy propyl methyl cellulose acetate succinate. When such dispersions are dissolved in an aqueous buffer solution, the dispersions provide superior aqueous concentration of drug relative to dispersions formed from other methods.

Nakamichi, et al., U.S. Pat. No. 5,456,923 disclose solid dispersions formed by twin-screw extrusion of low solubility drugs and various polymers.

Jensen et al., U.S. Pat. No. 5,460,823 discloses particles of a hydrophobic substance, such as a drug, and hydrocolloids having a size not exceeding 10 μm.

EP 0 988 863 A2 discloses water-insoluble complexes of poorly soluble compounds molecularly dispersed in water-insoluble ionic polymers. The compounds are molecularly dispersed in the ionic polymers in the amorphous form.

Yano, et al., In Vitro Stability and in Vivo Absorption Studies of Colloidal Particles Formed from a Solid Dispersion System, Chem. Pharm. Bull. 44(12) 2309-2313 (1996) disclose dispersions of a poorly soluble drug YM022 in hydroxypropyl methyl cellulose and polyoxyethylene hydrogenated castor oil. Colloidal particles having a mean diameter of 160 nm and containing 67%-77% of the drug YM022 were produced when the dispersion was administered to an aqueous solution. On oral administration to rats, good absorption was observed for the colloidal solution.

Nevertheless, there remains a need to improve the aqueous concentration and bioavailability of low-solubility drugs, and for compositions comprising a drug that are capable of providing enhanced concentration of the drug in aqueous solution relative to the equilibrium concentration of the drug, that maintain the concentration of the drug in such a solution over time or at least reduces the rate at which the drug concentration decreases from the enhanced concentration to the equilibrium concentration, that may be easily and cheaply prepared and that ultimately enhance the bioavailability of poorly soluble drugs (when dosed orally). These needs and others that will become apparent to one of ordinary skill are met by the present invention, which is summarized and described in detail below.

### BRIEF SUMMARY OF THE INVENTION

The present invention provides polymer/drug assemblies which greatly enhance the concentration of a low-solubility drug in aqueous solution. The invention provides aqueous solutions containing such polymer/drug assemblies, methods for forming solutions containing such assemblies, compositions comprising solid aggregated polymer/drug assemblies, and methods for forming such compositions.

In a first aspect of the invention, an aqueous solution comprises a low-solubility drug and an amphiphilic polymer that is at least partially dissolved in the aqueous solution. By “at least partially dissolved” is meant that not all of the polymer present in the solution must be completely dissolved, in the sense that it is entirely solvated. Some of the polymer may be present as polymer aggregates ranging from two or three molecules up to large macroscopic particles. A portion of the drug and a portion of the polymer are each present in the solution in the form of amorphous polymer/drug assemblies having a diameter of from 20 nm to 5 μm. The solution has a total dissolved drug concentration of at least 2.0-fold that of an equilibrium concentration of the drug. By “equilibrium concentration” is meant the drug concentration provided by a control composition comprising an equivalent amount of the drug in crystalline form but free from the polymer. The solution also has a free drug concentration of at least 1.5-fold that of the equilibrium concentration provided by the control composition.

In another aspect of the invention, a method is provided for forming an aqueous solution containing polymer/drug assemblies. The drug is administered to the solution in a fashion so as to achieve a concentration of drug in solution that at least temporarily exceeds the equilibrium concentration of the drug. An amphiphilic polymer is also at least partially dissolved in the solution in a sufficient amount so as to form polymer/ drug assemblies having a diameter of from 20 nm to 5000 nm.

In another aspect of the invention, a solid pharmaceutical composition is provided comprising a solid aggregated polymer/drug assembly comprising an amorphous, low-solubility drug and an amphiphilic polymer.

Finally, the invention provides a method for forming solid aggregated polymer/drug assemblies from aqueous solutions containing polymer/drug assemblies. A first solution of a low-solubility drug and an amphiphilic polymer is formed. A portion of the drug and a portion of the polymer are each present in the form of polymer/drug assemblies having a diameter of from 20 nm to 5000 nm. Solid aggregated polymer/drug assemblies are isolated from the first solution, the solid aggregated polymer/drug assemblies comprising the low-solubility drug in amorphous form and the amphiphilic polymer.

As described below in greater detail, a “polymer/drug assembly” refers to a collection of polymer molecules and drug molecules which are physically associated to form an assembly or aggregate that is sufficiently small that it remains “suspended” in solution (as described below) and which is “labile,” meaning that drug molecules may rapidly convert to free drug and free drug may rapidly associate with the polymer/drug assemblies.

As used herein, the term “free drug” refers to drug molecules which are dissolved in the aqueous solution and are generally either monomeric or clusters of no more than 100 molecules. Thus, by free drug we mean that the drug is not present in the form of a polymer/drug assembly or other species of aggregated drug, where the drug species or particle is sufficiently large that its solubility is less than 1.25-fold that of bulk crystalline drug. This generally means that “free drug” refers to that portion of any drug clusters present that are made up of no more than about 100 molecules.

As used herein, the term “total dissolved drug” refers to the total amount of drug dissolved in the aqueous solution, and includes drug present in any form less than about 5000 nm in size and includes drug in the form of free drug, micelles, and polymer/drug assemblies. Specifically, this means that total dissolved drug may be determined by separating out any undissolved drug by centrifugation or filtration and then measuring the amount of drug remaining in the supernatant or filtrate.

The present invention provides several advantages over prior methods for enhancing the concentration and bioavailability of low-solubility drugs. Polymer/drug assemblies, when present in an aqueous solution, dramatically increase the amount of free drug present in the solution. The polymer/drug assemblies greatly enhance the concentration of free drug in solution with respect to the concentration provided by a control composition of pure drug in either the crystalline or amorphous form. The polymer/drug assemblies also function as a reservoir of drug that: (1) is mobile (may diffuse rapidly); (2) is labile; and (3) provides a high free drug concentration. In combination, these properties greatly enhance the rate and extent of drug absorption (e.g., bioavailability). Thus, the compositions of the present invention result in higher relative bioavailability of drugs formulated to form such polymer/drug assemblies in solution compared to conventional formulations.

The foregoing and other objectives, features, and advantages of the invention will be more readily understood upon consideration of the following detailed description of the invention.

### BRIEF DESCRIPTION OF THE SEVERAL VIEWS OF THE DRAWINGS

FIG. 1 shows the results of a lability assay for the polymer/drug assemblies of Example 1.

FIG. 2 shows the results of a lability assay for the polymer/drug assemblies of Example 30.

FIG. 3 shows the results of a lability assay for the polymer/drug assemblies of Example 31.

FIG. 4 shows Differential Scanning Calorimetry (DSC) scans for the solid aggregated polymer/drug assemblies of Example 36, a 25% Drug 2/HPMCAS-MF solid amorphous dispersion, and a physical mixture of Drug 2 and HPMCAS-MF.

FIG. 5 shows DSC scans for the solid aggregated polymer/drug assemblies of Example 55 and the solid amorphous dispersion of Control C11.

FIG. 6 shows a scanning electron micrograph of the solid aggregated polymer/drug assemblies of Example 56.

FIG. 7 shows a scanning electron micrograph of the solid amorphous dispersion of Control C11.

FIG. 8 shows a scanning electron micrograph of the solid aggregated polymer/drug assemblies of Example 65.

FIG. 9 shows the powder X-ray diffraction patterns for (1) crystalline ziprasidone free-base, (2) the polymer/drug assemblies of Example 64, (3) the polymer/drug assemblies of Example 65, (4) the polymer/drug assemblies of Example 66, and (5) the solid amorphous dispersion of Control C11.

### DETAILED DESCRIPTION OF THE PREFERRED EMBODIMENTS

The present invention relates to polymer/drug assemblies that improve the concentration of low-solubility drugs in aqueous solution, and that provide improved bioavailability. The present invention arises out of the investigation by the inventors into the ability of certain solid, amorphous dispersions of drug and polymer to dramatically improve the aqueous concentration of a low-solubility drug in a use environment relative to conventional dosage formulations. The inventors observed that the solid, amorphous spray-dried dispersions of a low-solubility drug and the polymer hydroxypropyl methyl cellulose succinate acetate disclosed in Curatolo et al. EP 0 901 786 A2 provided greatly improved concentration of dissolved drug compared to other dosage formulations. During the course of investigating the properties of the aqueous solutions formed by administering these solid, amorphous dispersions to a use environment, the present inventors discovered the presence of small polymer/drug assemblies in the resulting aqueous solutions. These polymer/drug assemblies comprise small assemblies of polymer and amorphous drug, on the order of 5000 nm in diameter or smaller, which are present in the aqueous solution. The inventors believe that these assemblies play a significant role in improving the concentration of dissolved drug as well as free drug in the aqueous solution.

The ability of the polymer/drug assemblies to increase drug concentration and bioavailablity is a surprising result. Contrary to the conventional methods for improving drug concentration and absorption of drug, the present inventors have determined that the free drug concentration of a low-solubility drug may be improved by increasing the amount of drug present in the form of other drug containing species (the polymer/drug assemblies), rather than by improving the dissolution rate of the drug or even by attempting to increase directly the concentration or solubility of free drug by addition of a solvent or other “solubilizing” agents. This is a significant departure from conventional methods used to increase drug concentration which seek to directly increase the free drug concentration.

While not wishing to be bound by a particular theory, the present inventors believe that the polymer/drug assemblies of the present invention improve drug concentration in aqueous solution by raising the free energy of the drug while at the same time lowering the total free energy of the polymer and drug system. For low-solubility drugs, the lowest free energy state of the drug alone is the crystalline or amorphous form. Methods which succeed in generating in solution concentrations of free drug above the solubility of the crystalline or amorphous forms generally have limited success as the dissolved drug usually precipitates from solution as the crystalline or amorphous form. Thus, for example, if a more soluble salt form of a basic drug is formed and isolated as a crystalline material and subsequently administered to an aqueous use environment, the drug often initially dissolves in the solution but quickly converts to the free-base form of the drug and precipitates from solution as either the amorphous or crystalline free-base drug. The free energy of the drug in the polymer/drug assemblies is greater than the free energy of drug in a pure crystalline or amorphous phase (i.e., no polymer present). Surprisingly, the total free energy of the system decreases as the low-solubility drug and amphiphilic polymer partition from the aqueous solution to form polymer/drug assemblies. Thus, the driving force for formation of polymer/drug assemblies is a lowering of polymer free energy that exceeds the increase in free energy of the drug, so that the overall free energy of the system (drug and polymer) decreases.

When polymer and drug are added to an aqueous use environment that is either the GI tract of an animal, or an in vitro use environment that simulates the GI tract of an animal, it is believed that at least four different drug forms are formed: (1) free drug; (2) drug present within bile salt micelles that are either naturally occurring or synthetic that are present in the GI tract or test solution; (3) polymer/drug assemblies; and (4) precipitate. “Precipitate” is a general term for any relatively large particulates that form and fall out of solution. Any drug present in such precipitate is termed “not dissolved drug.” Such precipitate may comprise: (1) crystalline drug; (2) amorphous drug; or (3) a mixture of drug and polymer that is present as particles that are sufficiently large so as to drop out of solution (greater than about 5 to 10 microns in average diameter). It is desired to increase the free drug concentration because, in general, primarily free drug is directly absorbed from the GI tract into the blood. The absorption rate of a drug from the GI tract to the blood is therefore generally proportional to the free drug concentration at the intestinal membrane surface. Drug present in the other three phases generally must first convert to the free drug form in order to be absorbed.

It is believed that the polymer/drug assemblies of the present invention enhance the drug absorption rate, and therefore relative bioavailability, by one or more of the following mechanisms. The polymer/drug assemblies provide a higher free drug concentration that is sustained, particularly in the GI tract of a mammal, for physiologically relevant time, that is for 30 minutes to 16 hours or even longer. The polymer/drug assemblies provide a drug containing material that can rapidly release drug from the polymer/drug assembly to replace free drug as it is absorbed into the blood and removed from the solution. This rapid equilibration, termed “lability,” and hence replacement of free drug, allows the polymer/drug assemblies to function as a reservoir of drug that is available for conversion to free drug and then absorption. The ability of the polymer/drug assemblies to rapidly equilibrate with the free drug is due primarily to the small size of the polymer/drug assemblies, resulting in a high surface area to volume ratio, and high mobility of drug in the polymer/drug assemblies relative to other drug phases, such as crystalline drug or amorphous drug or even large polymer/drug particulates such as may be present as precipitate.

Owing to the fact that the presence of polymer/drug assemblies provides an enhanced free drug concentration in solutions when micelles are present, the assemblies may also provide a higher concentration of drug that is incorporated into micelles. Generally, for a given concentration of micelles, the amount of drug that partitions into the micelles will be roughly proportional to the free drug concentration. Thus, by increasing the amount of drug in the free drug state, the amount of drug in micelles may also be proportionally increased. Drug in micelles is particularly mobile (rapid diffusion rate) and labile (rapid dissociation rate) such that drug in micelles is particularly bioavailable (relative, for example, to any of the species present as precipitate).

Both drug-containing micelles and polymer/drug assemblies have sufficient mobility and lability that they can transport drug through the unstirred water layer (including the glycocalyx and mucus that covers the intestinal wall) thereby raising the free drug concentration at the intestinal wall which in turn can raise the drug absorption rate.

Finally, the conversion of much of the free drug that would otherwise be present at a concentration that greatly exceeds the equilibrium drug concentration to polymer/drug assemblies prevents or retards crystallization or precipitation of much of the drug as a low-solubility form, such as the lowest energy crystalline from of the drug or pure amorphous drug. The presence of polymer that interacts with the drug surface is also believed to prevent any drug clusters that may nucleate from growing into large amorphous particles or crystals by adsorbing to the drug-cluster surface.

In total, these effects may serve to increase the bioavailability of a low-solubility drug by at least 1.25-fold to more than 100-fold. The relative bioavailability provided by the polymer/drug assemblies is at least 1.25-fold to 10-fold or more the relative bioavailability of a control composition comprised of a composition containing an equivalent amount of drug but which does not form such polymer/drug assemblies.

The inventors have determined that polymer/drug assemblies may be formed through a variety of methods in addition to administering a solid, amorphous dispersion of a low-solubility drug and polymer to an aqueous solution. In addition, the inventors have found that a certain class of polymers, namely amphiphilic polymers, is preferred. The polymer/drug assemblies find utility any time it is desired either to raise the concentration of a low-solubility drug in an aqueous solution, increase the rate at which drug is absorbed from the lumen of the gastrointestinal tract, decrease the amount of drug that is dosed, raise the fraction of drug absorbed when a given dose is given orally, or a combination thereof. The polymer/drug assemblies, drugs, amphiphilic polymers which may be used, and methods for creating the polymer/drug assemblies are discussed in more detail below.

### Polymer/Drug Assemblies

The polymer/drug assemblies of the present invention comprise an amphiphilic polymer and a low-solubility drug. Such polymer/drug assemblies may be formed anytime a low-solubility drug and an amphiphilic polymer are at least both partially dissolved in sufficient amounts in an aqueous solution. The low-solubility drug must be dosed in a form and dosed at a high enough level to achieve at least temporarily a dissolved drug concentration that exceeds the equilibrium concentration of the drug provided by the lowest energy crystalline or amorphous form of the drug in the use environment. As described in more detail below, any method that results in providing an initially enhanced concentration of drug exceeding the equilibrium concentration, and which also provides in the solution at least partially dissolved polymer, may be used.

The aqueous solution may be any solution containing a significant amount of water, such as greater than about 20 wt %. More typically, the aqueous solution is a solution that contains from about 40 wt % up to near 100 wt % water. One subset of aqueous solutions are use environments. As used herein, a “use environment” can be either the in vivo environment of the GI tract, subdermal, intranasal, buccal, intrathecal, ocular, intraaural, subcutaneous spaces, vaginal tract, arterial and venous blood vessels, pulmonary tract or intramuscular tissue of an animal, such as a mammal and particularly a human, or the in vitro environment of a test solution, such as phosphate buffered saline (PBS) or a Model Fasted Duodenal (MFD) solution. An appropriate PBS solution is an aqueous solution comprising 20 mM sodium phosphate, 47 mM potassium phosphate, 87 mM NaCl and 0.2 mM KCl, adjusted to pH 6.5. An appropriate MFD solution is the same PBS solution wherein additionally is present 7.3 mM sodium taurocholic acid and 1.4 mM of 1-palmitoyl-2-oleyl-sn-glycero-3-phosphocholine. A composition or method of the invention can be tested in vivo or, more conveniently, in vitro to ascertain whether it is within the scope of the invention.

The polymer/drug assemblies are believed to be very small structures consisting of drug and polymer present in the solution. Although the drug may be present in extremely small clusters and may be to some extent ordered (such as the order that exists in micelles) the drug is non-crystalline in nature. While not wishing to be bound by a particular theory, the polymer/drug assemblies are thought to consist of micelle-like structures in which portions of the polymer and drug are in relatively close proximity, organizing so as to form one or more hydrophobic regions that are shielded from aqueous solution and one or more hydrophillic regions that are in contact with the aqueous solution.

The polymer/drug assemblies are small enough so as to remain suspended in solution without the application of mechanical stirring. By “suspended” is meant that the polymer/drug assemblies do not significantly precipitate or settle out of solution due to the influence of gravity. Following formation, at least 25% of the polymer/drug assemblies that are in solution at their maximum level remain suspended in solution upon standing with no stirring for at least ninety (90) minutes. More preferably, at least 50% of the maximum level remain suspended in solution upon standing with no stirring for at least ninety (90) minutes. Polymer/drug assemblies range generally from about 20 nm to 5000 nm in average diameter. For some polymer/drug combinations, this size range will be narrower, with the majority of the polymer/drug assemblies having a mean diameter of less than about 2 μm, and in some cases less than about 1 μm and typically fall within a narrower distribution of from 100 to 800 nm in average diameter. In general, smaller assemblies, that is, those less than about 1 μm in average diameter, are preferred, because smaller assemblies remain suspended longer, diffuse more rapidly, and are more labile relative to larger assemblies.

The amount of drug and polymer contained in an individual polymer/drug assembly varies depending on the nature of the polymer and drug, as well as the size of the assembly, but generally is in the range of from 5 wt % drug to 95 wt % drug. In general, for a given drug, the smaller the polymer/drug assembly, the smaller the fraction of drug in the polymer/drug assembly.

The small size of the polymer/drug assemblies means that they are highly mobile. Generally, the diffusion rate of particles is inversely related to their size. Thus, polymer/drug assemblies that are on the order of 100 nm in average diameter will generally diffuse more rapidly than, for example, particles of crystalline or amorphous drug that are greater than a few microns in diameter. Specifically, the polymer/drug assemblies of this invention will have diffusion coefficients in an aqueous solution such as PBS solution that are greater than about 1×10⁻¹⁰ cm²/sec. The polymer/drug assemblies can therefore rapidly diffuse through the unstirred aqueous layer adjacent to the lipid bilayer membrane of the epithelium and can rapidly release drug to the aqueous layer adjacent to the lipid wall of the intestine, thereby acting as a shuttle for the drug. This is particularly important for drug with relatively low aqueous solubility dosed at a level where a majority of the drug is not in the form of dissolved free drug. In such cases, the polymer/drug assemblies can shuttle drug to the intestinal wall and thereby maintain the concentration of free drug at the intestinal wall closer to that in the bulk intestinal lumen, thereby enhancing the rate and extent of drug absorption.

The polymer/drug assemblies are also stable but labile when present in a use environment. By stable is meant that in the absence of drug absorption as would occur in the GI tract, the concentration of the so-formed polymer/drug assemblies is relatively constant over extended periods of time, e.g., several hours. Generally, a majority of drug initially present in a solution in the form of such assemblies, when the total dissolved drug reaches its maximum value, remains suspended in solution, in the absence of any absorption, for at least ninety (90) minutes and preferably at least 240 minutes. Thus, the fraction of drug present in polymer/drug assemblies that remains in solution for at least 90 minutes is at least about 25% that of the maximum level and preferably at least about 50% of its maximum level.

By labile is meant that both polymer and drug molecules may rapidly dissociate and associate with the polymer/drug assemblies. The disassociation rate, or rate at which drug interconverts between a polymer/drug assembly and free drug, is very fast. The disassociation rate is believed to be roughly first order with respect to the concentration of the polymer/drug assemblies, and thus, a quantitative measure of the dissociation rate is the “half-life” or t_{½} of the dissociation of drug from the polymer/drug assembly. The “half-life” of the disassociation of drug from the polymer/drug assembly, termed t_{½} is defined as the time for the light-scattering signal of the polymer/drug assemblies to drop half way from an initial level to a final level upon a sudden change in conditions such as the rapid absorption of drug from solution. The value of t_{½} is typically less than about 1000 sec, and preferably less than about 200 sec. The fast disassociation time constant means that the drug in the polymer/drug assemblies is capable of quickly converting to free drug, and vice versa. Fast disassociation time constants are preferred, as this allows the drug in the polymer/drug assemblies to rapidly convert to free drug, which may then be absorbed.

Dissociation rates and disassociation time constants may be measured by any conventional method that distinguishes between free drug and drug in the polymer/drug assemblies. For example, free drug may be rapidly removed from solution by adding a material that binds the free drug, such as cyclodextrin, or adding a phase in which the drug is preferentially soluble such as an emulsified oil or a micelle-forming material. The rate at which the polymer/drug assemblies dissociate under these conditions to release free drug may then be measured by, for example, monitoring the decrease in the light-scattering signal, to determine the rate at which the drug in the polymer/drug assemblies disassociates to regenerate the free drug concentration.

The existence or presence of polymer/drug assemblies may be determined by any analytical method capable of measuring the presence of small molecular assemblies in solution. One method for determining the presence of the polymer/drug assemblies is through dynamic and static light scattering measurements. In combination, these techniques can assess the amount and size distributions of particles in solution, particularly those in the 20 nm to 5000 nm size range. The intensity of the light scattering signal from each method is roughly proportional to the concentration of polymer/drug assemblies for equivalent size assemblies. In addition, the distribution of assembly sizes is calculated from the light-scattering signal. For “dynamic light scattering,” the size and relative amount of assemblies is determined for assemblies in the 10 nm to 1000 nm range. The size this technique yields is termed the “hydrodynamic radius,” which is the effective radius of the polymer/drug assembly based on its rate of diffusion in solution. For “static light scattering” or “StLS,” the size and relative amount of assemblies is determined for assemblies generally in the 200 nm to 5000 nm size range. (The technique measures particles larger than 5000 nm as well.) The size this technique yields is termed the “diameter of gyration,” which is the average diameter of a sphere defined by the assembly tumbling in solution. Since the amount of light scattered is quite large for particles greater than about 5000 nm in size, such particles, termed precipitate, are generally removed via either filtration or centrifugation as described elsewhere, prior to analysis of the solutions via either light scattering method. Either technique may be used to evaluate whether a test solution is within the scope of this invention. However, dynamic light scattering is most appropriate for evaluating assemblies in the 20 nm to 1000 nm size range and static light scattering is most appropriate for evaluating assemblies in the 200 nm to 5000 nm size range. Thus, it is best to utilize both techniques when evaluating solutions. Because each technique measures a fundamentally different physical property, values obtained from the two methods will not normally be equivalent. However, within the scope of this invention are solutions or compositions that meet the size criteria given in the claims when evaluated by either or both techniques. It has been found that calculation of the concentration of drug present in polymer/drug assemblies by subtracting the free drug concentration from the total dissolved drug concentration for various solutions yields values that are roughly proportional to the magnitude of the light-scattering signal. This demonstrates that the drug in solution that is not present as free drug is present in the form of 20 nm to 5000 nm particles.

In addition, the presence of drug in the form of polymer/drug assemblies may be inferred from a combination of total dissolved drug and free drug concentration measurements. In solutions where polymer/drug assemblies are present, the concentration of free drug at a time that is at least 90 minutes following formation of the polymer/drug assemblies is at least 1.5-fold, preferably at least 2-fold, and more preferably at least 3-fold the equilibrium concentration of drug provided by a control composition comprising an equivalent amount of crystalline drug alone. The total dissolved drug concentration in the solution where polymer/drug assemblies are present at a time that is at least 90 minutes following formation of the polymer/drug assemblies is at least 2-fold, more preferably at least 4-fold, and even more preferably at least 10-fold the equilibrium concentration of drug provided by a control composition comprising an equivalent quantity of drug in the crystalline form alone.

Free drug may be quantified using any analytical technique capable of measuring the concentration of free drug but not drug in the form of polymer/drug assemblies. For example, a nuclear magnetic resonance (NMR) technique may be used, since the NMR measurement only yields a well-resolved signal for species that are sufficiently small or mobile that they may rapidly (<millisec.) rotate. In particular, the NMR signal has been found to be proportional to the amount of free drug and any drug that may be present in a mobile, solvated non-aggregated state such as in micelles but not drug present in polymer/drug assemblies. Free drug may also be quantified through permeation analysis in which the rate of drug transport through a dialysis membrane is proportional to the free drug concentration. The amount of drug present in polymer/drug assemblies may be calculated by subtracting the amount of free drug from the concentration of total dissolved drug.

As used herein, the term “total dissolved drug concentration” refers to drug that may be dissolved in the form of free drug, polymer/drug assemblies, or any other drug-containing submicron structure, assembly, aggregate, colloid, or micelle. It will be appreciated by one of ordinary skill that this definition of “total dissolved drug” encompasses not only monomeric solvated drug molecules but also a wide range of species such as polymer/drug assemblies that have submicron dimensions such as drug aggregates, aggregates of mixtures of polymer and drug, micelles, polymeric micelles, colloidal particles or nanocrystals, polymer/drug complexes, and other such drug-containing species that are present in the filtrate or supernatant in the specified dissolution test.

The concentration of total dissolved drug in a dissolution test is typically measured by sampling the test medium and analyzing for the dissolved drug concentration. To avoid relatively large drug particulates which would give an erroneous determination, the test solution is either filtered or centrifuged. Total dissolved drug is typically taken as that material that remains suspended (e.g., does not precipitate) in solution for a period of at least 1 hour without agitation. To speed analysis in in vitro tests, total dissolved drug can be taken to be that material that either passes a syringe filter or alternatively the material that remains in the supernatant following centrifugation. In cases where the polymer/drug assembly size is substantially less than 400 nm, filtration can be conducted using a 13 mm, 0.45 μm polyvinylidine difluoride syringe filter sold by Scientific Resources under the trademark TITAN®. In cases where larger suspended polymer/drug assemblies are present, filters with pore-size ratings of about 5000 nm to 10 μm may be used. Centrifugation is typically carried out in a polypropylene microcentrifuge tube by centrifuging at about 13,000 G for about 60 seconds. Other similar filtration or centrifugation methods can be employed and useful results obtained. For example, using other types of microfilters or other centrifugation speeds and thus within the specified ranges above may yield values higher or lower than that obtained with the filter or centrifugation conditions specified above but will still allow identification of preferred compositions. However, centrifugation for times longer than about 5 minutes at G levels greater than about 13,000 G may yield erroneously low results as the polymer/drug assemblies themselves may be removed.

### The Drug

The present invention is useful with any drug capable of being administered to a solution in a manner such that the concentration of dissolved drug exceeds the equilibrium concentration of the drug at least temporarily, as described below.

The term “drug” is conventional, denoting a compound having beneficial prophylactic and/or therapeutic properties when administered to an animal, especially humans. The drug does not need to be a low-solubility drug in order to benefit from this invention, although low-solubility drugs represent a preferred class for use with the invention. Even a drug that nonetheless exhibits appreciable solubility in the desired environment of use can benefit from the increased solubility/bioavailability made possible by this invention if the addition of the concentration-enhancing polymer can reduce the size of the dose needed for therapeutic efficacy or increase the rate of drug absorption in cases where a rapid onset of the drug's effectiveness is desired.

Preferably, the drug is a “low-solubility drug,” meaning that the drug may be either “substantially water-insoluble,” which means that the drug has a minimum aqueous solubility at physiologically relevant pH (e.g., pH 1-8) of less than 0.01 mg/mL, “sparingly water-soluble,” that is, has an aqueous solubility up to about 1 to 2 mg/mL, or even low to moderate aqueous-solubility, having an aqueous-solubility from about 1 mg/mL to as high as about 20 to 40 mg/mL. The invention finds greater utility as the solubility of the drug decreases. Thus, compositions of the present invention are preferred for low-solubility drugs having a solubility of less than 10 mg/mL, more preferred for low-solubility drugs having a solubility of less than 1 mg/mL, and even more preferred for low-solubility drugs having a solubility of less than 0.1 mg/mL. In general, it may be said that the drug has a dose-to-aqueous solubility ratio greater than 10 mL, and more typically greater than 100 mL, where the drug solubility (in mg/mL) is the minimum value observed in any physiologically relevant aqueous solution (e.g., those with pH values between 1 and 8) including USP simulated gastric and intestinal buffers, and dose is in mg. The dose-to-aqueous-solubility ratio may be calculated by dividing the dose (in mg) by the solubility (in mg/mL).

Preferred classes of drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, cholesterol-reducing agents, antiobesity agents, autoimmune disorder agents, anti-impotence agents, antibacterial and anti-fungal agents, hypnotic agents, anti-Parkinsonism agents, anti-Alzheimer's disease agents, antibiotics, anti-depressants, antiviral agents, anti-atherosclerotic agents, glycogen phosphorylase inhibitors, and cholesterol ester transfer protein inhibitors.

Each named drug should be understood to include the neutral form of the drug, pharmaceutically acceptable salts, as well as prodrugs. Specific examples of antihypertensives include prazosin, nifedipine, amlodipine besylate, trimazosin and doxazosin; specific examples of a blood glucose-lowering agent are glipizide and chlorpropamide; a specific example of an anti-impotence agent is sildenafil and sildenafil citrate; specific examples of antineoplastics include chlorambucil, lomustine and echinomycin; a specific example of an imidazole-type antineoplastic is tubulazole; a specific example of an anti-hypercholesterolemic is atorvastatin calcium; specific examples of anxiolytics include hydroxyzine hydrochloride and doxepin hydrochloride; specific examples of anti-inflammatory agents include betamethasone, prednisolone, aspirin, piroxicam, valdecoxib, carprofen, celecoxib, flurbiprofen and (+)-N-{4-[3-(4-fluorophenoxy)phenoxy]-2-cyclopenten-1-yl}-N-hyroxyurea; a specific example of a barbiturate is phenobarbital; specific examples of antivirals include acyclovir, nelfinavir, and virazole; specific examples of vitamins/nutritional agents include retinol and vitamin E; specific examples of beta blockers include timolol and nadolol; a specific example of an emetic is apomorphine; specific examples of a diuretic include chlorthalidone and spironolactone; a specific example of an anticoagulant is dicumarol; specific examples of cardiotonics include digoxin and digitoxin; specific examples of androgens include 17-methyltestosterone and testosterone; a specific example of a mineral corticoid is desoxycorticosterone; a specific example of a steroidal hypnotic/anesthetic is alfaxalone; specific examples of anabolic agents include fluoxymesterone and methanstenolone; specific examples of antidepression agents include sulpiride, [3,6-dimethyl-2-(2,4,6-trimethyl-phenoxy)-pyridin-4-yl]-(1-ethylpropyl)-amine, 3,5-dimethyl-4-(3′-pentoxy)-2-(2′,4,6′-trimethylphenoxy)pyridine, pyroxidine, fluoxetine, paroxetine, venlafaxine and sertraline; specific examples of antibiotics include carbenicillin indanylsodium, bacampicillin hydrochloride, troleandomycin, doxycyline hyclate, ampicillin and penicillin G; specific examples of anti-infectives include benzalkonium chloride and chlorhexidine; specific examples of coronary vasodilators include nitroglycerin and mioflazine; a specific example of a hypnotic is etomidate; specific examples of carbonic anhydrase inhibitors include acetazolamide and chlorzolamide; specific examples of antifungals include econazole, terconazole, fluconazole, voriconazole, and griseofulvin; a specific example of an antiprotozoal is metronidazole; specific examples of anthelmintic agents include thiabendazole and oxfendazole and morantel; specific examples of antihistamines include astemizole, levocabastine, cetirizine, loratadine, decarboethoxy-loratadine and cinnarizine; specific examples of antipsychotics include ziprasidone, olanzepine, thiothixene hydrochloride, fluspirilene, risperidone and penfluridole; specific examples of gastrointestinal agents include loperamide and cisapride; specific examples of serotonin antagonists include ketanserin and mianserin; a specific example of an anesthetic is lidocaine; a specific example of a hypoglycemic agent is acetohexamide; a specific example of an anti-emetic is dimenhydrinate; a specific example of an antibacterial is cotrimoxazole; a specific example of a dopaminergic agent is L-DOPA; specific examples of anti-Alzheimer's Disease agents are THA and donepezil; a specific example of an anti-ulcer agent/H2 antagonist is famotidine; specific examples of sedative/hypnotic agents include chlordiazepoxide and triazolam; a specific example of a vasodilator is alprostadil; a specific example of a platelet inhibitor is prostacyclin; specific examples of ACE inhibitor/antihypertensive agents include enalaprilic acid and lisinopril; specific examples of tetracycline antibiotics include oxytetracycline and minocycline; specific examples of macrolide antibiotics include erythromycin, clarithromycin, and spiramycin; a specific example of an azalide antibiotic is azithromycin, specific examples of glycogen phosphorylase inhibitors include [R-(R*S*)]-5-chloro-N-[2-hydroxy-3-{methoxymethylamino}-3-oxo-1-(phenylmethyl)propyl-1H-indole-2-carboxamide and 5-chloro-1H-indole-2-carboxylic acid [(1S)-benzyl-(2R)-hydroxy-3-((3R,4S)-dihydroxy-pyrrolidin-1-yl-)-3-oxypropyl]amide; specific examples of cholesterol esterase transfer protein inhibitors include [2R,4S]-4-[3,5-bis-trifluoromethyl-benzyl)-methoxycarbonyl-amino]-2-ethyl-6-trifluoromethyl-3,4-dihydro-2H-quinoline-1-carboxylic acid ethyl ester and [2R,4S]-4-[acetyl-(3,5-bis-trifluoromethyl-benzyl)-amino]-2-ethyl-6-trifluoromethyl-3,4-dihydro-2H-quinoline-1-carboxylic acid isopropyl ester.

The invention finds particular utility with cholesterol ester transfer protein (CETP) inhibitors. The inventors have recognized a subclass of CETP inhibitors that are essentially aqueous insoluble, highly hydrophobic, and are characterized by a set of physical properties for which the invention is particularly useful. This subclass exhibits dramatic enhancements in aqueous concentration and bioavailability when formulated using the compositions and methods of the present invention.

The first property of this subclass of essentially insoluble, hydrophobic CETP inhibitors is extremely low aqueous solubility. By extremely low aqueous solubility is meant that the minimum aqueous solubility at physiologically relevant pH (pH of 1 to 8) is less than about 10 μg/ml and preferably less than about 1 μg/ml.

A second property is a very high dose-to-solubility ratio. Extremely low solubility often leads to poor or slow absorption of the drug from the fluid of the gastrointestinal tract, when the drug is dosed orally in a conventional manner. For extremely low solubility drugs, poor absorption generally becomes progressively more difficult as the dose (mass of drug given orally) increases. Thus, a second property of this subclass of essentially insoluble, hydrophobic CETP inhibitors is a very high dose (in mg) to solubility (in mg/ml) ratio (ml). By “very high dose-to-solubility ratio,” is meant that the dose-to-solubility ratio has a value of at least 1000 ml, and preferably at least 5,000 ml, and more preferably at least 10,000 ml.

A third property of this subclass of essentially insoluble, hydrophobic CETP inhibitors is that they are extremely hydrophobic. By extremely hydrophobic is meant that the Clog P value of the drug, has a value of at least 4.0, preferably a value of at least 5.0, and more preferably a value of at least 5.5.

A fourth property of this subclass of essentially insoluble CETP inhibitors is that they have a low melting point. Generally, drugs of this subclass will have a melting point of about 150° C. or less, and preferably about 140° C. or less.

Primarily, as a consequence of some or all of these four properties, CETP inhibitors of this subclass typically have very low absolute bioavailabilities. Specifically, the absolute bioavailibility of drugs in this subclass when dosed orally in their undispersed (e.g., crystalline) state is less than about 10% and more often less than about 5%.

Turning now to the chemical structures of specific CETP inhibitors, one class of CETP inhibitors that finds utility with the present invention consists of oxy substituted 4-carboxyamino-2-methyl-1,2,3,4-tetrahydroquinolines having the Formula I

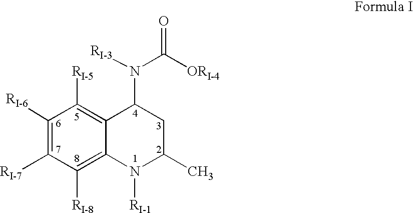

and pharmaceutically acceptable salts, enantiomers, or stereoisomers of said compounds;

wherein R_{I-1} is hydrogen, Y_{I}, W_{I}—X_{I}, W_{I}—Y_{I};

wherein W_{I} is a carbonyl, thiocarbonyl, sulfinyl or sulfonyl; X_{I} is —O—Y_{I}, —S—Y_{I}—N(H) —Y_{I} or —N—(Y_{I})₂;

wherein Y_{I} for each occurrence is independently Z_{I}, or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{I};

wherein Z_{I} is a partially saturated, fully saturated or fully unsaturated three to eight membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{I} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino, said (C₁-C₆)alkyl substituent is also optionally substituted with from one to nine fluorines;

R_{I-3} is hydrogen or Q_{I};

wherein Q_{I} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with VI;

wherein V_{I} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{I} substituent is optionally mono-, di-, tri-, or tetra-substituted independently with halo, (C₁-C₆)alkyl, (C₂-C₆)alkenyl, hydroxy, (C₁-C₆)alkoxy, ((C₁-C₄)alkylthio, amino, nitro, cyano, oxo, carbamoyl, mono-N- or di-N,N-(C₁-C₆) alkylcarbamoyl, carboxyl, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino, said (C₁-C₆)alkyl or (C₂-C₆)alkenyl substituents are also optionally substituted with from one to nine fluorines;

R_{I}-₄ is Q_{I-1} or V_{I-1}

wherein Q_{I-1} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{I-1};

wherein V_{I-1} is a partially saturated, fully saturated or fully unsaturated three to six membered ring optionally having one to two heteroatoms selected independently from oxygen, sulfur and nitrogen;

wherein said V_{I-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C6)alkylamino wherein said ((C₁-C₆)alkyl substituent is optionally mono-substituted with oxo, said (C₁-C₆)alkyl substituent is also optionally substituted with from one to nine fluorines;

wherein either R_{I-3} must contain V_{I} or R_{I-4} must contain V_{I-1}; and

R_{I-5} R_{I-6} R_{I-7} and R_{I-8} are each independently hydrogen, hydroxy or oxy wherein said oxy is substituted with T_{I} or a partially saturated, fully saturated or fully unsaturated one to twelve membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with T_{I};

wherein T_{I} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said TI substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines.

Compounds of Formula I are disclosed in commonly assigned pending U.S. patent application Ser. No. 09/390,731, the complete disclosure of which is herein incorporated by reference.

In a preferred embodiment, the CETP inhibitor is selected from one of the following compounds of Formula I:

[2R,4S] 4-[(3,5-dichloro-benzyl)-methoxycarbonyl-amino]-6,7-dimethoxy-2-methyl-3,4-dihydro-2H-quinoline-1-carboxylic acid ethyl ester;

[2R,4S] 4-[(3,5-dinitro-benzyl)-methoxycarbonyl-amino]-6,7-dimethoxy-2-methyl-3,4-dihydro-2H-quinoline-1-carboxylic acid ethyl ester;

[2R,4S] 4-[(2,6-dichloro-pyridin-4-ylmethyl)-methoxycarbonyl-amino]-6,7-dimethoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-6-methoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-7-methoxy-2-methyl-3,4-dihydro-2H-quinoline-1-carboxylic acid ethyl ester,

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid isopropyl ester;

[2R,4S] 4-[(3,5-bis-trifluoromethyl-benzyl)-ethoxycarbonyl-amino]-6,7-dimethoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid 2,2,2-trifluoro-ethylester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid propyl ester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid tert-butyl ester;

[2R,4S] 4-[(3,5-bis-trifluoromethyl-benzyl)-methoxycarbonyl-amino]-2-methyl-6-trifluoromethoxy-3,4-dihydro-2H-quinoline-1-carboxylic acid ethyl ester,

[2R,4S] (3,5-bis-trifluoromethyl-benzyl)-(l-butyryl-6,7-dimethoxy-2-methyl-1,2,3,4-tetrahydro-quinolin-4-yl)-carbamic acid methyl ester;

[2R,4S] (3,5-bis-trifluoromethyl-benzyl)-(l-butyl-6,7-dimethoxy-2-methyl-1,2,3,4-tetrahydro-quinolin-4-yl)-carbamic acid methyl ester;

[2R,4S] (3,5-bis-trifluoromethyl-benzyl)-[1-(2-ethyl-butyl)-6,7-dimethoxy-2-methyl-1,2,3,4-tetrahydro-quinolin-4-yl]-carbamic acid methyl ester, hydrochloride

Another class of CETP inhibitors that finds utility with the present invention consists of 4-carboxyamino-2-methyl-1,2,3,4,-tetrahydroquinolines, having the Formula II

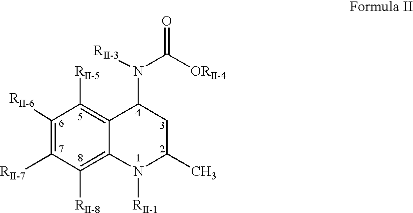

and pharmaceutically acceptable salts, enantiomers, or stereoisomers of said compounds;

wherein R_{II-1} is hydrogen, Y_{II}, W_{II}—X_{II}, W_{II}—Y_{II}; wherein W_{II} is a carbonyl, thiocarbonyl, sulfinyl or sulfonyl; X_{II} is —O—Y_{II}—S—Y_{II}, —N(H) —Y_{II}or —N—(Y_{II})₂;

wherein Y_{II} for each occurrence is independently Z_{II} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{II};

Z_{II} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{II} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C6)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C6)alkyloxycarbonyl, mono-N- or di-N,N-(C₁-C₆)alkylamino, said (C₁-C₆)alkyl is also optionally substituted with from one to nine fluorines;

R_{II-3} is hydrogen or Q_{II};

wherein Q_{II} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{II};

wherein V_{II} is a partially saturated, fully saturated or fully unsaturated three to twelve membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said V_{II} substituent is optionally mono-, di-, tri-, or tetra-substituted independently with halo, (C₁-C₆)alkyl, (C₂-C₆)alkenyl, hydroxy, (C1-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino or said (C₁-C₆)alkyl or (C₂-C₆)alkenyl substituents are optionally substituted with from one to nine fluorines;

R_{II-4} is Q_{II-1} or V_{II-1}

wherein Q_{II-1} a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{II-1};

wherein V_{II-1} is a partially saturated, fully saturated or fully unsaturated three to six membered ring optionally having one to two heteroatoms selected independently from oxygen, sulfur and nitrogen;

wherein said V_{II-1} substituent is optionally mono-, di-, tri-, or tetra-substituted independently with halo, (C₁-C₆) alkyl, (C₁-C₆)alkoxy, amino, nitro, cyano, (C₁-C₆) alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is optionally substituted with from one to nine fluorines;

wherein either R_{II-3} must contain V_{II} or R_{II-4} must contain V_{II-1}; and

R_{II-5}, R_{II-6} R_{II-7} and R_{II-8} are each independently hydrogen, a bond, nitro or halo wherein said bond is substituted with T_{II} or a partially saturated, fully saturated or fully unsaturated (C₁-C₁₂)straight or branched carbon chain wherein carbon may optionally be replaced with one or two heteroatoms selected independently from oxygen, sulfur and nitrogen wherein said carbon atoms are optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon is optionally mono-substituted with T_{II};

wherein T_{II} is a partially saturated, fully saturated or fully unsaturated three to twelve membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said TI substituent is optionally mono-, di- or tri-substituted independently with halo, (C₁-C₆)alkyl, (C₂-C₆) alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆) alkylamino, said (C₁-C₆)alkyl substituent is also optionally substituted with from one to nine fluorines; provided that at least one of substituents R_{II-5} R_{II-6}, R_{II-7} and R_{II-8} is not hydrogen and is not linked to the quinoline moiety through oxy.

Compounds of Formula II are disclosed in commonly assigned pending U.S. patent application Ser. No. 09/391,273 the complete disclosure of which is herein incorporated by reference.

In a preferred embodiment, the CETP inhibitor is selected from one of the following compounds of Formula II:

[2R,4S] 4-[(3,5-Bis-trifluoromethyl-benzyl)-methoxycarbonyl-amino]-2-methyl-7-trifluoromethyl-3,4-dihydro-2H-quinoline-1-carboxylic acid ethyl ester;

[2R,4S] 4-[(3,5-Bis-trifluoromethyl-benzyl)-methoxycarbonyl-amino]-7-chloro-2-methyl-3,4-dihydro-2H-quinoline-1-carboxylic acid ethyl ester;

[2R,4S] 4-[(3, 5-Bis-trifluoromethyl-benzyl)-methoxycarbonyl-amino]-6-chloro-2-methyl-3,4-dihydro-2H-quinoline-1-carboxylic acid ethyl ester;

[2R,4S] 4-[(3,5-Bis-trifluoromethyl-benzyl)-methoxycarbonyl-amino]-2,6,7-trimethyl-3,4-dihydro-2H-quinoline-1-carboxylic acid ethyl ester

[2R,4S] 4-[(3,5-Bis-trifluoromethyl-benzyl)-methoxycarbonyl-amino]-6,7-diethyl-2-methyl-3,4-dihydro-2H-quinoline-1-carboxylic acid ethyl ester;

[2R,4S] 4-[(3,5-Bis-trifluoromethyl-benzyl)-methoxycarbonyl-amino]-6-ethyl-2-methyl-3,4-dihydro-2H-quinoline-1-carboxylic acid ethyl ester;

[2R,4S] 4-[(3,5-Bis-trifluoromethyl-benzyl)-methoxycarbonyl-amino]-2-methyl-6-trifluoromethyl-3,4-dihydro-2H-quinoline-1-carboxylic acid ethyl ester.

[2R,4S] 4-[(3,5-bis-trifluoromethyl-benzyl)-methoxycarbonyl-amino]-2-methyl-6-trifluoromethyl-3,4-dihydro-2H-quinoline-1-carboxylic acid isopropyl ester.

Another class of CETP inhibitors that finds utility with the present invention consists of annulated 4-carboxyamino-2-methyl-1,2,3,4,-tetrahydroquinolines, having the Formula III

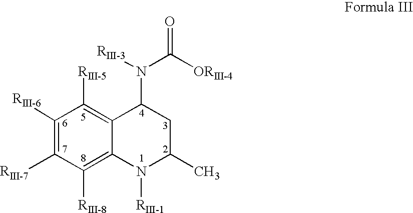

and pharmaceutically acceptable salts, enantiomers, or stereoisomers of said compounds;

wherein R_{II} I-₁ is hydrogen, Y_{II}, W_{II}—X_{III}, W_{III} —Y_{III};

wherein W_{III} is a carbonyl, thiocarbonyl, sulfinyl or sulfonyl;

X_{III} is —O—Y_{III} —S—Y_{III}, —N—(H) —Y_{III} or —N—(Y_{III})₂;

Y_{III} for each occurrence is independently Z_{III} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{III};

wherein Z_{III} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{II} I substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl optionally substituted with from one to nine fluorines;

R_{III-3} is hydrogen or Q_{III};

wherein Q_{III} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{III};

wherein V_{III} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{III} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄) alkylthio, amino, nitro, cyano, oxo, carboxy, (C₁-C₆)alkyloxycarbonyl, mono-N- or di-N,N-(C₁-C₆)alkylamino or said (C₁-C₆)alkyl or (C₂-C₆)alkenyl are optionally substituted with from one to nine fluorines;

R_{III-4} is Q_{III-1} or V_{III-1};

wherein Q_{III-1} a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{III-1};

wherein V_{III-1} is a partially saturated, fully saturated or fully unsaturated three to six membered ring optionally having one to two heteroatoms selected independently from oxygen, sulfur and nitrogen;

wherein said V_{III-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent optionally having from one to nine fluorines;

wherein either R_{III-3} must contain V_{III} or R_{III-4} must contain V_{III-1}; and

R_{III-5} and R_{III-6}, or R_{III-6} and R_{III-7}, and/or R_{III-7} and R_{III-8} are taken together and form at least one four to eight membered ring that is partially saturated or fully unsaturated optionally having one to three heteroatoms independently selected from nitrogen, sulfur and oxygen;

wherein said ring or rings formed by R_{III-5} and R_{III-6}, or R_{III-6} and R_{III-7}, and/or R_{III-7} and R_{III-8} are optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆) alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent optionally having from one to nine fluorines;

provided that the R_{III-5}, R_{III-6}, R_{III-7} and/or R_{III-8}, as the case may be, that do not form at least one ring are each independently hydrogen, halo, (C₁-C₆)alkoxy or (C₁-C₆)alkyl, said (C₁-C₆)alkyl optionally having from one to nine fluorines.

Compounds of Formula III are disclosed in commonly assigned pending U.S. patent application Ser. No. 09/390,738 the complete disclosure of which is herein incorporated by reference.

In a preferred embodiment, the CETP inhibitor is selected from one of the following compounds of Formula III:

[2R, 4S] 4-[(3,5-bis-trifluoromethyl-benzyl)-methoxycarbonylamino]-2-methyl-2,3,4,6,7,8-hexahydrocyclopenta[g]quinoline-1-carboxylic acid ethyl ester;

[6R, 8S] 8-[(3,5-bis-trifluoromethyl-benzyl)-methoxycarbonylamino]-6-methyl-3,6,7,8-tetrahydro-1H-2-thia-5-azacyclopenta[b]naphthalene-5-carboxylic acid ethylester;

[6R, 8S] 8-[(3,5-bis-trifluoromethyl-benzyl)-methoxycarbonylamino]-6-methyl-3,6,7,8-tetrahydro-2H-furo[2,3-g]quinoline-5-carboxylic acid ethyl ester;

[2R,4S] 4-[(3,5-bis-trifluoromethyl-benzyl)-methoxycarbonylamino]-2-methyl-3,4,6,8-tetrahydro-2H-furo[3,4-g]quinoline-1-carboxylic acid ethyl ester;

[2R,4S] 4-[(3,5-bis-trifluoromethyl-benzyl)-methoxycarbonylamino]-2-methyl-3,4,6,7,8,9-hexahydro-2H-benzo[g]quinoline-1-carboxylic acid propyl ester;

[7R,9S] 9-[(3,5-bis-trifluoromethyl-benzyl)-methoxycarbonylamino]-7-methyl-1,2,3,7,8,9-hexahydro-6-azacyclopenta[a]naphthalene-6-carboxylic acid ethyl ester; and

[6S,8R] 6-[(3,5-bis-trifluoromethyl-benzyl)-methoxycarbonylamino]-8-methyl-1,2,3,6,7,8-hexahydro-9-azacyclopenta[a]naphthalene-9-carboxylic acid ethyl ester.

Another class of CETP inhibitors that finds utility with the present invention consists of 4-carboxyamino-2-substituted-1,2,3,4,-tetrahydroquinolines, having the Formula IV

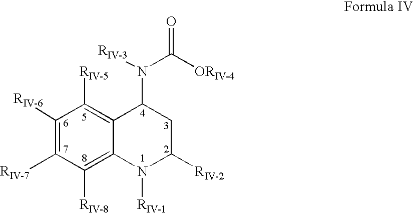

and pharmaceutically acceptable salts, enantiomers, or stereoisomers of said compounds;

wherein R_{IV-1} is hydrogen, Y_{IV}, W_{IV}—X_{IV} or W_{IV}—Y_{IV};

wherein W_{IV} is a carbonyl, thiocarbonyl, sulfinyl or sulfonyl;

X_{IV} is —O—Y_{IV}, —S—Y_{IV}, —N(H)—Y_{IV} or —N—(Y_{IV})₂;

wherein Y_{IV} for each occurrence is independently Z_{IV} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{IV};

wherein Z_{IV} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{IV} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines; R_{IV-2} is a partially saturated, fully saturated or fully unsaturated one to six membered straight or branched carbon chain wherein the carbons, other than the connecting carbon, may optionally be replaced with one or two heteroatoms selected independently from oxygen, sulfur and nitrogen wherein said carbon atoms are optionally mono-, di- or tri-substituted independently with halo, said carbon is optionally mono-substituted with oxo, said carbon is optionally mono-substituted with hydroxy, said sulfur is optionally mono- or di-substituted with oxo, said nitrogen is optionally mono- or di-substituted with oxo; or said R_{IV-2} is a partially saturated, fully saturated or fully unsaturated three to seven membered ring optionally having one to two heteroatoms selected independently from oxygen, sulfur and nitrogen, wherein said R_{IV-2} ring is optionally attached through (C₁-C₄) alkyl;

wherein said R_{IV-2} ring is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, oxo or (C₁-C₆)alkyloxycarbonyl;

with the proviso that R_{IV-2} is not methyl;

R_{IV-3} is hydrogen or Q_{IV};

wherein Q_{IV} is a fully saturated, partially unsaturated or fully unsaturated one to six membered straight or branched carbon chain wherein the carbons other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{IV};

wherein V_{IV} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{IV} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl or (C₂-C₆)alkenyl substituents are also optionally substituted with from one to nine fluorines;

R_{IV-4} is Q_{IV-1} or V_{IV-1};

wherein Q_{IV-1} a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{IV-1};

wherein V_{IV-1} is a partially saturated, fully saturated or fully unsaturated three to six membered ring optionally having one to two heteroatoms selected independently from oxygen, sulfur and nitrogen;

wherein said V_{IV-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is also optionally substituted with from one to nine fluorines;

wherein either R_{IV-3} must contain V_{IV} or R_{IV-4} must contain V_{IV-1};

R_{IV-5}, R_{IV-6}, R_{IV-7} and R_{IV-8} are each independently hydrogen, a bond, nitro or halo wherein said bond is substituted with T_{IV} or a partially saturated, fully saturated or fully unsaturated (C₁-C₁₂) straight or branched carbon chain wherein carbon, may optionally be replaced with one or two heteroatoms selected independently from oxygen, sulfur and nitrogen wherein said carbon atoms are optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon is optionally mono-substituted with T_{IV};

wherein T_{IV} is a partially saturated, fully saturated or fully unsaturated three to eight membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said T_{IV} substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines; and

wherein R_{IV-5} and R_{IV-6}, or R_{IV-6} and R_{IV-7}, and/or R_{IV-7} and R_{IV-8} may also be taken together and can form at least one four to eight membered ring that is partially saturated or fully unsaturated optionally having one to three heteroatoms independently selected from nitrogen, sulfur and oxygen;

wherein said ring or rings formed by R_{IV-5} and R_{IV-}6, or R_{IV-6} and R_{IV-7}, and/or R_{IV-7} and R_{IV-8} are optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines;

with the proviso that when R_{IV-2} is carboxyl or (C₁-C₄)alkylcarboxyl, then R_{IV-1} is not hydrogen.

Compounds of Formula IV are disclosed in commonly assigned pending U.S. patent application Ser. No. 09/391,152 the complete disclosure of which is herein incorporated by reference.

In a preferred embodiment, the CETP inhibitor is selected from one of the following compounds of Formula IV:

[2S,4S] 4-[(3,5-bis-trifluoromethyl-benzyl)-methoxycarbonylamino]-2-isopropyl-6-trifluoromethyl-3,4-dihydro-2H-quinoline-1-carboxylic acid isopropyl ester;

[2S,4S] 4-[(3,5-bis-trifluoromethyl-benzyl)-methoxycarbonylamino]-6-chloro-2-cyclopropyl-3,4-dihydro-2H-quinoline-1-carboxylic acid isopropyl ester;

[2S,4S] 2-cyclopropyl-4-[(3,5-dichloro-benzyl)methoxycarbonyl-amino]-6-trifluoromethyl-3,4-dihydro-2H-quinoline-1-carboxylic acid isopropyl ester;

[2S,4S] 4-[(3,5-bis-trifluoromethyl-benzyl)-methoxycarbonylamino]-2-cyclopropyl-6-trifluoromethyl-3,4-dihydro-2H-quinoline-1-carboxylic acid tert-butyl ester;

[2R,4R] 4-[(3,5-bis-trifluoromethyl-benzyl)methoxycarbonyl-amino]-2-cyclopropyl-6-trifluoromethyl-3,4-dihydro-2H-quinaline-1-carboxylic acid isopropyl ester;

[2S,4S] 4-[(3,5-bis-trifluoromethyl-benzyl)-methoxycarbonylamino]-2-cyclopropyl-6-trifluoromethyl-3,4-dihydro-2H-quinoline-1-carboxylic acid isopropyl ester;

[2S,4S] 4-[(3,5-bis-trifluoromethyl-benzyl)-methoxycarbonylamino]-2-cyclobutyl-6-trifluoromethyl-3,4-dihydro-2H-quinoline-1-carboxylic acid isopropyl ester,

[2R,4S] 4-[(3,5-bis-trifluoromethyl-benzyl)-methoxycarbonylamino]-2-ethyl-6-trifluoromethyl-3,4-dihydro-2H-quinoline-1-carboxylic acid isopropyl ester;

[2S,4S] 4-[(3,5-bis-trifluoromethyl-benzyl)-methoxycarbonylamino]-2-methoxymethyl-6-trifluoromethyl-3,4-dihydro-2H-quinoline-1-carboxylic acid isopropyl ester;

[2R,4S] 4-[(3,5-bis-trifluoromethyl-benzyl)-methoxycarbonylamino]-2-ethyl-6-trifluoromethyl-3,4-dihydro-2H-quinoline-1-carboxylic acid 2-hydroxy-ethyl ester;

[2S,4S] 4-[(3,5-bis-trifluoromethyl-benzyl)-methoxycarbonylamino]-2-cyclopropyl-6-trifluoromethyl-3,4-dihydro-2H-quinoline-1-carboxylic acid ethyl ester;

[2R,4S] 4-[(3,5-bis-trifluoromethyl-benzyl)-methoxycarbonylamino]-2-ethyl-6-trifluoromethyl-3,4-dihydro-2H-quinoline-1-carboxylic acid ethyl ester;

[2S,4S] 4-[(3,5-bis-trifluoromethyl-benzyl)-methoxycarbonylamino]-2-cyclopropyl-6-trifluoromethyl-3,4-dihydro-2H-quinoline-1-carboxylic acid propyl ester; and

[2R,4S] 4-[(3,5-bis-trifluoromethyl-benzyl)-methoxycarbonylamino]-2-ethyl-6-trifluoromethyl-3,4-dihydro-2H-quinoline-1-carboxylic acid propyl ester.

Another class of CETP inhibitors that finds utility with the present invention consists of 4-amino substituted-2-substituted-1,2,3,4,-tetrahydroquinolines, having the Formula V

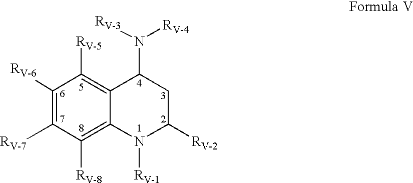

and pharmaceutically acceptable salts, enantiomers, or stereoisomers of said compounds;

wherein R_{V-1} is Y_{V}, W_{V}—X_{V} or W_{V}—Y_{V};

wherein W_{V} is a carbonyl, thiocarbonyl; sulfinyl or sulfonyl;

X_{V} is —O—Y_{V}, —S—Y_{V}, —N(H)—Y_{V} or —N—(Y_{V})₂;

wherein Y_{V} for each occurrence is independently Z_{V} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{V};

wherein Z_{V} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{V} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines;

R_{V-2} is a partially saturated, fully saturated or fully unsaturated one to six membered straight or branched carbon chain wherein the carbons, other than the connecting carbon, may optionally be replaced with one or two heteroatoms selected independently from oxygen, sulfur and nitrogen wherein said carbon atoms are optionally mono-, di- or tri-substituted independently with halo, said carbon is optionally mono-substituted with oxo, said carbon is optionally mono-substituted with hydroxy, said sulfur is optionally mono- or di-substituted with oxo, said nitrogen is optionally mono- or di-substituted with oxo; or said R_{V-2} is a partially saturated, fully saturated or fully unsaturated three to seven membered ring optionally having one to two heteroatoms selected independently from oxygen, sulfur and nitrogen, wherein said R_{V-2} ring is optionally attached through (C₁-C₄)alkyl;

wherein said R_{V-2} ring is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, oxo or (C₁-C₆) alkyloxycarbonyl;

R_{V-3} is hydrogen or Q_{V};

wherein Q_{V} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{V};

wherein V_{V} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{V} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl or (C₂-C₆)alkenyl substituents are also optionally substituted with from one to nine fluorines;

R_{V-4} is cyano, formyl, W_{V-1}Q_{V-1}, W_{V-1}V_{V-1}, (C₁-C₄)alkyleneV_{V-1} or V_{V-2};

wherein W_{V-1} is carbonyl, thiocarbonyl, SO or SO₂,

wherein Q_{V-1} a fully saturated, partially unsaturated or fully unsaturated one to six membered straight or branched carbon chain wherein the carbons may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{V-1};

wherein V_{V-1} is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{V-1} substituent is optionally mono-, di-, tri- or tetra-substituted independently with halo, (C₁-C₆)alkyl, (C₁-C₆)alkoxy, hydroxy, oxo, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is also optionally substituted with from one to nine fluorines;

wherein V_{V-2} is a partially saturated, fully saturated or fully unsaturated five to seven membered ring containing one to four heteroatoms selected independently from oxygen, sulfur and nitrogen;

wherein said V_{V-2} substituent is optionally mono-, di- or tri-substituted independently with halo, (C₁-C₂)alkyl, (C₁-C₂)alkoxy, hydroxy, or oxo wherein said (C₁-C₂)alkyl optionally has from one to five fluorines; and

wherein R_{V-4} does not include oxycarbonyl linked directly to the C⁴ nitrogen;

wherein either R_{V-3} must contain V_{V} or R_{V-4} must contain V_{V-1};

R_{V-5}, R_{V-6}, R_{V-7} and R_{V-8} are independently hydrogen, a bond, nitro or halo wherein said bond is substituted with T_{V} or a partially saturated, fully saturated or fully unsaturated (C₁-C₁₂) straight or branched carbon chain wherein carbon may optionally be replaced with one or two heteroatoms selected independently from oxygen, sulfur and nitrogen, wherein said carbon atoms are optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with T_{V};

wherein T_{V} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said T_{V} substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆) alkoxy, (C₁-C₄) alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent also optionally has from one to nine fluorines;

wherein R_{V-5} and R_{V-6}, or R_{V-6} and R_{V-7}, and/or R_{V-7} and R_{V-8} may also be taken together and can form at least one ring that is a partially saturated or fully unsaturated four to eight membered ring optionally having one to three heteroatoms independently selected from nitrogen, sulfur and oxygen;

wherein said rings formed by R_{V-5} and R_{V-6}, or R_{V-6} and R_{V-7}, and/or R_{V-7} and R_{V-8} are optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent also optionally has from one to nine fluorines.

Compounds of Formula V are disclosed in commonly assigned pending U.S. patent application Ser. No. 09/391,313 the complete disclosure of which is herein incorporated by reference.

In a preferred embodiment, the CETP inhibitor is selected from one of the following compounds of Formula V:

[2S,4S] 4-[(3,5-bis-trifluoromethyl-benzyl)-formyl-amino]-2-cyclopropyl-6-trifluoromethyl-3,4-dihydro-2H-quinoline-1-carboxylic acid isopropyl ester;

[2S,4S] 4-[(3,5-bis-trifluoromethyl-benzyl)-formyl-amino]-2-cyclopropyl-6-trifluoromethyl-3,4-dihydro-2H-quinoline-1-carboxylic acid propyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid tert-butyl ester;

[2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester;

[2R,4S] 4-[acetyl-(3,5-bis-trifluoromethyl-benzyl)-amino]-2-methyl-6-trifluoromethyl-3,4-dihydro-2H-quinoline-1-carboxylic acid ethyl ester,

[2S,4S] 4-[1-(3,5-bis-trifluoromethyl-benzyl)-ureido]-2-cyclopropyl-6-trifluoromethyl-3,4-dihydro-2H-quinoline-1-carboxylic acid isopropyl ester;

[2R,4S] 4-[acetyl-(3,5-bis-trifluoromethyl-benzyl)-amino]-2-ethyl-6-trifluoromethyl-3,4-dihydro-2H-quinoline-1-carboxylic acid ethyl ester;

[2S,4S] 4-[acetyl-(3,5-bis-trifluoromethyl-benzyl)-amino]-2-methoxymethyl-6-trifluoromethyl-3,4-dihydro-2H-quinoline-1-carboxylic acid isopropyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid propyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid ethyl ester;

[2R,4S] 4-[(3,5-bis-trifluoromethyl-benzyl)-formyl-amino]-2-ethyl-6-trifluoromethyl-3,4-dihydro-2H-quinoline-1-carboxylic acid isopropyl ester,

[2R,4S] 4-[(3,5-bis-trifluoromethyl-benzyl)-formyl-amino]-2-methyl-6-trifluoromethyl-3,4-dihydro-2H-quinoline-1-carboxylic acid ethyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid isopropyl ester;

[2R,4S] 4-[(3,5-bis-trifluoromethyl-benzyl)-formyl-amino]-2-ethyl-6-trifluoromethyl-3,4-dihydro-2H-quinoline-1-carboxylic acid ethyl ester;

[2S,4S] 4-[(3,5-bis-trifluoromethyl-benzyl)-formyl-amino]-2-cyclopropyl-6-trifluoromethyl-3,4-dihydro-2H-quinoline-1-carboxylic acid ethyl ester;

[2R,4S] 4-[(3,5-bis-trifluoromethyl-benzyl)-formyl-amino]-2-methyl-6-trifluoromethyl-3,4-dihydro-2H-quinoline-1-carboxylic acid isopropyl ester; and

[2R,4S] 4-[acetyl-(3,5-bis-trifluoromethyl-benzyl)-amino]-2-methyl-6-trifluoromethyl-3,4-dihydro-2H-quinoline-1-carboxylic acid isopropyl ester.

Another class of CETP inhibitors that finds utility with the present invention consists of cycloalkano-pyridines having the Formula VI

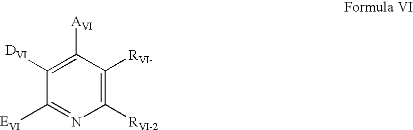

and pharmaceutically acceptable salts, enantiomers, or stereoisomers of said compounds;

in which

A_{VI} denotes an aryl containing 6 to 10 carbon atoms, which is optionally substituted with up to five identical or different substituents in the form of a halogen, nitro, hydroxyl, trifluoromethyl, trifluoromethoxy or a straight-chain or branched alkyl, acyl, hydroxyalkyl or alkoxy containing up to 7 carbon atoms each, or in the form of a group according to the formula

—BNR_{VI-3}R_{VI-4},

wherein

R_{VI-3} and R_{VI-4} are identical or different and denote a hydrogen, phenyl or a straight-chain or branched alkyl containing up to 6 carbon atoms,

D_{VI} denotes an aryl containing 6 to 10 carbon atoms, which is optionally substituted with a phenyl, nitro, halogen, trifluoromethyl or trifluoromethoxy, or a radical according to the formula

R_{VI-5}-L_{VI}-, 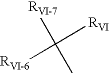
R_{VI-9}-T_{VI}—V_{VI}—X_{VI},

wherein

R_{VI-5}, R_{VI-6} and R_{VI-9} denote, independently from one another, a cycloalkyl containing 3 to 6 carbon atoms, or an aryl containing 6 to 10 carbon atom or a 5- to 7-membered, optionally benzo-condensed, saturated or unsaturated, mono-, bi- or tricyclic heterocycle containing up to 4 heteroatoms from the series of S, N and/or O, wherein the rings are optionally substituted, in the case of the nitrogen-containing rings also via the N function, with up to five identical or different substituents in the form of a halogen, trifluoromethyl, nitro, hydroxyl, cyano, carboxyl, trifluoromethoxy, a straight-chain or branched acyl, alkyl, alkylthio, alkylalkoxy, alkoxy or alkoxycarbonyl containing up to 6 carbon atoms each, an aryl or trifluoromethyl-substituted aryl containing 6 to 10 carbon atoms each, or an optionally benzo-condensed, aromatic 5- to 7-membered heterocycle containing up to 3 heteoatoms from the series of S, N and/or O, and/or in the form of a group according to the formula

BOR_{VI-10}, —SR_{VI-11}, —SO₂R_{VI-12} or BNR_{VI-13}R_{VI-14},

wherein

R_{VI-10}, R_{VI-11} and R_{VI-12} denote, independently from one another, an aryl containing 6 to 10 carbon atoms, which is in turn substituted with up to two identical or different substituents in the form of a phenyl, halogen or a straight-chain or branched alkyl containing up to 6 carbon atoms,

R_{VI-13} and R_{VI-14} are identical or different and have the meaning of R_{VI-3} and R_{VI-4} given above, or

R_{VI-5} and/or R_{VI-6} denote a radical according to the formula

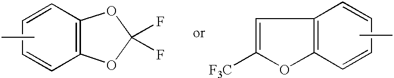

R_{VI-7} denotes a hydrogen or halogen, and

R_{VI-8} denotes a hydrogen, halogen, azido, trifluoromethyl, hydroxyl, trifluoromethoxy, a straight-chain or branched alkoxy or alkyl containing up to 6 carbon atoms each, or a radical according to the formula

—NR_{VI-15}R_{VI-16},

wherein

R_{VI-15} and R_{VI-16} are identical or different and have the meaning of R_{VI-3} and R_{VI-4} given above, or

R_{VI-7} and R_{VI-8} together form a radical according to the formula

═O or ═NR_{VI-17},

wherein

R_{VI-17} denotes a hydrogen or a straight-chain or branched alkyl, alkoxy or acyl containing up to 6 carbon atoms each,

L_{VI} denotes a straight-chain or branched alkylene or alkenylene chain containing up to 8 carbon atoms each, which are optionally substituted with up to two hydroxyl groups,

T_{VI} and X_{VI} are identical or different and denote a straight-chain or branched alkylene chain containing up to 8 carbon atoms, or

T_{VI} or X_{VI} denotes a bond,

V_{VI} denotes an oxygen or sulfur atom or an BNR_{VI-18} group, wherein

R_{VI-18} denotes a hydrogen or a straight-chain or branched alkyl containing up to 6 carbon atoms or a phenyl,

E_{VI} denotes a cycloalkyl containing 3 to 8 carbon atoms, or a straight-chain or branched alkyl containing up to 8 carbon atoms, which is optionally substituted with a cycloalkyl containing 3 to 8 carbon atoms or a hydroxyl, or a phenyl, which is optionally substituted with a halogen or trifluoromethyl,

R_{VI-1} and R_{VI-2} together form a straight-chain or branched alkylene chain containing up to 7 carbon atoms, which must be substituted with a carbonyl group and/or a radical according to the formula

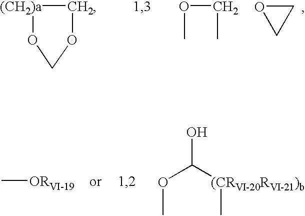

wherein

a and b are identical or different and denote a number equaling 1, 2 or 3,

R_{VI-19} denotes a hydrogen atom, a cycloalkyl containing 3 to 7 carbon atoms, a straight-chain or branched silylalkyl containing up to 8 carbon atoms, or a straight-chain or branched alkyl containing up to 8 carbon atoms, which is optionally substituted with a hydroxyl, a straight-chain or a branched alkoxy containing up to 6 carbon atoms or a phenyl, which may in turn be substituted with a halogen, nitro, trifluoromethyl, trifluoromethoxy or phenyl or tetrazole-substituted phenyl, and an alkyl that is optionally substituted with a group according to the formula

BOR_{VI-22},

wherein

R_{VI-22} denotes a straight-chain or branched acyl containing up to 4 carbon atoms or benzyl, or

R_{VI-19} denotes a straight-chain or branched acyl containing up to 20 carbon atoms or benzoyl, which is optionally substituted with a halogen, trifluoromethyl, nitro or trifluoromethoxy, or a straight-chain or branched fluoroacyl containing up to 8 carbon atoms,

R_{VI-20} and R_{VI-21} are identical or different and denote a hydrogen, phenyl or a straight-chain or branched alkyl containing up to 6 carbon atoms, or

R_{VI-20} and R_{VI-21} together form a 3- to 6-membered carbocyclic ring, and a the carbocyclic rings formed are optionally substituted, optionally also geminally, with up to six identical or different substituents in the form of trifluoromethyl, hydroxyl, nitrile, halogen, carboxyl, nitro, azido, cyano, cycloalkyl or cycloalkyloxy containing 3 to 7 carbon atoms each, a straight-chain or branched alkoxycarbonyl, alkoxy or alkylthio containing up to 6 carbon atoms each, or a straight-chain or branched alkyl containing up to 6 carbon atoms, which is in turn substituted with up to two identical or different substituents in the form of a hydroxyl, benzyloxy, trifluoromethyl, benzoyl, a straight-chain or branched alkoxy, oxyacyl or carboxyl containing up to 4 carbon atoms each and/or a phenyl, which may in turn be substituted with a halogen, trifluoromethyl or trifluoromethoxy, and/or the carbocyclic rings formed are optionally substituted, also geminally, with up to five identical or different substituents in the form of a phenyl, benzoyl, thiophenyl or sulfonylbenzyl, which in turn are optionally substituted with a halogen, trifluoromethyl, trifluoromethoxy or nitro, and/or optionally in the form of a radical according to the formula

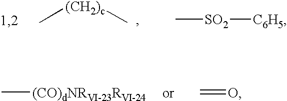

wherein

c is a number equaling 1, 2, 3 or 4,

d is a number equaling 0 or 1,

R_{VI-23} and R_{VI-24} are identical or different and denote a hydrogen, cycloalkyl containing 3 to 6 carbon atoms, a straight-chain or branched alkyl containing up to 6 carbon atoms, benzyl or phenyl, which is optionally substituted with up to two identical or different substituents in the form of halogen, trifluoromethyl, cyano, phenyl or nitro, and/or the carbocyclic rings formed are optionally substituted with a spiro-linked radical according to the formula

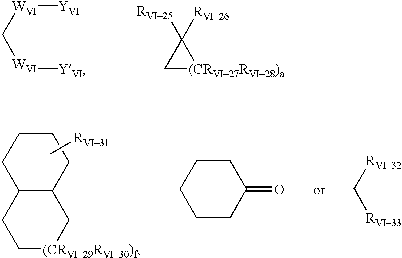

wherein

W_{VI} denotes either an oxygen atom or a sulfur atom,

Y_{VI} and Y=_{VI} together form a 2- to 6-membered straight-chain or branched alkylene chain,

e is a number equaling 1, 2, 3, 4, 5, 6 or 7,

f is a number equaling 1 or 2,

R_{VI-25}, R_{VI-26}, R_{VI-27}, R_{VI-28}, R_{VI-29}, R_{VI-30} and R_{VI-31} are identical or different and denote a hydrogen, trifluoromethyl, phenyl, halogen or a straight-chain or branched alkyl or alkoxy containing up to 6 carbon atoms each, or

R_{VI-25} and R_{VI-26} or R_{VI-27} and R_{VI-28} each together denote a straight-chain or branched alkyl chain containing up to 6 carbon atoms or

R_{VI-25} and R_{VI-26} or R_{VI-27} and R_{VI-28} each together form a radical according to the formula

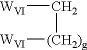

wherein

W_{VI} has the meaning given above,

g is a number equaling 1, 2, 3, 4, 5, 6 or 7,

R_{VI-32} and R_{VI-33} together form a 3- to 7-membered heterocycle, which contains an oxygen or sulfur atom or a group according to the formula SO, SO₂ or BNR_{VI-34}, wherein

R_{VI-34} denotes a hydrogen atom, a phenyl, benzyl, or a straight-chain or branched alkyl containing up to 4 carbon atoms, and salts and N oxides thereof, with the exception of 5(6H)-quinolones, 3-benzoyl-7,8-dihydro-2,7,7-trimethyl-4-phenyl.

Compounds of Formula VI are disclosed in European Patent Application No. EP 818448 A1, the complete disclosure of which is herein incorporated by reference.

In a preferred embodiment, the CETP inhibitor is selected from one of the following compounds of Formula VI:

2-cyclopentyl-4-(4-fluorophenyl)-7,7-dimethyl-3-(4-trifluoromethylbenzoyl)-4,6,7,8-tetrahydro-1H-quinolin-5-one;

2-cyclopentyl-4-(4-fluorophenyl)-7,7-dimethyl-3-(4-trifluoromethylbenzoyl)-7,8-dihydro-6H-quinolin-5-one;

[2-cyclopentyl-4-(4-fluorophenyl)-5-hydroxy-7,7-dimethyl-5,6,7,8-tetrahydroquinolin-3-yl]-(4-trifluoromethylphenyl)-methanone;

[5-(t-butyldimethylsilanyloxy)-2-cyclopentyl-4-(4-fluorophenyl)-7,7-dimethyl-5,6,7,8-tetrahydroquinolin-3-yl]-(4-trifluoromethylphenyl)-methanone;

[5-(t-butyldimethylsilanyloxy)-2-cyclopentyl-4-(4-fluorophenyl)-7,7-dimethyl-5,6,7,8-tetrahydroquinolin-3-yl]-(4-trifluoromethylphenyl)-methanol;

5-(t-butyldimethylsilanyloxy)-2-cyclopentyl-4-(4-fluorophenyl)-3-[fluoro-(4-trifluoromethylphenyl)-methyl]-7,7-dimethyl-5,6,7,8-tetrahydroquinoline;

2-cyclopentyl-4-(4-fluorophenyl)-3-[fluoro-(4-trifluoromethylphenyl)-methyl]-7,7-dimethyl-5,6,7,8-tetrahydroquinolin-5-ol.

Another class of CETP inhibitors that finds utility with the present invention consists of substituted-pyridines having the Formula VII

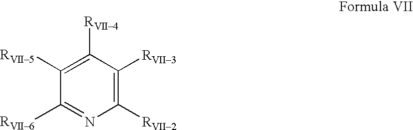

or a pharmaceutically acceptable salt or tautomer thereof, wherein

R_{VII-2} and R_{VII-6} are independently selected from the group consisting of hydrogen, hydroxy, alkyl, fluorinated alkyl, fluorinated aralkyl, chlorofluorinated alkyl, cycloalkyl, heterocyclyl, aryl, heteroaryl, alkoxy, alkoxyalkyl, and alkoxycarbonyl; provided that at least one of R_{VII-2} and R_{VII-6} is fluorinated alkyl, chlorofluorinated alkyl or alkoxyalkyl;

R_{VII-3} is selected from the group consisting of hydroxy, amido, arylcarbonyl, heteroarylcarbonyl, hydroxymethyl —CHO, —CO₂R_{VII-7}, wherein R_{VII-7} is selected from the group consisting of hydrogen, alkyl and cyanoalkyl; and

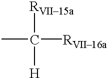

wherein R_{VII-15a} is selected from the group consisting of hydroxy, hydrogen, halogen, alkylthio, alkenylthio, alkynylthio, arylthio, heteroarylthio, heterocyclylthio, alkoxy, alkenoxy, alkynoxy, aryloxy, heteroaryloxy and heterocyclyloxy, and

R_{VII-16a} is selected from the group consisting of alkyl, haloalkyl, alkenyl, haloalkenyl, alkynyl, haloalkynyl, aryl, heteroaryl, and heterocyclyl, arylalkoxy, trialkylsilyloxy;

R_{VII-4} is selected from the group consisting of hydrogen, hydroxy, halogen, alkyl, alkenyl, alkynyl, cycloalkyl, cycloalkenyl, haloalkyl, haloalkenyl, haloalkynyl, aryl, heteroaryl, heterocyclyl, cycloalkylalkyl, cycloalkenylalkyl, aralkyl, heteroarylalkyl, heterocyclylalkyl, cycloalkylalkenyl, cycloalkenylalkenyl, aralkenyl, hetereoarylalkenyl, heterocyclylalkenyl, alkoxy, alkenoxy, alkynoxy, aryloxy, heteroaryloxy, heterocyclyloxy, alkanoyloxy, alkenoyloxy, alkynoyloxy, aryloyloxy, heteroaroyloxy, heterocyclyloyloxy, alkoxycarbonyl, alkenoxycarbonyl, alkynoxycarbonyl, aryloxycarbonyl, heteroaryloxycarbonyl, heterocyclyloxycarbonyl, thio, alkylthio, alkenylthio, alkynylthio, arylthio, heteroarylthio, heterocyclylthio, cycloalkylthio, cycloalkenylthio, alkylthioalkyl, alkenylthioalkyl, alkynylthioalkyl, arylthioalkyl, heteroarylthioalkyl, heterocyclylthioalkyl, alkylthioalkenyl, alkenylthioalkenyl, alkynylthioalkenyl, arylthioalkenyl, heteroarylthioalkenyl, heterocyclythioalkenyl, alkylamino, alkenylamino, alkynylamino, arylamino, heteroarylamino, heterocyclylamino, aryldialkylamino, diarylamino, diheteroarylamino, alkylarylamino, alkylheteroarylamino, arylheteroarylamino, trialkylsilyl, trialkenylsilyl, triarylsilyl, —CO (O)N (R_{VII-8a}R_{VII-8b}), wherein R_{VII-8a} and R_{VII-8b} are independently selected from the group consisting of alkyl, alkenyl, alkynyl, aryl, heteroaryl and heterocyclyl, —SO₂R_{VII-9}, wherein R_{VII-9} is selected from the group consisting of hydroxy, alkyl, alkenyl, alkynyl, aryl, heteroaryl and heterocyclyl, —OP(O)(OR_{VII-10a}) (OR_{VII-10b}), wherein R_{VII-10a} and R_{VII-10b} are independently selected from the group consisting of hydrogen, hydroxy, alkyl, alkenyl, alkynyl, aryl, heteroaryl and heterocyclyl, and —OP(S) (OR_{VII-11a}) (OR_{VII-11b}), wherein R_{VII-11a} and R_{VII-11b} are independently selected from the group consisting of alkyl, alkenyl, alkynyl, aryl, heteroaryl and heterocyclyl;

R_{VII-5} is selected from the group consisting of hydrogen, hydroxy, halogen, alkyl, alkenyl, alkynyl, cycloalkyl, cycloalkenyl, haloalkyl, haloalkenyl, haloalkynyl, aryl, heteroaryl, heterocyclyl, alkoxy, alkenoxy, alkynoxy, aryloxy, heteroaryloxy, heterocyclyloxy, alkylcarbonyloxyalkyl, alkenylcarbonyloxyalkyl, alkynylcarbonyloxyalkyl, arylcarbonyloxyalkyl, heteroarylcarbonyloxyalkyl, heterocyclylcarbonyloxyalkyl, cycloalkylalkyl, cycloalkenylalkyl, aralkyl, heteroarylalkyl, heterocyclylalkyl, cycloalkylalkenyl, cycloalkenylalkenyl, aralkenyl, heteroarylalkenyl, heterocyclylalkenyl, alkylthioalkyl, cycloalkylthioalkyl, alkenylthioalkyl, alkynylthioalkyl, arylthioalkyl, heteroarylthioalkyl, heterocyclylthioalkyl, alkylthioalkenyl, alkenylthioalkenyl, alkynylthioalkenyl, arylthioalkenyl, heteroarylthioalkenyl, heterocyclylthioalkenyl, alkoxyalkyl, alkenoxyalkyl, alkynoxylalkyl, aryloxyalkyl, heteroaryloxyalkyl, heterocyclyloxyalkyl, alkoxyalkenyl, alkenoxyalkenyl, alkynoxyalkenyl, aryloxyalkenyl, heteroaryloxyalkenyl, heterocyclyloxyalkenyl, cyano, hydroxymethyl, —CO₂R_{VII-14}, wherein R_{VII-14} is selected from the group consisting of alkyl, alkenyl, alkynyl, aryl, heteroaryl and heterocyclyl;

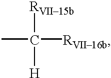

wherein R_{VII-15b} is selected from the group consisting of hydroxy, hydrogen, halogen, alkylthio, alkenylthio, alkynylthio, arylthio, heteroarylthio, heterocyclylthio, alkoxy, alkenoxy, alkynoxy, aryloxy, heteroaryloxy, heterocyclyloxy, aroyloxy, and alkylsulfonyloxy, and

R_{VII-16b} is selected form the group consisting of alkyl, alkenyl, alkynyl, aryl, heteroaryl, heterocyclyl, arylalkoxy, and trialkylsilyloxy;

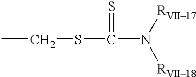

wherein R_{VII-17} and R_{VII-18} are independently selected from the group consisting of alkyl, cycloalkyl, alkenyl, alkynyl, aryl, heteroaryl and heterocyclyl;

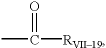

wherein R_{VII-19} is selected from the group consisting of alkyl, cycloalkyl, alkenyl, alkynyl, aryl, heteroaryl, heterocyclyl, —SR_{VII-20}, —OR_{VII-21}, and BR_{VII-22}CO₂R_{VII-23}, wherein

R_{VII-20} is selected from the group consisting of alkyl, alkenyl, alkynyl, aryl, heteroaryl, heterocyclyl, aminoalkyl, aminoalkenyl, aminoalkynyl, aminoaryl, aminoheteroaryl, aminoheterocyclyl, alkylheteroarylamino, arylheteroarylamino,

R_{VII-21} is selected from the group consisting of alkyl, alkenyl, alkynyl, aryl, heteroaryl, and heterocyclyl,

R_{VII-22} is selected from the group consisting of alkylene or arylene, and

R_{VII-23} is selected from the group consisting of alkyl, alkenyl, alkynyl, aryl, heteroaryl, and heterocyclyl;

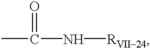

wherein R_{VII-24} is selected from the group consisting of hydrogen, alkyl, cycloalkyl, alkenyl, alkynyl, aryl, heteroaryl, heterocyclyl, aralkyl, aralkenyl, and aralkynyl;

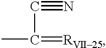

wherein R_{VII-25} is heterocyclylidenyl;

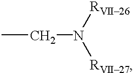

wherein R_{VII-26} and R_{VII-27} are independently selected from the group consisting of hydrogen, alkyl, cycloalkyl, alkenyl, alkynyl, aryl, heteroaryl, and heterocyclyl;

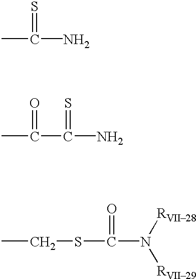

wherein R_{VII-28} and R_{VII-29} are independently selected from the group consisting of hydrogen, alkyl, cycloalkyl, alkenyl, alkynyl, aryl, heteroaryl, and heterocyclyl;

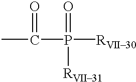

wherein R_{VII-30} and R_{VII-31} are independently alkoxy, alkenoxy, alkynoxy, aryloxy, heteroaryloxy, and heterocyclyloxy; and

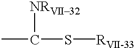

wherein R_{VII-32} and R_{VII-33} are independently selected from the group consisting of hydrogen, alkyl, cycloalkyl, alkenyl, alkynyl, aryl, heteroaryl, and heterocyclyl;

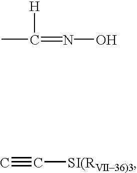

wherein R_{VII-36} is selected from the group consisting of alkyl, alkenyl, aryl, heteroaryl and heterocyclyl;

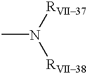

wherein R_{VII-37} and R_{VII-38} are independently selected from the group consisting of hydrogen, alkyl, cycloalkyl, alkenyl, alkynyl, aryl, heteroaryl, and heterocyclyl;

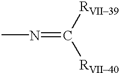

wherein R_{VII-39} is selected from the group consisting of hydrogen, alkoxy, alkenoxy, alkynoxy, aryloxy, heteroaryloxy, heterocyclyloxy, alkylthio, alkenylthio, alkynylthio, arylthio, heteroarylthio and heterocyclylthio, and

R_{VII-40} is selected from the group consisting of haloalkyl, haloalkenyl, haloalkynyl, haloaryl, haloheteroaryl, haloheterocyclyl, cycloalkyl, cycloalkenyl, heterocyclylalkoxy, heterocyclylalkenoxy, heterocyclylalkynoxy, alkylthio, alkenylthio, alkynylthio, arylthio, heteroarylthio and heterocyclylthio;

—N═R_{VII-41},

wherein R_{VII-41} is heterocyclylidenyl;

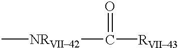

wherein R_{VII-42} is selected from the group consisting of hydrogen, alkyl, alkenyl, alkynyl, aryl, heteroaryl, and heterocyclyl, and

R_{VII-43} is selected from the group consisting of hydrogen, alkyl, alkenyl, alkynyl, aryl, heteroaryl, heterocyclyl, cycloalkyl, cycloalkenyl, haloalkyl, haloalkenyl, haloalkynyl, haloaryl, haloheteroaryl, and haloheterocyclyl;

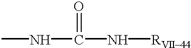

wherein R_{VII-44} is selected from the group consisting of hydrogen, alkyl, cycloalkyl, alkenyl, alkynyl, aryl, heteroaryl and heterocyclyl;

—N═S═O;

—N═C═S;

—N═C═O;

—N₃;

—SR_{VII-45}

wherein R_{VII-45} is selected from the group consisting of hydrogen, alkyl, alkenyl, alkynyl, aryl, heteroaryl, heterocyclyl, haloalkyl, haloalkenyl, haloalkynyl, haloaryl, haloheteroaryl, haloheterocyclyl, heterocyclyl, cycloalkylalkyl, cycloalkenylalkyl, aralkyl, heteroarylalkyl, heterocyclylalkyl, cycloalkylalkenyl, cycloalkenylalkenyl, aralkenyl, heteroarylalkenyl, heterocyclylalkenyl, alkylthioalkyl, alkenylthioalkyl, alkynylthioalkyl, arylthioalkyl,heteroarylthioalkyl, heterocyclylthioalkyl, alkylthioalkenyl, alkenylthioalkenyl, alkynylthioalkenyl, arylthioalkenyl, heteroarylthioalkenyl, heterocyclylthioalkenyl, aminocarbonylalkyl, aminocarbonylalkenyl, aminocarbonylalkynyl, aminocarbonylaryl, aminocarbonylheteroaryl, and aminocarbonylheterocyclyl,

—SR_{VII-46}, and —CH₂R_{VII-47},

wherein R_{VII-46} is selected from the group consisting of alkyl, alkenyl, alkynyl, aryl, heteroaryl and heterocyclyl, and

R_{VII-47} is selected from the group consisting of hydrogen, alkyl, alkenyl, alkynyl, aryl, heteroaryl and heterocyclyl; and

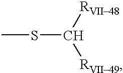

wherein R_{VII-48} is selected from the group consisting of hydrogen, alkyl, cycloalkyl, alkenyl, alkynyl, aryl, heteroaryl and heterocyclyl, and

R_{VII-49} is selected from the group consisting of alkoxy, alkenoxy, alkynoxy, aryloxy, heteroaryloxy, heterocyclyloxy, haloalkyl, haloalkenyl, haloalkynyl, haloaryl, haloheteroaryl and haloheterocyclyl;

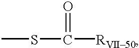

wherein R_{VII-50} is selected from the group consisting of hydrogen, alkyl, cycloalkyl, alkenyl, alkynyl, aryl, heteroaryl, heterocyclyl, alkoxy, alkenoxy, alkynoxy, aryloxy, heteroaryloxy and heterocyclyloxy;

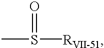

wherein R_{VII-51} is selected from the group consisting of alkyl, alkenyl, alkynyl, aryl, heteroaryl, heterocyclyl, haloalkyl, haloalkenyl, haloalkynyl, haloaryl, haloheteroaryl and haloheterocyclyl; and

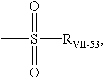

wherein R_{VII-53} is selected from the group consisting of alkyl, alkenyl, alkynyl, aryl, heteroaryl and heterocyclyl;

provided that when R_{VII-5} is selected from the group consisting of heterocyclylalkyl and heterocyclylalkenyl, the heterocyclyl radical of the corresponding heterocyclylalkyl or heterocyclylalkenyl is other than δ-lactone; and

provided that when R_{VII-4} is aryl, heteroaryl or heterocyclyl, and one of R_{VII-2} and R_{VII-6} is trifluoromethyl, then the other of R_{VII-2} and R_{VII-6} is difluoromethyl.

Compounds of Formula VII are disclosed in WO 9941237-A1, the complete disclosure of which is incorporated by reference.

In a preferred embodiment, the CETP inhibitor is selected from the following compounds of Formula VII: Dimethyl 5,5-dithiobis[2-difluoromethyl-4-(2-methylpropyl)-6-(trifluoromethyl)-3-pyridine-carboxylate].

Another class of CETP inhibitors that finds utility with the present invention consists of substituted biphenyls having the Formula VIII

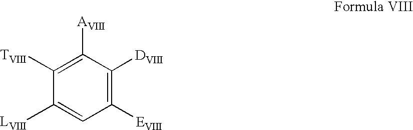

or a pharmaceutically acceptable salt, enantiomers, or stereoisomers thereof,

in which

A_{VIII} stands for aryl with 6 to 10 carbon atoms, which is optionally substituted up to 3 times in an identical manner or differently by halogen, hydroxy, trifluoromethyl, trifluoromethoxy, or by straight-chain or branched alkyl, acyl, or alkoxy with up to 7 carbon atoms each, or by a group of the formula

—NR_{VIII-1}R_{VIII-2},

wherein

R_{VIII-1} and R_{VIII-2} are identical or different and denote hydrogen, phenyl, or straight-chain or branched alkyl with up to 6 carbon atoms,

D_{VIII} stands for straight-chain or branched alkyl with up to 8 carbon atoms, which is substituted by hydroxy,

E_{VIII} and L_{VIII} are either identical or different and stand for straight-chain or branched alkyl with up to 8 carbon atoms, which is optionally substituted by cycloalkyl with 3 to 8 carbon atoms, or stands for cycloalkyl with 3 to 8 carbon atoms, or

E_{VIII} has the above-mentioned meaning and

L_{VIII} in this case stands for aryl with 6 to 10 carbon atoms, which is optionally substituted up to 3 times in an identical manner or differently by halogen, hydroxy, trifluoromethyl, trifluoromethoxy, or by straight-chain or branched alkyl, acyl, or alkoxy with up to 7 carbon atoms each, or by a group of the formula

—NR_{VIII-3}R_{VIII-4},

wherein

R_{VIII-3} and R_{VIII-4} are identical or different and have the meaning given above for R_{VIII-1} and R_{VIII-2}, or

E_{VIII} stands for straight-chain or branched alkyl with up to 8 carbon atoms, or stands for aryl with 6 to 10 carbon atoms, which is optionally substituted up to 3 times in an identical manner or differently by halogen, hydroxy, trifluoromethyl, trifluoromethoxy, or by straight-chain or branched alkyl, acyl, or alkoxy with up to 7 carbon atoms each, or by a group of the formula

—NR_{VIII-5}R_{VIII-6},

wherein

R_{VIII-5} and R_{VIII-6} are identical or different and have the meaning given above for R_{VIII-1} and R_{VIII-2}, and

L_{VIII} in this case stands for straight-chain or branched alkoxy with up to 8 carbon atoms or for cycloalkyloxy with 3 to 8 carbon atoms,

T_{VIII} stands for a radical of the formula

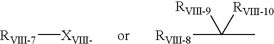

R_{VIII-7} and R_{VIII-8} are identical or different and denote cycloalkyl with 3 to 8 carbon atoms, or aryl with 6 to 10 carbon atoms, or denote a 5- to 7-member aromatic, optionally benzo-condensed, heterocyclic compound with up to 3 heteroatoms from the series S, N and/or O, which are optionally substituted up to 3 times in an identical manner or differently by trifluoromethyl, trifluoromethoxy, halogen, hydroxy, carboxyl, by straight-chain or branched alkyl, acyl, alkoxy, or alkoxycarbonyl with up to 6 carbon atoms each, or by phenyl, phenoxy, or thiophenyl, which can in turn be substituted by halogen, trifluoromethyl, or trifluoromethoxy, and/or the rings are substituted by a group of the formula

—NR_{VIII-11}R_{VIII-12},

wherein

R_{VIII-11} and R_{VIII-12} are identical or different and have the meaning given above for R_{VIII-1} and R_{VIII-2},

X_{VIII} denotes a straight or branched alkyl chain or alkenyl chain with 2 to 10 carbon atoms each, which are optionally substituted up to 2 times by hydroxy,

R_{VIII-9} denotes hydrogen, and

R_{VIII-10} denotes hydrogen, halogen, azido, trifluoromethyl, hydroxy, mercapto, trifluoromethoxy, straight-chain or branched alkoxy with up to 5 carbon atoms, or a radical of the formula

—NR_{VIII-13}R_{VIII-14},

wherein

R_{VIII-13} and R_{VIII-14} are identical or different and have the meaning given above for R_{VIII-1} and R_{VIII-2}, or

R_{VIII-9} and R_{VIII-10} form a carbonyl group together with the carbon atom.

Compounds of Formula VIII are disclosed in WO 9804528, the complete disclosure of which is incorporated by reference.

Another class of CETP inhibitors that finds utility with the present invention consists of substituted 1,2,4-triazoles having the Formula IX

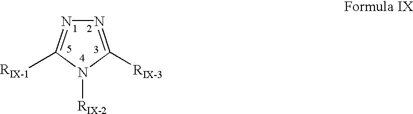

or a pharmaceutically acceptable salt or tautomer thereof;

wherein R_{IX-1} is selected from higher alkyl, higher alkenyl, higher alkynyl, aryl, aralkyl, aryloxyalkyl, alkoxyalkyl, alkylthioalkyl, arylthioalkyl, and cycloalkylalkyl;

wherein R_{IX-2} is selected from aryl, heteroaryl, cycloalkyl, and cycloalkenyl, wherein R_{IX-2} is optionally substituted at a substitutable position with one or more radicals independently selected from alkyl, haloalkyl, alkylthio, alkylsulfinyl, alkylsulfonyl, alkoxy, halo, aryloxy, aralkyloxy, aryl, aralkyl, aminosulfonyl, amino, monoalkylamino and dialkylamino; and

wherein R_{IX-3} is selected from hydrido, —SH and halo; provided R_{IX-2}cannot be phenyl or 4-methylphenyl when R_{IX-1} is higher alkyl and when R_{IX-3} is BSH.

Compounds of Formula IX are disclosed in WO 9914204, the complete disclosure of which is incorporated by reference.

In a preferred embodiment, the CETP inhibitor is selected from the following compounds of Formula IX:

2,4-dihydro-4-(3-methoxyphenyl)-5-tridecyl-3H-1,2,4-triazole-3-thione;

2,4-dihydro-4-(2-fluorophenyl)-5-tridecyl-3H-1,2,4-triazole-3-thione;

2,4-dihydro-4-(2-methylphenyl)-5-tridecyl-3H-1,2,4-triazole-3-thione;

2,4-dihydro-4-(3-chlorophenyl)-5-tridecyl-3H-1,2,4-triazole-3-thione;

2,4-dihydro-4-(2-methoxyphenyl)-5-tridecyl-3H-1,2,4-triazole-3-thione;

2,4-dihydro-4-(3-methylphenyl)-5-tridecyl-3H-1,2,4-triazole-3-thione;

4-cyclohexyl-2,4-dihydro-5-tridecyl-3H-1,2,4-triazole-3-thione;

2,4-dihydro-4-(3-pyridyl)-5-tridecyl-3H-1,2,4-triazole-3-thione;

2,4-dihydro-4-(2-ethoxyphenyl)-5-tridecyl-3H-1,2,4-triazole-3-thione;

2,4-dihydro-4-(2,6-dimethylphenyl)-5-tridecyl-3H-1,2,4-triazole-3-thione;

2,4-dihydro-4-(4-phenoxyphenyl)-5-tridecyl-3H-1,2,4-triazole-3-thione;

4-(1,3-benzodioxol-5-yl)-2,4-dihydro-5-tridecyl-3H-1,2,4-triazole-3-thione;

4-(2-chlorophenyl)-2,4-dihydro-5-tridecyl-3H-1,2,4-triazole-3-thione;

2,4-dihydro-4-(4-methoxyphenyl)-5-tridecyl-3H-1,2,4-triazole-3-thione;

2,4-dihydro-5-tridecyl-4-(3-trifluoromethylphenyl)-3H-1,2,4-triazole-3-thione;

2,4-dihydro-5-tridecyl-4-(3-fluorophenyl)-3H-1,2,4-triazole-3-thione;

4-(3-chloro-4-methylphenyl)-2,4-dihydro-5-tridecyl-3H-1,2,4-triazole-3-thione;

2,4-dihydro-4-(2-methylthiophenyl)-5-tridecyl-3H-1,2,4-triazole-3-thione;

4-(4-benzyloxyphenyl)-2,4-dihydro-5-tridecyl-3H-1,2,4-triazole-3-thione;

2,4-dihydro-4-(2-naphthyl)-5-tridecyl-3H-1,2,4-triazole-3-thione;

2,4-dihydro-5-tridecyl-4-(4-trifluoromethylphenyl)-3H-1,2,4-triazole-3-thione;

2,4-dihydro-4-(1-naphthyl)-5-tridecyl-3H-1,2,4-triazole-3-thione;

2,4-dihydro-4-(3-methylthiophenyl)-5-tridecyl-3H-1,2,4-triazole-3-thione;

2,4-dihydro-4-(4-methylthiophenyl)-5-tridecyl-3H-1,2,4-triazole-3-thione;

2,4-dihydro-4-(3,4-dimethoxyphenyl)-5-tridecyl-3H-1,2,4-triazole-3-thione;

2,4-dihydro-4-(2,5-dimethoxyphenyl)-5-tridecyl-3H-1,2,4-triazole-3-thione;

2,4-dihydro-4-(2-methoxy-5-chlorophenyl)-5-tridecyl-3H-1,2,4-triazole-3-thione;

4-(4-aminosulfonylphenyl)-2,4-dihydro-5-tridecyl-3H-1,2,4-triazole-3-thione;

2,4-dihydro-5-dodecyl-4-(3-methoxyphenyl)-3H-1,2,4-triazole-3-thione;

2,4-dihydro-4-(3-methoxyphenyl)-5-tetradecyl-3H-1,2,4-triazole-3-thione;

2,4-dihydro-4-(3-methoxyphenyl)-5-undecyl-3H-1,2,4-triazole-3-thione; and

2,4-dihydro-(4-methoxyphenyl)-5-pentadecyl-3H-1,2,4-triazole-3-thione.

A_{X} represents a radical of the formula

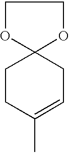

D_{X} represents an aryl having 6 to 10 carbon atoms, that is optionally substituted by phenyl, nitro, halogen, trifluormethyl or trifluormethoxy, or it represents a radical of the formula

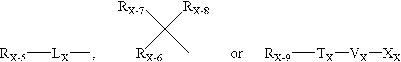

in which

R_{X-5}, R_{X-6} and R_{X-10} independently of one another denote cycloalkyl having 3 to 6 carbon atoms, or an aryl having 6 to 10 carbon atoms or a 5- to 7-membered aromatic, optionally benzo-condensed saturated or unsaturated, mono-, bi-, or tricyclic heterocyclic ring from the series consisting of S, N and/or O, in which the rings are substituted, optionally, in case of the nitrogen containing aromatic rings via the N function, with up to 5 identical or different substituents in the form of halogen, trifluoromethyl, nitro, hydroxy, cyano, carbonyl, trifluoromethoxy, straight straight-chain or branched acyl, alkyl, alkylthio, alkylalkoxy, alkoxy, or alkoxycarbonyl each having up to 6 carbon atoms, by aryl or trifluoromethyl-substituted aryl each having 6 to 10 carbon atoms or by an, optionally benzo-condensed, aromatic 5- to 7-membered heterocyclic ring having up to 3 heteroatoms from the series consisting of S, N, and/or O, and/or substituted by a group of the formula

BOR_{X-10}, —SR_{X-11}, SO₂R_{X-12} or BNR_{X-13}R_{X-14},

in which

R_{X-10}, R_{X-11} and R_{X-12} independently from each other denote aryl having 6 to 10 carbon atoms, which is in turn substituted with up to 2 identical or different substituents in the form of phenyl, halogen or a straight-chain or branched alkyl having up to 6 carbon atoms,

R_{X-13} and R_{X-14} are identical or different and have the meaning of R_{X-3} and R_{X-4} indicated above, or

R_{X-5} and/or R_{X-6} denote a radical of the formula

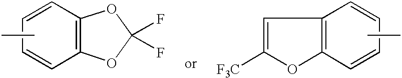

R_{X-7} denotes hydrogen or halogen, and

R_{X-8} denotes hydrogen, halogen, azido, trifluoromethyl, hydroxy, trifluoromethoxy, straight-chain or branched alkoxy or alkyl having up to 6 carbon atoms or a radical of the formula

BNR_{X-15}R_{X-16},

in which

R_{X-15} and R_{X-16} are identical or different and have the meaning of R_{X-3} and R_{X-4} indicated above, or

R_{X-7} and R_{X-8} together form a radical of the formula

=0 or=NR_{X-17},

in which

R_{X-17} denotes hydrogen or straight chain or branched alkyl, alkoxy or acyl having up to 6 carbon atoms,

L_{X} denotes a straight chain or branched alkylene or alkenylene chain having up to 8 carbon atoms, that are optionally substituted with up to 2 hydroxy groups,

T_{X} and X_{X} are identical or different and denote a straight chain or branched alkylene chain with up to 8 carbon atoms or

T_{X} or X_{X} denotes a bond,

V_{X} represents an oxygen or sulfur atom or an BNR_{X-18}-group, in which

R_{X-18} denotes hydrogen or straight chain or branched alkyl with up to 6 carbon atoms or phenyl,

E_{X} represents cycloalkyl with 3 to 8 carbon atoms, or straight chain or branched alkyl with up to 8 carbon atoms, that is optionally substituted by cycloalkyl with 3 to 8 carbon atoms or hydroxy, or represents a phenyl, that is optionally substituted by halogen or trifluoromethyl,

R_{X-1} and R_{X-2} together form a straight-chain or branched alkylene chain with up to 7 carbon atoms, that must be substituted by carbonyl group and/or by a radical with the formula

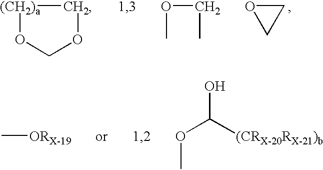

in which a and b are identical or different and denote a number equaling 1,2, or 3,

R_{X-19} denotes hydrogen, cycloalkyl with 3 up to 7 carbon atoms, straight chain or branched silylalkyl with up to 8 carbon atoms or straight chain or branched alkyl with up to 8 carbon atoms, that are optionally substituted by hydroxyl, straight chain or branched alkoxy with up to 6 carbon atoms or by phenyl, which in turn might be substituted by halogen, nitro, trifluormethyl, trifluoromethoxy or by phenyl or by tetrazole-substituted phenyl, and alkyl, optionally be substituted by a group with the formula

BOR_{X-22},

in which

R_{X-22} denotes a straight chain or branched acyl with up to 4 carbon atoms or benzyl, or

R_{X-19} denotes straight chain or branched acyl with up to 20 carbon atoms or benzoyl, that is optionally substituted by halogen, trifluoromethyl, nitro or trifluoromethoxy, or it denotes straight chain or branched fluoroacyl with up to 8 carbon atoms and 9 fluorine atoms,

R_{X-20} and R_{X-21} are identical or different and denote hydrogen, phenyl or straight chain or branched alkyl with up to 6 carbon atoms, or

R_{X-20} and R_{X-21} together form a 3- to 6-membered carbocyclic ring, and the carbocyclic rings formed are optionally substituted, optionally also geminally, with up to six identical or different substituents in the form of triflouromethyl, hydroxy, nitrile, halogen, carboxyl, nitro, azido, cyano, cycloalkyl or cycloalkyloxy with 3 to 7 carbon atoms each, by straight chain or branched alkoxycarbonyl, alkoxy or alkylthio with up to 6 carbon atoms each or by straight chain or branched alkyl with up to 6 carbon atoms, which in turn is substituted with up to 2 identically or differently by hydroxyl, benzyloxy, trifluoromethyl, benzoyl, straight chain or branched alkoxy, oxyacyl or carbonyl with up to 4 carbon atoms each and/or phenyl, which may in turn be substituted with a halogen, trifuoromethyl or trifluoromethoxy, and/or the formed carbocyclic rings are optionally substituted, also geminally, with up to 5 identical or different substituents in the form of phenyl, benzoyl, thiophenyl or sulfonylbenzyl, which in turn are optionally substituted by halogen, trifluoromethyl, trifluoromethoxy or nitro, and/or optionally are substituted by a radical with the formula 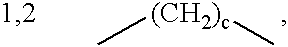 —SO₂—C₆H₅, —(CO)_{d}NR_{X-23}R_{X-24} or ═O,

in which

c denotes a number equaling 1, 2, 3, or 4,

d denotes a number equaling 0 or 1,

R_{X-23} and R_{X-24} are identical or different and denote hydrogen, cycloalkyl with 3 to 6 carbon atoms, straight chain or branched alkyl with up to 6 carbon atoms, benzyl or phenyl, that is optionally substituted with up to 2 identically or differently by halogen, trifluoromethyl, cyano, phenyl or nitro, and/or the formed carbocyclic rings are substituted optionally by a spiro-linked radical with the formula

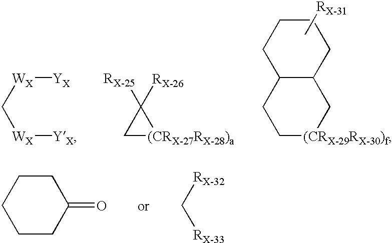

in which

W_{X} denotes either an oxygen or a sulfur atom

Y_{X} and Y′_{X} together form a 2 to 6 membered straight chain or branched alkylene chain,

e denotes a number equaling 1, 2, 3, 4, 5, 6, or 7,

f denotes a number equaling 1 or 2,

R_{X-25}, R_{X-26}, R_{X-27}, R_{X-28}, R_{X-29}, R_{X-30} and R_{X-31} are identical or different and denote hydrogen, trifluoromethyl, phenyl, halogen or straight chain or branched alkyl or alkoxy with up to 6 carbon atoms each, or

R_{X-25} and R_{X-26} or R_{X-27} and R_{X-28} respectively form together a straight chain or branched alkyl chain with up to 6 carbon atoms, or

R_{X-25} and R_{X-26} or R_{X-27} and R_{X-28} each together form a radical with the formula

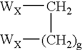

in which

W_{X} has the meaning given above,

g denotes a number equaling 1, 2, 3, 4, 5, 6, or 7,

R_{X-32} and R_{X-33} form together a 3- to 7-membered heterocycle, which contains an oxygen or sulfur atom or a group with the formula

SO, SO₂ or —NR_{X-34},

in which

R_{X-34} denotes hydrogen, phenyl, benzyl or straight or branched alkyl with up to 4 carbon atoms.

Compounds of Formula X are disclosed in WO 9914215, the complete disclosure of which is incorporated by reference.

In a preferred embodiment, the CETP inhibitor is selected from the following compounds of Formula X:

2-cyclopentyl-5-hydroxy-7,7-dimethyl-4-(3-thienyl)-3-(4-trifluoromethylbenxoyl)-5,6,7,8-tetrahydroquinoline;

2-cyclopentyl-3-[fluoro-(4-trifluoromethylphenyl)methyl]-5-hydroxy-7,7-dimethyl-4-(3-thienyl)-5,6,7,8-tetrahydroquinoline; and

2-cyclopentyl-5-hydroxy-7,7-dimethyl-4-(3-thienyl)-3-(trifluoromethylbenxyl)-5,6,7,8-tetrahydroquinoline.

Another class of CETP inhibitors that finds utility with the present invention consists of substituted tetrahydro naphthalines and analogous compound having the Formula XI

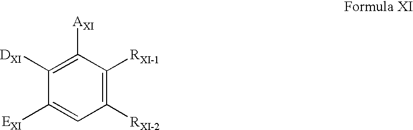

and stereoisomers, stereoisomer mixtures, and salts thereof,

in which

A_{XI} stands for cycloalkyl with 3 to 8 carbon atoms, or stands for aryl with 6 to 10 carbon atoms, or stands for a 5-to 7-membered, saturated, partially unsaturated or unsaturated, possibly benzocondensated, heterocycle with up to 4 heteroatoms from the series S, N and/or O, where aryl and the heterocyclic ring systems mentioned above are substituted up to 5-fold, identical or different, by cyano, halogen, nitro, carboxyl, hydroxy, trifluoromethyl, trifluoro- methoxy, or by straight-chain or branched alkyl, acyl, hydroxyalkyl, alkylthio, alkoxycarbonyl, oxyalkoxycarbonyl or alkoxy each with up to 7 carbon atoms, or by a group of the formula

—NR_{XI-3}R_{XI-4},

in which

R_{XI-3} and R_{XI-4} are identical or different and denote hydrogen, phenyl, or straight-chain or branched alkyl with up to 6 carbon atoms

D_{XI} stands for a radical of the formula

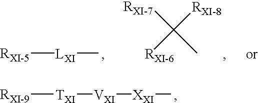

in which

R_{XI-5}, R_{XI-6} and R_{XI-9}, independent of each other, denote cycloalkyl with 3 to 6 carbon atoms, or denote aryl with 6 to 10 carbon atoms, or denote a 5- to 7-membered, possibly benzocondensated, saturated or unsaturated, mono-, bi- or tricyclic heterocycle with up to 4 heteroatoms of the series S, N and/or O, where the cycles are possibly substitutedCin the case of the nitrogen-containing rings also via the N-functionCup to 5-fold, identical or different, by halogen, trifluoromethyl. nitro, hydroxy, cyano, carboxyl, trifluoromethoxy, straight-chain or branched acyl, alkyl, alkylthio, alkylalkoxy, alkoxy or alkoxycarbonyl with up to 6 carbon atoms each. by aryl or trifluoromethyl substituted aryl with 6 to 10 carbon atoms each, or by a possibly benzocondensated aromatic 5- to 7-membered heterocycle with up to 3 heteroatoms of the series S, N and/or O, and/or are substituted by a group of the formula

—OR_{XI-10}, —SR_{XI-11}, —SO₂R_{XI-12} or —NR_{XI-13}R_{XI-}₁₄,

in which

R_{XI-10}, R_{XI-11}and R_{XI-12}, independent of each other, denote aryl with 6 to 10 carbon atoms, which itself is substituted up to 2-fold, identical or different, by phenyl, halogen, or by straight-chain or branched alkyl with up to 6 carbon atoms,

R_{XI-13} and R_{XI-14} are identical or different and have the meaning given above for R_{XI-3} and R_{XI-4}, or

R_{XI-5} and/or R_{XI-6} denote a radical of the formula

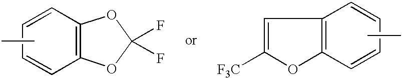

R_{XI-}₇ denotes hydrogen, halogen or methyl, and

R_{XI-}8 denotes hydrogen, halogen, azido, trifluoromethyl, hydroxy, trifluoromethoxy, straight-chain or branched alkoxy or alkyl with up to 6 carbon atoms each, or a radical of the formula

—NR_{XI-15}R_{XI-16},

in which

R_{XI-15} and R_{XI-16} are identical or different and have the meaning given above for R_{XI-3} and R_{XI-4}, or

R_{XI-7} and R_{XI-8} together form a radical of the formula

═O or —NR_{XI-17},

in which

R_{XI-17} denotes hydrogen or straight-chain or branched alkyl, alkoxy or acyl with up to 6 carbon atoms each,

L_{XI} denotes a straight-chain or branched alkylene- or alkenylene chain with up to 8 carbon atoms each, which is possibly substituted up to 2-fold by hydroxy,

T_{XI} and X_{XI} are identical or different and denote a straight-chain or branched alkylene chain with up to 8 carbon atoms, or

T_{XI} and X_{XI} denotes a bond,

V_{XI} stands for an oxygen- or sulfur atom or for an —NR_{XI-18} group,

in which

R_{XI-18} denotes hydrogen or straight-chain or branched alkyl with up to 6 carbon atoms, or phenyl,

E_{XI} stands for cycloalkyl with 3 to 8 carbon atoms, or stands for straight-chain or branched alkyl with up to 8 carbon atoms, which is possibly substituted by cycloalkyl with 3 to 8 carbon atoms or hydroxy, or stands for phenyl, which is possibly substituted by halogen or trifluoromethyl,

R_{XI-1} and R_{XI-2} together form a straight-chain or branched alkylene chain with up to 7 carbon atoms, which must be substituted by a carbonyl group and/or by a radical of the formula

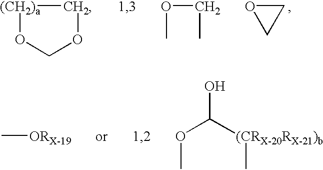

in which

a and b are identical or different and denote a number 1, 2 or 3

R_{XI-19} denotes hydrogen, cycloalkyl with 3 to 7 carbon atoms, straight-chain or branched silylalkyl with up to 8 carbon atoms, or straight-chain or branched alkyl with up to 8 carbon atoms, which is possibly substituted by hydroxy, straight-chain or branched alkoxy with up to 6 carbon atoms, or by phenyl, which itself can be substituted by halogen, nitro, trifluoromethyl, trifluoromethoxy or by phenyl substituted by phenyl or tetrazol, and alkyl is possibly substituted by a group of the formula

—OR_{XI-22},

in which

R_{XI-22} denotes straight-chain or branched acyl with up to 4 carbon atoms, or benzyl, or

R_{XI-19} denotes straight-chain or branched acyl with up to 20 carbon atoms or benzoyl, which is possibly substituted by halogen, trifluoromethyl, nitro or trifluoromethoxy, or denotes straight-chain or branched fluoroacyl with up to 8 carbon atoms and 9 fluorine atoms,

R_{XI-20} and R_{XI-21} are identical or different, denoting hydrogen, phenyl or straight-chain or branched alkyl with up to 6 carbon atoms, or

R_{XI-20} and R_{XI-21} together form a 3- to 6-membered carbocycle, and, possibly also geminally, the alkylene chain formed by R_{XI-1} and R_{XI-}2, is possibly substituted up to 6-fold, identical or different, by trifluoromethyl, hydroxy, nitrile, halogen, carboxyl, nitro, azido, cyano, cycloalkyl or cycloalkyloxy with 3 to 7 carbon atoms each, by straight-chain or branched alkoxycarbonyl, alkoxy or alkoxythio with up to 6 carbon atoms each, or by straight- chain or branched alkyl with up to 6 carbon atoms, which itself is substituted up to 2-fold, identical or different. by hydroxyl, benzyloxy, trifluoromethyl, benzoyl, straight-chain or branched alkoxy, oxyacyl or carboxyl with up to 4 carbon atoms each, and/or phenyl- which itself can be substituted by halogen, trifluoromethyl or trifluoromethoxy and/or the alkylene chain formed by R_{XI-1} and R_{XI-2} is substituted, also geminally, possibly up to 5-fold, identical or different, by phenyl, benzoyl, thiophenyl or sulfobenzyl—which themselves are possibly substituted by halogen, trifluoromethyl, trifluoromethoxy or nitro, and/or the alkylene chain formed by R_{XI-1} and R_{XI-2} is possibly substituted by a radical of the formula 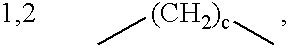 —SO₂—C₆H₅, —(CO)_{d}NR_{XI-23}R_{XI-24} or ═O,

in which

c denotes a number 1, 2, 3 or 4,

d denotes a number 0 or 1,

R_{XI-23} and R_{XI-24} are identical or different and denote hydrogen, cycloalkyl with 3 to 6 carbon atoms, straight-chain or branched alkyl with up to 6 carbon atoms, benzyl or phenyl, which is possibly substituted up to 2-fold. identical or different, by halogen, trifluoromethyl, cyano, phenyl or nitro, and/or the alkylene chain formed by R_{XI-1} and R_{XI-2} is possibly substituted by a spiro-jointed radical of the formula

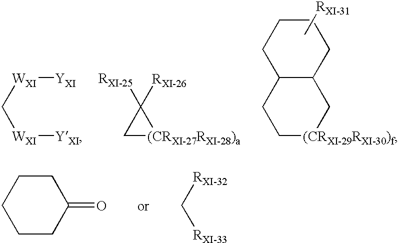

in which

W_{XI} denotes either an oxygen or a sulfur atom,

Y_{XI} and Y′_{XI} together form a 2- to 6-membered straight-chain or branched alkylene chain,

e is a number 1, 2, 3, 4, 5, 6 or 7,

f denotes a number 1 or 2,

R_{XI-25}, R_{XI-26}, R_{XI-}27, R_{XI-28}, R_{XI-29}, R_{XI-30} and R_{XI-31} are identical or different and denote hydrogen, trifluoromethyl, phenyl, halogen, or straight-chain or branched alkyl or alkoxy with up to 6 carbon atoms each, or

R_{XI-25} and R_{XI-26} or R_{XI-27} and R_{XI-28} together form a straight-chain or branched alkyl chain with up to 6 carbon atoms, or

R_{XI-25} and R_{XI-26} or R_{X-27} and R_{XI-28} together form a radical of the formula

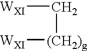

in which

W_{XI} has the meaning given above,

g is a number 1, 2, 3, 4, 5, 6 or 7,

R_{XI-32} and R_{XI-33} together form a 3- to 7-membered heterocycle that contains an oxygen- or sulfur atom or a group of the formula

SO, SO₂ or —NR_{XI-34},

in which

R_{XI-34} denotes hydrogen, phenyl, benzyl, or straight-chain or branched alkyl with up to 4 carbon atoms.

Compounds of Formula XI are disclosed in WO 9914174, the complete disclosure of which is incorporated by reference.

Another class of CETP inhibitors that finds utility with the present invention consists of 2-aryl-substituted pyridines having the Formula (XII)

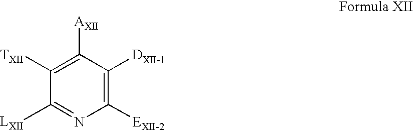

or pharmaceutically acceptable salts, enantiomers, or stereoisomers of said compounds,

in which

A_{XII} and E_{XII} are identical or different and stand for aryl with 6 to 10 carbon atoms which is possibly substituted, up to 5-fold identical or different, by halogen, hydroxy, trifluoromethyl, trifluoromethoxy, nitro or by straight-chain or branched alkyl, acyl, hydroxy alkyl or alkoxy with up to 7 carbon atoms each, or by a group of the formula

—NR_{XII-1}R_{XII-2},

where

R_{XII-1} and R_{XII-2} are identical or different and are meant to be hydrogen, phenyl or straight-chain or branched alkyl with up to 6 carbon atoms,

D_{XII} stands for straight-chain or branched alkyl with up to 8 carbon atoms, which is substituted by hydroxy,

L_{XII} stands for cycloalkyl with 3 to 8 carbon atoms or for straight-chain or branched alkyl with up to 8 carbon atoms, which is possibly substituted by cycloalkyl with 3 to 8 carbon atoms, or by hydroxy,

T_{XII} stands for a radical of the formula

R_{XII-3}—X_{XII}—or

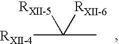

where

R_{XII-3} and R_{XII-4} are identical or different and are meant to be cycloalkyl with 3 to 8 carbon atoms, or aryl with 6 to 10 carbon atoms, or a 5-to 7-membered aromatic, possibly benzocondensated heterocycle with up to 3 heteroatoms from the series S, N and/or O, which are possibly substituted. up to 3-fold identical or different, by trifluoromethyl, trifluoromethoxy, halogen, hydroxy, carboxyl, nitro, by straight-chain or branched alkyl, acyl, alkoxy or alkoxycarbonyl with up to 6 carbon atoms each. or by phenyl, phenoxy or phenylthio which in turn can be substituted by halogen, trifluoromethyl or trifluoromethoxy, and/or where the cycles are possibly substituted by a group of the formula

—NR_{XII-7}R_{XII-8},

where

R_{XII-7} and R_{XII-8} are identical or different and have the meaning of R_{XII-1} and R_{XII-2} given above,

X_{XII} is a straight-chain or branched alkyl or alkenyl with 2 to 10 carbon atoms each, possibly substituted up to 2-fold by hydroxy or halogen,

R_{XII-5} stands for hydrogen, and

R_{XII-6} means to be hydrogen, halogen, mercapto, azido, trifluoromethyl, hydroxy, trifluoromethoxy, straight-chain or branched alkoxy with up to 5 carbon atoms, or a radical of the formula

BNR_{XII-9}R_{XII-10},

where

R_{XII-9} and R_{XII-10} are identical or different and have the meaning of R_{XII-1} and R_{XII-2} given above, or

R_{XII-5} and R_{XII-6} together with the carbon atom, form a carbonyl group.

Compounds of Formula XII are disclosed in EP 796846-A1, the complete disclosure of which is incorporated by reference.

In a preferred embodiment, the CETP inhibitor is selected from the following compounds of Formula XII:

4,6-bis-(p-fluorophenyl)-2-isopropyl-3-[(p-trifluoromethylphenyl)-(fluoro)-methyl]-5-(1-hydroxyethyl)pyridine;

2,4-bis-(4-fluorophenyl)-6-isopropyl-5-[4-(trifluoromethylphenyl)-fluoromethyl]-3-hydroxymethyl)pyridine; and

2,4-bis-(4-fluorophenyl)-6-isopropyl-5-[2-(3-trifluoromethylphenyl)vinyl]-3-hydroxymethyl)pyridine.

Another class of CETP inhibitors that finds utility with the present invention consists of compounds having the Formula (XIII)

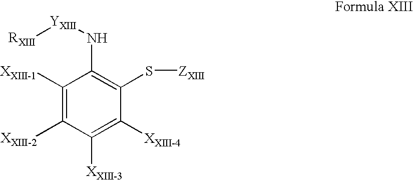

or pharmaceutically acceptable salts, enantiomers, stereoisomers, hydrates, or solvates of said compounds, in which

R_{XIII} is a straight chain or branched C₁₋₁₀ alkyl; straight chain or branched C₂₋₁₀ alkenyl; halogenated C₁₋₄ lower alkyl; C₃₋₁₀ cycloalkyl that may be substituted; C₅₋₈ cycloalkenyl that may be substituted; C₃₋₁₀ cycloalkyl C₁₋₁₀ alkyl that may be substituted; aryl that may be substituted; aralkyl that may be substituted; or a 5-or 6-membered heterocyclic group having 1 to 3 nitrogen atoms, oxygen atoms or sulfur atoms that may be substituted,

X_{XIII-1}, X_{XIII-2}, X_{XIII-3}, X_{XIII-4} may be the same or different and are a hydrogen atom; halogen atom; C₁₋₄ lower alkyl; halogenated C₁₋₄ lower alkyl; C₁₋₄ lower alkoxy; cyano group; nitro group; acyl; or aryl, respectively;

Y_{XIII} is —CO—; or BSO₂—; and

Z_{XIII} is a hydrogen atom; or mercapto protective group.

Compounds of Formula XIII are disclosed in WO 98/35937, the complete disclosure of which is incorporated by reference.

In a preferred embodiment, the CETP inhibitor is selected from the following compounds of Formula XIII:

N,N′-(dithiodi-2,1-phenylene)bis[2,2-dimethyl-propanamide];

N,N′-(dithiodi-2,1-phenylene)bis[1-methyl-cyclohexanecarboxamide];

N,N′-(dithiodi-2,1-phenylene)bis[1-(3-methylbutyl)-cyclopentanecarboxamide];

N,N′-(dithiodi-2,1-phenylene)bis[1-(3-methylbutyl)-cyclohexanecarboxamide];

N,N′-(dithiodi-2,1-phenylene)bis[1-(2-ethylbutyl)-cyclohexanecarboxamide];

N,N′-(dithiodi-2,1-phenylene)bis-tricyclo[3.3.1.1^{3,7}]decane-1-carboxamide;

propanethioic acid, 2-methyl-,S-[2 [[[1-(2-ethylbutyl)cyclohexyl]carbonyl]amino]phenyl] ester;

propanethioic acid, 2,2-dimethyl-, S-[2-[[[l-(2-ethylbutyl)cyclohexyl]carbonyl]amino]phenyl] ester; and

ethanethioic acid, S-[2-[[[1-(2-ethylbutyl)cyclohexyl]carbonyl]amino]phenyl] ester.

Another class of CETP inhibitors that finds utility with the present invention consists of polycyclic aryl and heteroaryl tertiary-heteroalkylamines having the Formula XIV

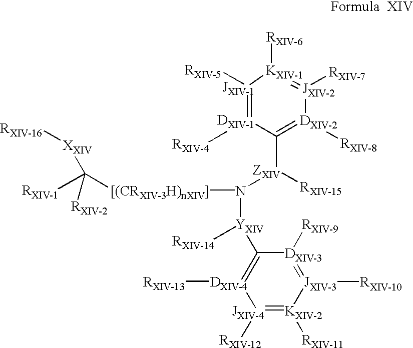

and pharmaceutically acceptable forms thereof, wherein:

n_{XIV} is an integer selected from 0 through 5;

R_{XIV-1} is selected from the group consisting of haloalkyl, haloalkenyl, haloalkoxyalkyl, and haloalkenyloxyalkyl;

X_{XIV} is selected from the group consisting of O, H, F, S, S(O),NH, N(OH), N(alkyl), and N(alkoxy);

R_{XIV-16} is selected from the group consisting of hydrido, alkyl, alkenyl, alkynyl, aryl, aralkyl, aryloxyalkyl, alkoxyalkyl, alkenyloxyalkyl, alkylthioalkyl, arylthioalkyl, aralkoxyalkyl, heteroaralkoxyalkyl, alkylsulfinylalkyl, alkylsulfonylalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxyalkyl, halocycloalkenyloxyalkyl, perhaloaryl, perhaloaralkyl, perhaloaryloxyalkyl, heteroaryl, heteroarylalkyl, monocarboalkoxyalkyl, monocarboalkoxy, dicarboalkoxyalkyl, monocarboxamido, monocyanoalkyl, dicyanoalkyl, carboalkoxycyanoalkyl, acyl, aroyl, heteroaroyl, heteroaryloxyalkyl, dialkoxyphosphonoalkyl, trialkylsilyl, and a spacer selected from the group consisting of a covalent single bond and a linear spacer moiety having from 1 through 4 contiguous atoms linked to the point of bonding of an aromatic substituent selected from the group consisting of R_{XIV-4}, R_{XIV-8}, R_{XIV-9}, and R_{XIV-13} to form a heterocyclyl ring having from 5 through 10 contiguous members with the provisos that said spacer moiety is other than a covalent single bond when R_{XIV-2} is alkyl and there is no R_{XIV-16} wherein X is H or F;

D_{XIV-1}, D_{XIV-2}, J_{XIV-1}, J_{XIV-2} and K_{XIV-1} are independently selected from the group consisting of C, N, O, S and a covalent bond with the provisos that no more than one of D_{XIV-1}, D_{XIV-2}, J_{XIV-1}, J_{XIV-2} and K_{XIV-1} is a covalent bond, no more than one of D_{XIV-1}, D_{XIV-2}, J_{XIV-1} J_{XIV-2} and K_{XIV-1} is O, no more than one of D_{XIV-1}, D_{XIV-2}, J_{XIV-1}, J_{XIV-2} and K_{XIV-1} is S, one of D_{XIV-1}, D_{XIV-2}, J_{XIV-1}, J_{XIV-2} and K_{XIV-1} must be a covalent bond when two of D_{XIV-1}, D_{XIV-2}₁, J_{XIV-1}, J_{XIV-2} and K_{XIV-1} are O and S, and no more than four of D_{XIV-1}, D_{XIV-2}, J_{XIV-1}, J_{XIV-2} and K_{XIV-1} are N;

D_{XIV-3}, D_{XIV-4}, J_{XIV-3}, J_{XIV-4} and K_{XIV-2} are independently selected from the group consisting of C, N, O, S and a covalent bond with the provisos that no more than one of D_{XIV-3}, D_{XIV-4}, J_{XIV-3}, J_{XIV-4} and K_{XIV-2} is a covalent bond, no more than one of D_{XIV-3}, D_{XIV-4}, J_{XIV-3}, J_{XIV-4} and K_{XIV-2} is O, no more than one of D_{XIV-3}, D_{XIV-4}, J_{XIV-3}, J_{XIV-4} and K_{XIV-2} is S, one of D_{XIV-3}, D_{XIV-4}, J_{XIV-3}, J_{XIV-4}; and K_{XIV-2} must be a covalent bond when two of D_{XIV-3}, D_{XIV-4}, J_{XIV-3}, J_{XIV-4} and K_{XIV-2} are O and S, and no more than four of D_{XIV-3}, D_{XIV-4}, J_{XIV-3}, J_{XIV-4} and K_{XIV-2} and K_{XIV-2} are N;

R_{XIV-2} is independently selected from the group consisting of hydrido, hydroxy, hydroxyalkyl, amino, aminoalkyl, alkylamino, dialkylamino, alkyl, alkenyl, alkynyl, aryl, aralkyl, aralkoxyalkyl, aryloxyalkyl, alkoxyalkyl, heteroaryloxyalkyl, alkenyloxyalkyl, alkylthioalkyl, aralkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, aloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, alkylsulfinylalkyl, alkylsulfonylalkyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, and diaralkoxyphosphonoalkyl;

R_{XIV-2} and R_{XIV-3} are taken together to form a linear spacer moiety selected from the group consisting of a covalent single bond and a moiety having from 1 through 6 contiguous atoms to form a ring selected from the group consisting of a cycloalkyl having from 3 through 8 contiguous members, a cycloalkenyl having from 5 through 8 contiguous members, and a heterocyclyl having from 4 through 8 contiguous members;

R_{XIV-3} is selected from the group consisting of hydrido, hydroxy, halo, cyano, aryloxy, hydroxyalkyl, amino, alkylamino, dialkylamino, acyl, sulfhydryl, acylamido, alkoxy, alkylthio, arylthio, alkyl, alkenyl, alkynyl, aryl, aralkyl, aryloxyalkyl, alkoxyalkyl, heteroarylthio, aralkylthio, aralkoxyalkyl, alkylsulfinylalkyl, alkylsulfonylalkyl, aroyl, heteroaroyl, aralkylthioalkyl, heteroaralkylthioalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, and diaralkoxyphosphonoalkyl;

Y_{XIV} is selected from a group consisting of a covalent single bond, (C(R_{XIV-14})₂)_{qXIV} wherein _{qXIV} is an integer selected from 1 and 2 and (CH (R_{XIV-14}) )_{g}_{XIV}13 W_{XIV}—(CH (R_{XIV-14}) )_{pXIV} wherein _{gXIV} and_{pXIV} are integers independently selected from 0 and 1;

R_{XIV-14} is independently selected from the group consisting of hydrido, hydroxy, halo, cyano, aryloxy, amino, alkylamino, dialkylamino, hydroxyalkyl, acyl, aroyl, heteroaroyl, heteroaryloxyalkyl, sulfhydryl, acylamido, alkoxy, alkylthio, arylthio, alkyl, alkenyl, alkynyl, aryl, aralkyl, aryloxyalkyl, aralkoxyalkylalkoxy, alkylsulfinylalkyl, alkylsulfonylalkyl, aralkylthioalkyl, heteroaralkoxythioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, diaralkoxyphosphonoalkyl, a spacer selected from a moiety having a chain length of 3 to 6 atoms connected to the point of bonding selected from the group consisting of R_{XIV-9} and R_{XIV-13} to form a ring selected from the group consisting of a cycloalkenyl ring having from 5 through 8 contiguous members and a heterocyclyl ring having from 5 through 8 contiguous members and a spacer selected from a moiety having a chain length of 2 to 5 atoms connected to the point of bonding selected from the group consisting of R_{XIV-4} and R_{XIV-8} to form a heterocyclyl having from 5 through 8 contiguous members with the proviso that, when Y_{XIV} is a covalent bond, an R_{XIV-14} substituent is not attached to Y_{XIV};

R_{XIV-14} and R_{XIV-14}, when bonded to the different atoms, are taken together to form a group selected from the group consisting of a covalent bond, alkylene, haloalkylene, and a spacer selected from a group consisting of a moiety having a chain length of 2 to 5 atoms connected to form a ring selected from the group of a saturated cycloalkyl having from 5 through 8 contiguous members, a cycloalkenyl having from 5 through 8 contiguous members, and a heterocyclyl having from 5 through 8 contiguous members;

R_{XIV-14} and R_{XIV-14}, when bonded to the same atom are taken together to form a group selected from the group consisting of oxo, thiono, alkylene, haloalkylene, and a spacer selected from the group consisting of a moiety having a chain length of 3 to 7 atoms connected to form a ring selected from the group consisting of a cycloalkyl having from 4 through 8 contiguous members, a cycloalkenyl having from 4 through 8 contiguous members, and a heterocyclyl having from 4 through 8 contiguous members;

W_{XIV} is selected from the group consisting of O, C(O), C (S), C (O) N (R_{XIV-14}), C (S) N (R_{XIV-14}), (R_{XIV-14}) NC (O), (R_{XIV-14}) NC (S), S, S (O), S (O)₂, S (O)₂N (R_{XIV-14}), (R_{XIV-14}) NS (O)₂, and N(R_{XIV-14}) with the proviso that R_{XIV-14} is selected from other than halo and cyano;

Z_{XIV} is independently selected from a group consisting of a covalent single bond, (C(R_{XIV-15})₂)_{qXIV-2} wherein _{qXIV-2} is an integer selected from 1 and 2, (CH (R_{XIV-15}))_{jXIV}—W—(CH(R_{XIV-15}) )ₖ_{XIV} wherein _{jXIV} and _{kXIV} are integers independently selected from 0 and 1 with the proviso that, when Z_{XIV} is a covalent single bond, an R_{XIV-15} substituent is not attached to Z_{XIV};

R_{XIV-15} is independently selected, when Z_{XIV} is (C(R_{XIV-15})₂)_{qXIV} wherein _{qXIV} is an integer selected from 1 and 2, from the group consisting of hydrido, hydroxy, halo, cyano, aryloxy, amino, alkylamino, dialkylamino, hydroxyalkyl, acyl, aroyl, heteroaroyl, heteroaryloxyalkyl, sulfhydryl, acylamido, alkoxy, alkylthio, arylthio, alkyl, alkenyl, alkynyl, aryl, aralkyl, aryloxyalkyl, aralkoxyalkyl, alkylsulfinylalkyl, alkylsulfonylalkyl, aralkylthioalkyl, heteroaralkylthioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, diaralkoxyphosphonoalkyl, a spacer selected from a moiety having a chain length of 3 to 6 atoms connected to the point of bonding selected from the group consisting of R_{XIV-4} and R_{XIV-8} to form a ring selected from the group consisting of a cycloalkenyl ring having from 5 through 8 contiguous members and a heterocyclyl ring having from 5 through 8 contiguous members, and a spacer selected from a moiety having a chain length of 2 to 5 atoms connected to the point of bonding selected from the group consisting of R_{XIV-9} and R_{XIV-13} to form a heterocyclyl having from 5 through 8 contiguous members;

R_{XIV-15} and R_{XIV-15}, when bonded to the different atoms, are taken together to form a group selected from the group consisting of a covalent bond, alkylene, haloalkylene, and a -spacer selected from a group consisting of a moiety having a chain length of 2 to 5 atoms connected to form a ring selected from the group of a saturated cycloalkyl having from 5 through 8 contiguous members, a cycloalkenyl having from 5 through 8 contiguous members, and a heterocyclyl having from 5 through 8 contiguous members;

R_{XIV-15} and R_{XIV-15}, when bonded to the same atom are taken together to form a group selected from the group consisting of oxo, thiono, alkylene, haloalkylene, and a spacer selected from the group consisting of a moiety having a chain length of 3 to 7 atoms connected to form a ring selected from the group consisting of a cycloalkyl having from 4 through 8 contiguous members, a cycloalkenyl having from 4 through 8 contiguous members, and a heterocyclyl having from 4 through 8 contiguous members;

R_{XIV-15} is independently selected, when Z_{XIV} is (CH (R_{XIV-15}) )_{jXIV}—W—(CH (R_{XIV-15}) ) _{kXIV} wherein _{jXIV} and _{kXIV} are integers independently selected from 0 and 1, from the group consisting of hydrido, halo, cyano, aryloxy, carboxyl, acyl, aroyl, heteroaroyl, hydroxyalkyl, heteroaryloxyalkyl, acylamido, alkoxy, alkylthio, arylthio, alkyl, alkenyl, alkynyl, aryl, aralkyl, aryloxyalkyl, alkoxyalkyl, heteroaryloxyalkyl, aralkoxyalkyl, heteroaralkoxyalkyl, alkylsulfonylalkyl, alkylsulfinylalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxyalkyl, carboalkoxy, carboxamide, carboxamidoalkyl, carboaralkoxy, dialkoxyphosphonoalkyl, diaralkoxyphosphonoalkyl, a spacer selected from a linear moiety having a chain length of 3 to 6 atoms connected to the point of bonding selected from the group consisting of R_{XIV-4} and R_{XIV-8} to form a ring selected from the group consisting of a cycloalkenyl ring having from 5 through 8 contiguous members and a heterocyclyl ring having from 5 through 8 contiguous members, and a spacer selected from a linear moiety having a chain length of 2 to 5 atoms connected to the point of bonding selected from the group consisting of R_{XIV-9} and R_{XIV-13} to form a heterocyclyl ring having from 5 through 8 contiguous members;

R_{XIV-4}, R_{XIV-5}, R_{XIV-6}, R_{XIV-7}, R_{XIV-8}, R_{XIV-9}, R_{XIV-10}, R_{XIV-11}, R_{XIV-12}, and R_{XIV-13} are independently selected from the group consisting of perhaloaryloxy, alkanoylalkyl, alkanoylalkoxy, alkanoyloxy, N-aryl-N-alkylamino, heterocyclylalkoxy, heterocyclylthio, hydroxyalkoxy, carboxamidoalkoxy, alkoxycarbonylalkoxy, alkoxycarbonylalkenyloxy, aralkanoylalkoxy, aralkenoyl, N-alkylcarboxamido, N-haloalkylcarboxamido, N-cycloalkylcarboxamido, N-arylcarboxamidoalkoxy, cycloalkylcarbonyl, cyanoalkoxy, heterocyclylcarbonyl, hydrido, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl; haloalkenyl, haloalkoxy, hydroxyhaloalkyl, hydroxyaralkyl, hydroxyaikyl, hydoxyheteroaralkyl, haloalkoxyalkyl, aryl, heteroaralkynyl, aryloxy, aralkoxy, aryloxyalkyl, saturated heterocyclyl, partially saturated heterocyclyl, heteroaryl, heteroaryloxy, heteroaryloxyalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl with the proviso that there are one to five non-hydrido ring substituents R_{XIV-4}, R_{XIV-5}, R_{XIV-6}, R_{XIV-7}, and R_{XIV-8} present, that there are one to five non-hydrido ring substituents R_{XIV-9}, R_{XIV-10}, R_{XIV-11}, R_{XIV-12}, and R_{XIV-13} present, and R_{XIV-4}, R_{XIV-5}, R_{XIV-6}, R_{XIV-7}, R_{XIV-8}, R_{XIV-9}, R_{XIV-10}, R_{XIV-11}, R_{XIV-12}, and R_{XIV-13} are each independently selected to maintain the tetravalent nature of carbon, trivalent nature of nitrogen, the divalent nature of sulfur, and the divalent nature of oxygen;

R_{XIV-4} and R_{XIV-5}, R_{XIV-5} and R_{XIV-6}, R_{XIV-6} and R_{XIV-7}, R_{XIV-7} and R_{XIV-8}, R_{XIV-8} and R_{XIV-9}, R_{XIV-9} and R_{XIV-10}, R_{XIV-10} and R_{XIV-11}, R_{XIV-11} and R_{XIV-12}, and R_{XIV-12} and R_{XIV-13} are independently selected to form spacer pairs wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the provisos that no more than one of the group consisting of spacer pairs R_{XIV-4} and R_{XIV-5}, R_{XIV-5} and R_{XIV-}6, R_{XIV-6} and R_{XIV-}7, and R_{XIV-7} and R_{XIV-8} are used at the same time and that no more than one of the group consisting of spacer pairs R_{XIV-9} and R_{XIV-10}, R_{XIV-10} and R_{XIV-11}, R_{XIV-11} and R_{XIV-12}, and R_{XIV-12} and R_{XIV-13} are used at the same time;

R_{XIV-4} and R_{XIV-9}, R_{XIV-4} and R_{XIV-13}, R_{XIV-8} and R_{XIV-9}, and R_{XIV-8} and R_{XIV-13} are independently selected to form a spacer pair wherein said spacer pair is taken together to form a linear moiety wherein said linear moiety forms a ring selected from the group consisting of a partially saturated heterocyclyl ring having from 5 through 8 contiguous members and a heteroaryl ring having from 5 through 6 contiguous members with the proviso that no more than one of the group consisting of spacer pairs R_{XIV-4} and R_{XIV-9}, R_{XIV-4} and R_{XIV-13}, R_{XIV-8} and R_{XIV-9}, and R_{XIV-8} and R_{XIV-13} is used at the same time.

Compounds of Formula XIV are disclosed in WO 00/18721, the entire disclosure of which is incorporated by reference.

In a preferred embodiment, the CETP inhibitor is selected from the following compounds of Formula XIV:

3-[[3-(3-trifluoromethoxyphenoxy)phenyl][[3-(1,1,2,2-tetrafluoroethoxy)-phenyl]methyl]amino]-1,1,1-trifluoro-2-propanol;

3-[[3-(3-isopropylphenoxy)phenyl][[3-(1,1,2,2-tetrafluoroethoxy)phenyl]-methyl]amino]-1,1,1-trifluoro-2-propanol;

3-[[3-(3-cyclopropylphenoxy)phenyl][[3-(1,1,2,2-tetrafluoroethoxy)phenyl]-methyl]amino]-1,1,1-trifluoro-2-propanol;

3-[[3-(3-(2-furyl)phenoxy)phenyl][[3-(1,1,2,2-tetrafluoroethoxy)phenyl]-methyl]amino]1,1,1-trifluoro-2-propanol;

3-[[3-(2,3-dichlorophenoxy)phenyl][[3-(1,1,2,2-tetrafluoroethoxy)phenyl]-methyl]amino]-1,1,1-trifluoro-2-propanol;

3-[[3-(4-fluorophenoxy)phenyl][[3-(1,1,2,2-tetrafluoroethoxy)phenyl]-methyl]amino]-1,1,1-trifluoro-2-propanol;

3-[[3-(4-methlylphenoxy)phenyl][[3-(1,1,2,2-tetrafluoroethoxy)phenyl]-methyl]amino]-1,1,1-trifluoro-2-propanol;

3-[[3-(2-fluoro-5-bromophenoxy)phenyl][[3-(1,1,2,2-tetrafluoroethoxy)phenyl]-methyl]amino]-1,1,1-trifluoro-2-propanol;

3-[[3-(4-chloro-3-ethylphenoxy)phenyl][[3-(1,1,2,2-tetrafluoroethoxy)phenyl]-methyl]amino]-1,1,1-trifluoro-2-propanol;

3-[[3-[3-(1,1,2,2-tetrafluoroethoxy)phenoxy]phenyl][[3-(1,1,2,2-tetrafluoro-ethoxy)phenyl]methyl]amino]-1,1,1-trifluoro-2-propanol;

3-[[3-[3-(pentafluoroethyl)phenoxy]phenyl][[3-(1,1,2,2-tetrafluoroethoxy)-phenyl]methyl]amino]-1,1,1-trifluoro-2-propanol;

3-[[3-(3,5-dimethylphenoxy)phenyl][[3-(1,1,2,2-tetrafluoroethoxy)phenyl]-methyl]amino]-1,1,1-trifluoro-2-propanol;

3-[[3-(3-ethylphenoxy)phenyl][[3-(1,1,2,2-tetrafluoroethoxy) phenyl]-methyl]amino]-1,1,1-trifluoro-2-propanol;

3-[[3-(3-t-butylphenoxy)phenyl][[3-(1,1,2,2-tetrafluoroethoxy)phenyl]-methyl]amino]1,1,1-trifluoro-2-propanol;

3-[[3-(3-methylphenoxy)phenyl][[3-(1,1,2,2-tetrafluoroethoxy)phenyl]-methyl]amino]-1,1,1-trifluoro-2-propanol;

3-[[3-(5,6,7,8-tetrahydro-2-naphthoxy)phenyl][[3-(1,1,2,2-tetrafluoroethoxy)phenyl]methyl]amino]-1,1,1-trifluoro-2-propanol;

3-[[3-(phenoxy)phenyl][[3-(1,1,2,2-tetrafluoroethoxy) phenyl]methyl]amino]-1,1,1-trifluoro-2-propanol;

3-[[3-[3-(N,N-dimethylamino)phenoxy]phenyl][[3-(1,1,2,2-tetrafluoroethoxy)phenyl]methyl]amino]-1,1,1-trifluoro-2-propanol;

3-[[[3-(1,1,2,2-tetrafluoroethoxy)phenyl]methyl][3-[[3-(trifluoromethoxy)-phenyl]methoxy]phenyl]amino]-1,1,1-trifluoro-2-propanol;

3-[[[3-(1,1,2,2-tetrafluoroethoxy)phenyl]methyl][3-[[3-(trifluoromethyl)-phenyl]methoxy]phenyl]amino]-1,1,1-trifluoro-2-propanol;

3-[[[3-(1,1,2,2-tetrafluoroethoxy)phenyl]methyl][3-[[3,5-dimethylphenyl]-methoxy]phenyl]amino]-1,1,1-trifluoro-2-propanol;

3-[[[3-(1,1,2,2-tetrafluoroethoxy)phenyl]methyl][3-[[3-(trifluoromethylthio)-phenyl]methoxy]phenyl]amino]-1,1,-trifluoro-2-propanol;

3-[[[3-(1,1,2,2-tetrafluoroethoxy)phenyl]methyl][3-[[3,5-difluorophenyl]-methoxy]phenyl]amino]-1,1,1-trifluoro-2-propanol;

3-[[[3-(1,1,2,2-tetrafluoroethoxy)phenyl]methyl][3-[cyclohexylmethoxy]-phenyl]amino]-1,1,l-trifluoro-2-propanol;

3-[[3-(2-difluoromethoxy-4-pyridyloxy)phenyl][[3-(1,1,2,2-tetrafluoroethoxy)-phenyl]methyl]amino]-1,1,1-trifluoro-2-propanol;

3-[[3-(2-trifluoromethyl-4-pyridyloxy)phenyl][[3-(1,1,2,2-tetrafluoroethoxy)-phenyl]methyl]amino]-1,1,1-trifluoro-2-propanol;

3-[[3-(3-difluoromethoxyphenoxy)phenyl][[3-(1,1,2,2-tetrafluoroethoxy)-phenyl]methyl]amino]-1,1,1-trifluoro-2-propanol;

3-[[[3-(3-trifluoromethylthio)phenoxy]phenyl][[3-(1,1,2,2-tetrafluoroethoxy)-phenyl]methyl]amino]-1,1,1-trifluoro-2-propanol;

3-[[3-(4-chloro-3-trifluoromethylphenoxy)phenyl][[3-(1,1,2,2-tetrafluoroethoxy)-phenyl]methyl]amino]-1,1,1,-trifluoro-2-propanol;

3-[[3-(3-trifluoromethoxyphenoxy)phenyl][[3-(pentafluoroethymethyl]amino]-1,1,1-trifluoro-2-propanol;

3-[[3-(3-isopropylphenoxy)phenyl][[3-(pentafluoroethyl) phenyl]methyl]-amino]-1,1,1-trifluoro-2-propanol;

3-[[3-(3-cyclopropylphenoxy)phenyl][[3-(pentafluoroethyl) phenyl]methyl]-amino]-1,1,1-trifluoro-2-propanol;

3-[[3-(3-(2-furyl)phenoxy)phenyl][[3-(pentafluoroethyl) phenyl]methyl]-amino]-1,1,1-trifluoro-2-propanol;

3-[[3-(2,3-dichlorophenoxy)phenyl][[3-(pentafluoroethyl) phenyl]methyl]-amino]-1,1,1-trifluoro-2-propanol;

3-[[3-(4-fluorophenoxy)phenyl][[3-(pentafluoroethyl) phenyl]methyl]amino]l1-1,1-trifluoro-2-propanol;

3-[[3-(4-methylphenoxy)phenyl][[3-(pentafluoroethyl) phenyl]methyl]amino]-1,1,1-trifluoro-2-propanol;

3-[[3-(2-fluoro-5-bromophenoxy)phenyl][[3-(pentafluoroethyl) phenyl]methyl]-amino]-1,1,1-trifluoro-2-propanol;

3-[[3-(4-chloro-3-ethylphenoxy)phenyl][[3-(pentafluoroethyl) phenyl]methyl]-amino]-1,1,1-trifluoro-2-propanol;

3-[[3-[3-(1,1,2,2-tetrafluoroethoxy)phenoxy]phenyl][[3-(pentafluoroethyl)-phenyl]methyl]amino]-1,1,1-trifluoro-2-propanol;

3-[[3-[3-(pentafluoroethyl)phenoxy]phenyl][[3-(pentafluoroethyl)phenyl]-methyl]amino]-1,1,1-trifluoro-2-propanol;

3-[[3-(3,5-dimethylphenoxy)phenyl][[3-(pentafluoroethyl) phenyl]methyl]-amino]-1,1,1-trifluoro-2-propanol;

3-[[3-(3-ethylphenoxy)phenyl][[3-(pentafluoroethyl) phenyl]methyl]amino]-1,1,1-trifluoro-2-propanol;

3-[[3-(3-t-butylphenoxy)phenyl][[3-(pentafluoroethyl) phenyl]methyl]amino]-1,1,1-trifluoro-2-propanol;

3-[[3-(3-methylphenoxy)phenyl][[3-pentafluoroethyl) phenyl]methyl]amino]-1,1,1-trifluoro-2-propanol;

3-[[3-(5,6,7,8-tetrahydro-2-naphthoxy)phenyl][[3-(pentafluoroethyl)phenyl]-methyl]amino]-1,1,1-trifluoro-2-propanol;

3-[[3-(phenoxy)phenyl][[3-(pentafluoroethyl)phenyl]methyl] amino]-1,1,1-trifluoro-2-propanol;

3-[[3-[3-(N,N-dimethylamino)phenoxy]phenyl][[3-(pentafluoroethyl)phenyl]-methyl]amino]-1,1,1-trifluoro-2-propanol;

3-[[[3-(pentafluoroethyl)phenyl]methyl][3-[[3-(trifluoromethoxy)phenyl]-methoxy]phenyl]amino]-1,1,1-trifluoro-2-propanol;

3-[[[3-(pentafluoroethyl)phenyl]methyl][3-[[3-(trifluoromethyl)phenyl]-methoxy]phenyl]amino]-1,1,1-trifluoro-2-propanol;

3-[[[3-(pentafluoroethyl)phenyl]methyl][3-[[3,5-dimethylphenyl]methoxy]-phenyl]amino]-1,1,1-trifluoro-2-propanol;

3-[[[3-(pentafluoroethyl)phenyl]methyl][3-[[3-(trifluoromethylthio)phenyl]-methoxy]phenyl]amino]-1,1,1-trifluoro-2-propanol;

3-[[[3-(pentafluoroethyl)phenyl]methyl][3-[[3,5-difluorophenyl]methoxy]-phenyl]amino]-1,1,1-trifluoro-2-propanol;

3-[[[3-(pentafluoroethyl)phenyl]methyl][3-[cyclohexylmethoxy]phenyl]-amino]-1,1,1-trifluoro-2-propanol;

3-[[3-(2-difluoromethoxy-4-pyridyloxy)phenyl][[3-(pentafluoroethyl)phenyl]-methyl]amino]-1,1,1-trifluoro-2-propanol;

3-[[3-(2-trifluoromethyl-4-pyridyloxy)phenyl][[3-(pentafluoroethyl)phenyl]-methyl]amino]-1,1,1-trifluoro-2-propanol;

3-[[3-(3-difluoromethoxyphenoxy)phenyl][[3-(pentafluoroethyl) phenyl]-methyl]amino]-1,1,1-trifluoro-2-propanol;

3-[[[3-(3-trifluoromethylthio)phenoxy]phenyl][[3-(pentafluoroethyl)phenyl]-methyl]amino]-1,1,1-trifluoro-2-propanol;

3-[[3-(4-chloro-3-trifluoromethylphenoxy)phenyl][[3-(pentafluoroethyl)-phenyl]methyl]amino]-1,1,1-trifluoro-2-propanol;

3-[[3-(3-trifluoromethoxyphenoxy)phenyl][[3-(heptafluoropropyl)phenyl]-methyl]amino]-1,1,1-trifluoro-2-propanol;

3-[[3-(3-isopropylphenoxy)phenyl][[3-(heptafluoropropyl) phenyl]methyl]-amino]-1,1,1-trifluoro-2-propanol;

3-[[3-(3-cyclopropylphenoxy)phenyl][[3-(heptafluoropropyl) phenyl]methyl]-amino]-1,1,1-trifluoro-2-propanol;

3-[[3-(3-(2-furyl)phenoxy)phenyl][[3-(heptafluoropropyl) phenyl]methyl]-amino]-1,1,1-trifluoro-2-propanol;

3-[[3-(2,3-dichlorophenoxy)phenyl][[3-(heptafluoropropyl) phenyl]methyl]-amino]-1,1,1-trifluoro-2-propanol;

3-[[3-(4-fluorophenoxy)phenyl][[3-(heptafluoropropyl) phenyl]methyl]amino]-1,1,1-trifluoro-2-propanol;

3-[[3-(4-methylphenoxy)phenyl][[3-(heptafluoropropyl) phenyl]methyl]amino]-1,1,1-trifluoro-2-propanol;

3-[[3-(2-fluoro-5-bromophenoxy)phenyl][[3-(heptafluoropropyl) phenyl]-methyl]amino]-1,1,1-trifiuoro-2-propanol;

3-[[3-(4-chloro-3-ethylphenoxy)phenyl][[3-(heptafluoropropyl) phenyl]methyl]-amino]-1,1,1-trifluoro-2-propanol;

3-[[3-[3-(1,1,2,2-tetrafluoroethoxy)phenoxy]phenyl][[3-(heptafluoropropyl)-phenyl]methyl]amino]-1,1,1-trifluoro-2-propanol;

3-[[3-[3-(pentafluoroethyl)phenoxy]phenyl][[3-(heptafluoropropyl)phenyl]-methyl]amino]-1,1,1-trifluoro-2-propanol;

3-[[3-(3,5-dimethylphenoxy)phenyl][[3-(heptafluoropropyl) phenyl]methyl]-amino]-1,1,1-trifluoro-2-propanol;

3-[[3-(3-ethylphenoxy)phenyl][[3-(heptafluoropropyl) phenyl]methyl]amino]-1,1,1-trifluoro-2-propanol;

3-[[3-(3-t-butylphenoxy)phenyl][[3-(heptafluoropropyl) phenyl]methyl]amino]-1,1,1-trifluoro-2-propanol;

3-[[3-(3-methylphenoxy)phenyl][[3-(heptafluoropropyl) phenyl]methyl]amino]-1,1,1-trifluoro-2-propanol;

3-[[3-(5,6,7,8-tetrahydro-2-naphthoxy)phenyl][[3-(heptafluoropropyl)phenyl]-methyl]amino]-1,1,1-trifluoro-2-propanol;

3-[[3-(phenoxy)phenyl][[3-(heptafluoropropyl)phenyl]methyl] amino]-1,1,1-trifluoro-2-propanol;

3-[[3-[3-(N,N-dimethylamino)phenoxy]phenyl][[3-(heptafluoropropyl)phenyl]-methyl]amino]-1,1,1-trifluoro-2-propanol;

3-[[[3-(heptafluoropropyl)phenyl]methyl][3-[[3-(trifluoromethoxy)phenyl]-methoxy]phenyl]amino]-1,1,1-trifluoro-2-propanol;

3-[[[3-(heptafluoropropyl)phenyl]methyl][3-[[3-(trifluoromethyl)phenyl]-methoxy]phenyl]amino]-1,1,1-trifluoro-2-propanol;

3-[[[3-(heptafluoropropyl)phenyl]methyl][3-[[3,5-dimethylphenyl]methoxy]-phenyl]amino]-1,1,1-trifluoro-2-propanol;

3-[[[3-(heptafluoropropyl)phenyl]methyl][3-[[3-(trifluoromethylthio)phenyl]-methoxy]phenyl]amino]-1,1,1-trifluoro-2-propanol;

3-[[[3-(heptafluoropropyl)phenyl]methyl][3-[[3,5-difluorophenyl]methoxy]-phenyl]amino]-1,1,1-trifluoro-2-propanol;

3-[[[3-(heptafluoropropyl)phenyl]methyl][3-[cyclohexylmethoxy]phenyl]-amino]-1,1,1-trifluoro-2-propanol;

3-[[3-(2-difluoromethoxy-4-pyridyloxy)phenyl][[3-(heptafluoropropyl)phenyl]-methyl]amino]-1,1,1-trifluoro-2-propanol;

3-[[3-(2-trifluoromethyl-4-pyridyloxy)phenyl][[3-(heptafluoropropyl)phenyl]-methyl]amino]-1,1,1-trifluoro-2-propanol;

3-[[3-(3-difluoromethoxyphenoxy)phenyl][[3-(heptafluoropropyl) phenyl]-methyl]amino]-1,1,1-trifluoro-2-propanol;

3-[[[3-(3-trifluoromethylthio)phenoxy]phenyl][[3-(heptafluoropropyl)phenyl]-methyl]amino]-1,1,1-trifluoro-2-propanol;

3-[[3-(4-chloro-3-trifluoromethylphenoxy)phenyl][[3-(heptafluoropropyl)-phenyl]-methyl]amino]-1,1,1-trifluoro-2-propanol;

3-[[3-(3-trifluoromethoxyphenoxy)phenyl][[2-fluoro-5-(trifluoromethyl)-phenyl]-methyl]amino]-1,1,1-trifluoro-2-propanol;

3-[[3-(3-isopropylphenoxy)phenyl][[2-fluoro-5-(trifluoromethyl)phenyl]-methyl]amino]-1,1,1-trifluoro-2-propanol;

3-[[3-(3-cyclopropylphenoxy)phenyl][[2-fluoro-5-(trifluoromethyl)phenyl]-methyl]amino]-1,1,1-trifluoro-2-propanol;

3-[[3-(3-(2-furyl)phenoxy)phenyl][[2-fluoro-5-(trifluoromethyl)phenyl]-methyl]amino]-1,1,1-trifluoro-2-propanol;

3-[[3-(2,3-dichlorophenoxy)phenyl][[2-fluoro-5-(trifluoromethyl)phenyl]-methyl]amino]-1,1,1-trifluoro-2-propanol;

3-[[3-(4-fluorophenoxy)phenyl][[2-fluoro-5-(trifluoromethyl) phenyl]-methyl]amino]-1,1,1-trifluoro-2-propanol;

3-[[3-(4-methylphenoxy)phenyl][[2-fluoro-5-(trifluoromethyl)phenyl]-methyl]amino]-1,1,1-trifluoro-2-propanol;

3-[[3-(2-fluoro-5-bromophenoxy)phenyl][[2-fluoro-5-(trifluoromethyl)-phenyl]methyl]amino]-1,1,1-trifluoro-2-propanol;

3-[[3-(4-chloro-3-ethylphenoxy)phenyl][[2-fluoro-5-(trifluoromethyl)-phenyl]methyl]amino]-1,1,1-trifluoro-2-propanol;

3-[[3-[3-(1,1,2,2-tetrafluoroethoxy)phenoxy]phenyl][[2-fluoro-5-(trifluoro-methyl)phenyl]methyl]amino]-1,1,1-trifluoro-2-propanol;

3-[[3-[3-(pentafluoroethyl)phenoxy]phenyl][[2-fluoro-5-(trifluoromethyl)-phenyl]-methyl]amino]-1,1,1-trifluoro-2-propanol;

3-[[3-(3,5-dimethylphenoxy)phenyl][[2-fluoro-5-(trifluoromethyl)phenyl]-methyl]amino]-1,1,1-trifluoro-2-propanol;

3-[[3-(3-ethylphenoxy)phenyl][[2-fluoro-5-(trifluoromethyl) phenyl]methyl]-amino]-1,1,1-trifluoro-2-propanol;

3-[[3-(3-t-butylphenoxy)phenyl][[2-fluoro-5-(trifluoromethyl) phenyl]methyl]-amino]-1,1,1-trifluoro-2-propanol;

3-[[3-(3-methylphenoxy)phenyl][[2-fluoro-5-(trifluoromethyl) phenyl]methyl]-amino]-1,1,1-trifluoro-2-propanol;

3-[[3-(5,6,7,8-tetrahydro-2-naphthoxy)phenyl][[2-fluoro-5-(trifluoromethyl)-phenyl]methyl]amino]-1,1,1-trifluoro-2-propanol;

3-[[3-(phenoxy)phenyl][[2-fluoro-5-(trifluoromethyl) phenyl]methyl]amino]-1,1,1-trifluoro-2-propanol;

3-[[3-[3-(N,N-dimethylamino)phenoxy]phenyl][[2-fluoro-5-(trifluoromethyl)-phenyl]methyl]amino]-1,1,1-trifluoro-2-propanol;

3-[[[2-fluoro-5-(trifluoromethyl)phenyl]methyl][3-[[3-(trifluoromethoxy)-phenyl]methoxy]phenyl]amino]-1,1,1-trifluoro-2-propanol;

3-[[[2-fluoro-5-(trifluoromethyl)phenyl]methyl][3-[[3-(trifluoromethyl)-phenyl]methoxy]phenyl]amino]-1,1,1-trifluoro-2-propanol;

3-[[[2-fluoro-5-(trifluoromethyl)phenyl]methyl][3-[[3,5-dimethylphenyl]-methoxy]phenyl]amino]-1,1,1-trifluoro-2-propanol;

3-[[[2-fluoro-5-(trifluoromethyl)phenyl]methyl][3-[[3-(trifluoromethylthio)-phenyl]methoxy]phenyl]amino]1,1,1-trifluoro-2-propanol;

3-[[[2-fluoro-5-(trifluoromethyl)phenyl]methyl][3-[[3,5-difluorophenyl]-methoxy]phenyl]amino]-1,1,1-trifluoro-2-propanol;

3-[[[2-fluoro-5-(trifluoromethyl)phenyl]methyl][3-[cyclohexylmethoxy]-phenyl]amino]-1,1,1-trifluoro-2-propanol;

3-[[3-(2-difluoromethoxy-4-pyridyloxy)phenyl][[2-fluoro-5-(trifluoromethyl)-phenyl]methyl]amino]-1,1,1-trifluoro-2-propanol;

3-[[3-(2-trifluoromethyl-4-pyridyloxy)phenyl][[2-fluoro-5-(trifluoromethyl)-phenyl]methyl]amino]-1,1,1-trifluoro-2-propanol;

3-[[3-(3-difluoromethoxyphenoxy)phenyl][[2-fluoro-5-(trifluoromethyl)-phenyl]methyl]amino]-1,1,1-trifluoro-2-propanol;

3-[[[3-(3-trifluoromethylthio)phenoxy]phenyl][[2-fluoro-5-(trifluoromethyl)-phenyl]methyl]amino]-1,1,1-trifluoro-2-propanol;

3-[[3-(4-chloro-3-trifluoromethylphenoxy)phenyl][[2-fluoro-5-(trifluoromethyl)phenyl]methyl]amino]1,1,1-trifluoro-2-propanol;

3-[[3-(3-trifluoromethoxyphenoxy)phenyl][[2-fluoro-4-(trifluoromethyl)-phenyl]methyl]amino]-1,1,1-trifluoro-2-propanol;

3-[[3-(3-isopropylphenoxy)phenyl][[2-fluoro-4-(trifluoromethyl)phenyl]-methyl]amino]-1,1,1-trifluoro-2-propanol;

3-[[3-(3-cyclopropylphenoxy)phenyl][[2-fluoro-4-(trifluoromethyl)phenyl]-methyl]amino]-1,1,1-trifluoro-2-propanol;

3-[[3-(3-(2-furyl)phenoxy)phenyl][[2-fluoro-4-(trifluoromethyl)phenyl]-methyl]amino]-1,1,1-trifluoro-2-propanol;

3-[[3-(2,3-dichlorophenoxy)phenyl][[2-fluoro-4-(trifluoromethyl)phenyl]-methyl]amino]-1,1,1-trifluoro-2-propanol;

3-[[3-(4-fluorophenoxy)phenyl][[2-fluoro-4-(trifluoromethyl) phenyl]-methyl]amino]-1,1,1-trifluoro-2-propanol;

3-[[3-(4-methylphenoxy)phenyl][[2-fluoro-4-(trifluoromethyl) phenyl]-methyl] amino]-1,1,1-trifluoro-2-propanol;

3-[[3-(2-fluoro-5-bromophenoxy)phenyl][[2-fluoro-4-(trifluoromethyl)-phenyl]methyl]amino]-1,1,1-trifluoro-2-propanol;

3-[[3-(4-chloro-3-ethylphenoxy)phenyl][[2-fluoro-4-(trifluoromethyl)-phenyl]methyl]amino]-1,1,1-trifluoro-2-propanol;

3-[[3-[3-(1,1,2,2-tetrafluoroethoxy)phenoxy]phenyl][[2-fluoro-4-(trifluoro-methyl)phenyl]methyl]amino]-1,1,1-trifluoro-2-propanol;

3-[[3-[3-(pentafluoroethyl)phenoxy]phenyl][[2-fluoro-4-(trifluoromethyl)-phenyl]methyl]amino]-1,1,1-trifluoro-2-propanol;

3-[[3-(3,5-dimethylphenoxy)phenyl][[2-fluoro-4-(trifluoromethyl)phenyl]-methyl]amino]-1,1,1-trifluoro-2-propanol;

3-[[3-(3-ethylphenoxy)phenyl][[2-fluoro-4-(trifluoromethyl) phenyl]methyl]-amino]-1,1,1-trifluoro-2-propanol;

3-[[3-(3-t-butylphenoxy)phenyl][[2-fluoro-4-(trifluoromethyl) phenyl]methyl]-amino]-1,1,1-trifluoro-2-propanol;

3-[[3-(3-methylphenoxy)phenyl][[2-fluoro-4-(trifluoromethyl) phenyl]methyl]-amino]-1,1,1-trifluoro-2-propanol;

3-[[3-(5,6,7,8-tetrahydro-2-naphthoxy)phenyl][[2-fluoro-4-(trifluoromethyl)-phenyl]methyl]amino]-1,1,1-trifluoro-2-propanol;

3-[[3-(phenoxy)phenyl][[2-fluoro-4-(trifluoromethyl) phenyl]methyl]amino]-1,1,1-trifluoro-2-propanol;

3-[[3-[3-(N,N-dimethylamino)phenoxy]phenyl][[2-fluoro-4-(trifluoromethyl)-phenyl]methyl]amino]-1,1,1-trifluoro-2-propanol;

3-[[[2-fluoro-4-(trifluoromethyl)phenyl]methyl][3-[[3-(trifluoromethoxy)-phenyl]methoxy]phenyl]amino]-1,1,1-trifluoro-2-propanol;

3-[[[2-fluoro-4-(trifluoromethyl)phenyl]methyl][3-[[3-(trifluoromethyl)-phenyl]methoxy]phenyl]amino]-1,1,1-trifluoro-2-propanol;

3-[[[2-fluoro-4-(trifluoromethyl)phenyl]methyl][3-[[3,5-dimethylphenyl]-methoxy]phenyl]amino]-1,1,1-trifluoro-2-propanol;

3-[[[2-fluoro-4-(trifluoromethyl)phenyl]methyl][3-[[3-(trifluoromethylthio)-phenyl]methoxy]phenyl]amino]-1,1,1-trifluoro-2-propanol;

3-[[[2-fluoro-4-(trifluoromethyl)phenyl]methyl][3-[[3,5-difluorophenyl]-methoxy]phenyl]amino]-1,1,1-trifluoro-2-propanol;

3-[[[2-fluoro-4-(trifluoromethyl)phenyl]methyl][3-[cyclohexylmethoxy]-phenyl]amino]-1,1,1-trifluoro-2-propanol;

3-[[3-(2-difluoromethoxy-4-pyridyloxy)phenyl][[2-fluoro-4-(trifluoromethyl)-phenyl]methyl]amino]-1,1,1-trifluoro-2-propanol;

3-[[3-(2-trifluoromethyl-4-pyridyloxy)phenyl][[2-fluoro-4-(trifluoromethyl)-phenyl]methyl]amino]-1,1,1-trifluoro-2-propanol;

3-[[3-(3-difluoromethoxyphenoxy)phenyl][[2-fluoro-4-(trifluoromethyl)-phenyl]methyl]amino]-1,1,1-trifluoro-2-propanol;

3-[[[3-(3-trifluoromethylthio)phenoxy]phenyl][[2-fluoro-4-(trifluoromethyl)-phenyl]methyl]amino]-1,1,1-trifluoro-2-propanol; and

3-[[3-(4-chloro-3-trifluoromethylphenoxy)phenyl][[ 2-fluoro-4-(trifluoro-methyl)phenyl]methyl]amino]-1,1,1-trifluoro-2-propanol.

Another class of CETP inhibitors that finds utility with the present invention consists of substitued N-Aliphatic-N-Aromatic tertiary-Heteroalkylamines having the Formula XV

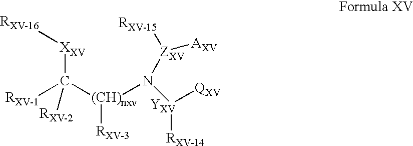

and pharmaceutically acceptable forms thereof, wherein:

n_{XV} is an integer selected from 1 through 2;

A_{XV} and Q_{XV} are independently selected from the group consisting of —CH₂(CR_{XV-37}R_{XV-38})_{vXV}—(CR_{XV-33}R_{XV-34})_{uXV}-T_{XV}—(CR_{XV-35}R_{XV-36})w_{XV-}H,

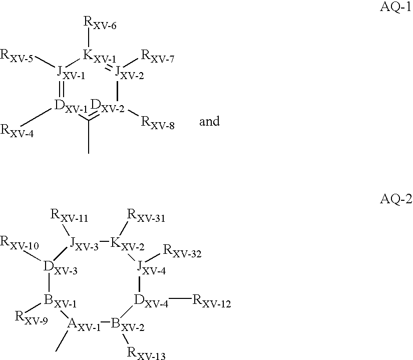

with the provisos that one of A_{XV} and Q_{XV} must be AQ-1 and that one of A_{XV} and Q_{XV} must be selected from the group consisting of AQ-2 and —CH₂ (CR_{XV-37}R_{XV-38})_{vXV}—(CR_{XV-33}R_{XV-34})ᵤ_{XV}-T_{XV} —(CR_{XV-35}R_{XV-36})w_{XV} —H;

T_{XV} is selected from the group consisting of a single covalent bond, O, S, S(O), S(O)₂, C(R_{XV-33})═C(R_{XV-35}), and C≡C;

v_{XV} is an integer selected from 0 through 1 with the proviso that v_{XV} is 1 when any one of R_{XV-33}, R_{XV-34}, R_{XV-35}, and R_{XV-36} is aryl or heteroaryl;

u_{XV} and w_{XV} are integers independently selected from 0 through 6;

A_{XV-1} is C(R_{XV-30}) ;

D_{XV-1}, D_{XV-2}, J_{XV-1}, J_{XV-2}, and K_{XV-1} are independently selected from the group consisting of C, N, O, S and a covalent bond with the provisos that no more than one of D_{XV-1}, D_{XV-2}, J_{XV-1}, J_{XV-}2, and K_{XV-1} is a covalent bond, no more than one of D_{XV-1}, D_{XV-2}, J_{XV-1}, J_{XV-2}, and K_{XV-1} is O, no more than one of D_{XV-1}, D_{XV-2}, J_{XV-1}, J_{XV-2}, and K_{XV-1} is S, one of D_{XV-1}, D_{XV-2}, J_{XV-1}, J_{XV-2}, and K_{XV-1} must be a covalent bond when two of D_{XV-1}, D_{XV-2}, J_{XV-1}, J_{XV-2}, and K_{XV-1} are O and S, and no more than four of D_{XV-1}, D_{XV-2}, J_{XV-1}, J_{XV-2}, and K_{XV-1}, are N;

B_{XV-1}, B_{XV-2}, D_{XV-3}, D_{XV-4}, J_{XV-3}, J_{XV-4}, and K_{XV-2} are independently selected from the group consisting of C, C(R_{XV-30}), N, O, S and a covalent bond with the provisos that no more than 5 of B_{XV-1}, B_{XV-2}, D_{XV-3}, D_{XV-3}, J_{XV-4}, and K_{XV-2} are a covalent bond, no more than two of B_{XV-1}, B_{XV-2}, D_{XV-3}, D_{XV-4}, J_{XV-3}, J_{XV-4}, and K_{XV-2} are O, no more than two of B_{XV-1}, B_{XV-2}, D_{XV- 3}, D_{XV-4}, J_{XV-3}, J_{XV-4}, and K_{XV-2} are S, no more than two of B_{XV-1}, B_{XV-2}, D_{XV-3}, D_{XV-4}, J_{XV-3}, J_{XV-4}, and K_{XV-2} are simultaneously O and S, and no more than two of B_{XV-1}, B_{XV-2}, D_{XV-3}, D_{XV-4}, J_{XV-3}, J_{XV-4}, and K_{XV-2} are N;

B_{XV-1} and D_{XV-3}, D_{XV-3} and J_{XV-3}, J_{XV-3} and K_{XV-2}, K_{XV-2} and J_{XV-4}, J_{XV-4} and D_{XV-4}, and D_{XV-4} and B_{XV-2} are independently selected to form an in-ring spacer pair wherein said spacer pair is selected from the group consisting of C(R_{XV-33})═C(R_{XV-35}) and N═N with the provisos that AQ-2 must be a ring of at least five contiguous members, that no more than two of the group of said spacer pairs are simultaneously C(R_{XV-33})═C(R_{XV-35}) and that no more than one of the group of said spacer pairs can be N═N unless the other spacer pairs are other than C(R_{XV-33})═C(R_{XV-35}), O, N, and S;

R_{XV-1} is selected from the group consisting of haloalkyl and haloalkoxymethyl;

R_{XV-2} is selected from the group consisting of hydrido, aryl, alkyl, alkenyl, haloalkyl, haloalkoxy, haloalkoxyalkyl, perhaloaryl, perhaloaralkyl, perhaloaryloxyalkyl and heteroaryl;

R_{XV-3} is selected from the group consisting of hydrido, aryl, alkyl, alkenyl, haloalkyl, and haloalkoxyalkyl;

Y_{XV} is selected from the group consisting of a covalent single bond, (CH₂)_{q} wherein q is an integer selected from 1 through 2 and (CH₂)ⱼ—O—(CH₂)ₖ wherein j and k are integers independently selected from 0 through 1;

Z_{XV} is selected from the group consisting of covalent single bond, (CH₂)_{q} wherein q is an integer selected from 1 through 2, and (CH₂)ⱼ—O—(CH₂)ₖ wherein j and k are integers independently selected from 0 through 1;

R_{XV-4}, R_{XV-8}, R_{XV-9} and R_{XV-13} are independently selected from the group consisting of hydrido, halo, haloalkyl, and alkyl;

R_{XV-30} is selected from the group consisting of hydrido, alkoxy, alkoxyalkyl, halo, haloalkyl, alkylamino, alkylthio, alkylthioalkyl, alkyl, alkenyl, haloalkoxy, and haloalkoxyalkyl with the proviso that R_{XV-30} is selected to maintain the tetravalent nature of carbon, trivalent nature of nitrogen, the divalent nature of sulfur, and the divalent nature of oxygen;

R_{XV-30}, when bonded to A_{XV}, is taken together to form an intra-ring linear spacer connecting the A_{XV-1}-carbon at the point of attachment of R_{XV-30} to the point of bonding of a group selected from the group consisting of R_{XV-10}, R_{XV-11}, R_{XV-12}, R_{XV 31}, and R_{XV-32} wherein said intra-ring linear spacer is selected from the group consisting of a covalent single bond and a spacer moiety having from 1 through 6 contiguous atoms to form a ring selected from the group consisting of a cycloalkyl having from 3 through 10 contiguous members, a cycloalkenyl having from 5 through 10 contiguous members, and a heterocyclyl having from 5 through 10 contiguous members;

R_{XV-30}, when bonded to A_{XV-1} is taken together to form an intra-ring branched spacer connecting the A_{XV-1}-carbon at the point of attachment of R_{XV-30} to the points of bonding of each member of any one of substituent pairs selected from the group consisting of substituent pairs R_{XV-10} and R_{XV-11}, R_{XV 10} and R_{XV-31}, R_{XV 10} and R_{XV-32}, R_{XV-10} and R_{XV-12}, R_{XV-11} and R_{XV-31}, R_{XV-11} and R_{XV-32}, R_{XV-11} and R_{XV-12}, R_{XV-31} and R_{XV-32}, R_{XV-31} and R_{XV-12}, and R_{XV-32} and R_{XV-12} and wherein said intra-ring branched spacer is selected to form two rings selected from the group consisting of cycloalkyl having from 3 through 10 contiguous members, cycloalkenyl having from 5 through 10 contiguous members, and heterocyclyl having from 5 through 10 contiguous members;

R_{XV-4}, R_{XV-5}, R_{XV-6}, R_{XV-7}, R_{XV-8}, R_{XV-9}, R_{XV-10}, R_{XV-11}, R_{XV-12}, R_{XV-13}, R_{XV-31}, R_{XV-32}, R_{XV-33}, R_{XV-34}, R_{XV-35}, and R_{XV-36} are independently selected from the group consisting of hydrido, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, hydoxyheteroaralkyl, haloalkoxyalkyl, aryl, heteroaralkynyl, aryloxy, aralkoxy, aryloxyalkyl, saturated heterocyclyl, partially saturated heterocyclyl, heteroaryl, heteroaryloxy, heteroaryloxyalkyl, arylalkenyl, heteroarylalkenyl, carboxyalkyl, carboalkoxy, alkoxycarboxamido, alkylamidocarbonylamido, alkylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl with the provisos that R_{XV-4}, R_{XV-5}, R_{XV-6}, R_{XV-7}, R_{XV-8}, R_{XV-9}, R_{XV-10}, R_{XV-11}, R_{XV-12}, R_{XV-13}, R_{XV-31}, R_{XV-32}, R_{XV-33}, R_{XV-34}, R_{XV-35}, and R_{XV-36} are ea independently selected to maintain the tetravalent nature of carbon, trivalent nature of nitrogen, the divalent nature of sulfur, and the divalent nature of oxygen, that no more than three of the R_{XV-33} and R_{XV-34} substituents are simultaneously selected from other than the group consisting of hydrido and halo, and that no more than three of the R_{XV-35} and R_{XV-36} substituents are simultaneously selected from other than the group consisting of hydrido and halo;

R_{XV-9}, R_{XV-10}, R_{XV-11}, R_{XV-12}, R_{XV-13}, R_{XV-31}, and R_{XV-32} are independently selected to be oxo with the provisos that B_{XV-1}, B_{XV-2}, D_{XV-3}, D_{XV-4}, J_{XV-3}, J_{XV-4}, and K_{XV-2} are independently selected from the group consisting of C and S, no more than two of R_{XV-9}, R_{XV-10}, R_{XV-11}, R_{XV-12}, R_{XV-13}, R_{XV-31}, and R_{XV-32} are simultaneously oxo, and that R_{XV-9}, R_{XV-10}, R_{XV-11}, R_{XV-12}, R_{XV-13}, R_{XV-31}, and R_{XV-32} are each independently selected to maintain the tetravalent nature of carbon, trivalent nature of nitrogen, the divalent nature of sulfur, and the divalent nature of oxygen;

R_{XV-4} and R_{XV-5}, R_{XV-5} and R_{XV-6}, R_{XV-6} and R_{XV-7}, R_{XV-7} and R_{XV-8}, R_{XV-9} and R_{XV-10}, R_{XV-10} and R_{XV-11}, R_{XV-11} and R_{XV-31}, R_{XV-31} and R_{XV-32}, R_{XV-32} and R_{XV-12}, and R_{XV-12} and R_{XV-13} are independently selected to form spacer pairs wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the provisos that no more than one of the group consisting of spacer pairs R_{XV-4} and R_{XV-5}, R_{XV-5} and R_{XV-6}, R_{XV-6} and R_{XV-7}, R_{XV-7} and R_{XV-8} is used at the same time and that no more than one of the group consisting of spacer pairs R_{XV-9} and R_{XV-10}, R_{XV-10} and R_{XV-11}, R_{XV-11} and R_{XV-31}, R_{XV-31} and R_{XV-32}, R_{XV-32} and R_{XV-12}, and R_{XV-12} and R_{XV-13} are used at the same time;

R_{XV-9} and R_{XV-11}, R_{XV-9} and R_{XV-12}, R_{XV-9} and R_{XV-13} R_{XV-9} and R_{XV-31}, R_{XV-9} and R_{XV-32}, R_{XV-10} and R_{XV-12}, R_{XV-10} and R_{XV-13}, R_{XV-10} and R_{XV-31}, R_{XV-10} and R_{XV-32}, R₁₁ and R_{XV-12}, R_{XV-11} and R_{XV-13}, R_{XV-11} and R_{XV-32}, R_{XV-12} and R_{XV-31}, R_{XV-13} and R_{XV-31}, and R_{XV-13} and R_{XV-32} are independently selected to form a spacer pair wherein said spacer pair is taken together to form a linear spacer moiety selected from the group consisting of a covalent single bond and a moiety having from 1 through 3 contiguous atoms to form a ring selected from the group consisting of a cycloalkyl having from 3 through 8 contiguous members, a cycloalkenyl having from 5 through 8 contiguous members, a saturated heterocyclyl having from 5 through 8 contiguous members and a partially saturated heterocyclyl having from 5 through 8 contiguous members with the provisos that no more than one of said group of spacer pairs is used at the same time;

R_{XV-37} and R_{XV-38} are independently selected from the group consisting of hydrido, alkoxy, alkoxyalkyl, hydroxy, amino, thio, halo, haloalkyl, alkylamino, alkylthio, alkylthioalkyl, cyano, alkyl, alkenyl, haloalkoxy, and haloalkoxyalkyl.

Compounds of Formula XV are disclosed in WO 00/18723, the entire disclosure of which is incorporated by reference.

In a preferred embodiment, the CETP inhibitor is selected from the following compounds of Formula XV:

3-[[3-(4-chloro-3-ethylphenoxy)phenyl] (cyclohexylmethyl)amino]-1,1,1-trifluoro-2-propanol;

3-[[3-(4-chloro-3-ethylphenoxy)phenyl] (cyclopentylmethyl)amino]-1,1,1-trifluoro-2-propanol;

3-[[3-(4-chloro-3-ethylphenoxy)phenyl] (cyclopropylmethyl)amino]-1,1,1-trifluoro-2-propanol;

3-[[3-(4-chloro-3-ethylphenoxy)phenyl][(3-trifiuoromethyl)cyclohexyl-methyl]amino]-1,1,1-trifluoro-2-propanol;

3-[[3-(4-chloro-3-ethylphenoxy)phenyl][(3-pentafluoroethyl) cyclohexyl-methyl]amino]-1,1,1-trifluoro-2-propanol;

3-[[3-(4-chloro-3-ethylphenoxy)phenyl][(3-trifluoromethoxy) cyclohexyl-methyl]amino]-1,1,1-trifluoro-2-propanol;

3-[[3-(4-chloro-3-ethylphenoxy)phenyl][[3-(1,1,2,2-tetrafluoroethoxy)cyclo-hexylmethyl]amino]-1,1,1-trifluoro-2-propanol;

3-[[3-(3-trifluoromethoxyphenoxy)phenyl] (cyclohexylmethyl)amino]-1,1,1-trifluoro-2-propanol;

3-[[3-(3-trifluoromethoxyphenoxy) phenyl] (cyclopentylmethyl)amino]amino-1,1,1-trifluoro-2-propanol;

3-[[3-(3-trifluoromethoxyphenoxy)phenyl] (cyclopropylmethyl)amino]-1,1,1-trifluoro-2-propanol;

3-[[3-(3-trifluoromethoxyphenoxy)phenyl][(3-trifluoromethyl)cyclohexyl-methyl]amino-1,1,1-trifluoro-2-propanol;

3-[[3-(3-trifluoromethoxyphenoxy)phenyl](3-pentafluoroethyl)cyclohexyl-methyl]amino]-1,1,1-trifluoro-2-propanol;

3-[[3-(3-trifluoromethoxyphenoxy)phenyl][(3-trifluoromethoxy)cyclohexyl-methyl]amino]-1,1,1-trifluoro-2-propanol;

3-[[3-(3-trifluoromethoxyphenoxy)phenyl][[3-(1,1,2,2-tetrafluoroethoxy)cyclohexyl-methyl]amino]-1,1,1-trifluoro-2-propanol;

3-[[3-(3-isopropylphenoxy)phenyl] (cyclohexylmethyl]amino]-1,1,1-trifiuoro-2-propanol:

3-[[3-(3-isopropylphenoxy)phenyl] (cyclopentylmethyl]amino]-1,1,1-trifluoro-2-propanol;

3-[[3-(3-isopropylphenoxy)phenyl] (cyclopropylmethyl)amino]-1,1,1-trifluoro-2-propanol;

3-[[3-(3-isopropylphenoxy)phenyl][(3-trifluoromethyl) cyclohexyl-methyl]amino]-1,1,1-trifluoro-2-propanol;

3-[[3-(3-isopropylphenoxy)phenyl][(3-pentafluoroethyl) cyclohexyl-methyl]amino]-1,1,1-trifluoro-2-propanol;

3-[[3-(3-isopropylphenoxy)phenyl][(3-trifluoromethoxy) cyclohexyl-methyl]amino]-1,1,1-trifluoro-2-propanol;

3-[[3-(3-isopropylphenoxy)phenyl][3-(1,1,2,2-tetrafluoroethoxy)cyclohexyl-methyl]amino]-1,1,1-trifluoro-2-propanol;

3-[[3-(2,3-dichlorophenoxy)phenyl] (cyclohexylmethyl)amino]-1,1,1-trifluoro-2-propanol;

3-[[3-(2,3-dichlorophenoxy)phenyl] (cyclopentylmethyl) amino]-1,1,1-trifluoro-2-propanol;

3-[[3-(2,3-dichlorophenoxy)phenyl] (cyclopropylmethy)amino]-1,1,1-trifluoro-2-propanol;

3-[[3-(2,3-dichlorophenoxy)phenyl][(3-trifluoromethyl) cyclohexyl-methyl]amino]-1,1,1-trifluoro-2-propanol;

3-[[3-(2,3-dichlorophenoxy)phenyl][(3-pentafluoroethyl) cyclohexyl-methyl]amino]-1,1,1-trifluoro-2-propanol;

3-[[3-(2,3-dichlorophenoxy)phenyl][(3-trifluoromethoxy) cyclohexyl-methyl]amino]-1,1,1-trifluoro-2-propanol;

3-[[3-(2,3-dichlorophenoxy)phenyl][3-(1,1,2,2-tetrafluoroethoxy)cyclo-hexyl-methyl]amino]-1,1,1-trifluoro-2-propanol;

3-[[3-(4-fluorophenoxy)phenyl] (cyclohexylmethyl)amino]-1,1,1-trifluoro-2-propanol;

3-[[3-(4-fluorophenoxy)phenyl] (cyclopentylmethyl)amino]-1,1,1-trifluoro-2-propanol;

3-[[3-(4-fluorophenoxy)phennyl](cyclopropylmethyl)amino]-1,1,1-triflouro-2-propanol;

3-[[3-(4-fluorophenoxy)phenyl][(3-trifluoromethyl) cyclohexyl-methyl]amino]-1,1,1-trifluoro-2-propanol;

3-[[3-(4-fluorophenoxy)phenyl][(3-pentafluoroethyl) cyclohexyl-methyl]amino]-1,1,1-trifluoro-2-propanol;

3-[[3-(4-fluorophenoxy)phenyl][(3-trifluoromethoxy) cyclohexyl-methyl]amino]-1,1,1-trifluoro-2-propanol;

3-[[3-(4-fluorophenoxy)phenyl][[3-(1,1,2,2-tetrafluoroethoxy)cyclohexyl-methyl]amino]-1,1,1-trifluoro-2-propanol;

3-[[3-(3-trifluoromethoxybenzyloxy]phenyl] (cyclohexylmethyl)amino]-1,1,1-trifluoro-2-propanol;

3-[[3-(3-trifluoromethoxybenzyloxy)phenyl] (cyclopentylmethyl)amino]-1,1,1-trifluoro-2-propanol;

3-[[3-(3-trifluoromethoxybenzyloxy)phenyl] (cyclopropylmethyl]amino]-1,1,1-trifluoro-2-propanol;

3-[[3-(3-trifluoromethoxybenzyloxy)phenyl][(3-trifluoromethyl)cyclohexyl-methyl]amino]-1,1,1-trifluoro-2-propanol;

3-[[3-(3-trifluoromethoxybenzyloxy)phenyl][(3-pentafluoroethyl)cyclohexyl-methyl]amino]-1,1,1-trifluoro-2-propanol;

3-[[3-(3-trifluoromethoxybenzyloxy]phenyl][(3-trifluoromethoxy)cyclohexyl-methyl]amino]-1,1,1-trifluoro-2-propanol;

3-[[3-(3-trifluoromethoxybenzyloxy)phenyl][3-(1,1,2,2-tetrafluoroethoxy)-cyclohexylmethyl]amino]-1,1,1-trifluoro-2-propanol;

3-[[3-(3-trifluoromethylbenzyloxy)phenyl] (cyclohexylmethyl)amino]-1,1,1-trifluoro-2-propanol;

3-[[3-(3-trifluoromethylbenzyloxy)phenyl] (cyclopentylmethyl)amino]-1,1,1-trifluoro-2-propanol;

3-[[3-(3-trifluoromethylbenzyloxy)phenyl] (cyclopropylmethyl)amino]-1,1,1-trifluoro-2-propanol;

3-[[3-(3-trifluoromethylbenzyloxy)phenyl][(3-trifluoromethyl)cyclohexyl-methyl]amino]-1,1,1-trifluoro-2-propanol;

3-[[3-(3-trifluoromethylbenzyloxy)phenyl][(3-pentafluoroethyl)cyclohexyl-methyl]amino]-1,1,1-trifluoro-2-propanol;

3-[[3-(3-trifluoromethylbenzyloxy)phenyl][(3-trifluoromethoxy)cyclohexyl-methyl]amino]-1,1,1-trifluoro-2-propanol;

3-[[3-(3-trifluoromethylbenzyloxy)phenyl][3-(1,1,2,2-tetrafluoroethoxy)cyclohexyl-methyl]amino]-1,1,1-trifluoro-2-propanol;

3-[[[(3-trifluoromethyl)phenyl]methyl](cyclohexyl)amino]-1,1,1-trifluoro-2-propanol;

3-[[[(3-pentafluoroethyl)phenyl]methyl](cyclohexyl)amino]-1,1,1-trifluoro-2-propanol;

3-[[[(3-trifluoromethoxy)phenyl]methyl](cyclohexyl)amino]-1,1,1-trifluoro-2-propanol;

3-[[[3-(1,1,2,2-tetrafluoroethoxy)phenyl] methyl](cyclohexyl)amino]-1,1,1-trifluoro-2-propanol;

3-[[[(3-trifluoromethyl)phenyl]methyl] (4-methylcyclohexyl)amino]-1,1,1-trifluoro-2-propanol;

3-[[[(3-pentafluoroethyl)phenyl]methyl] (4-methylcyclohexyl)amino]-1,1,1-trifluoro-2-propanol;

3-[[[(3-trifluoromethoxy)phenyl]methyl] (4-methylcyclohexyl)amino]-1,1,1-trifluoro-2-propanol;

3-[[[3-(1,1,2,2-tetrafluoroethoxy)phenyl]methyl](4-methylcyclohexyl)amino]-1,1,1-trifluoro-2-propanol;

3-[[[(3-trifluoromethyl]phenyl]methyl](3-trifluoromethylcyclohexyl)amino]-1,1,1-trifluoro-2-propanol;

3-[[[(3-pentafluoroethyl)phenyl]methyl](3-trifluoromethylcyclohexyl)amino]-1,1,1-trifluoro-2-propanol;

3-[[[(3-trifluoromethoxy)phenyl]methyl](3-trifluoromethylcyclohexyl)amino]-1,1,1-trifluoro-2-propanol;

3-[[[3-(1,1,2,2-tetrafluoroethoxy)phenyl]methyl](3-trifluoromethylcyclohexyl)amino]-1,1,1-trifluoro-2-propanol;

3-[[[(3-trifluoromethyl)phenyl]methyl][3-(4-chloro-3-ethylphenoxy)cyclo-hexyl]amino]-1,1,1-trifluoro-2-propanol;

3-[[[(3-pentafluoroethyl)phenyl]methyl][3-(4-chloro-3-ethylphenoxy)cyclo-hexyl]amino]-1,1,1-trifluoro-2-propanol;

3-[[[(3-trifluoromethoxy)phenyl]methyl][3-(4-chloro-3-ethylphenoxy)cyclo-hexyl]amino]-1,1,1-trifluoro-2-propanol;

3-[[[3-(1,1,2,2-tetrafluoroethoxy)phenyl]methyl][3-(4-chloro-3-ethylphenoxy)-cyclohexyl]amino]-1,1,1-trifluoro-2-propanol;

3-[[[(3-trifluoromethyl]phenyl]methyl](3-phenoxycyclohexyl)amino]-1,1,1-trifluoro-2-propanol;

3-[[[(3-pentafluoroethyl)phenyl]methyl](3-phenoxycyclohexyl)amino]-1,1,1-trifluoro-2-propanol;

3-[[[(3-trifluoromethoxy)phenyl]methyl](3-phenoxycyclohexyl)amino]-1,1,1-trifluoro-2-propanol;

3-[[[3-(1,1,2,2-tetrafluoroethoxy)phenyl]methyl](3-phenoxycyclohexyl)amino]-1,1,1-trifluoro-2-propanol;

3-[[[(3-trifloromethyl)phenyl]methyl](3-isopropoxycyclohexyl)amino]-1,1,1-trifluoro-2-propanol;

3-[[[(3-pentafluoroethyl)phenyl]methyl](3-isopropoxycyclohexyl)amino]-1,1,1-trifluoro-2-propanol;

3-[[[(3-trifluoromethoxy)phenyl]methyl](3-isopropoxycyclohexyl)amino]-1,1,1-trifluoro-2-propanol;

3-[[[3-(1,1,2,2-tetrafluoroethoxy)phenyl]methyl](3-isopropoxycyclohexyl)-amino]-1,1,1-trifluoro-2-propanol;

3-[[[(3-trifluoromethyl)phenyl]methyl](3-cyclopentyloxycyclohexyl]amino]-1,1,1-trifluoro-2-propanol;

3-[[[(3-pentafluoroethyl]phenyl]methyl](3-cyclopentyloxycyclohexyl)amino]-1,1,1-trifluoro-2-propanol;

3-[[[(3-trifluoromethoxy)phenyl]methyl](3-cyclopentyloxycyclohexyl)amino]-1,1,1-trifluoro-2-propanol;

3-[[[3-(1,1,2,2-tetrafluoroethoxy)phenyl]methyl](3-cyclopentyloxycyclohexyl)-amino]-1,1,1-trifluoro-2-propanol;

3-[[[(2-trifluoromethyl)pyrid-6-yl]methyl](3-isopropoxycyclohexyl)amino]-1,1,1-trifluoro-2-propanol;

3-[[[(2-trifluoromethyl)pyrid-6-yl]methyl](3-cyclopentyloxycyclohexyl)-amino]-1,1,1-trifluoro-2-propanol;

3-[[[(2-trifluoromethyl)pyrid-6-yl]methyl](3-phenoxycyclohexyl)amino]-1,1,1-trifluoro-2-propanol;

3-[[[(2-trifluoromethyl)pyrid-6-yl]methyl](3-trifluoromethylcyclohexyl)amino]-1,1,1-trifluoro-2-propanol;

3-[[[(2-trifluoromethyl)pyrid-6-yl]methyl][3-(4-chloro-3-ethylphenoxy)cyclo-hexyl]amino]-1,1,1-trifluoro-2-propanol;

3-[[[(2-trifluoromethyl)pyrid-6-yl]methyl][3-(1,1,2,2-tetrafluoroethoxy)cyclo-hexyl]amino]-1,1,1-trifluoro-2-propanol;

3-[[[(2-trifluoromethyl)pyrid-6-yl]methyl](3-pentafluoroethylcyclohexyl)-amino]-1,1,1-trifluoro-2-propanol;

3-[[[(2-trifluoromethyl)pyrid-6-yl]methyl](3-trifluoromethoxycyclohexyl)-amino]-1,1,1-trifluoro-2-propanol;

3-[[[(3-trifluoromethyl)phenyl]methyl][3-(4-chloro-3-ethylphenoxy)propyl]-amino]-1,1,1-trifluoro-2-propanol;

3-[[[(3-pentafluoroethyl)phenyl]methyl][3-(4-chloro-3-ethylphenoxy)propyl]-amino]-1,1,1-trifluoro-2-propanol;

3-[[[(3-trifluoromethoxy)phenyl]methyl][3-(4-chloro-3-ethylphenoxy)propyl]-amino]-1,1,1-trifluoro-2-propanol;

3-[[[3-(1,1,2,2-tetrafluoroethoxy)phenyl]methyl][3-(4-chloro-3-ethylphenoxy)-propyl]amino]-1,1,1-trifluoro-2-propanol;

3-[[[(3-trifluoromethyl)phenyl]methyl][3-(4-chloro-3-ethylphenoxy)-2,2,-di-fluropropyl]amino]-1,1,1-trifluoro-2-propanol;

3-[[[(3-pentafluoroethyl)phenyl]methyl][3-(4-chloro-3-ethylphenoxy)-2,2-di-fluropropyl]amino]-1,1,1-trifluoro-2-propanol;

3-[[[(3-trifluoromethoxy)phenyl]methyl][3-(4-chloro-3-ethylphenoxy)-2,2,-di-fluropropyl]amino]-1,1,1-trifluoro-2-propanol;

3-[[[3-(1,1,2,2-tetrafluoroethoxy)phenyl]methyl][3-(4-chloro-3-ethylphenoxy)-2,2,-difluropropyl]amino]-1,1,1-trifluoro-2-propanol;

3-[[[(3-trifluoromethyl)phenyl]methyl][3-(isopropoxy)propyl]amino]-1,1,1-trifluoro-2-propanol;

3-[[[(3-pentafluoroethyl)phenyl]methyl][3-(isopropoxy)propyl]amino]-1,1,1-trifluoro-2-propanol;

3-[[[(3-trifluoromethoxy)phenyl]methyl][3-(isopropoxy)propyl]amino]-1,1,1-trifluoro-2-propanol;

3-[[[3-(1,1,2,2-tetrafluoroethoxy)phenyl]methyl]]3-(isopropoxy)propyl]amino]-1,1,1-trifluoro-2-propanol; and

3-[[[3-(1,1,2,2-tetrafluoroethoxy)phenyl]methyl][3-(phenoxy)propyl]amino]-1,1,1-trifluoro-2-propanol.

Another class of CETP inhibitors that finds utility with the present invention consists of (R)-chiral halogenated 1-substituted amino-(n+1)-alkanols having the Formula XVI

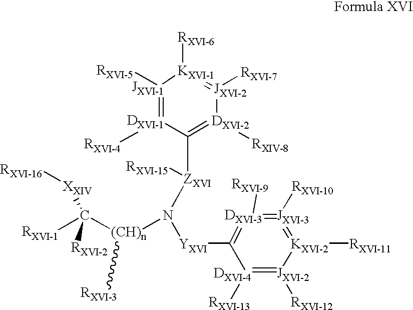

and pharmaceutically acceptable forms thereof, wherein:

n_{XVI} is an integer selected from 1 through 4;

X_{XVI} oxy;

R_{XVI-1} is selected from the group consisting of haloalkyl, haloalkenyl, haloalkoxymethyl, and haloalkenyloxymethyl with the proviso that R_{XVI-1} has a higher Cahn-Ingold-Prelog stereochemical system ranking than both R_{XVI-2} and (CHR_{XVI-3})ₙ—N(A_{XVI})Q_{XVI} wherein A_{XVI} is Formula XVI-(II) and Q is Formula XVI-(III);

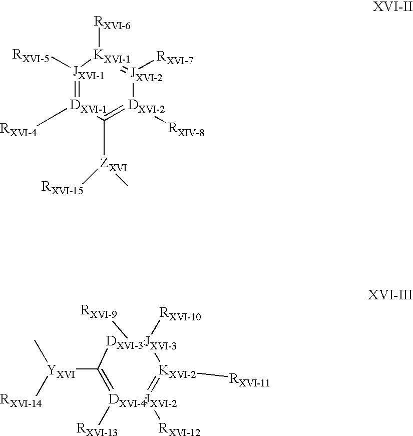

R_{XVI-16} is selected from the group consisting of hydrido, alkyl, acyl, aroyl, heteroaroyl, trialkylsilyl, and a spacer selected from the group consisting of a covalent single bond and a linear spacer moiety having a chain length of 1 to 4 atoms linked to the point of bonding of any aromatic substituent selected from the group consisting of R_{XVI-4}, R_{XVI-8}, R_{XVI-9}, and R_{XVI-13} to form a heterocyclyl ring having from 5 through 10 contiguous members;

D_{XVI-1}, D_{XVI-2}, J_{XVI-1}, J_{XVI-2} and K_{XVI-1}, are independently selected from the group consisting of C, N, O, S and covalent bond with the provisos that no more than one of D_{XVI-1}, D_{XVI-2}, J_{XVI-1}, J_{XVI-2} and K_{XVI-1}, is a covalent bond, no more than one D_{XVI-1}, D_{XVI-2}, J_{XVI-1}, J_{XVI-2} and K_{XVI-1}, is be O, no more than one of D_{XVI-1}, D_{XVI-2}, J_{XVI-1}, J_{XVI-2} and K_{XVI-1} is S, one of D_{XVI-1}, D_{XVI-2}, J_{XVI-1}, J_{XVI-2} and K_{XVI-1}, must be a covalent bond when two of D_{XVI-1}, D_{XVI-2}, J_{XVI-1}, J_{XVI-2} and K_{XVI-1}, are O and S, and no more than four of D_{XVI-1}, D_{XVI-2}, J_{XVI-1}, J_{XVI-2} and K_{XVI-1} is N;

D_{XVI-3}, D_{XVI-4}, J_{XVI-3}, J_{XVI-4} and K_{XVI-2} are independently selected from the group consisting of C, N, O, S and covalent bond with the provisos that no more than one is a covalent bond, no more than one of D_{XVI-3}, D_{XVI-4}, J_{XVI-3}, J_{XVI-4} and K_{XVI-2} is O, no more than one of D_{XVI-3}, D_{XVI-4}, J_{XVI-3}, J_{XVI}₄ and K_{XVI-2} is S, no more than two of D_{XVI-3}, D_{XVI-4}, J_{XVI-3}, J_{XVI-4} and K_{XVI-2} is O and S, one of D_{XVI-3}, D_{XVI-4}, J_{XVI-3}, J_{XVI-4} and K_{XVI-2} must be a covalent bond when two of D_{XVI-3}, D_{XVI-4}, J_{XVI-3}, J_{XVI-4} and K_{XVI-2} are O and S, and no more than four of D_{XVI-3}, D_{XVI-4}, J_{XVI-3}, J_{XVI-4} and K_{XVI-2} are N;

R_{XVI-2} is selected from the group consisting of hydrido, aryl, aralkyl, alkyl, alkenyl, alkenyloxyalkyl, haloalkyl, haloalkenyl, halocycloalkyl, haloalkoxy, haloalkoxyalkyl, haloalkenyloxyalkyl, halocycloalkoxy, halocycloalkoxyalkyl, perhaloaryl, perhaloaralkyl, perhaloaryloxyalkyl, heteroaryl, dicyanoalkyl, and carboalkoxycyanoalkyl, with the proviso that R_{XVI-2} has a lower Cahn-Ingold-Prelog system ranking than both R_{XV-1} and (CHR_{XVI-3})ₙ—N(A_{XVI})Q_{XVI};

R_{XVI-3} is selected from the group consisting of hydrido, hydroxy, cyano, aryl, aralkyl, acyl, alkoxy, alkyl, alkenyl, alkoxyalkyl, heteroaryl, alkenyloxyalkyl, haloalkyl, haloalkenyl, haloalkoxy, haloalkoxyalkyl, haloalkenyloxyalkyl, monocyanoalkyl, dicyanoalkyl, carboxamide, and carboxamidoalkyl, with the provisos that (CHR_{XVI-3})ₙ—N(A_{XVI})Q_{XVI} has a lower Cahn-Ingold-Prelog stereochemical system ranking than R_{XVI-1} and a higher Cahn-Ingold-Prelog stereochemical system ranking than R_{XVI-2};

Y_{XVI} is selected from a group consisting of a covalent single bond, (C(R_{XVI-14})₂)_{q} wherein q is an integer selected from 1 and 2 and (CH(R_{XVI-14}) )_{g}-W_{XVI}—(CH (R_{XVI-14}))ₚ wherein g and p are integers independently selected from 0 and 1;

R_{XVI-14} is selected from the group consisting of hydrido, hydroxy, cyano, hydroxyalkyl, acyl, alkoxy, alkyl, alkenyl, alkynyl, alkoxyalkyl, haloalkyl, haloalkenyl, haloalkoxy, haloalkoxyalkyl, haloalkenyloxyalkyl, monocarboalkoxyalkyl, monocyanoalkyl, dicyanoalkyl, carboalkoxycyanoalkyl, carboalkoxy, carboxamide, and carboxamidoalkyl;

Z_{XVI} is selected from a group consisting of a covalent single bond, (C(R_{XVI-15})₂)_{q}, wherein q is an integer selected from 1 and 2, and (CH(R_{XVI-15}))ⱼ-W_{XVI}—(CH(R_{XVI-15}))ₖ wherein j and k are integers independently selected from 0 and 1;

W_{XVI} is selected from the group consisting of O, C(O), C(S), C(O)N(R_{XVI-14}), C(S)N(R_{XVI-14}), (R_{XVI-14})NC(O), (R_{XVI-14})NC (S), S, S (O), S(O), S(O)₂N (R_{XVI-14}), (R_{XVI-14})NS(O)₂, and N (R_{XVI-14}) with the proviso that R_{XVI-14} is other than cyano;

R_{XVI-15} is selected, from the group consisting of hydrido, cyano, hydroxyalkyl, acyl, alkoxy, alkyl, alkenyl, alkynyl, alkoxyalkyl, haloalkyl, haloalkenyl, haloalkoxy, haloalkoxyalkyl, haloalkenyloxyalkyl, monocarboalkoxyalkyl, monocyanoalkyl, dicyanoalkyl, carboalkoxycyanoalkyl, carboalkoxy, carboxamide, and carboxamidoalkyl;

R_{XVI-4}, R_{XVI-5} R_{XVI-6}, R_{XVI-7}, R_{XVI-8}, R_{XVI-9}, R_{XVI-10}, R_{XVI-11}, R_{XVI-12}, and R_{XVI-13} are independently selected from the group consisting of hydrido, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroaralkyl, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl, amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, hydoxyheteroaralkyl, haloalkoxyalkyl, aryl, heteroaralkynyl, aryloxy, aralkoxy, aryloxyalkyl, saturated heterocyclyl, partially saturated heterocyclyl, heteroaryl, heteroaryloxy, heteroaryloxyalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl with the proviso that R_{XVI-4}, R_{XVI-5}, R_{XVI-6}, R_{XVI-7}, R_{XVI-8}, R_{XVI-9}, R_{XVI-10}, R_{XVI-11}, R_{XVI-12}, and R_{XVI-13} are each independently selected to maintain the tetravalent nature of carbon, trivalent nature of nitrogen, the divalent nature of sulfur, and the divalent nature of oxygen;

R_{XVI-4} and R_{XVI-5}, R_{XVI-5} and R_{XVI-6}, R_{XVI-6} and R_{XVI-7}, R_{XVI-7} and R_{XVI-8}, R_{XVI-9} and R_{XVI-10}, R_{XVI-10} and R_{XV-111}, R_{XVI-11} and R_{XVI-12}, R_{XVI-12} and R_{XIV-13} are independently selected to form spacer pairs wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the provisos that no more than one of the group consisting of spacer pairs R_{XVI-4} and R_{XVI-5}, R_{XVI-5} and R_{XVI-6}, R_{XVI-6} and R_{XVI-7}, and R_{XVI-7} and R_{XVI-8} is used at the same time and that no more than one of the group consisting of spacer pairs R_{XIV-9} and R_{XVI-10}, R_{XVI-10} and R_{XVI-11}, R_{XVI-11} and R_{XVI-12}, and R_{XVI-12} and R_{XVI-13} can be used at the same time;

R_{XVI-4} and R_{XVI-9} R_{XVI-4} and R_{XVI-13}, R_{XVI-8} and R_{XVI-9}, and R_{XVI-8} and R_{XVI-13} is independently selected to form a spacer pair wherein said spacer pair is taken together to form a linear moiety wherein said linear moiety forms a ring selected from the group consisting of a partially saturated heterocyclyl ring having from 5 through 8 contiguous members and a heteroaryl ring having from 5 through 6 contiguous members with the proviso that no more than one of the group consisting of spacer pairs R_{XVI-4} and R_{XVI-9}, R_{XVI-4} and R_{XVI-13}, R_{XVI-8} and R_{XVI-9}, and R_{XVI-8} and R_{XVI-13} is used at the same time.

Compounds of Formula XVI are disclosed in WO 00/18724, the entire disclosure of which is incorporated by reference.

In a preferred embodiment, the CETP inhibitor is selected from the following compounds of Formula XVI:

(2R)-3-[[3-(3-trifluoromethoxyphenoxy)phenyl][[3-(1,1,2,2-tetrafluoroethoxy)phenyl]methyl]amino]-1,1,1-trifluoro-2-propanol;

(2R)-3-[[3-(3-isopropylphenoxy)phenyl][[3-(1,1,2,2-tetrafluoroethoxy)phenyl]-methyl]amino]-1,1,1-trifluoro-2-propanol;

(2R)-3-[[3-(3-cyclopropylphenoxy)phenyl][[3-(1,1,2,2-tetrafluoroethoxy)phenyl]-methyl]amino]-1,1,1-trifluoro-2-propanol;

(2R)-3-[[3-(3-(2-furyl)phenoxy)phenyl][[3-(1,1,2,2-tetrafluoroethoxy)phenyl]-methyl]amino]-1,1,1-trifluoro-2-propanol;

(2R)-3-[[3-(2,3-dichlorophenoxy)phenyl][[3-(1,1,2,2-tetrafluoroethoxy)phenyl]-methyl]amino]-1,1,1-trifluoro-2-propanol;

(2R)-3-[[3-(4-fluorophenoxy)phenyl][[3-(1,1,2,2-tetrafluoroethoxy)phenyl]-methyl]amino]-1,1,1-trifluoro-2-propanol;

(2R)-3-[[3-(4-methylphenoxy)phenyl][[3-(1,1,2,2-tetrafluoroethoxy)phenyl]-methyl]amino]-1,1,1-trifluoro-2-propanol;

(2R)-3-[[3-(2-fluoro-5-bromophenoxy)phenyl][[3-(1,1,2,2-tetrafluoroethoxy)phenyl]-methyl]amino]-1,1,1-trifluoro-2-propanol;

(2R)-3-[[3-(4-chloro-3-ethylphenoxy)phenyl][[3-(1,1,2,2-tetrafluoroethoxy)phenyl]-methyl]amino]-1,1,1-trifluoro-2-propanol;

(2R)-3-[[3-[3-(1,1,2,2-tetrafluoroethoxy)phenoxy]phenyl] [[3-(1,1,2,2-tetrafluoro-ethoxy)phenyl]methyl]amino]-1,1,1 -trifluoro-2-propanol;

(2R)-3-[[3-[3-(pentafluoroethyl)phenoxy]phenyl][[3-(1,1,2,2-tetrafluoroethoxy)-phenyl]methyl]amino]-1,1,1-trifluoro-2-propanol;

(2R)-3-[[3-(3,5-dimethylphenoxy)phenyl][[3-(1,1,2,2-tetrafluoroethoxy)phenyl]-methyl]amino]-1,1,1-trifluoro-2-propanol;

(2R)-3-[[3-(3-ethylphenoxy)phenyl][[3-(1,1,2,2-tetrafluoroethoxy)phenyl]-methyl]amino]-1,1,1-trifluoro-2-propanol;

(2R)-3-[[3-(3-t-butylphenoxy)phenyl][[3-(1,1,2,2-tetrafluoroethoxy)phenyl]-methyl]amino]-1,1,1-trifluoro-2-propanol:

(2R)-3-[[3-(3-methylphenoxy)phenyl][[3-(1,1,2,2-tetrafluoroethoxy)phenyl]-methyl]amino]-1,1,1-trifluoro-2-propanol;

(2R)-3-[[3-(5,6,7,8-tetrahydro-2-naphthoxy)phenyl][[3-(1,1,2,2-tetrafluoro-ethoxy) phenyl]methyl]amino]-1,1,-trifluoro-2-propanol;

(2R)-3-[[3-(phenoxy)phenyl][[3-(1,1,2,2-tetrafluoroethoxy)phenyl]methyl]amino]-1,1,1-trifluoro-2-propanol;

(2R)-3-[[3-[3-(N,N-dimethylamino)phenoxy]phenyl][[3-(1,1,2,2-tetrafluoro-ethoxy)phenyl]methyl]amino]-1,1,1-trifluoro-2-propanol;

(2R)-3-[[[3-(1,1,2,2,-tetrafluoroethoxy)phenyl]methyl][3-[[3-(trifluoromethoxy)-phenyl]methoxy]phenyl]amino]-1,1,1-trifluoro-2-propanol;

(2R)-3-[[[3-(1,1,2,2-tetrafluoroethoxy)phenyl]methyl][3-[[3-(trifluoro-methyl)phenyl]methoxy]phenyl]amino]-1,1,1-trifluoro-2-propanol;

(2R)-3-[[[3-(1,1,2,2-tetrafluoroethoxy)phenyl]methyl][3-[[3,5-dimethylphenyl]-methoxy]phenyl]amino]-1,1,1-trifluoro-2-propanol;

(2R)-3-[[[3-(1,1,2,2-tetrafluoroethoxy)phenyl]methyl][3-[[3-(trifluoromethylthio)-phenyl]methoxy]phenyl]amino]-1,1,1-trifluoro-2-propanol;

(2R)-3-[[[3-(1,1,2,2-tetrafluoroethoxy)phenyl]methyl][3-[[3,5-difluorophenyl]-methoxy]phenyl]amino]-1,1,1-trifluoro-2-propanol;

(2R)-3-[[[3-(1,1,2,2-tetrafluoroethoxy)phenyl]methyl][3-[cyclohexylmethoxy]-phenyl]amino]-1,1,1-trifluoro-2-propanol;

(2R)-3-[[3-(2-difluoromethoxy-4-pyridyloxy)phenyl][[3-(1,1,2,2-tetrafluoroethoxy)-phenyl]methyl]amino]-1,1,1-trifluoro-2-propanol;

(2R)-3-[[3-(2-trifluoromethyl-4-pyridyloxy)phenyl][[3-(1,1,2,2-tetrafluoroethoxy)-phenyl]methyl]amino]-1,1,1-trifluoro-2-propanol;

(2R)-3-[[3-(3-difluoromethoxyphenoxy)phenyl][[3-(1,1,2,2-tetrafluoroethoxy)-phenyl]methyl]amino]-1,1,1-trifluoro-2-propanol;

(2R)-3-[[[3-(3-trifuoromethylthio)phenoxy]phenyl][[3-(1,1,2,2-tetrafluoroethoxy)-phenyl]methyl]amino]-1,1,1-trifluoro-2-propanol;

(2R)-3-[[3-(4-chloro-3-trifluoromethylphenoxy)phenyl][[3-(1,1,2,2-tetrafluoroethoxy)-phenyl]methyl]amino]-1,1,1-trifluoro-2-propanol;

(2R)-3-[[3-(3-trifluoromethoxyphenoxy)phenyl][[3-(pentafluoroethyl)phenyl]-methyl]amino]-1,1,1,-trifluoro-2-propanol;

(2R)-3-[[3-(3-isopropylphenoxy)phenyl][[3-(pentafluoroethyl)phenyl]methyl]-amino]-1,1,1-trifluoro-2-propanol;

(2R)-3-[[3-(3-cyclopropylphenoxy)phenyl][[3-(pentafluoroethyl)phenyl]methyl]-amino]-1,1,1-trifluoro-2-propanol;

(2R)-3-[[3-(3-(2-furyl)phenoxy)phenyl][[3-(pentafluoroethyl)phenyl]methyl]-amino]-1,1,1-trifluoro-2-propanol;

(2R)-3-[[3-(2,3-dichlorophenoxy)phenyl][[3-(pentafluoroethyl)phenyl]methyl]-amino]-1,1,1-trifluoro-2-propanol;

(2R)-3-[[3-(4-fluorophenoxy)phenyl][[3-(pentafluoroethyl)phenyl]methyl]amino]-1,1,1-trifluoro-2-propanol;

(2R)-3-[[3-(4-methylphenoxy)phenyl][[3-(pentafluoroethyl)phenyl]methyl]amino]-1,1,1-trifluoro-2-propanol;

(2R)-3-[[3-(2-fluoro-5-bromophenoxy)phenyl][[3-(pentafluoroethyl)phenyl]methyl]-amino]-1,1,1-trifluoro-2-propanol;

(2R)-3-[[3-(4-chloro-3-ethylphenoxy)phenyl][[3-(pentafluoroethyl)phenyl]methyl]-amino]-1,1,1-trifluoro-2-propanol;

(2R)-3-[[3-[3-(1,1,2,2-tetrafluoroethoxy)phenoxy]phenyl][[3-(pentafluoroethyl)-phenyl]methyl]amino]-1,1,1-trifluoro-2-propanol;

(2R)-3-[[3-[3-(pentafluoroethyl)phenoxy]phenyl][[3-(pentafluoroethyl)phenyl]-methyl]amino]-1,1,1-trifluoro-2-propanol;

(2R)-3-[[3-(3,5-dimethylphenoxy)phenyl][[3-(pentafluoroethyl) phenyl]methyl]-amino]-1,1,1-trifluoro-2-propanol;

(2R)-3-[[3-(3-ethylphenoxy)phenyl][[3-(pentafluoroethyl) phenyl]methyl]amino]-1,1,1-trifluoro-2-propanol;

(2R)-3-[[3-(3-t-butylphenoxy)phenyl][[3-(pentafluoroethyl) phenyl]methyl]amino]-1,1,1-trifluoro-2-propanol;

(2R)-3-[[3-(3-methylphenoxy)phenyl][[3-(pentafluoroethyl) phenyl]methyl]amino]-1,1,1-trifluoro-2-propanol;

(2R)-3-[[3-(5,6,7,8-tetrahydro-2-naphthoxy)phenyl][[3-(pentafluoroethyl)phenyl]-methyl]amino]-1,1,1-trifluoro-2-propanol;

(2R)-3-[[3-(phenoxy)phenyl][[3(pentafluoroethyl) phenyl]methyl]amino]-1,1,1-trifluoro-2-propanol;

(2R)-3-[[3-[3-(N,N-dimethylamino)phenoxy]phenyl][[3-(pentafluoroethyl)phenyl]-methyl]amino]-1,1,1-trifluoro-2-propanol;

(2R)-3-[[[3-(pentafluoroethyl)phenyl]methyl][3-[[3-(trifluoromethoxy)phenyl]-methoxy]phenyl]amino]-1,1,1-trifluoro-2-propanol;

(2R)-3-[[[3-(pentafluoroethyl)phenyl]methyl][3-[[3-(trifluoromethyl)-phenyl]-methoxy]phenyl]amino]-1,1,1-trifluoro-2-propanol;

(2R)-3-[[[3-(pentafluoroethyl)phenyl]methyl][3-[[3,5-dimethylphenyl]methoxy]-phenyl]amino]-1,1,1-trifluoro-2-propanol;

(2R)-3-[[[3-(pentafluoroethyl)phenyl]methyl][3-[[3-(trifluoromethylthio)phenyl]-methoxy]phenyl]amino]-1,1,1-trifluoro-2-propanol;

(2R)-3-[[[3-(pentafluoroethyl)phenyl]methyl][3-[[3,5-difluorophenyl]methoxy]-phenyl]amino]-1,1,1-trifluoro-2-propanol;

(2R)-3-[[[3-(pentafluoroethyl)phenyl]methyl][3-[cyclohexylmethoxy]phenyl]-amino]-1,1,1-trifluoro-2-propanol;

(2R)-3-[[3-(2-difluoromethoxy-4-pyridyloxy)phenyl][[3-(pentafluoroethyl)phenyl]-methyl]amino]-1,1,1-trifluoro-2-propanol;

(2R)-3-[[3-(2-trifluoromethyl-4-pyridyloxy)phenyl][[3-(pentafluoroethyl)phenyl]-methyl]amino]-1,1,1-trifluoro-2-propanol;

(2R)-3-[[3-(3-difluoromethoxyphenoxy)phenyl][[3-(pentafluoroethyl)phenyl]-methyl]amino]-1,1,1-trifluoro-2-propanol;

(2R)-3-[[[3-(3-trifluoromethylthio)phenoxy]phenyl][[3-(pentafluoroethyl)phenyl]-methyl]amino]-1,1,1-trifluoro-2-propanol;

(2R)-3-[[3-(4-chloro-3-trifluoromethylphenoxy)phenyl][[3-(pentafluoroethyl)-phenyl]methyl]amino]-1,1,1-trifluoro-2-propanol;

(2R)-3-[[3-(3-trifluoromethoxyphenoxy)phenyl][[3-(heptafluoropropyl)phenyl]-methyl]amino]-1,1,1-trifluoro-2-propanol;

(2R)-3-[[3-(3-isopropylphenoxy)phenyl][[3-(heptafluoropropyl)phenyl]methyl]-amino]-1,1,1-trifluoro-2-propanol;

(2R)-3-[[3-(3-cyclopropylphenoxy)phenyl][[3-(heptafluoropropyl)phenyl]methyl]-amino]-1,1,1-trifluoro-2-propanol;

(2R)-3-[[3-(3-(2-furyl)phenoxy)phenyl][[3-(heptafluoropropyl) phenyl]methyl]-amino]-1,1,1-trifluoro-2-propanol;

(2R)-3-[[3-(2,3-dichlorophenoxy)phenyl][[3-(heptafluoropropyl) phenyl]methyl]-amino]-1,1,1-trifluoro-2-propanol;

(2R)-3-[[3-(4-fluorophenoxy)phenyl][[3-(heptafluoropropyl) phenyl]methyl]amino]-1,1,1-trifluoro-2-propanol;

(2R)-3-[[3-(4-methylphenoxy)phenyl][[3-(heptafluoropropyl) phenyl]methyl]amino]-1,1,1,-trifluoro-2-propanol;

(2R)-3-[[3-(2-fluoro-5-bromophenoxy)phenyl][[3-(heptafluoropropyl)phenyl]-methyl]amino]-1,1,1-trifluoro-2-propanol;

(2R)-3-[[3-(4-chloro-3-ethylphenoxy)phenyl][[3-(heptafluoropropyl)phenyl]methyl]-amino]-1,1,1-trifluoro-2-propanol;

(2R)-3-[[3-[3-(1,1,2,2-tetrafluoroethoxy)phenoxy]phenyl][[3-(heptafluoropropyl)-phenyl]methyl]amino]-1,1,1-trifluoro-2-propanol;

(2R)-3-[[3-[3-(pentafluoroethyl)phenoxy]phenyl][[3-(heptafluoropropyl)phenyl]-methyl]amino]-1,1,1-trifluoro-2-propanol;

(2R)-3-[[3-(3,5-dimethylphenoxy)phenyl][[3-(heptafluoropropyl) phenyl]methyl]-amino]-1,1,1-trifluoro-2-propanol;

(2R)-3-[[3-(3-ethylphenoxy)phenyl][[3-(heptafluoropropyl) phenyl]methyl]amino]-1,1,1-trifluoro-2-propanol;

(2R)-3-[[3-(3-t-butylphenoxy)phenyl][[3-(heptafluoropropyl) phenyl]methyl]amino]-1,1,1-trifluoro-2-propanol;

(2R)-3-[[3-(3-methylphenoxy)phenyl][[3-(heptafluoropropyl) phenyl]methyl]amino]-1,1,1-trifluoro-2-propanol;

(2R)-3-[[3-(5,6,7,8-tetrahydro-2-naphthoxy)phenyl][[3-(heptafluoropropyl)phenyl]-methyl]amino]-1,1,1-trifluoro-2-propanol;

(2R)-3-[[3-(phenoxy)phenyl][[3-(heptafluoropropyl) phenyl]methyl]amino]-1,1,1-trifluoro-2-propanol;

(2R)-3-[[3-[3-(N,N-dimethylamino)phenoxy]phenyl][[3-(heptafluoropropyl)phenyl]-methyl]amino]-1,1,1-trifluoro-2-propanol;

(2R)-3-[[[3-(heptafluoropropyl)phenyl]methyl][3-[[3-(trifluoromethoxy)phenyl]-methoxy]phenyl]amino]-1,1,1-trifluoro-2-propanol;

(2R)-3-[[[3-(heptafluoropropyl)phenyl]methyl][3-[[3-(trifluoromethyl)phenyl]-methoxy]phenyl]amino]-1,1,1-trifluoro-2-propanol;

(2R)-3-[[[3-(heptafluoropropyl)phenyl]methyl][3-[[3,5-dimethylphenyl]methoxy]-phenyl]amino]-1,1,1-trifluoro-2-propanol;

(2R)-3-[[[3-(heptafluoropropyl)phenyl]methyl][3-[[3-(trifluoromethylthio)phenyl]-methoxy]phenyl]amino]-1,1,1-trifluoro-2-propanol;

(2R)-3-[[[3-(heptafluoropropyl)phenyl]methyl][3-[[3,5-difluorophenyl]methoxy]-phenyl]amino]-1,1,1-trifluoro-2-propanol;

(2R)-3-[[[3-(heptafluoropropyl)phenyl]methyl][3-[cyclohexylmethoxy]phenyl]-amino]-1,1,1-trifluoro-2-propanol;

(2R)-3-[[3-(2-difluoromethoxy-4-pyridyloxy)phenyl][[3-(heptafluoropropyl)phenyl]-methyl]amino]-1,1,1-trifluoro-2-propanol;

(2R)-3-[[3-(2-trifluoromethyl-4-pyridyloxy)phenyl][[3-(heptafluoropropyl)phenyl]-methyl]amino]-1,1,1-trifluoro-2-propanol;

(2R)-3-[[3-(3-difluoromethoxyphenoxy)phenyl][[3-(heptafluoropropyl)phenyl]-methyl]amino]-1,1,1-trifluoro-2-propanol;

(2R)-3-[[[3-(3-trifluoromethylthio)phenoxy]phenyl][[3-(heptafluoropropyl)phenyl]-methyl]amino]-1,1,1-trifluoro-2-propanol;

(2R)-3-[[3-(4-chloro-3-trifluoromethylphenoxy)phenyl][[3-(heptafluoropropyl)-phenyl]methyl]amino]-1,1,1-trifluoro-2-propanol;

(2R)-3-[[3-(3-trifluoromethoxyphenoxy)phenyl][[2-fluoro-5-(trifluoromethyl)-phenyl]methyl]amino]-1,1,1-trifluoro-2-propanol;

(2R)-3-[[3-(3-isopropylphenoxy)phenyl][[2-fluoro-5-(trifluoromethyl )phenyl]-methyl]amino]-1,1,1-trifluoro-2-propanol;

(2R)-3-[[3-(3-cyclopropylphenoxy)phenyl][[2-fluoro-5-(trifluoromethyl)phenyl]-methyl]amino]-1,1,1-trifluoro-2-propanol;

(2R)-3-[[3-(3-(2-furyl)phenoxy)phenyl][[2-fluoro-5-(trifluoromethyl)phenyl]-methyl]amino]-1,1,1-trifluoro-2-propanol;

(2R)-3-[[3-(2,3-dichlorophenoxy)phenyl][[2-fluoro-5-(trifluoromethyl)phenyl]-methyl]amino]-1,1,1-trifluoro-2-propanol;

(2R)-3-[[3-(4-fluorophenoxy)phenyl][[2-fluoro-5-(trifluoromethyl)phenyl]-methyl]amino]-1,1,1-trifluoro-3-propanol;

(2R)-3-[[3-(4-methylphenoxy)phenyl][[2-fluoro-5-(trifluoromethyl)phenyl]-methyl]amino]-1,1,1-trifluoro-2-propanol;

(2R)-3-[[3-(2-fluoro-5-bromophenoxy)phenyl][[2-fluoro-5-(trifluoromethyl)-phenyl]methyl]amino]-1,1,1-trifluoro-2-propanol;

(2R)-3-[[3-(4-chloro-3-ethylphenoxy)phenyl][[2-fluoro-5-(trifluoromethyl)-phenyl]methyl]amino]-1,1,1-trifluoro-2-propanol;

(2R)-3-[[3-[3-(1,1,2,2-tetrafluoroethoxy)phenoxy]phenyl][[2-fluoro-5-(trifluoro-methyl)phenyl]methyl]amino]-1,1,1-trifluoro-2-propanol;

(2R)-3-[[3-[3-(pentafluoroethyl)phenoxy]phenyl][[2-fluoro-5-(trifluoromethyl)-phenyl]methyl]amino]-1,1,1-trifluoro-2-propanol;

(2R)-3-[[3-(3,5-dimethylphenoxy)phenyl][[2-fluoro-5-(trifluoromethyl)phenyl]-methyl]amino]-1,1,1-trifluoro-2-propanol;

(2R)-3-[[3-(3-ethylphenoxy)phenyl][[2-fluoro-5-(trifluoromethyl)phenyl]methyl]-amino]-1,1,1-trifluoro-2-propanol;

(2R)-3-[[3-(3-t-butylphenoxy)phenyl][[2-fluoro-5-(trifluoromethyl)phenyl]methyl]-amino]-1,1,1-trifluoro-2-propanol;

(2R)-3-[[3-(3-methylphenoxy)phenyl][[2-fluoro-5-(trifluoromethyl)phenyl]methyl]-amino]-1,1,1-trifluoro-2-propanol;

(2R)-3-[[3-(5,6,7,8-tetrahydro-2-naphthoxy)phenyl][[2-fluoro-5-(trifluoromethyl)-phenyl]methyl]amino]-1,1,1-trifluoro-2-propanol;

(2R)-3-[[3-(phenoxy)phenyl][[2-fluoro-5-(trifluoromethyl) phenyl]methyl]amino]-1,1,1-trifluoro-2-propanol;

(2R)-3-[[3-[3-(N,N-dimethylamino,phenoxy]phenyl][[2-fluoro-5-(trifluoromethyl)-phenyl]methyl]amino]-1,1,1-trifluoro-2-propanol;

(2R)-3-[[[2-fluoro-5-(trifluoromethyl)phenyl]methyl][3-[[3-(trifluoromethoxy)-phenyl]methoxy]phenyl]amino]-1,1,1-trifluoro-3-propanol;

(2R)-3-[[[2-fluoro-5-(trifluoromethyl)phenyl]methyl][3-[[3-(trifluoromethyl)-phenyl]methoxy]phenyl]amino]-1,1,1-trifluoro-2-propanol;

(2R)-3-[[[2-fluoro-5-(trifluoromethyl)phenyl]methyl][3-[[3,5-dimethylphenyl]-methoxy]phenyl]amino]-1,1,1-trifluoro-2-propanol;

(2R)-3-[[[2-fluoro-5-(trifluoromethyl)phenyl]methyl][3-[[3-(trifluoromethylthio)-phenyl]methoxy]phenyl]amino]-1,1,1-trifluoro-2-propanol;

(2R)-3-[[[2-fluoro-5-(trifluoromethyl)phenyl]methyl][3-[[3,5-difluorophenyl]-methoxy]phenyl]amino]-1,1,1-trifluoro-2-propanol;

(2R)-3-[[[2-fluoro-5-(trifluoromethyl)phenyl]methyl][3-[cyclohexylmethoxyl-phenyl]amino]-1,1,1-trifluoro-2-propanol;

(2R)-3-[[3-(2-difluoromethoxy-4-pyridyloxy)phenyl][[2-fluoro-5-(trifluoromethyl)-phenyl]methyl]amino]-1,1,1-trifluoro-2-propanol;

(2R)-3-[[3-(2-trifluoromethyl-4-pyridyloxy)phenyl][[2-fluoro-5-(trifluoromethyl)-phenyl]methyl]amino]-1,1,1-trifluoro-2-propanol;

(2R)-3-[[3-(3-difluoromethoxyphenoxy)phenyl][[2-fluoro-5-(trifluoromethyl)-phenyl]methyl]amino]-1,1,1-trifluoro-2-propanol;

(2R)-3-[[[3-(3-trifluoromethylthio)phenoxy]phenyl][[2-fluoro-5-(trifluoromethyl)-phenyl]methyl]amino]-1,1,1-trifluoro-2-propanol;

(2R)-3-[[3-(4-chloro-3-trifluoromethylphenoxy)phenyl][[2-fluoro-5-(trifluoro-methyl)phenyl]methyl]amino]-1,1,1-trifluoro-2-propanol;

(2R)-3-[[3-(3-trifluoromethoxyphenoxy)phenyl][[2-fluoro-4-(trifluoromethyl)-phenyl]methyl]amino]-1,1,1-trifluoro-2-propanol;

(2R)-3-[[3-(3-isopropylphenoxy)phenyl][[2-fluoro-4-(trifluoromethyl)phenyl]-methyl]amino]-1,1,1-trifluoro-2-propanol;

(2R)-3-[[3-(3-cyclopropylphenoxy)phenyl][[2-flouro-4-(trifluoromethyl)phenyl]-methyl]amino]-1,1,1-trifluoro-2-propanol;

(2R)-3-[[3-(3-(2-furyl)phenoxy)phenyl][[2-fluoro-4-(trifluoromethyl)phenyl]-methyl]amino]-1,1,1-trifluoro-2-propanol;

(2R)-3-[[3-(2,3-dichlorophenoxy)phenyl][[2-fluoro-4-(trifluoromethyl)phenyl]-methyl]amino]-1,1,1-trifluoro-2-propanol;

(2R)-3-[[3-(4-fluorophenoxy)phenyl][[2-fluoro-4-(trifluoromethyl)phenyl]-methyl]amino]-1,1,1-trifluoro-2-propanol;

(2R)-3-[[3-(4-methylphenoxy)phenyl][[2-fluoro-4-(trifluoromethyl)phenyl]-methyl]amino]-1,1,1-trifluoro-2-propanol;

(2R)-3-[[3-(2-fluoro-5-bromophenoxy)phenyl][[2-fluoro-4-(trifluoromethyl)-phenyl]methyl]amino]-1,1,1-trifluoro-2-propanol;

(2R)-3-[[3-(4-chloro-3-ethylphenoxy)phenyl][[2-fluoro-4-(trifluoromethyl)-phenyl]methyl]amino]-1,1,1-trifluoro-2-propanol;

(2R)-3-[[3-[3-(1,1,2,2-tetrafluoroethoxy)phenoxy]phenyl][[2-fluoro-4-(trifluoromethyl)phenyl]methyl]amino]-1,1,1-trifluoro-2-propanol;

(2R)-3-[[3-[3-(pentafluoroethyl)phenoxy]phenyl][[2-fluoro-4-(trifluoromethyl)-phenyl]methyl]amino]-1,1,1-trifluoro-2-propanol;

(2R)-3-[[3-(3,5-dimethylphenoxy)phenyl][[2-fluoro-4-(trifluoromethyl)phenyl]-methyl]aminol-1,1,1-trifluoro-2-propanol;

(2R)-3-[[3-(3-ethylphenoxy)phenyl][[2-fluoro-4-(trifluoromethyl)phenyl]methyl]-amino]-1,1,1-trifluoro-2-propanol;

(2R)-3-[[3-(3-t-butylphenoxy)phenyl][[2-fluoro-4-(trifluoromethyl)phenyl]methyl]-amino]-1,1,1-trifluoro-2-propanol;

(2R)-3-[[3-(3-methylphenoxy)phenyl][[2-fluoro-4-(trifluoromethyl)phenyl]methyl]-amino]-1,1,1-trifluoro-2-propanol;

(2R)-3-[[3-(5,6,7,8-tetrahydro-2-naphthoxy)phenyl][[2-fluoro-4-(trifluoromethyl)-phenyl]methyl]amino]-1,1,1-trifluoro-2-propanol;

(2R)-3-[[3-(phenoxy)phenyl][[2-fluoro-4-(trifluoromethyl) phenyl]methyl]amino]-1,1,1-trifluoro-2-propanol;

(2R)-3-[[3-[3-(N,N-dimethylamino)phenoxy]phenyl][[2-fluoro-4-(trifluoromethyl)-phenyl]methyl]amino]-1,1,1-trifluoro-2-propanol;

(2R)-3-[[[2-fluoro-4-(trifluoromethyl)phenyl]methyl][3-[[3-(trifluoromethoxy)phenyl]methoxy]phenyl]amino]-1,1,1-trifluoro-2-propanol;

(3R)-3-[[[2-fluoro-4-(trifluoromethyl)phenyl]methyl][3-[[3-(trifluoromethyl)phenyl]methoxy]phenyl]amino]-1,1,1-trifluoro-2-propanol;

(2R)-3-[[[2-fluoro-4-(trifluoromethyl)phenyl]methyl][3-[[3,5-dimethylphenyl]-methoxy]phenyl]amino]-1,1,1-trifluoro-2-propanol;

(2R)-3-[[[2-fluoro-4-(trifluoromethyl)phenyl]methyl][3-[[3-(trifluoromethylthio)-phenyl]methoxy]phenyl]amino]-1,1,1-trifluoro-2-propanol;

(2R)-3-[[[2-fluoro-4-(trifluoromethyl)phenyl]methyl][3-[[3,5-difluorophenyl]-methoxy]phenyl]amino]-1,1,1-trifluoro-2-propanol;

(2R)-3-[[[2-fluoro-4-(trifluoromethyl)phenyl]methyl][3-[cyclohexylmethoxy]-phenyl]amino]-1,1,1-trifluoro-2-propanol;

(2R)-3-[[3-(2-difluoromethoxy-4-pyridyloxy)phenyl][[2-fluoro-4-(trifluoromethyl)-phenyl]methyl]amino]-1,1,1-trifluoro-2-propanol;

(2R)-3-[[3-(2-trifluoromethyl-4-pyridyloxy)phenyl][[2-fluoro-4-(trifluoromethyl)-phenyl]methyl]amino]-1,1,1-trifluoro-2-propanol;

(2R)-3-[[3-(3-difluoromethoxyphenoxy)phenyl][[2-fluoro-4-(trifluoromethyl)-phenyl]methyl]amino]-1,1,1-trifluoro-2-propanol;

(2R)-3-[[[3-(3-trifluoromethylthio)phenoxy]phenyl][[2-fluoro-4-(trifluoromethyl)-phenyl]methyl]amino]-1,1,1-trifluoro-2-propanol; and

(2R)-3-[[3-(4-chloro-3-trifluoromethylphenoxy)phenyl][[2-fluoro-4-(trifluoromethyl)phenyl]methyl]amino]-1,1,1-trifluoro-2-propanol.

Another class of CETP inhibitors that finds utility with the present invention consists of quinolines of Formula XVII

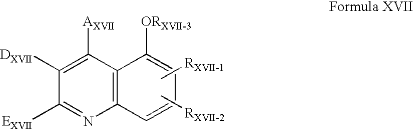

and pharmaceutically acceptable forms thereof, wherein:

A_{XVII} denotes an aryl containing 6 to 10 carbon atoms, which is optionally substituted with up to five identical or different substituents in the form of a halogen, nitro, hydroxyl, trifluoromethyl, trifluoromethoxy or a straight-chain or branched alkyl, acyl, hydroxyalkyl or alkoxy containing up to 7 carbon atoms each, or in the form of a group according to the formula —NR_{XVII-4}R_{XVII-5}, wherein

R_{XVII-4} and R_{XVII-5} are identical or different and denote a hydrogen, phenyl or a straight-chain or branched alkyl containing up to 6 carbon atoms,

D_{XVII} denotes an aryl containing 6 to 10 carbon atoms, which is optionally substituted with a phenyl, nitro, halogen, trifluoromethyl or trifluoromethoxy, or a radical according to the formula

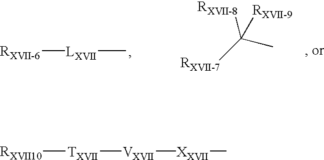

wherein

R_{XVII-6} R_{XVII-7} R_{XVII-10} denote, independently from one another, a cycloalkyl containing 3 to 6 carbon atoms, or an aryl containing 6 to 10 carbon atom or a 5- to 7-membered, optionally benzo-condensed, saturated or unsaturated, mono-, bi- or tricyclic heterocycle containing up to 4 heteroatoms from the series of S, N and/or O, wherein the rings are optionally substituted, in the case of the nitrogen-containing rings also via the N function, with up to five identical or different substituents in the form of a halogen, trifluoromethyl, nitro, hydroxyl, cyano, carboxyl, trifluoromethoxy, a straight-chain or branched acyl, alkyl, alkylthio, alkylalkoxy, alkoxy or alkoxycarbonyl containing up to 6 carbon atoms each, an aryl or trifluoromethyl-substituted aryl containing 6 to 10 carbon atoms each, or an optionally benzo-condensed, aromatic 5- to 7-membered heterocycle containing up to 3 heteoatoms from the series of S, N and/or O, and/or in the form of a group according to the formula

—OR_{XVII-11}, —SR_{XVII-12}, —SO₂R_{XVII-13}, or —NR_{XVII-14}R_{XVII-15};

R_{XVII-11}, R_{XVII-12} and R_{XVII-13} denote, independently from one another, an aryl containing 6 to 10 carbon atoms, which is in turn substituted with up to two identical or different substituents in the form of a phenyl, halogen or a straight-chain or branched alkyl containing up to 6 carbon atoms,

R_{XVII-14} and R_{XVII-15} are identical or different and have the meaning of R_{XVII-4} and R_{XVII-5} given above, or

R_{XVII-6} and/or R_{XVII-7} denote a radical according to the formula

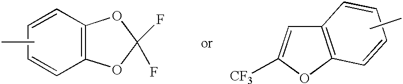

R_{XVII-8} denotes a hydrogen or halogen, and

R_{XVII-9} denotes a hydrogen, halogen, azido, trifluoromethyl, hydroxyl, trifluoromethoxy, a straight-chain or branched alkoxy or alkyl containing up to 6 carbon atoms each, or a radical according to the formula NR_{XVII-16}R_{XVII-17};

R_{XVII-16} and R_{XVII-17} are identical or different and have the meaning of R_{XVII-4} and R_{XVII-5} above; or

R_{XVII-8} and R_{XVII-9} together form a radical according to the formula

═O or ═NR_{XVII-18};

R_{XVII-18} denotes a hydrogen or a straight-chain or branched alkyl, alkoxy or acyl containing up to 6 carbon atoms each;

L_{XVII} denotes a straight-chain or branched alkylene or alkenylene chain containing up to 8 carbon atoms each, which are optionally substituted with up to two hydroxyl groups;

T_{XVII} and X_{XVII} are identical or different and denote a straight-chain or branched alkylene chain containing up to 8 carbon atoms; or

T_{XVII} and X_{XVII} denotes a bond;

V_{XVII} denotes an oxygen or sulfur atom or —NR_{XVII-19};

R_{XVII-19} denotes a hydrogen or a straight-chain or branched alkyl containing up to 6 carbon atoms or a phenyl;

E_{XVII} denotes a cycloalkyl containing 3 to 8 carbon atoms, or a straight-chain or branched alkyl containing up to 8 carbon atoms, which is optionally substituted with a cycloalkyl containing 3 to 8 carbon atoms or a hydroxyl, or a phenyl, which is optionally substituted with a halogen or trifluoromethyl;

R_{XVII-1} and R_{XVII-2} are identical or different and denote a cycloalkyl containing 3 to 8 carbon atoms, hydrogen, nitro, halogen, trifluoromethyl, trifluoromethoxy, carboxy, hydroxy, cyano, a straight-chain or branched acyl, alkoxycarbonyl or alkoxy with up to 6 carbon atoms, or NR_{XVII-20}R_{XVII-21};

R_{XVII-20} and R_{XVII-21} are identical or different and denote hydrogen, phenyl, or a straight-chain or branched alkyl with up to 6 carbon atoms; and or

R_{XVII-1} and/or R_{XVII-2} are straight-chain or branched alkyl with up to 6 carbon atoms, optionally substituted with halogen, trifluoromethoxy, hydroxy, or a straight-chain or branched alkoxy with up to 4 carbon atoms, aryl containing 6-10 carbon atoms optionally substituted with up to five of the same or different substituents selected from halogen, cyano, hydroxy, trifluoromethyl, trifluoromethoxy, nitro, straight-chain or branched alkyl, acyl, hydroxyalkyl, alkoxy with up to 7 carbon atoms and NR_{XVII-22}R_{XVII-23};

R_{XVII-22} and R_{XVII-23} are identical or different and denote hydrogen, phenyl or a straight-chain or branched akyl up to 6 carbon atoms; and/or

R_{XVII-1} and R_{XVII-2} taken together form a straight-chain or branched alkene or alkane with up to 6 carbon atoms optionally substituted with halogen, trifluoromethyl, hydroxy or straight-chain or branched alkoxy with up to 5 carbon atoms;

R_{XVII-3} denotes hydrogen, a straight-chain or branched acyl with up to 20 carbon atoms, a benzoyl optionally substituted with halogen, trifluoromethyl, nitro or trifluoromethoxy, a straight-chained or branched fluoroacyl with up to 8 carbon atoms and 7 fluoro atoms, a cycloalkyl with 3 to 7 carbon atoms, a straight chained or branched alkyl with up to 8 carbon atoms optionally substituted with hydroxyl, a straight-chained or branched alkoxy with up to 6 carbon atoms optionally substituted with phenyl which may in turn be substituted with halogen, nitro, trifluoromethyl, trifluoromethoxy, or phenyl or a tetrazol substitued phenyl, and/or an alkyl that is optionally substituted with a group according to the formula

—OR_{XVII-24};

R_{XVII-24} is a straight-chained or branched acyl with up to 4 carbon atoms or benzyl.

Compounds of Formula XVII are disclosed in WO 98/39299, the entire disclosure is incorporated by reference.

Another class of CETP inhibitors that finds utility with the present invention consists of 4-Phenyltetrahydroquinolines of Formula XVIII

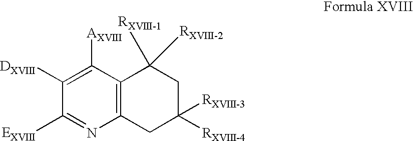

N oxides thereof, and pharmaceutically acceptable forms thereof, wherein:

A_{XVIII} denotes a phenyl optionally substituted with up to two identical or different substituents in the form of halogen, trifluoromethyl or a straight-chain or branched alkyl or alkoxy containing up to three carbon atoms;

D_{XVIII} denotes the formula

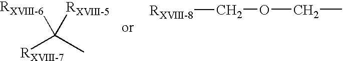

R_{XVIII-5} and R_{XVIII-6} are taken together to form ═O; or

R_{XVIII-5} denotes hydrogen and R_{XVIII-6} denotes halogen or hydrogen; or

R_{XVIII-5} and R_{XVIII-6} denote hydrogen;

R_{XVIII-7} and R_{XVIII-8} are identical or different and denote phenyl, naphthyl, benzothiazolyl, quinolinyl, pyrimidyl or pyridyl with up to four identical or different substituents in the form of halogen, trifluoromethyl, nitro, cyano, trifluoromethoxy, —SO₂—CH₃ or NR_{XVIII-9}R_{XVIII-10};

R_{XVIII-9} and R_{XVIII-10} are identical or different and denote hydrogen or a straight-chained or branched alkyl of up to three carbon atoms;

E_{XVIII} denotes a cycloalkyl of from three to six carbon atoms or a straight-chained or branched alkyl of up to eight carbon atoms;

R_{XVIII-1} denotes hydroxy;

R_{XVIII-2} denotes hydrogen or methyl;

R_{XVIII-3} and R_{XVIII-4} are identical or different and denote straight-chained or branched alkyl of up to three carbon atoms; or

R_{XVIII-3} and R_{XVIII-4} taken together form an alkenylene made up of between two and four carbon atoms.

Compounds of Formula XVIII are disclosed in WO 99/15504, the entire disclosure of which is incorporated by reference.

### Amphiphilic Polymers

Amphiphilic polymers suitable for use in the present invention should be pharmaceutically acceptable, and have at least some solubility in aqueous solution at physiologically relevant pHs (e.g., 1-8). The polymer may be neutral (non-ionizable) or ionizable, and should have an aqueous-solubility of at least 0.1 mg/mL over at least a portion of the pH range of 1-8. Amphiphilic polymers suitable for use with the present invention may be cellulosic or non-cellulosic. The polymers may be neutral or ionizable in aqueous solution. Of these, those with the greatest degree of amphiphilicity are preferred. Many such highly amphiphilic polymers are ionizable cellulosic polymers.

By “amphiphilic” is meant that the polymer has hydrophobic and hydrophilic portions. The hydrophobic portion may comprise groups such as aliphatic or aromatic hydrocarbon groups. The hydrophilic portion may comprise either ionizable or non-ionizable groups that are capable of polar or hydrogen-bond donor or acceptor interactions with water or other molecules. Examples of such groups are hydroxyls, carboxylic acids, esters, ethers, amines or amides.

Amphiphilic, and preferably ionizable, polymers are preferred because it is believed that such polymers may tend to have simultaneously both relatively strong interactions with the drug and relatively strong interactions with water in aqueous solutions. These interactions may promote the formation of the various types of polymer/drug assemblies in the aqueous use environment as described previously. In addition, the repulsion of the like charges of the ionized groups of such polymers may serve to limit the size of the polymer/drug assemblies to the nanometer or submicron scale. For example, while not wishing to be bound by a particular theory, such polymer/drug assemblies may comprise hydrophobic drug clusters surrounded by the polymer with the polymer's hydrophobic regions turned inward towards the drug and the hydrophilic regions of the polymer turned outward toward the aqueous environment. Alternatively, depending on the specific chemical nature of the drug, the ionized functional groups of the polymer may associate, for example, via ion pairing or hydrogen bonds, with ionic or polar groups of the drug. In the case of ionizable polymers, the hydrophilic regions of the polymer would include the ionized functional groups. Such polymer/drug assemblies in solution may well resemble polymeric micellar-like structures. In any case, regardless of the mechanism of action, the inventors have observed that such amphiphilic polymers, particularly ionizable cellulosic polymers, have been shown to form polymer/drug assemblies in aqueous solution and result in high levels of free drug and total dissolved drug relative to control compositions free from such polymers.

One class of amphiphilic polymers suitable for use with the present invention comprises neutral (non-ionizable) non-cellulosic polymers. Exemplary neutral non-cellulosic polymers include: vinyl polymers and copolymers having at least one substituent selected from the group comprising hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyethylene glycol polypropylene glycol copolymers; polyvinyl pyrrolidone (also known as povidone or PVP; polyethylene polyvinyl alcohol copolymers; and polyoxyethylene-polyoxypropylene block copolymers.

Vinyl homopolymers, those with only one type of vinyl repeat unit, may be somewhat amphiphilic. For example, polyvinyl pyrrolidone is somewhat amphiphilic in that the pendant cyclic amido groups are relatively hydrophilic and the remainder of the polymer, including the backbone itself, is relatively hydrophobic, consisting of methylene groups.

Generally, copolymers of a relatively hydrophilic repeat unit and a relatively hydrophobic repeat unit will be more amphiphilic than most homopolymers. Exemplary amphiphilic copolymers are polyvinyl alcohol/polyvinyl acetate copolymers and polyethylene polyvinyl alcohol copolymers.

A preferred class of neutral non-cellulosic polymers are comprised of vinyl copolymers of at least one hydrophilic, hydroxyl-containing repeat unit and at least one hydrophobic, alkyl- or aryl-containing repeat unit. Such neutral vinyl copolymers are termed “amphiphilic hydroxyl-functional vinyl copolymers.” Amphiphilic hydroxyl-functional vinyl copolymers are believed to provide high concentration enhancements due to the amphiphilicity of these copolymers which provide both sufficient hydrophobic groups to interact with the hydrophobic, low-solubility drugs and also sufficient hydrophilic groups to have sufficient aqueous solubility for good dissolution. The copolymeric structure of the amphiphilic hydroxyl-functional vinyl copolymers also allows their hydrophilicity and hydrophobicity to be adjusted to maximize performance with a specific low-solubility drug.

The preferred copolymers have the general structure:

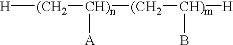

where A and B represent “hydrophilic, hydroxyl-containing” and “hydrophobic” substituents, respectively, and n and m represent the average number of hydrophilic vinyl repeat units and average number of hydrophobic vinyl repeat units respectively per polymer molecule. Copolymers may be block copolymers, random copolymers or they may have structures anywhere between these two extremes. The sum of n and m is generally from about 50 to about 20,000 and therefore the polymers have molecular weights from about 2,500 to about 1,000,000 daltons.

The hydrophilic, hydroxyl-containing repeat units, “A,” may simply be hydroxyl (—OH) or it may be any short-chain, 1 to 6 carbon, alkyl with one or more hydroxyls attached thereto. The hydroxyl-substituted alkyl may be attached to the vinyl backbone via carbon-carbon or ether linkages. Thus, exemplary “A” structures include, in addition to hydroxyl itself, hydroxymethyl, hydroxyethyl, hydroxypropyl, hydroxymethoxy, hydroxyethoxy and hydroxypropoxy.

The hydrophobic substituent, “B,” may simply be: hydrogen (—H), in which case the hydrophobic repeat unit is ethylene; an alkyl or aryl substituent with up to 12 carbons attached via a carbon-carbon bond such as methyl, ethyl or phenyl; an alkyl or aryl substituent with up to 12 carbons attached via an ether linkage such as methoxy, ethoxy or phenoxy; an alkyl or aryl substituent with up to 12 carbons attached via an ester linkage such as acetate, propionate, butyrate or benzoate. The amphiphilic hydroxyl-functional vinyl copolymers of the present invention may be synthesized by any conventional method used to prepare substituted vinyl copolymers. Some substituted vinyl copolymers such as polyvinyl alcohol/polyvinyl acetate are well known and commercially available.

A particularly convenient subclass of amphiphilic hydroxyl-functional vinyl copolymers to synthesize are those where the hydrophobic substituent “B” comprises the hydrophilic substituent “A” to which an alkylate or arylate group is attached via an ester linkage to one or more of the hydroxyls of A. Such copolymers may be synthesized by first forming the homopolymer of the hydrophobic vinyl repeat unit having the substituent B, followed by hydrolysis of a portion of the ester groups to convert a portion of the hydrophobic repeat units to hydrophilic, hydroxyl-containing repeat units having the substituent A. For example, partial hydrolysis of the homopolymer, polyvinylbutyrate, yields the copolymer, vinylalcohol/vinylbutyrate copolymer for which A is hydroxyl (—OH) and B is butyrate (—OOC—CH₂—CH₂—CH₃).

For all types of copolymers, the value of n must be sufficiently large relative to the value of m that the resulting copolymer is at least partially water soluble. Although the value of the ratio, n/m varies depending on the identity of A and B, it is generally at least about 1 and more commonly about 2 or more. The ratio n/m can be as high as 200. When the copolymer is formed by hydrolysis of the hydrophobic homopolymer, the relative values of n and m are typically reported in “percent hydrolysis,” which is the fraction (expressed as a percent) of the total repeat units of the copolymer that are in the hydrolyzed or hydroxyl form. The percent hydrolysis, H, is given as

$$H = 100 * ( \frac{n}{n + m} )$$

Thus, vinylbutyrate/vinylalcohol copolymer (formed by hydrolysis of a portion of the butyrate groups) having a percent hydrolysis of 75% has an n/m ratio of 3.

One family of amphiphilic hydroxyl-functional vinyl copolymers are those where A is hydroxyl and B is acetate. Such copolymers are termed vinylacetate/vinylalcohol copolymers. Some commercial grades are also sometimes referred to simply as polyvinylalcohol. However, the true homopolymer, polyvinylalcohol is not amphiphilic, is almost entirely water insoluble. Preferred vinylacetate/vinylalcohol copolymers are those where H is between about 67% and 99.5%, or n/m has a value between about 2 and 200. The preferred average molecular weight is between about 2500 and 1,000,000 daltons and more preferably between about 3000 and about 100,000 daltons.

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; high molecular weight proteins such as gelatin and albumin; and carboxylic acid functionalized starches such as starch glycolate.

Such polymers may be amphiphilic, particularly when two or more types of repeat units with different types of hydrophilicity are present. Thus, one preferred class of non-cellulosic polymers that are amphiphilic are copolymers of a relatively hydrophilic and a relatively hydrophobic monomer. For example, copolymers of methacrylic acid and methylmethacrylate, hydrophilic and hydrophobic repeat units, respectively, are amphiphilic and useful in this invention. Their degree of amphiphilicity is much greater than, for example, the corresponding hydrophilic homopolymer polyacrylic acid or the hydrophobic homopolymer poly(methylmethacrylate). Examples of such copolymers are Eudragit L100 and Eudragit S100. Another example of an amphiphilic ionizable vinyl copolymer is the ternary copolymer of butylmethacrylate, methylmethacrylate and 2-dimethyl-amino ethyl methacrylate. One example of this class of polymers is Eudragit E100. Note that for copolymers of two or more repeat units, the relative fraction of hydrophilic and hydrophobic repeat units is chosen to have some, but not too much, aqueous solubility. Generally, good results are obtained with copolymers that have aqueous solubilities from about 0.1 mg/ml up to about 100 mg/ml over at least a portion of the pH range of 1-8. Best results are often obtained with polymers that have aqueous solubilities in the 0.5 mg/ml to 40 mg/ml range. Some such amphiphilic copolymers do not completely dissolve, but tend to form cloudy solutions. Such polymer solutions are sometimes referred to as “hydrocolloid” solutions.

A preferred class of polymers comprises ionizable and neutral (or non-ionizable) cellulosic polymers with at least one ester- and/or ether-linked substituent in which the polymer has a degree of substitution of at least 0.05 for each substituent. It should be noted that in the polymer nomenclature used herein, ether-linked substituents are recited prior to “cellulose” as the moiety attached to the ether group; for example, “ethylbenzoic acid cellulose” has ethyoxy- benzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.05 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulose polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic substituents include ether- or ester-linked nonionizable groups such as the hydroxy alkyl groups hydroxy ethyl, hydroxy propyl, and the alkyl ether groups such as ethoxyethoxy or methoxyethoxy. Another class of hydrophilic substituents are those that are ether- or ester-linked to the cellulose and, following substitution have ionizable groups such as carboxylic acids, thiocarboxylic acids, substituted phenoxy groups, amines, phosphates or sulfonates. Examples of such ionizable hydrophilic substituents that are ester linked include succinate, citrate, phthalate, trimellitate and glycolate. Examples of such ionizable hydrophilic substituents that are ether linked include carboxymethyl, carboxyethyl, and ethoxybenzoic acid.

Thus, in the case of cellulosic polymers, “amphiphilic polymers” (or specifically “amphiphilic cellulosic polymers”) constitutes any cellulosic polymer that has one or more ester or ether linked substituent chosen from the group consisting of hydrophilic substituents and hydrophobic substituents. A preferred class of amphiphilic cellulosic polymers are those that have at least one hydrophilic substituent and at least one hydrophobic substituent.

Hydrophilic substituents fall into 5 classes:

1. unsubstituted hydroxyls,

2. ether-linked non-ionizable substituents,

3. ether-linked ionizable substituents,

4. ester-linked non-ionizable substituents, and

5. ester-linked ionizable substituents.

Hydrophobic substituents fall into 2 classes:

1. ether-linked non-ionizable substituents, and

2. ester-linked non-ionizable substituents.

In some cases, substituents may, to some extent, be both hydrophilic and hydrophobic. Thus, for example, although ionizable substituents are generally referred to as hydrophilic, it is recognized that in the case of a substituent such as phthalate, that the aromatic ring portion of the substituent is somewhat hydrophobic.

Exemplary hydrophilic ether-linked non-ionizable substituents include the hydroxyalkyl substituents such as hydroxymethyl, hydroxyethyl, hydroxypropyl, etc. and the alkyl ether groups such as ethoxyethoxy or methoxyethoxy.

Exemplary hydrophilic ether-linked ionizable substituents include: carboxylic acids, such as acetic acid, propionic acid, benzoic acid, carboxymethoxy (commonly referred to as carboxymethyl), carboxyethoxy (commonly referred to as carboxyethyl), carboxypropoxy (commonly referred to as carboxypropyl), and carboxyphenoxy (commonly referred to as carboxyphenyl), salicylic acid (attached to the cellulosic polymer via the phenolic hydroxyl), alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as ethoxy phosphate; and sulfonates, such as ethoxy sulphonate.

Exemplary hydrophilic ester-linked non-ionizable substituents include hydroxyacetate, hydroxypropionate, and hydroxybutyrate.

Exemplary hydrophilic ester-linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate.

Exemplary hydrophobic substituents include ether-linked non-ionizable groups such as methyl, ethyl, propyl, butyl, phenyl, etc.; or ester-linked non-ionizable groups such as acetate, propionate, butyrate, benzoate, or phenylate.

The inventors have found that the amphiphilic polymer hydroxypropyl methyl cellulose acetate succinate works well in forming the polymer/drug assemblies of the present invention. As disclosed in Curatolo et al. (EP 0 901 786 A2), when dispersions of low-solubility drugs and HPMCAS are introduced to an aqueous use environment, the resulting aqueous solution has an enhanced concentration of the low solubility drug. While not specifically disclosed by Curatolo et al., it is believed this concentration enhancement is closely related to the presence of polymer/drug assemblies as disclosed herein.

The present inventors have found that the concentration enhancement and polymer/drug assembly-forming properties of hydroxypropyl methyl cellulose acetate succinate (HPMCAS) are not unique but are displayed by other amphiphilic cellulosic polymers. Indeed, Yano, et al., in Chem. Pharm. Bull. 44(12)2309-2313 (1996) present data that show that small colloidal particles may have formed when a dispersion of the poorly soluble drug YM022 and the amphiphilic polymer hydroxypropyl methyl cellulose and polyoxyethylene hydrogenated castor oil was administered to an aqueous solution. As a result, such polymers are also effective in forming polymer/drug assemblies.

In one embodiment, the polymer comprises an ionizable cellulosic polymer, provided that the polymer is not solely hydroxy propyl methyl cellulose acetate succinate.

In another embodiment, the polymer comprises a non-ionizable cellulosic polymer, with the proviso that the polymer is not solely hydroxypropyl methyl cellulose.

Examples of suitable amphiphilic cellulosic polymers that have at least one hydrophobic substituent and at least one hydrophilic substituent include hydroxypropyl cellulose acetate succinate, hydroxypropyl methyl cellulose acetate, hydroxypropyl methyl cellulose succinate, hydroxypropyl methyl cellulose acetate succinate, hydroxypropyl methyl cellulose phthalate, hydroxypropyl methyl cellulose acetate phthalate, hydroxypropyl methyl cellulose, hydroxyethyl methyl cellulose, hydroxyethyl methyl cellulose succinate, hydroxyethyl cellulose acetate succinate, hydroxyethyl methyl cellulose acetate succinate, hydroxyethyl methyl cellulose acetate phthalate, hydroxyethyl cellulose acetate, hydroxyethyl ethyl cellulose, carboxymethyl ethyl cellulose, cellulose acetate phthalate, hydroxypropyl cellulose acetate phthalate, methyl cellulose acetate phthalate, ethyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Examples of amphiphilic cellulosic polymers that have one or more ester- or ether-linked substituent chosen from the group consisting of hydrophilic substituents and hydrophobic substituents include the polymers listed above, as well as carboxyethyl cellulose, carboxymethyl cellulose, hydroxypropyl cellulose, and methyl cellulose.

While, as listed above, a wide range of polymers may be used to form the polymer/drug assemblies of the present invention, the inventors have found that polymers that are highly amphiphilic and are relatively hydrophobic in that they have some, but limited, solubility, have shown the best performance as demonstrated by the formation of stable, small polymer/drug assemblies and therefore showing high values for: (1) total dissolved drug; (2) free drug concentration; and (3) a high ratio of free drug to total dissolved drug. In particular, cellulosic polymers that are aqueous insoluble in their nonionized state but are only somewhat aqueous soluble in their ionized state perform particularly well. Analogous to the previous discussion, amphiphilic ionizable cellulosic polymers that have a solubility, over at least a portion of the physiologically relevant pH range (1-8), of between about 0.1 mg/ml and up to about 100 mg/ml are preferred, with those amphiphilic cellulosic polymers with aqueous solubility between about 0.5 mg/ml and 40 mg/ml being more preferred. Such polymers may be hydrocolloids and only partially dissolved, forming cloudy solutions.

For ionizable polymers, the polymer may be present in either the acidic, basic or a neutralized salt form thereof. In addition, while specific polymers have been discussed as being suitable for use in the various embodiments of the present invention, blends of such polymers may also be suitable. Thus, the term “amphiphilic polymer” is intended to include blends of polymers in addition to a single species of polymer.

### Methods for Forming Solutions Containing Polymer/Drug Assemblies

The polymer/drug assemblies of the present invention may be formed by a wide variety of methods. Essentially any method that provides an aqueous solution comprising (1) a total dissolved drug concentration that is at least temporarily greater than the equilibrium concentration of the drug in that solution provided by the lowest energy crystalline or amorphous form of the drug alone, and (2) an amphiphilic, aqueous-soluble polymer in a sufficient amount to maintain the dissolved drug concentration greater than the equilibrium concentration, results in the formation of polymer/drug assemblies. Exemplary methods for forming an aqueous solution containing substantial amounts of polymer/drug assemblies follows.

The drug may be administered to the aqueous solution using any method, dosage form or drug formulation which results in a total dissolved drug concentration in the aqueous solution that exceeds the equilibrium concentration of the drug in that solution at least temporarily. The “equilibrium concentration of drug” is the concentration provided by the lowest energy crystalline form of the drug, or if a crystalline form is unknown, by the amorphous form of the drug. The reported or measured solubility of the drug in the solution may be taken as the equilibrium concentration. Exemplary dosage forms and drug formulations which provide, at least temporarily, a dissolved drug concentration that exceeds the equilibrium concentration include: a solid amorphous dispersion of the drug in the amphiphilic polymer; a solid amorphous dispersion of the drug in a matrix material other than the amphiphilic polymer; a solubility-improved form of the drug; and, a soluble complex of the drug and a complexing agent (such as a compound that forms a coordinate bond with the drug, such as a cyclodextrin). Solubility-improved forms include crystalline highly soluble salt forms of the drug, high-energy crystalline forms of the drug (such as polymorphs), amorphous drug (where the drug may also exist in crystalline form), a mixture of the drug and a solubilizing agent, and drug predissolved in a solution. Examples of such solubility-improved forms are more fully described in commonly assigned pending patent application titled Pharmaceutical Compositions Providing Enhanced Drug Concentrations, Ser. No. 09/742,785, filed Dec. 20, 2000, which claims priority to provisional patent application Serial No. 60/171,841, filed Dec. 23, 1999, the disclosure of which is incorporated by reference, and Pharmaceutical Compositions Comprising Drug and Concentration-Enhancing Polymers, Ser. No. 60/300,314, filed Jun. 22, 2001, which is also hereby incorporated by reference.

Because the resulting solution must have a sufficiently high drug concentration, it is necessary to administer a sufficient quantity of drug to the aqueous solution. Good results are generally obtained when the total amount of drug present in the aqueous solution would result in a total dissolved drug concentration that exceeds the solubility of the amorphous form of the drug by at least 1.5-fold and more preferably by at least 2-fold. For example, for a drug having a solubility of 10 μg/ml in amorphous form, at least 15 μg of drug, and more preferably at least 20 μg of drug, would be added to 1 ml of aqueous solution to form polymer/drug assemblies.

The amphiphilic polymer may be added to the solution either with the drug or separate therefrom. Thus, the polymer may be mixed with the drug, may be dissolved in the aqueous solution before or after adding the drug, or may be a separate composition from a drug-containing composition. The amount of polymer is preferably equal to the total amount of drug in solution, and more preferably is at least 2-fold the amount of total drug administered to the solution. Thus, where 10 μg of drug is administered to a solution, preferably at least 10 μg, and even more preferably at least 20 μg, of amphiphilic polymer is also administered to the solution. Good results may be obtained where the amount of polymer administered is even greater, such as from 3-fold to 10-fold the amount of drug.

Turning now to specific methods for forming solutions containing polymer/drug assemblies, one preferred method is to administer a solid amorphous dispersion of a drug and amphiphilic polymer to an aqueous solution. Formulations of a solid amorphous dispersion of drug and amphiphilic polymer may be formed using any conventional method. While the drug in its pure state may be crystalline or amorphous, at least a major portion of the drug in the dispersion is amorphous. By “amorphous” is meant simply that the drug is in a non-crystalline state. As used herein, the term “a major portion” of the drug means that at least 60% of the drug in the dispersion is in the amorphous form, rather than the crystalline form. It has been found that the aqueous concentration of the drug in a use environment tends to improve as the amount of amorphous drug present in the dispersion increases. Preferably, the drug in the dispersion is “substantially amorphous.” As used herein, “substantially amorphous” means that the amount of the drug in amorphous form is at least 75%. More preferably, the drug in the dispersion is “almost completely amorphous” meaning that the amount of drug in the amorphous form is at least 90%. Amounts of crystalline drug may be measured by powder X-ray diffraction, Scanning Electron Microscope (SEM) analysis, differential scanning calorimetry (DSC), or any other standard quantitative measurement.

The amorphous drug in the dispersion can exist as a pure phase, as a solid solution of drug homogeneously distributed throughout the polymer or any combination of these states or those states that lie intermediate between them. To maximize the concentration enhancement provided by the dispersion, the dispersion is preferably substantially homogeneous so that the amorphous drug is dispersed as homogeneously as possible throughout the polymer. As used herein, “substantially homogeneous” means that the drug present in relatively pure amorphous domains within the solid dispersion is relatively small, and is less than 20%, and preferably less than 10%, of the total amount of drug. While the dispersion may have some drug-rich domains, it is preferred that the dispersion itself have a single glass transition temperature (T_{g}) which demonstrates that the dispersion is substantially homogeneous. This contrasts with a simple physical mixture of pure amorphous drug particles and pure amorphous polymer particles which generally display two distinct T_{g}S, one that of the drug and one that of the polymer. T_{g} as used herein is the characteristic temperature where a glassy material, upon gradual heating, undergoes a relatively rapid (e.g., 10 to 100 seconds) physical change from a glass state to a rubber state. Dispersions that are substantially homogeneous generally are more physically stable and have improved concentration-enhancing properties and, in turn, improved bioavailability, relative to nonhomogeneous dispersions.

Dispersions of the drug and polymer may be made according to any known process which results in at least a major portion of the drug in the dispersion being in the amorphous state. Such processes include mechanical, thermal and solvent processes. Exemplary mechanical processes include milling and extrusion; melt processes include high temperature fusion, solvent modified fusion and melt-congeal processes; and solvent processes include non-solvent precipitation, spray coating and spray-drying. See, for example, U.S. Pat. No. 5,456,923, U.S. Pat. No. 5,939,099 and U.S. Pat. No. 4,801,460 which describe formation of dispersions via extrusion processes; U.S. Pat. No. 5,340,591 and U.S. Pat. No. 4,673,564 which describe forming dispersions by milling processes; and U.S. Pat. No. 5,684,040, U.S. Pat. No. 4,894,235 and U.S. Pat. No. 5,707,646 which describe the formation of dispersions via melt/congeal processes, the disclosures of which are incorporated by reference.

In particular, when either the polymer or the drug has a relatively low melting point, typically less than about 200° C. and preferably less than about 160° C., extrusion or melt-congeal processes that provide heat and/or mechanical energy are often suitable for forming almost completely amorphous dispersions. Often, when the drug has significant solubility in the dispersion material, such methods may also make substantially homogeneous dispersions. For example, 10 wt % drug and 90 wt % of a suitable polymer may be dry blended, with or without the addition of water, and the blend fed to a twin-screw extrusion device. The processing temperature may vary from about 50° C. up to about 200° C. depending on the melting point of the drug and polymer, which is a function of the polymer grade chosen and the amount of water, if any, added. Generally, the higher the melting point of the drug and polymer, the higher the processing temperature. Generally, the lowest processing temperature that produces a satisfactory dispersion (almost completely amorphous and substantially homogeneous) is chosen.

Another method for forming dispersions is “solvent processing,” which consists of dissolution of the drug and one or more polymers in a common solvent. The term “solvent” is used broadly and includes mixtures of solvents. “Common” here means that the solvent, which can be a mixture of compounds, will simultaneously dissolve the drug and the polymer(s).

After both the drug and polymer(s) have been dissolved, the solvent is rapidly removed by evaporation or by mixing with a non-solvent. Exemplary processes are spray-drying, spray-coating (pan-coating, fluidized bed coating, etc.), vacuum evaporation, and precipitation by rapid mixing of the polymer and drug solution with CO₂, water, or some other non-solvent. Preferably, removal of the solvent results in a solid dispersion which is substantially homogeneous. In substantially homogeneous dispersions, the drug is dispersed as homogeneously as possible throughout the polymer and can be thought of as a solid solution of drug dispersed in the polymer(s). When the resulting dispersion constitutes a solid solution of drug in polymer, the dispersion may be thermodynamically stable, meaning that the concentration of drug in the polymer is at or below its equilibrium value, or it may be considered a supersaturated solid solution where the drug concentration in the dispersion polymer(s) is above its equilibrium value.

The solvent may be removed through the process of spray-drying. The term spray-drying is used conventionally and broadly refers to processes involving breaking up liquid mixtures into small droplets (atomization) and rapidly removing solvent from the mixture in a container (spray-drying apparatus) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This is accomplished by either (1) maintaining the pressure in the spray-drying apparatus at a partial vacuum (e.g., 0.01 to 0.50 atm); (2) mixing the liquid droplets with a warm drying gas; or (3) both. In addition, at least a portion of the heat required for evaporation of solvent may be provided by heating the spray solution.

Solvents suitable for spray-drying can be any organic compound in which the drug and polymer are mutually soluble. Preferably, the solvent is also volatile with a boiling point of 150° C. or less. In addition, the solvent should have relatively low toxicity and be removed from the dispersion to a level that is acceptable according to The International Committee on Harmonization (ICH) guidelines. Removal of solvent to this level may require a processing step such as tray-drying subsequent to the spray-drying or spray-coating process. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethyl acetamide or dimethylsulfoxide can also be used. Mixtures of solvents, such as 50% methanol and 50% acetone, can also be used, as can mixtures with water as long as the polymer and drug are sufficiently soluble to make the spray-drying process practicable. As described previously, addition of at least a few percent water is often preferred.

Generally, the temperature and flow rate of the drying gas is chosen so that the polymer/drug-solution droplets are dry enough by the time they reach the wall of the apparatus that they are essentially solid, and so that they form a fine powder and do not stick to the apparatus wall. The actual length of time to achieve this level of dryness depends on the size of the droplets. Droplet sizes generally range from 1 μm to 500 μm in diameter, with 5 to 100 μm being more typical. The large surface-to-volume ratio of the droplets and the large driving force for evaporation of solvent leads to actual drying times of a few seconds or less, and more typically less than 0.1 second. This rapid drying is often critical to the particles maintaining a uniform, homogeneous dispersion instead of separating into drug-rich and polymer-rich phases. As above, to get large enhancements in concentration and bioavailability it is often necessary to obtain as homogeneous of a dispersion as possible. Solidification times should be less than 100 seconds, preferably less than a few seconds, and more preferably less than 1 second. In general, to achieve this rapid solidification of the drug/polymer solution, it is preferred that the size of droplets formed during the spray-drying process are less than about 100 μm in diameter. The resultant solid particles thus formed are generally less than about 100 μm in diameter.

Following solidification, the solid powder typically stays in the spray-drying chamber for about 5 to 60 seconds, further evaporating solvent from the solid powder. The final solvent content of the solid dispersion as it exits the dryer should be low, since this reduces the mobility of drug molecules in the dispersion, thereby improving its stability. Generally, the solvent content of the dispersion as it leaves the spray-drying chamber should be less than 10 wt % and preferably less than 2 wt %. In some cases, it may be preferable to spray a solvent or a solution of a polymer or other excipient into the spray-drying chamber to form granules, so long as the dispersion is not adversely affected.

Spray-drying processes and spray-drying equipment are described generally in Perry's Chemical Engineers' Handbook, Sixth Edition (R. H. Perry, D. W. Green, J. O. Maloney, eds.) McGraw-Hill Book Co. 1984, pages 20-54 to 20-57. More details on spray-drying processes and equipment are reviewed by Marshall “Atomization and Spray-Drying,” 50 Chem. Eng. Prog. Monogr. Series 2 (1954).

The amount of polymer relative to the amount of drug present in the solid amorphous dispersions depends on the drug and polymer and may vary widely from a drug-to-polymer weight ratio of from 0.01 to about 4 (e.g., 1 wt % drug to 80 wt % drug). However, in most cases it is preferred that the drug-to-polymer ratio is greater than about 0.05 (4.8 wt % drug) and less than about 2.5 (71 wt % drug).

Thus, solutions containing polymer/drug assemblies can be formed by administering a solid amorphous dispersion of a drug and amphiphilic polymer, such as those described above, to an aqueous solution.

Another method to form polymer/drug assemblies is to administer a solid amorphous drug/matrix dispersion mixed with the amphiphilic polymer to an aqueous solution. The drug/matrix dispersion may be formed using any of the methods described above for forming solid amorphous dispersions. A solution containing polymer/drug assemblies may be formed by either (1) combining the solid drug/matrix dispersion with the amphiphilic polymer and then adding the mixture to the aqueous solution; (2) adding the drug/matrix dispersion to an aqueous solution that already contains amphiphilic polymer; or (3) adding the drug/matrix dispersion to the aqueous solution and then adding the amphiphilic polymer to the aqueous solution. It is preferred that the amphiphilic polymer be either present in the solution, or administered with or shortly after the drug is administered to the solution. In forming solutions of this invention by utilizing a drug/matrix dispersion, the drug/matrix dispersion should have sufficient energy, and be added in sufficient quantity that, at least temporarily a drug concentration that is at least 1.25-fold the equilibrium concentration is achieved. Compositions comprising drug/matrix dispersions and concentration-enhancing polymer are more fully disclosed in commonly assigned co-pending patent application Ser. No. 60/300,261, entitled Pharmaceutical Compositions of Dispersions of Amorphous Drug Mixed With Polymers Jun. 22, 2001, the disclosure of which is hereby incorporated by reference.

The matrix may comprise a single component or it may be a mixture of two or more components. The components may be intimately mixed to form a single phase or molecular dispersion or they may exist as two or more distinct phases with differing compositions.

At least a portion of the matrix is either water swellable, dispersible, or soluble in aqueous solution at physiologically relevant pH (e.g., pH 1-8). The matrix as a whole should be a solid at room temperature, and remain substantially solid up to a temperature of about 40° C., preferably up to a temperature of about 60° C., and more preferably up to a temperature of about 70° C. In order to achieve this, the matrix should be comprised of at least one or more components with a melting point above about 40° C., preferably above about 60° C., and more preferably above about 70° C. The matrix should also be “inert,” meaning not undesirably reactive or bioactive, and should be biologically inert or non-toxic in the sense that it is acceptable for administration to or injection into an animal such as a human.

The amount of matrix relative to the amount of drug present in the dispersion depends on the drug and matrix and may vary widely from a drug-to-matrix weight ratio of from 0.01 to about 4 (e.g., 1 wt % drug to 80 wt % drug). This will vary dependent on the dose of the drug. When the dose is low, less than about 50 mg, the drug-to-matrix weight ratio can be quite small, even less than 0.01. In general, when the dose is relatively high, that is greater than about 50 mg, the drug-to-matrix ratio may be as high as 4.

The components used in the matrix may be polymeric or non-polymeric, and may comprise a mixture of several components. Thus, the matrix may comprise a mixture of polymeric components, a mixture of non-polymeric components, or a mixture of polymeric and non-polymeric components.

The term “polymeric” is used conventionally, meaning a compound that is made of monomers connected together to form a larger molecule. A polymeric component generally consists of at least about 20 monomers. Thus, the molecular weight of a polymeric component will generally be about 2000 daltons or more. Polymeric matrix components generally will result in dispersions with improved concentration enhancement relative to non-polymeric matrix components. Exemplary polymeric components for use as the matrix include polyethylene glycols, polyoxyethylene glycols, polyethylene-propylene glycol copolymers, polyethylene oxides, polyvinyl pyrrolidinone (also referred to as polyvinylpyrolidone or PVP), polyvinyl alcohol, polyethylene-vinyl alcohol copolymers, polyvinyl alcohol polyvinyl acetate copolymers, xanthan gum, carrageenan, hydroxypropyl cellulose, hydroxypropyl methyl cellulose, carboxy methyl cellulose, carboxylic acid-functionalized polymethacrylates, amine-functionalized polymethacrylates, chitosan, chitin, polydextrose, dextrin and starch. Also included within this definition are high molecular weight proteins such as gelatin and albumin.

By “non-polymeric” is meant that the component is not polymeric. Exemplary non-polymeric materials for use as a matrix component include: alcohols, such as stearyl alcohol and cetyl alcohol; organic acids, such as stearic acid, citric acid, fumaric acid, tartaric acid, and malic acid; organic bases, such as glucosamine, N-methylglucamine, tris(hydroxymethyl)amino methane, and dodecylamine, salts such as sodium chloride, potassium chloride, lithium chloride, calcium chloride, magnesium chloride, sodium sulfate, potassium sulfate, sodium carbonate, and magnesium sulfate; amino acids such as alanine and glycine; sugars such as glucose, sucrose, xylitol, fructose, lactose, mannitol, sorbitol, and maltitol; fatty acid esters such as glyceryl (mono- and di-) stearates, glyceryl (mono- and di-) behenates, triglycerides, sorbitan monostearate, saccharose monostearate, glyceryl (palmitic stearic) ester, polyoxyethylene sorbitan fatty-acid esters; waxes, such as microcrystalline wax, paraffin wax, beeswax, synthetic wax, castor wax, and carnauba wax; alkyl sulfates such as sodium lauryl sulfate and magnesium lauryl sulfate; and phospholipids, such as lecithin.

Thus, solutions containing polymer/drug assemblies can be formed by administering a solid amorphous drug/matrix dispersion mixed with an amphiphilic polymer, such as those described above, to an aqueous solution.

Yet another method to form polymer/drug assemblies is to administer the drug in a solid solubility-improved form to an aqueous solution with amphiphilic polymer. A solid solubility-improved form of the drug may be a crystalline highly soluble salt form of the drug, high-energy crystalline form of the drug (such as polymorphs), amorphous drug (where the drug may also exist in crystalline form), a mixture of the drug and a solubilizing agent, or a soluble complex of the drug and a complexing agent. Such solubility-improved forms are capable of providing, at least temporarily, a concentration of dissolved drug that exceeds the equilibrium concentration of drug. An aqueous solution containing polymer/drug assemblies may be formed from such solubility-improved forms by any of the following methods. The drug in the solubility improved form and polymer may be added separately to the aqueous solution. The drug may be added prior to the polymer, at the same time, or after the polymer has been added to the solution. Alternatively, the drug and polymer may first be combined together, such as by mixing or by formulation into a single dosage form, and then added to the aqueous solution.

Finally, polymer/drug assemblies may be formed by predissolving the drug in a solution, and then adding the solution of predissolved drug along with an amphiphilic polymer to an aqueous solution. For example, the drug may be dissolved in an organic, preferably water-miscible solvent, and then the resulting drug solution mixed with an aqueous solution in which the polymer is dissolved. The aqueous solution may contain various solutes, particularly those that render the amphiphilic polymer soluble, such as acids, bases or buffers. Alternatively, the amphiphilic polymer may be dissolved in a water-miscible solvent in which both the drug and polymer are soluble to form a solution of drug and polymer. The solution may then be mixed with sufficient aqueous solution such that the drug concentration exceeds its equilibrium concentration in the resulting aqueous solution.

The manner in which these pre-dissolution methods are conducted has a significant effect on the type of polymer/drug assemblies that are formed. In general, a significant fraction of the total drug in such solutions is in the form of the small polymer/drug assemblies only when the total drug administered to the solution exceeds the equilibrium solubility of the drug in the combined solutions but in the absence of polymer. It is also generally observed that when the drug is administered to the solution at extremely high levels, it may interact with the polymer to form large polymer/drug assemblies that are greater than about 5 μm in size. Such large polymer/drug assemblies tend to precipitate. Although in some cases, solutions in which a large fraction of the drug is in the form of a precipitate may function well, it is generally preferred to combine the solutions in the above methods so as to avoid having most of the drug be present as a precitate. Thus, in general, it is preferred to combine the solutions in the above methods such that the final drug concentration in the solution is greater than about 2-fold the equilibrium solubility of the drug in the final solution but less than about 10 mg/ml. When the solubility of the drug is less than about 10 μg/ml in the final solution, it is often preferable to have the final drug concentration be less than about 2 mg/ml.

It is also generally preferred to combine the solutions such that the two solutions, once contacted, rapidly become completely mixed. Thus, it is often preferred to agitate or mix the solutions as they are being combined or immediately following their combination. Alternatively, the solutions may be combined within pipes, tubes or conduits and be pumped through static or dynamic means to cause mixing such as being pumped through an in-line mixer. Yet another alternative is to slowly add one to the other while the combined solutions are being agitated or mixed.

### Solid Aggregated Polymer/Drug Assemblies

Another separate aspect of the invention comprises compositions of solid aggregated polymer/drug assemblies which, when administered to an aqueous solution, provide enhanced drug concentration. By “solid aggregated polymer/drug assembly” is meant a solid composition of drug and polymer which has been separated from a solution containing polymer/drug assemblies. Such solid aggregated polymer/drug assemblies are capable of providing significantly enhanced dissolved drug concentration in aqueous solution. In fact, such solid polymer/drug assemblies generally are capable of providing even higher concentrations of total dissolved drug than solid amorphous dispersions of the same drug and polymer.

Specifically, when a solid amorphous dispersion of a drug in a concentration-enhancing polymer is formed and subsequently dosed to an aqueous solution, it provides an enhanced total dissolved drug concentration relative to dosing the drug in crystalline or amorphous form in the absence of the polymer. When the amount of dispersion dosed to the solution is increased, the total amount of dissolved drug generally increases. However, the fraction of drug dosed that dissolves generally decreases. Thus, for example, if a solid amorphous dispersion of drug in HPMCAS is dosed at 0.5 mg/ml, 1 mg/ml, and 2 mg/ml to a PBS solution, and the maximum total dissolved drug obtained is 0.48, 0.65, and 0.95 mg/ml, respectively, then the fraction of dosed drug that dissolves, expressed as a percent, is 96%, 65%, and 48%, respectively. When solid aggregated polymer/drug assemblies are formed by, for example, lyophilizing the supernatant from an aqueous solution formed by dissolution of the same dispersion they provide a greater amount of total dissolved drug, and a greater fraction of dissolved drug relative to the amount of dosed drug. For example, if solid aggregated polymer/drug assemblies of drugs and HPMCAS were dosed as a dry powder to a PBS solution at a dose of 2.0 mg/ml, the maximum total dissolved drug would be greater than 0.95 mg/ml, and the fraction of the drug dissolved would be greater than 48%. The solid aggregated polymer/drug assemblies provide a maximum total dissolved drug concentration in a use environment that is preferably at least 1.1-fold higher than that provided by the precursor solid amorphous dispersion of drug and amphiphilic polymer. In general, such improvements are greater at higher doses where the fraction of total dissolved drug for the dispersion is only about 60% or less of the dose. This improvement in performance indicates that the solid aggregated polymer/drug assemblies are different in physical state than the corresponding solid amorphous dispersions of the same drug and amphiphilic polymer.

When forming the solid aggregated polymer/drug assemblies from an aqueous solution containing polymer/drug assemblies, the resulting solid particles are relatively small but usually are larger than the polymer/drug assemblies which were present in the solution from which the solid particles were formed. The solid assemblies often loosely aggregate to form particles larger than 5 μm in diameter. However, upon administering the solid assemblies to an aqueous solution, a substantial portion, and often most, of the polymer and drug return to the smaller size of the polymer/drug assemblies present in solution.

While not wishing to be bound by any particular mechanism for this difference, the following distinctions have generally been observed for the solid aggregated polymer/drug assemblies which demonstrate their unique physical form relative to previously known forms of polymer/drug compositions. Some or all of the following differences have been observed for solid aggregated polymer/drug assemblies relative to solid dispersions of the same drug and polymer:

1. enhanced fraction of total dissolved drug relative to that dosed;

2. a shift in the value of, or the absence of, the glass-transition temperature;

3. a shift or the absence of an exotherm in the DSC trace indicating an inhibition of crystallization and an improved physical stability;

4. the appearance of broad peaks in the powder x-ray diffraction pattern indicating an increase in order of the drug.

These differences in physical properties indicate that, although composed of the same components, solid aggregated polymer/drug assemblies have a physical form distinct and preferred over other known forms such as solid amorphous dispersions, physical mixtures of amorphous drug and polymer or physical mixtures of crystalline drug and polymer.

The unique state of drug within the solid aggregated polymer/drug assembly may be termed a “semi-ordered state.” By “semi-ordered state” is meant that, in contrast to a solid amorphous dispersion, the drug displays one or more characteristics that indicate the drug has become more ordered. However, this semi-ordered state is also distinct from the crystalline state in that the compositions do not display substantial melting exotherms or powder x-ray diffraction patterns characteristic of bulk crystalline drug. Each of the three properties, in addition to enhanced total dissolved drug discussed above, that distinguish the “semi-ordered state” of drug within the solid aggregated polymer/drug assemblies are described below.

Differential scanning calorimetry is a standard method for assessing the physical state of materials. Amorphous materials generally display a characteristic change in heat capacity upon heating near the temperature where they change from the “glassy state” to the rubbery state. The temperature at which this transition occurs is known as the “glass transition temperature” or T_{g}. See, for example Moynihan, et al. (279 Ann. N.Y. Acad. Sci. 15-35 (1976)) for a description of this physical property and how it may be measured. A common method involves subjecting a sample of a material to differential scanning calorimetry analysis. For solid amorphous dispersions in which the drug and polymer that make up the dispersion are homogeneously mixed, the dispersion will generally show a single T_{g} value intermediate between that of the amorphous drug T_{g}. and the amorphous polymer T_{g}. In contrast, a corresponding solid aggregated polymer/drug assembly will generally show either a weak T_{g}. shifted to a temperature significantly different from that of the dispersion, or alternatively, the T_{g} will be absent altogether from the DSC heat-flow curve.

Another physical transition that may be observed by DSC analysis is the crystallization of the amorphous drug present in the sample. Generally, when amorphous drug alone or amorphous drug dispersed in a polymer is heated above the T_{g} of the material, an exothermic heat flow is observed due to the crystallization of the drug. This transition is normally observed at a temperature of 10° C. to 70° C. above the T_{g}. A general characteristic of solid aggregated polymer/drug assemblies that distinguishes them from amorphous drug alone or a solid amorphous dispersion of drug in polymer is that upon DSC analysis the crystallization exothermic transition is either (1) observed at a higher temperature than that observed for either the amorphous drug alone or a solid dispersion of drug in polymer or (2) the crystallization exotherm is absent altogether from the DSC heat-flow curve. These changes in the crystallization exotherm indicate that crystallization is inhibited by the solid aggregated polymer/drug assembly state and that this state is a more physically stable amorphous state relative to amorphous drug alone or the solid amorphous dispersion state.

Finally, powder X-ray diffraction analysis of solid aggregated polymer/drug assemblies generally yield diffraction patterns distinct from any of the known physical states of drug including:

1. the crystalline drug state (which shows many sharp diffraction lines);

2. the amorphous drug alone (which shows one or two extremely broad scattering bands); and

3. the solid amorphous dispersion (which also typically shows one or two extremely broad scattering bands).

Specifically, the solid aggregated polymer/drug assemblies may show several broad scattering lines that are much broader than crystalline drug but more numerous, sharper and more distinct than either amorphous state. This suggests that the drug in the solid aggregated polymer/drug assemblies is more ordered than is the drug in the normal amorphous state or in solid amorphous dispersions but less ordered than in the crystalline state.

In summary, the solid aggregated polymer/drug assemblies of the present invention constitute a novel, unique and preferred state of polymer and drug that has the advantages of improved dissolution in an aqueous use environment, and improved physical stability.

Solid aggregated polymer/drug assemblies are comprised of amorphous drug and amphiphilic polymer. Such solid aggregated polymer/drug assemblies may be composed of any of the drugs and polymers described above for the polymer/drug assemblies that exist in solution. The composition of such assemblies can generally range from about 1.0 wt % drug up to about 98 wt % drug; the remainder being polymer as well as any of the additives previously described. Generally, the drug concentration in the assemblies will be less than about 90 wt % and more than about 5 wt %. The solid aggregated polymer/drug assemblies may contain up to about 40 wt % additives such as water, solvents, plasticizers, surfactants, buffers, acids, bases or micelle-forming materials that may improve the performance of the assemblies when reconstituted or may improve the stability of the assemblies. Surfactants are a particularly preferred additive.

Solid aggregated polymer/drug assemblies may be formed by various methods. In one method, a solution containing polymer/drug assemblies in a solvent is first prepared, and then the polymer/drug assemblies are isolated from the solution. Solutions containing polymer/drug assemblies may be formed by any of the methods discussed above. The polymer/drug assemblies may then be isolated by a variety of methods, such as by removal of the solvent. The solvent may be aqueous or organic, and may comprise a mixture of materials. However, in general, the solvent is preferably aqueous meaning that it contains at least some water. Specifically, the solvent should comprise at least about 20 wt % water and water levels of about 40 wt % or more are even more preferred. It is believed that it is preferred to have an aqueous solvent as water promotes the hydrophillic and lipophilic interactions that lead to the formation of polymer/drug assemblies. The solvent may also contain substantial quantities of other solutes or additives to aid in forming the polymer/drug assemblies. The solvent may be removed by centrifugation followed by decantation, evaporation by for example rotary evaporation, spray-drying or lyophilization. Alternatively, the polymer/drug assemblies may be isolated from the aqueous solution by filtration such as microfiltration or ultrafiltration, and then dried, or centrifugation followed by separating the solids from the supernatant followed by drying the solids. Substantial quantities of organic solvent or water may remain in the solid and it may still perform satisfactory.

In another method, a dry composition of polymer/drug assemblies is formed and then preferred sizes are selected. Selection may occur by any conventional method that separates particles by weight or size. For example, the solution may be centrifuged prior to removal of solvent, thus preferentially removing larger or denser polymer/drug assemblies from solution. Solvent may then be removed from the resulting solution of smaller or less dense polymer/drug assemblies. Yet another method for selecting for particular kinds of polymer/drug assemblies is to screen the resulting dry composition by size. One preferred size is less than about 1 μm to less than about 10 μm in diameter. The inventors have found that these methods allow for the selection of polymer/drug assemblies that are smaller, and when reconstituted in solution provide for a higher total concentration of dissolved drug relative to that dosed.

Solid aggregated polymer/drug assemblies may also be formed by combining solutions at a commercial scale in either a batch or continuous process. An exemplary batch process may comprise first forming in a large (100 L to 10,000 L) stirred, temperature-controlled vessel, a solution of drug in an organic, water miscible solvent such as acetone, propanol, N-methylpyrrolidone, or the like. Generally the concentration of drug in the solution should be below its solubility limit in the solvent but at least about 10-fold its aqueous solubility. A second solution is prepared by combining the amphiphilic polymer that will form the assemblies with water in a second large, stirred, temperature-controlled vessel. Often it is desirable to add acid, base, or preferably a buffer to the solution such that the pH of the solution is near neutrality; that is between about pH 4 and 10 and preferably between about pH 5 and 9. This is particularly important when the polymer, drug or both are ionizable. In many cases it is preferred, when the solutions are combined, for the drug and polymer to be substantially in the ionic state they are normally in at the pH of the duodenum or small intestines. It is also often desirable to add a surfactant to one or both solutions as well. In one embodiment the so formed organic solution of drug is then pumped into the vessel containing the aqueous solution of polymer while vigorously stirring the solution in the vessel. Generally the solution is added over 1 to,1000 minutes. However, it is generally preferred to add the drug solution relatively slowly; typically over a period of at least 10 minutes. Following combination of the two solutions, the solution is typically stirred for an additional 1 to 100 minutes and then the polymer/drug assemblies so formed are separated from the solvent. This may be accomplished by filtration of various types to yield a wet concentrated mass that may be further dried by processing in a tray dryer or a fluid-bed dryer by passing a dry gas over the material. Alternatively, vacuum or microwave drying processes may be used.

An alternate method for separating the solid aggregated polymer/drug assemblies from the solvent mixture is via an evaporative process such as spray drying. Thus the solution mixture may be delivered to a commercial spray dryer along with the drying gas such as nitrogen or air. The solution mixture is “atomized” to form small droplets and the solvents (organic solvent and water) are evaporated rapidly to yield a dry powdered product. Typically, residual solvent and water is present in the dry powdered product which may be removed in a subsequent drying step utilizing, for example, a tray dryer, drum dryer, a vacuum dryer or a fluid-bed dryer. The resulting dry powder typically will consist of particles about 0.5 to about 200 μm in diameter. Such particles, may be further processed to form granules via any granulation process know in the pharmaceutical arts such as dry granulation or wet granulation.

An alternate method for forming solid aggregated polymer/drug assemblies by combining solutions may be conducted by bringing together preformed solutions by pumping through an “in line mixer.” Thus, (1) an aqueous solution of polymer, preferably with a pH from about 4 to about 10, and (2) an organic solution of drug (as described above) are pumped at a controlled rate to an “in-line mixer” such that the solutions are rapidly and completely mixed. The mixture, including the polymer/drug assemblies formed upon mixing may be delivered to a tank for storage until further processing is convenient or may be fed directly to a process to separate the polymer/drug assemblies from the mixed solvent. Any of the processes described above such as filtration or evaporative processes such as spray drying may be employed.

The solid aggregated polymer/drug assemblies, when administered in a sufficient amount, improve the concentration of the drug in a use environment relative to a control composition. At a minimum, the solid aggregated polymer/drug assemblies provide concentration-enhancement relative to a control composition comprising crystalline drug alone. Thus, when a composition comprising the solid aggregated polymer/drug assemblies is administered to a use environment, the composition provides improved drug concentration (as described more fully below) relative to a control consisting of an equivalent amount of crystalline drug but with no polymer present. Preferably, the compositions containing solid aggregated polymer/drug assemblies of the present invention provide concentration-enhancement relative to a control composition containing an equivalent amount of amorphous drug but with no polymer. Even more preferably, the solid aggregated polymer/drug assemblies provide a total dissolved drug concentration that is enhanced relative to a control composition consisting of a solid amorphous dispersion of the same polymer and drug. Even more preferably, the maximum fraction of dosed drug that dissolves upon dosing the solid aggregated polymer/drug assemblies to an aqueous solution is at least 1.1-fold that observed when a solid amorphous dispersion consisting of the same polymer and drug is dosed at a level that yields, for the dispersion, a total dissolved drug fraction that is 60% or less of that dosed to the solution.

A composition containing solid aggregated polymer/drug assemblies of the present invention provides a Maximum Drug Concentration (MDC) in a use environment that is at least 1.25-fold the MDC of at least one of the control compositions. In other words, if the MDC provided by the control composition is 100 μg/mL, then a composition of the present invention containing polymer/drug assemblies provides an MDC of at least 125 μg/mL. More preferably, the MDC of drug achieved with the compositions containing solid aggregated polymer/drug assemblies of the present invention are at least 2-fold, and even more preferably at least 3-fold, that of at least one of the control compositions.

Alternatively, the compositions containing solid aggregated polymer/drug assemblies of the present invention provide in an aqueous use environment a concentration versus time Area Under The Curve (AUC), for any period of at least 90 minutes between the time of introduction into the use environment and about 270 minutes following introduction to the use environment that is at least 1.25-fold that of at least one of the control compositions. More preferably, the AUC achieved with the compositions of the present invention are at least 2-fold and more preferably at least 3-fold that of at least one of the control compositions.

Alternatively, the compositions of the present invention containing solid aggregated polymer/drug assemblies, when dosed orally to a human or other animal, provide an AUC in drug concentration in the blood plasma or serum that is at least 1.25-fold that observed when one of the control compositions is dosed. More preferably, the AUC in the blood plasma or serum is at least 2-fold and more preferably at least 3-fold that observed when one of the control compositions is dosed. Thus, the compositions of the present invention can be evaluated in either an in vitro or in vivo test, or both.

A typical test to evaluate enhanced drug concentration can be conducted by (1) adding a sufficient quantity of test composition (e.g., the composition containing solid aggregated polymer/drug assemblies) to a test medium (such as PBS or MFD solution), such that if all of the drug dissolved, the theoretical concentration of drug would exceed the equilibrium concentration of the drug in the test medium by a factor of at least 2; (2) adding an appropriate amount of control composition to an equivalent amount of test medium, and (3) determining whether the measured MDC and/or AUC of the test composition in the test medium is at least 1.25-fold that of the MDC and/or AUC provided by the control composition. In conducting such a dissolution test, the amount of test composition used is an amount such that if all of the drug dissolved, the drug concentration would be at least 2-fold to 100-fold that of the equilibrium concentration of the drug. The concentration of dissolved drug is typically measured as a function of time by sampling the test medium and plotting drug concentration in the test medium vs. time so that the MDC and/or AUC can be ascertained.

To avoid drug particulates which would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. As previously discussed, other similar filtration or centrifugation methods can be employed and useful results obtained.

Alternatively, the compositions of the present invention provide improved relative bioavailability. Relative bioavailability of the drug in the compositions of the present invention can be tested in vivo in animals or humans using conventional methods for making such a determination. An in vivo test, such as a crossover study, may be used to determine whether a test composition provides an enhanced relative bioavailability compared with a control composition. In an in vivo crossover study a “test composition” of solid aggregated polymer/drug assemblies is dosed to half a group of test subjects and, after an appropriate washout period (e.g., one week) the same subjects are dosed with a “control composition.” The “control composition” may be any of the control compositions described earlier. The other half of the group is dosed with the control composition first, followed by the test composition. The relative bioavailability is measured as the concentration in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC can be made by plotting the serum or plasma concentration of drug along the ordinate (y-axis) against time along the abscissa (x-axis). Generally, the values for relative bioavailability represent a number of values taken from all of the subjects in a patient test population averaged over the entire test population.

A preferred embodiment of the invention is one in which the relative bioavailability of the test composition is at least 1.25 relative to at least one of the control compositions. (That is, the AUC in the blood provided by the test composition is at least 1.25-fold the AUC provided by the control composition.) An even more preferred embodiment of the invention is one in which the relative bioavailability of the test composition is at least 2.0 relative to at least one of the control compositions. The determination of AUCs is a well-known procedure and is described, for example, in Welling, “Pharmacokinetics Processes and Mathematics,” ACS Monograph 185 (1986).

Despite the acceptability of such tests, as stated previously, preferred solid aggregated polymer/drug assemblies show superior dissolution properties relative to solid amorphous dispersions of the same polymer and drug. Thus, any of the above dissolution or bioavailability tests may be used to compare a solid aggregated polymer/drug assembly and a control composition comprising a solid amorphous dispersion of the same drug and polymer. Both the solid aggregated polymer/drug assemblies and control dispersion should be dosed at a high enough level that the performance of the control is substantially less than the theoretical maximum. Preferred compositions of the present invention are those that perform at least 1.1-fold that of the control.

In some embodiments, the drug has improved physical stability in the solid aggregated polymer/drug assemblies. The physical stability of the drug in the assemblies may be evaluated by measuring the rate of change in the physical state of the drug from non-crystalline to crystalline in the assemblies and comparing the rate to the corresponding rate of change provided by a control composition consisting of undispersed amorphous drug alone. More preferably, the solid aggregated polymer/drug assemblies show improved physical stability relative to a control composition consisting of a solid amorphous dispersion of an equivalent amount of the amphiphilic polymer and an equivalent amount of drug. The rate of change may be measured by determining the fraction of drug in the crystalline state in the assemblies or control over time. This may be measured by any standard physical measurement, such as X-ray diffraction, DSC, solid state NMR or Scanning Electron Microscope (SEM) analysis. Physically stable compositions of the present invention will crystallize at a slower rate than a control composition. Preferably, the rate of crystallization of the drug in the assemblies is less than 90%, and more preferably less than 80%, of the rate of crystallization of a control composition.

Although the key ingredients present in the compositions of the present invention are simply the solid aggregated polymer/drug assemblies, the inclusion of other excipients in the composition may be useful. These excipients may be utilized with solid aggregated polymer/drug assemblies in order to formulate the mixture into tablets, capsules, suspensions, powders for suspension, creams, transdermal patches, depots, and the like.

One very useful class of excipients is surfactants. Suitable surfactants include fatty acid and alkyl sulfonates; commercial surfactants such as benzethanium chloride (HYAMINE® 1622, available from Lonza, Inc., Fairlawn, N.J.); DOCUSATE SODIUM (available from Mallinckrodt Spec. Chem., St. Louis, Mo.); polyoxyethylene sorbitan fatty acid esters (TWEEN®, available from ICI Americas Inc., Wilmington, Del.); LIPOSORB® P-20 (available from Lipochem Inc., Patterson N.J.); CAPMUL® POE-0 (available from Abitec Corp., Janesville, Wis.), and natural surfactants such as sodium taurocholic acid, 1-palmitoyl-2-oleoyl-sn-glycero-3-phosphocholine, lecithin, and other phospholipids and mono- and diglycerides. Such materials can advantageously be employed to increase the rate of dissolution by facilitating wetting, thereby increasing the maximum dissolved concentration, and also to inhibit crystallization or precipitation of drug by interacting with the dissolved drug by mechanisms such as complexation, formation of inclusion complexes, formation of micelles or adsorbing to the surface of solid drug. These surfactants may comprise up to 5 wt % of the composition.

The addition of pH modifiers such as acids, bases, or buffers may also be beneficial, retarding the dissolution of the composition (e.g., acids such as citric acid or succinic acid when the polymer is anionic) or, alternatively, enhancing the rate of dissolution of the composition (e.g., bases such as sodium acetate or amines when the polymer is anionic).

Other conventional formulation excipients may be employed in the compositions of this invention, including those excipients well-known in the art (e.g., as described in Remington's Pharmaceutical Sciences (16th ed. 1980). Generally, excipients such as fillers, disintegrating agents, pigments, binders, lubricants, glidants, flavorants, and so forth may be used for customary purposes and in typical amounts without adversely affecting the properties of the compositions. These excipients may be utilized after the drug/polymer composition has been formed, in order to formulate the composition into tablets, capsules, suspensions, powders for suspension, creams, transdermal patches, and the like.

Examples of other matrix materials, fillers, or diluents include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers such as polyethylene oxide, and hydroxypropyl methyl cellulose.

Examples of surface active agents include sodium lauryl sulfate and polysorbate 80.

Examples of drug complexing agents or solubilizers include the polyethylene glycols, caffeine, xanthene, gentisic acid and cylodextrins.

Examples of disintegrants include sodium starch glycolate, sodium carboxymethyl cellulose, calcium carboxymethyl cellulose, croscarmellose sodium, crospovidone (polyvinylpolypyrrolidone), methyl cellulose, microcrystalline cellulose, powdered cellulose, starch, pregelatinized starch, and sodium alginate.

Examples of tablet binders include acacia, alginic acid, carbomer, carboxymethyl cellulose sodium, dextrin, ethylcellulose, gelatin, guar gum, hydrogenatetd vegetable oil, hydroxyethyl cellulose, hydroxypropyl cellulose, hydroxypropyl methyl cellulose, methyl cellulose, liquid glucose, maltodextrin, polymethacrylates, povidone, pregelatinized starch, sodium alginate, starch, sucrose, tragacanth, and zein.

Examples of lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate.

Examples of glidants include silicon dioxide, talc and cornstarch.

Compositions of this invention containing solid aggregated polymer/drug assemblies may be used in a wide variety of dosage forms for administration of drugs. Exemplary dosage forms are powders or granules that may be taken orally either dry or reconstituted by addition of water to form a paste, slurry, suspension or solution; tablets; capsules; multiparticulates; and pills. Various additives may be mixed, ground, or granulated with the compositions of this invention to form a material suitable for the above dosage forms.

In some cases, the overall dosage form or particles, granules or beads that make up the dosage form may have superior performance if coated with an enteric polymer to prevent or retard dissolution until the dosage form leaves the stomach. Exemplary enteric coating materials include HPMCAS, HPMCP, CAP, CAT, carboxymethylethyl cellulose, carboxylic acid-functionalized polymethacrylates, and carboxylic acid-functionalized polyacrylates.

Compositions of this invention may be administered in a controlled release dosage form. In one such dosage form, the composition of the solid polymer/drug assemblies is incorporated into an erodible controlled-release matrix device. By an erodible controlled-release matrix is meant aqueous-erodible or water-swellable or aqueous-soluble in the sense of being either erodible or swellable or dissolvable in pure water or requiring the presence of an acid or base to ionize the erodible controlled-release matrix sufficiently to cause erosion or dissolution. When contacted with the aqueous environment of use, the erodible controlled-release matrix imbibes water and forms an aqueous-swollen gel or “matrix” that entraps the solid aggregated polymer/drug assemblies. The aqueous-swollen controlled-release matrix gradually erodes, swells, disintegrates or dissolves in the environment of use, thereby controlling the release of the drug to the environment of use.

Alternatively, the compositions of the present invention may be administered by or incorporated into a non-erodible controlled-release matrix device.

Alternatively, the solid aggregated polymer/drug assemblies of the invention may be delivered via a coated osmotic or hydrogel controlled release dosage form. This dosage form has two components: (a) the core which contains an osmotic agent and the solid aggregated polymer/drug assemblies and (b) a coating surrounding the core, the coating controlling the influx of water to the core from an aqueous environment of use so as to cause drug release by extrusion of some or all of the core to the environment of use. The osmotic agent contained in the core of this device may be an aqueous-swellable hydrophilic polymer, osmogen, or osmagent. The coating is preferably polymeric, aqueous-permeable, and has at least one delivery port.

Alternatively, the solid aggregated polymer/drug assemblies of the invention may be delivered via a coated osmotic or hydrogel controlled release dosage form having three components: (a) a drug-containing composition containing the solid aggregated polymer/drug assemblies (b) a water-swellable composition wherein the water-swellable composition is in a separate region within a core formed by the drug-containing composition and the water-swellable composition, and (c) a coating around the core that is water-permeable, and has at least one delivery port therethrough. In use, the core imbibes water through the coating, swelling the water-swellable composition and increasing the pressure within the core, and fluidizing the drug-containing composition. Because the coating remains intact, the drug-containing composition is extruded out of the delivery port into an environment of use.

In addition to the above additives or excipients, use of any conventional materials and procedures for preparation of suitable dosage forms using the compositions of this invention known by those skilled in the art are potentially useful.

### EXAMPLES

### Examples 1 and 2

Amorphous solid dispersions of the low-solubility drug 5-chloro-1H-indole-2-carboxylic acid [(1S)-benzyl-3-((3R, 4S)-dihydroxypyrroldin-1-yl-)-(2R)-hydroxy-3-oxypropyl] amide (Drug 1) and the amphiphilic polymer hydroxypropyl methyl cellulose acetate succinate were prepared. Crystalline Drug 1 has an aqueous solubility of from 60 to 80 μg/mL. Drug 1 was mixed in a solvent together with a “medium fine” (AQUOT-MF) grade of HPMCAS (manufactured by Shin Etsu) to form a spray solution. For Example 1, the spray solution comprised 1.25 wt % Drug 1, 3.75 wt % HPMCAS, and 95 wt % acetone. For Example 2, the spray solution comprised 7.5 wt % Drug 1, 7.5 wt % HPMCAS, 4.25 wt % water, and 80.75 wt % acetone. These solutions were then spray-dried by directing an atomizing spray using a two-fluid external-mix spray nozzle at 2.7 bar (Example 1) or 3.0 bar (Example 2) at a feed rate of 200 g/min into the stainless-steel chamber of a Niro spray-dryer, using nitrogen as the drying gas, maintained at a temperature of 180° C. (Example 1) or 195° C. (Example 2) at the inlet at a flow rate of about 1900 gm/min; the drying gas and evaporated solvent exited the dryer at 70° C.

The resulting amorphous solid dispersions were collected via a cyclone and then dried in a Gruenberg solvent tray-dryer by spreading the spray-dried particles onto polyethylene-lined trays to a depth of not more than 1 cm and then drying them at 40° C. for at least 8 hours. After drying, the solid dispersions of Example 1 contained 25 wt % Drug 1, and the solid dispersions of Example 2 contained 50 wt % Drug 1.

Control C1 consisted of the polymer HPMCAS-MF alone, as received from the manufacturer.

### Example 3

The solid dispersions of Examples 1 and 2, as well as Control C1, were administered to respective aqueous solutions, and the resulting aqueous solutions were evaluated using light-scattering methods to demonstrate the presence of polymer/drug assemblies in solution. For dynamic light scattering (DLS) analysis, 400 mg of the solid dispersion of Example 1 and 200 mg of Example 2 were each added to respective tubes containing 50 mL PBS and then equilibrated to 37° C. for two hours. In both of these solutions, the total amount of active Drug 1 in PBS would have been 2000 μg/mL if all of the drug had dissolved. After 2 hours, 2 mL of solution was removed and centrifuged at 13,000 G for one minute, to remove precipitated material, leaving free drug, free polymer, and polymer/drug assemblies in solution. Dynamic light-scattering (based on diffusion of particles) of the supernatant of each of the centrifuged solutions was measured using a PSS-NICOMP 380 Submicron Particle Sizer, and the size of any drug and polymer particles in the solution was calculated. The mean particle sizes (hydrodynamic radius) for the bulk of particles in solution are shown in Table 1. (The value reported is a volume-weighted mean, assuming a gaussian size distribution, with approximately 85% of the particle volume being within about 30% of the reported size.)

For static light scattering (StLS) analysis, the solid dispersions of Examples 1 and 2 were each added to PBS equilibrated to 37° C. for two hours. In both of the resulting solutions, the total amount of active Drug 1 in PBS would have been 2000 μg/mL if all of the drug had dissolved. Two hours after adding the solid dispersions to PBS solution, the supernatant was centrifuged for 5 minutes at 13,000 G and analyzed. Light-scattering was measured using a Horiba LA-910. The particle size distribution (radius of gyration) for StLS was calculated based on the angular dependency of scattered light from two separate light sources. The StLS measurement was more sensitive to light scattering by a few large particles than the DLS measurement, so the median particle size is used rather than the mean. Results are also shown below in Table 1.

TABLE 1

| | DLS Mean | StLS Median |
|---|---|---|
| | Particle Size | Particle Size |
| Example | (nm) | (nm) |
| 1 | 79 | 80 |
| 2 | 83 | * |
| C1 | 12 | no data |

*large particles were present, which disproportionately scatter light and skew the calculated particle size to larger diameters; this suggests the aggregation of the smaller particles into larger groups.

When no drug was present (Control 1), small particles about 10 to 20 nm in size were present due to aggregation of the polymer (HPMCAS-MF) with itself, likely as a result of its amphiphilicity, which renders the polymer only sparingly water soluble. For solutions containing Drug 1 (Examples 1 and 2), particles were present with an average size of about 80 nm. This demonstrates the formation of polymer/drug assemblies in solution.

### Example 4

To determine the concentration and nature of the polymer/drug assemblies formed in solution, aqueous solutions prepared using the solid dispersions of Example 1 and Example 2 were analyzed using HPLC and NMR. Solid dispersions were added to PBS at 37° C. and mixed using a vortex mixer to form the polymer/drug assemblies. A sufficient amount of dispersion was added so that the total amount of Drug 1 in PBS would have been 500 μ/mL if all of the drug had dissolved. Ninety minutes after the addition of the solid dispersions, samples were centrifuged (13,000 G for 5 minutes). The concentrations of free drug and free polymer in the supernatant were determined by NMR. HPLC was used to determine the amount of total dissolved drug in the supernatant following centrifugation that consists of free drug and drug in polymer/drug assemblies. The centrifuged precipitate was collected and then dissolved in DMSO and analyzed by NMR to obtain the concentrations of drug and polymer in the precipitate. The results are shown in Table 2 below. The amount of drug contained in the polymer/drug assemblies was calculated by subtracting the concentration of free drug in the supernatant from the total dissolved drug, and the amount of polymer contained in the polymer/drug assemblies was calculated by subtracting the free polymer and the polymer in the precipitate from the total polymer added to solution (contained in the solid dispersion).

TABLE 2

| | | | NMR | NMR | | | | | |
|---|---|---|---|---|---|---|---|---|---|
| | Added | Added | Free | Free | HPLC | NMR | NMR | | |
| | Total | HPMCAS- | Drug 1 | Polymer | Total | Drug 1 | Polymer in | Calculated | Calculated |
| | Drug 1 | MF | Conc. in | Conc. in | Dissolved | in Pre- | Precip- | Drug 1 in | Polymer in |
| Ex. | Conc. | Conc. | Solution | Solution | Drug 1 | cipitate | itate | Assemblies | Assemblies |
| No. | (μg/mL) | (μg/mL) | (μg/mL) | (μg/mL) | (μg/mL) | (μg/mL) | (μg/mL) | (μg/mL) | (μg/mL) |
| 1 | 500 | 1500 | 340 | 750 | 490 | 0 | 0 | 150 | 750 |
| 2 | 500 | 500 | 320 | 250 | 500 | 30 | 40 | 180 | 210 |

These results show that for solutions formed by administering the solid dispersion of Example 1, virtually all of the drug remained in solution, either as free drug or as polymer/drug assemblies. The dispersions of Example 1 (25 wt % Drug 1) formed solutions containing polymer/drug assemblies in PBS which contained 17 wt % Drug 1. The dispersions of Example 2 (50 wt % Drug 1) formed solutions containing polymer/drug assemblies in PBS which contained 46 wt % Drug 1.

The data in Table 2 also show that at 90 minutes following administration of the dispersion to aqueous solution, the free drug concentration for both Examples 1 and 2 was about 3-fold the equilibrium solubility of the crystalline Drug 1 (about 120 μg/mL in PBS) and about 2-fold the solubility of amorphous Drug 1 (about 190 μg/mL in PBS). The total dissolved drug concentration for both Examples 1 and 2 was about 4.1-fold and 4.2-fold, respectively, the solubility of crystalline drug. The ratio of free drug to total dissolved drug was 0.69 and 0.64, respectively, for solutions formed from the dispersions of Examples 1 and 2.

### Example 5

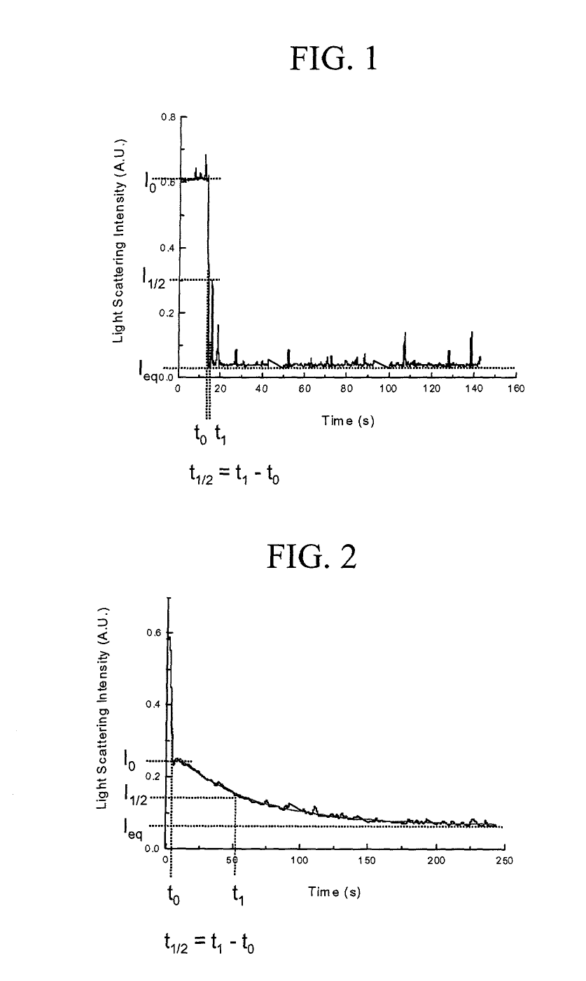

This example describes a “lability assay” that demonstrates that polymer/drug assemblies are a labile drug reservoir capable of replenishing the free drug concentration as it is depleted from solution. In this example, a concentrated bile salt/phospholipid mixture that forms micelles in which Drug 1 is highly soluble was used to rapidly absorb the free drug from solution. The solid dispersions of Examples 1 and 2 were added to PBS at 37° C. and allowed to equilibrate for 90 minutes. The total amount of active Drug 1 in solution would have been 2000 μg/mL if all of the drug had dissolved. Samples were centrifuged for 1 minute at 13,000 G and 1 mL was transferred to a stirred UV cuvette. Light-scattering was measured using a helium/neon laser incident beam at 90° relative to a detector connected to an oscilloscope, which was linked to a computer. After equilibration of the light scattering signal, 1 mL of another solution containing the micelle-forming mixture of bile salt and phospholipid, 100 mg/mL sodium taurocholic acid and 1-palmitoyl-2-oleyl-sn-glycero-3-phosphocholine (NaTC/POPC, with a 4/1 weight ratio) was added to the sample while stirring. The decay of the light-scattering signal was measured as the drug partitioned from the polymer/drug assemblies to the micelles, and as a result, a portion of the polymer/drug assemblies disintegrated, thus leading to a decrease in the light-scattering signal. The decay data was fit to a monoexponential decay model by computer and the time for transfer of the drug from polymer/drug assemblies to micelles in solution was calculated. In lability measurements with polymer/drug assemblies formed in solutions containing solid dispersions of Examples 1 and 2, the decay was extremely rapid (drug from the polymer/drug assemblies rapidly partitioned into the micelles and the polymer/drug assemblies rapidly disintegrated). The time for this to occur was reported as a t_{½} or half-life value, and is given as <1 second (which was at the limit of detection of the experiment). The decay data is shown for Example 1 in FIG. 1, and the results are summarized in Table 3.

TABLE 3

| Example | Lability |
|---|---|
| No. | (sec) |
| 1 | <1 |
| 2 | <1 |

The results show that upon absorption of drug (in this case into micelles), the polymer/drug assemblies formed using the solid dispersions of Examples 1 and 2 can rapidly dissociate to release free drug.

### Example 6

This example demonstrates formation of solid aggregated polymer/drug assemblies. A solid dispersion of 25 wt % Drug 1 and HPMCAS was formed in the manner described in Example 1 (except that the nozzle pressure was 2.9 bar) and aqueous buffer solution was prepared by adding 2 g ammonium carbonate to 200 mL HPLC-grade water, and adjusting the pH to 6.5 using glacial acetic acid. 400 mg of the solid dispersion was added to 50 mL buffer. The resulting solution was stirred for 90 minutes at 37° C., then centrifuged 1 minute at 13,000 G. The supernatant was lyophilized overnight to isolate the polymer/drug assemblies in solid powdered form. The solid aggregated polymer/drug assemblies contained 22.7 wt % Drug 1.

Control 2. Control 2 (C2) consisted of the original 25% Drug 1/HPMCAS-MF dispersion.

### Example 7

This example shows that solid aggregated polymer/drug assemblies provide concentration-enhancement. 15.9 mg of the solid aggregated polymer/drug assemblies of Example 6, or 14.4 mg of C2, was added to a microcentrifuge tube. A sufficient amount of each material was added so that the concentration of drug would have been 2000 μg/mL, if all of the drug had dissolved. The tubes were placed in a 37° C. temperature-controlled chamber, and 1.8 mL PBS at pH 6.5 and 290 mOsm/kg was added to each respective tube. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by high-performance liquid chromatography (HPLC). The contents of each respective tube were mixed on the vortex mixer and allowed to stand undisturbed at 37° C. until the next sample was taken. Samples were collected at 4, 10, 20, 40, 90, and 1200 minutes. The results are shown in Table 4.

TABLE 4

| | | Drug 1 | |
|---|---|---|---|
| | Time | Concentration | AUC |
| Example | (min) | (μg/mL) | (min*μg/mL) |
| 6 | 0 | 0 | 0 |
| | 4 | 1813 | 3,600 |
| | 10 | 1848 | 14,600 |
| | 20 | 1937 | 33,500 |
| | 40 | 1923 | 72,100 |
| | 90 | 1923 | 168,300 |
| | 1200 | 1956 | 2,321,100 |
| C2 | 0 | 0 | 0 |
| | 4 | 661 | 1300 |
| | 10 | 652 | 5,300 |
| | 20 | 711 | 12,100 |
| | 40 | 776 | 26,900 |
| | 90 | 837 | 67,300 |
| | 1200 | 1185 | 1,189,500 |

The concentrations of drug obtained in these samples were used to determine the maximum concentration of drug in Cₘₐₓ₉₀ and the area under the concentration-versus-time curve during the initial ninety minutes (“AUC₉₀”). The results are shown in Table 5.

TABLE 5

| | | | AUC₉₀ |
|---|---|---|---|
| Example | Cₘₐₓ₉₀(μg/mL) | (Cₘₐₓ₉₀/C_{dose}*100 | (min*μg/mL) |
| 6 | 1937 | 97 | 168,300 |
| C2 | 837 | 42 | 67,300 |

As can be seen from the data, the solid aggregated polymer/drug assemblies of Example 6 provided greater concentration-enhancement than the original dispersion, with the Cₘₐₓ₉₀ for the test composition being 2.3-fold that of the control and the AUC₉₀ being 2.5-fold that of the control. The higher ratio of Cₘₐₓ₉₀/C_{dose} obtained for the composition of the invention shows the higher fraction of drug in solubilized form.

### Examples 8-12

The formation of polymer/drug assemblies in solution was conducted by adding a mixture of amorphous drug particles formed by spray-drying and polymer particles to an aqeuous solution. Varying amounts of amorphous Drug 1 and the polymer, HPMCAS-MF, were added to PBS. The amorphous Drug 1 particles were formed by spray-drying a solution of 7 wt % Drug 1 in a solvent of 95/5 (v/v) acetone/water. For Examples 8-12, 0.2, 2.0, 20.0, 50.0, and 100.0 mg, respectively, of amorphous Drug 1 were added to a mortar with 200 mg of HPMCAS-MF particles with an average particle size of 4 μm, and the two types of particles were mixed using a spatula. Each mixture of drug and polymer particles was then added to 100 mL of PBS and then equilibrated to 37° C. for two hours.

Control 3 (C3) consisted of the polymer HPMCAS-MF alone in solution (that is, no drug added).

### Example 13

This example demonstrates that polymer/drug assemblies form only when drug is added to a solution in a form and amount sufficient to generate a drug concentration in excess of the equilibrium solubility of the drug. Two hours after the drug and polymer of Examples 8-12 (and Control 3) were added to PBS, 30 mL of each solution was removed and centrifuged at 13,000 G for five minutes. Dynamic light-scattering of the supernatant of each of the centrifuged solutions was measured as described in Example 3, and the size of any drug and polymer particles in the solution was calculated. Concentrations of drug and polymer in solution, and the corresponding mean particle size for the bulk of particles in solution are shown in Table 6.

TABLE 6

| | | | Mean |
|---|---|---|---|
| | Drug 1 | HPMCAS-MF | Particle |
| Example | Concentration | Concentration | Size |
| No. | (mg/mL)* | (mg/mL) | (nm) |
| 8 | 0.002 | 2.0 | 18 |
| 9 | 0.02 | 2.0 | 16 |
| 10 | 0.2 | 2.0 | 14 |
| 11 | 0.5 | 2.0 | 84 |
| 12 | 1.0 | 2.0 | 83 |
| C3 | 0 | 2.0 | 12 |

*assuming all drug in solution dissolved

When no drug is present (Control 3), small particles about 10 to 20 nm in size are present due to aggregation of the polymer (HPMCAS-MF) alone. At low concentrations of amorphous Drug 1 (0.002 to 0.2 mg/mL), light-scattering shows only small particles in solution (about 10 to 20 nm in size), like that present for polymer alone. For concentrations of amorphous Drug 1 (≧0.5 mg/mL) greater than the equilibrium solubility of amorphous Drug 1 (190 μg/mL in PBS), particles were present with an average size of about 80 to 85 nm. This demonstrates the formation of polymer/drug assemblies in solution, and shows that the amount of drug required for formation of polymer/drug assemblies is approximately equal to or greater than the amorphous drug solubility.

### Example 14

To determine the composition of polymer/drug assemblies for the solutions of Examples 10, 11, and 12 above (Table 6), the solutions were analyzed using HPLC and NMR, as described in Example 4. The results are shown in Table 7 below.

TABLE 7

| | | | NMR | NMR | | | | | |
|---|---|---|---|---|---|---|---|---|---|
| | Added | Added | Free | Free | HPLC | NMR | NMR | | |
| | Total | HPMCAS- | Drug 1 | Polymer | Total | Drug 1 | Polymer in | Calculated | Calculated |
| | Drug 1 | MF | Conc. in | Conc. in | Dissolved | in Pre- | Precip- | Drug 1 in | Polymer in |
| Ex. | Conc. | Conc. | Solution | Solution | Drug 1 | cipitate | itate | Assemblies | Assemblies |
| No. | (μg/mL) | (μg/mL) | (μg/mL) | (μg/mL) | (μg/mL) | (μg/mL) | (μg/mL) | (μg/mL) | (μg/mL) |
| 10 | 200 | 2000 | 170 | 1770 | 200 | 0 | 0 | 30 | 230 |
| 11 | 500 | 2000 | 270 | 1370 | 460 | 50 | 90 | 190 | 540 |
| 12 | 1000 | 2000 | 300 | 1000 | 540 | 380 | 540 | 240 | 460 |

The results show that for solutions of Examples 11 and 12, there is sufficient amount of amphiphilic polymer for the total dissolved drug to exceed the maximum concentration provided by amorphous drug alone (about 190 μg/mL in PBS). In addition, the enhancement is sustained for a long period of time.

The data in Table 7 also show that for drug concentrations exceeding the solubility limit of Drug 1 (Examples 11 and 12), a significant amount of the total dissolved drug is contained in polymer/drug assemblies (>40%). The free drug concentration for Example 11 is about 2.25-fold the solubility of the crystalline Drug 1 (about 120 μg/mL in PBS) and about 1.4-fold the solubility of amorphous Drug 1 (about 190 μg/mL in PBS). In addition, the free drug concentration for Example 12 is about 2.5-fold the solubility of the crystalline Drug 1 and about 1.6-fold the solubility of amorphous Drug 1.

### Example 15

This example shows the concentration-enhancement provided by the formation of polymer/drug assemblies in solution is sustained for a long period of time. In a dissolution test, amorphous Drug 1 was added to PBS at 37° C., with varying concentrations of HPMCAS-MF. First, HPMCAS-MF was dissolved in PBS with stirring at 37° C. and 1.5 mL of this solution was placed in a microcentrifuge tube. Next, 15 mg of amorphous Drug 1 was added to this solution and mixed, centrifuged, and sampled as described in Example 7. The control was amorphous Drug 1 in PBS solution without HPMCAS-MF. The amounts of Drug 1 and HPMCAS used in each dissolution test, and the resulting drug concentrations measured at 1.5 hours and 20 hours, are shown in Table 8.

TABLE 8

| | | HPLC Total | HPLC Total |
|---|---|---|---|
| Drug 1 | HPMCAS-MF | Dissolved | Dissolved |
| Concentration | Concentration | Drug 1 | Drug 1 |
| Added | Added | at 90 min. | at 1200 min. |
| (mg/mL) | (mg/mL) | (μg/mL) | (μg/mL) |
| 10 | 20 | 8099 | 7451 |
| 10 | 10 | 7453 | 5431 |
| 10 | 5 | 4928 | 1550 |
| 10 | 1 | 487 | 203 |
| 10 | 0.5 | 447 | 289 |
| 10 | 0 | 224 | 196 |
| (amorphous | | | |
| drug alone) | | | |

These Examples show that when high concentrations of amorphous drug are added to solutions of the amphiphilic polymer HPMCAS that high concentrations of total dissolved drug are obtained and are sustained for at least 1200 minutes. In addition, the total dissolved drug increases with increasing concentrations of polymer, reaching a value of more than 7400 μg/mL at 20 mg/mL polymer. This value is more than 37-fold that obtained at 1200 minutes in the absence of the polymer.

### Examples 16-17

These samples demonstrate the use of different polymer grades. The solid dispersion of Example 16 was made using HPMCAS-LF, and the solid dispersion of Example 17 was made using HPMCAS-HF, using the following procedures. These polymer grades, all made by Shin-Etsu, have different levels of substitution, as shown, along with the HPMCAS-MF grade (used in Examples 1 to 15) in Table 8A.

TABLE 8A

| | Substitution* (%) | | | |
|---|---|---|---|---|
| HPMCAS Grade | Hydroxypropyl | Methyl | Acetate | Succinate |
| HPMCAS-MF | 5.0-9.0 | 21.0-25.0 | 7.0-11.0 | 10.0-14.0 |
| HPMCAS-HF | 6.0-10.0 | 22.0-26.0 | 10.0-14.0 | 4.0-8.0 |
| HPMCAS-LF | 5.0-9.0 | 20.0-24.0 | 5.0-9.0 | 14.0-18.0 |

* Substitution per manufacturer's specification

Examples 16 and 17 were prepared by first forming solutions containing 2.5 wt % Drug 1, 2.5 wt % polymer, 90 wt % acetone, and 5 wt % water, which were then spray-dried by pumping the solution into a spray-dryer apparatus at a rate of 1.3 mL/min. The spray solution was metered using a Cole Parmer 74900 series rate-controlling syringe pump. The solution was pumped into a Spraying Systems Co. two-fluid nozzle, model number SULA, with nitrogen as the atomizing gas. The nitrogen was heated to a temperature of 100° C. at a flow rate of about 1 scfm. The solution was sprayed from the top of an 11-centimeter diameter, 46 cm tall, stainless steel cylindrical chamber. The resulting solid amorphous dispersion was collected on Whatman® 1 filter paper, dried under vacuum, and stored in a dessicator.

### Example 18

Aqueous solutions were prepared from the solid dispersions of Example 16 and Example 17, and analyzed using NMR, using the same procedure described in Example 4. A sufficient amount of the solid dispersions was added to PBS at 37° C. so that the concentration of drug would have been 2000 μg/mL, if all of the drug had dissolved. The concentrations of free drug in the supernatant of the resulting solutions were determined by NMR. The results are shown in Table 9.

TABLE 9

| | NMR Free Drug 1 |
|---|---|
| | Concentration |
| Example | in Solution |
| No. | (μg/mL) |
| 16 | 450 |
| 17 | 324 |

The data in Table 9 show the Drug 1 enhancement in free drug concentration provided by the presence of the polymer/drug assemblies. The NMR free drug concentration for Example 16 is 3.8-fold the solubility of the crystalline Drug 1 (120 μg/mL), and the free drug concentration for Example 17 is 2.7-fold the solubility of the crystalline Drug 1.

### Example 19

This example demonstrates polymer/drug assemblies formed from a blend of polymers. A 25 wt % Drug 1 solid dispersion was formed with a blend of the polymers HPMCAS-MF and HPMC. These dispersions were added to aqueous solutions to form polymer/drug assemblies. To form the dispersion, a spray solution containing 1.2 wt % Drug 1, 2.4 wt % HPMC E3 Prem LV, 1.2 wt % HPMCAS-MF, 85.7 wt % methanol, and 9.5 wt % water was spray-dried using a Niro spray-dryer. The solution was spray-dried by directing an atomizing spray using a two-fluid external-mix spray nozzle at 2.7 bar at a feed rate of 105 g/min into the stainless-steel chamber of the spray-dryer. Nitrogen drying gas was introduced to the dryer at a temperature of 123° C. and a flow rate of about 1900 gm/min; drying gas and evaporated solvent exited the dryer at a temperature of 47° C. The resulting solid dispersion was collected via a cyclone and then dried in a Gruenberg solvent tray-dryer at 40° C. for at least 8 hours.

### Example 20

Solutions formed from the dispersions of Example 19 were analyzed using DLS and NMR, as described in Examples 3 and 4. Samples of the solid dispersion of Example 19 were added to PBS in a sufficient amount so that the total amount of dissolved drug would have been 2000 μg/mL, if all of the drug dissolved. The solution was equilibrated at 37° C. for 60 minutes. After 60 minutes, the sample tubes were centrifuged for 1 minute at 13,000 G and supernatant was diluted 1:6 in methanol. The drug concentration was then analyzed by NMR as in Example 4. The results are shown in Table 10.

TABLE 10

| | | NMR Free Drug 1 |
|---|---|---|
| | DLS Mean | Concentration |
| Example | Particle Size | in Solution |
| No. | (nm) | (μg/mL) |
| 19 | 496 | 345 |

The solution contained polymer/drug assemblies of about 500 nm. In addition, the polymer/drug assemblies provided a free drug concentration for the solution formed from the dispersion of Example 19 that was 2.9-fold the solubility of the crystalline Drug 1.

### Examples 26-28

These examples demonstrate polymer/drug assemblies formed by dissolving solid amorphous dispersions of drug in non-cellulosic polymers in aqueous solutions. Solid dispersions were made with Drug 1 using the amphiphilic, hydroxyl-functional vinyl copolymer, vinyl acetate/vinyl alcohol copolymer (VAVAC). Three grades of VAVAC were used to form dispersions: (1) 80% hydrolyzed (20% of vinyl alcohol repeat units acetylated) (“VAVAC-20%”), average molecular weight 9,000-10,000 daltons (Aldrich Chem. Co., #36,062-7); (2) 87-89% hydrolyzed (about 12% of vinyl alcohol repeat units acetylated) (“VAVAC-12%”), average molecular weight 13,000-23,000 daltons (Aldrich Chem. Co., #36,317-0); and (3) 98% hydrolyzed (2% of vinyl alcohol repeat units acetylated) (“VAVAC-2%”), average molecular weight 13,000-23,000 daltons (Aldrich Chem. Co., #34,840-6). To form the solid dispersions of Examples 26-28, solutions containing Drug 1 and polymer in a solvent were spray-dried by pumping each solution into a “mini” spray-drier apparatus as described in Examples 16 and 17. Table 12 summarizes the variables for the dispersions of Examples 26-28.

TABLE 12

| | | | | Spray | |
|---|---|---|---|---|---|
| | | Drug | | Solution | |
| Example | | Conc. | | Solids | Spray |
| No. | Drug | (%) | Polymer | (%) | Solvent |
| 26 | 1 | 25 | VAVAC-20% | 1.3 | 4/1 MeOH/H2O |
| 27 | 1 | 25 | VAVAC-12% | 1.0 | 1.8/1 MeOH/H2O |
| 28 | 1 | 25 | VAVAC-2% | 1.5 | 1/1 MeOH/H2O |

### Example 29

The solid dispersions of Examples 26-28, and Controls 5 and 6 (consisting of VAVAC-20% or VAVAC-12% alone), were added to PBS equilibrated to 37° C. and then analyzed by dynamic light scattering (DLS). A sufficient amount of the dispersions of Examples 26-28 were added so that the total amount of Drug 1 in PBS would have been 2000 mg/mL if all of the drug had dissolved. Two hours after the solid dispersions and Controls were added to PBS, 1 mL of solution was removed and centrifuged at 13,000 G for five minutes. Dynamic light-scattering of the supernatant of each of the centrifuged solutions was measured as described in Example 3, and the size of any drug and polymer particles in the solution was calculated. The mean particle sizes for the bulk of particles in solution are shown in Table 13.

TABLE 13

| | DLS Mean |
|---|---|
| | Particle Size |
| Example No. | (nm) |
| 26 | 20 |
| (Drug 1/VAVAC-20%) | |
| 27 | 574 |
| (Drug 1/VAVAC-12%) | |
| 28 | 885 |
| (Drug 1/VAVAC-2%) | |
| C5 | 12 |
| (VAVAC-20%) | |
| C6 | 5 |
| (VAVAC-12%) | |

When no drug is present (Controls 5 and 6), small particles about 10 nm in size were present due to aggregation of the polymer, likely as a result of its amphiphilicity. For solutions containing Drug 1 (formed from the dispersions of Examples 26-28), larger particles were present. This demonstrates the formation of polymer/drug assemblies in solution.

### Examples 30 and 31

These examples demonstrate polymer/drug assemblies formed by dissolution of dispersions of a second drug in polymer. Amorphous solid dispersions of 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (Drug 2) and HPMCAS-MF were made by first mixing Drug 2 in a solvent together with HPMCAS-MF to form a spray solution. For Example 30, the spray solution comprised 1 wt % Drug 2, 9 wt % HPMCAS, and 90 wt % acetone. For Example 31, the spray solution comprised 2.5 wt % Drug 2, 7.5 wt % HPMCAS, and 90 wt % acetone. These spray solutions were then spray-dried by directing an atomizing spray using a two-fluid external-mix spray nozzle at 2.7 bar at a feed rate of 150 g/min into the stainless-steel chamber of a Niro spray-dryer. Nitrogen drying gas at a temperature of 155° C. and about 1900 gm/min was introduced to the dryer; drying gas and evaporated solvent exited the dryer at a temperature of 70° C.

The resulting solid dispersions were collected via a cyclone and then dried in a Gruenberg solvent tray-dryer at 40° C. for at least 8 hours. After drying, the dispersions of Example 30 contained 10 wt % Drug 2, and the dispersions of Example 31 contained 25 wt % Drug 2.

### Example 32

The solutions formed from solid dispersions of Examples 30 and 31 were evaluated using light-scattering methods to demonstrate the formation of polymer/drug assemblies in solution. For DLS analysis, 500 mg of the dispersions of Example 30 or 200 mg of Example 31 were each added to 50 mL PBS equilibrated to 37° C. for two hours. In both of these experiments, the total amount of active Drug 2 in PBS would have been 1000 μg/mL, if all of the drug had dissolved. Light-scattering was measured using a PSS-NICOMP 380 Submicron Particle Sizer as described in Example 3. The mean particle sizes (hydrodynamic radius) for the bulk of particles in solution are shown in Table 14.

For static light scattering (StLS) analysis, the solid dispersions of Examples 30 and 31 were each added to PBS equilibrated to 37° C for two hours. After centrifuging for 1 minute at 13,000 G, light-scattering was measured using a Horiba LA-910 as described in Example 3. The particle size distribution (radius of gyration) for StLS is calculated and results are shown below in Table 14.

TABLE 14

| | DLS Mean | StLS Median |
|---|---|---|
| | Particle Size | Particle Size |
| Example | (nm) | (nm) |
| 30 | 674 | 100 |
| 31 | 419 | 90 |
| C1 | 12 | —* |
| (from Table 1) | | |

*value is less than detection limit

The data in Table 14 shows particles were present with an average size larger than polymer alone, C1. This demonstrates the formation of polymer/drug assemblies in solution.

### Example 33

To determine the composition of the polymer/drug assemblies, solutions formed from the dispersions of Example 30 and Example 31 in PBS were analyzed using HPLC and NMR, as described in Example 4. Because of the low solubility of Drug 2 (approximately 0.010 μg/ml), it was not possible to directly observe free drug by NMR measurements in PBS without the presence of NaTC/POPC, which form micelles in which Drug 2 is highly soluble. To measure the free drug concentration using NMR, 2 wt % NaTC/POPC was added to the solution. By measuring the amount of drug that partitioned into NaTC/POPC micelles, the concentration of free drug in PBS was calculated using the equation:

D_{M}=Kₚ×D_{f}×V_{M}

where D_{M} is the concentration of drug in micelles (measured by NMR), Kₚ is the partition coefficient for drug partitioning between water and micelles (30,000), D_{f} is the free drug concentration in PBS, and V_{M} is the volume ratio occupied by micelles in the PBS solution (cm³ micelles/cm³ solution=0.021). Drug 2 solid dispersions of Examples 30 and 31 were added to solution to give a total of 1,000 μg/mL Drug 2 (if all of the Drug 2 dissolved). The results are shown in Table 15 below. The amount of polymer contained in the polymer/drug assemblies was calculated by subtracting the free polymer and the polymer in the precipitate from the total polymer added to solution (contained in the solid dispersions). The solution formed from the dispersions of Example 30 (10 wt % Drug 2) formed polymer/drug assemblies in PBS which contained 81 wt % Drug 2. The solutions formed from the dispersions of Example 31 (25 wt % Drug 2) formed polymer/drug assemblies in PBS which contained 55 wt % Drug 2.

TABLE 15

| | | | NMR | NMR | | | | | |
|---|---|---|---|---|---|---|---|---|---|
| | Added | Added | Free | Free | HPLC | NMR | NMR | Calculated | |
| | Total | HPMCAS- | Drug 2 | Polymer | Total | Drug 2 | Polymer in | Drug 2 in | Calculated |
| | Drug 2 | MF | Conc. in | Conc. in | Dissolved | in Pre- | Precip- | Assem- | Polymer in |
| Ex. | Conc. | Conc. | Solution | Solution | Drug 2 | cipitate | itate | blies | Assemblies |
| No. | (μg/mL) | (μg/mL) | (μg/mL) | (μg/mL) | (μg/mL) | (μg/mL) | (μg/mL) | (μg/mL) | (μg/mL) |
| 30 | 1000 | 9000 | 0.085 | 8770 | 920 | 80 | 10 | 920 | 220 |
| 31 | 1000 | 3000 | 0.085 | 2180 | 730 | 270 | 220 | 730 | 600 |

The data in Table 15 show that essentially all of the total dissolved drug is contained in polymer/drug assemblies. In addition, the free drug concentration in PBS for both Examples 30 and 31 is about 8.5-fold the solubility of the crystalline Drug 2 (0.010 g/mL).

### Example 34

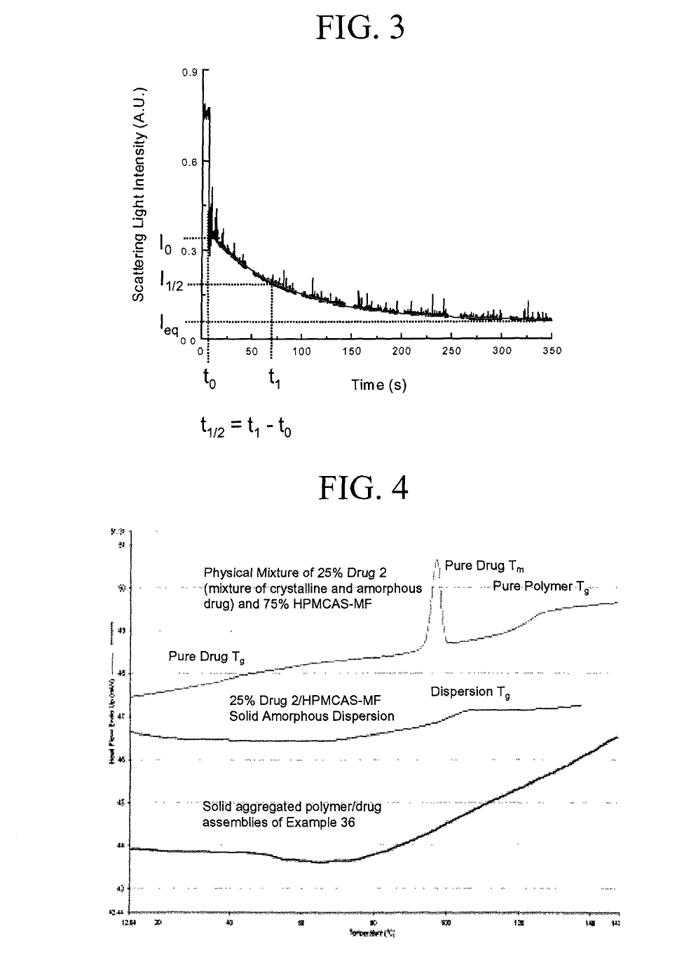

A “lability assay” was performed using the solutions formed from the dispersions of Examples 30 and 31 to show that polymer/drug assemblies could rapidly dissociate and release free drug as it was depleted by partitioning of drug into bile salt/phospholipid micelles, similar to that described in Example 5. A concentrated bile salt/phospholipid mixture was chosen to provide a micellular phase in which Drug 2 is highly soluble as described in Example 5. A sufficient amount of the dispersions of Examples 30 and 31 was added to solution such that if all of the drug dissolved, a total concentration of 1000 μg/mL would have resulted. Light scattering intensity as a function of time is shown in FIGS. 2 and 3, The lability or t_{½} calculated for each experiment is shown in Table 16. The lability or t_{½} was calculated by determining the time required for the light scattering to drop halfway from its value, following addition of the bile salt/phospholipid solution (that is, I₀ at the value at t₀ shown in FIGS. 2 and 3) to its equilibrium value.

The results indicate that the polymer/drug assemblies were very labile in that the time required for half of the drug to dissociate is only about 60 seconds.

TABLE 16

| | Lability |
|---|---|
| Example No. | (sec) |
| 30 | 60 |
| 31 | 60 |

### Example 3

This example demonstrates the free drug concentration enhancement provided by polymer/drug assemblies by measuring the amount of free drug that partitions into micelles. The dispersions of Examples 30 and 31 were added to PBS with varying amounts of NaTC/POPC, and the concentration of drug in NaTC/POPC micelles was measured by NMR spectroscopy. For these experiments, the dispersions were added to solution in a sufficient amount so that the total concentration of Drug 2 would have been 2,000 μ/mL if all of the drug had dissolved. Crystalline Drug 2 (Control 7, or C7) was also added to PBS with varying amounts of NaTC/POPC for comparison. The results are shown in Table 17.

TABLE 17

| | Drug 2 Concentration (g/mL) | | | |
|---|---|---|---|---|
| | NaTC/POPC | NaTC/POPC | NaTC/POPC | NaTC/POPC |
| | Conc. | Conc. | Conc. | Conc. |
| Example | 0 wt % | 0.5 wt % | 1.0 wt % | 2.0 wt % |
| 30 | 0.085* | 12 | 22 | 53 |
| 31 | 0.085* | 12 | 28 | 48 |
| C7 | 0.010** | 2 | 4 | 6 |

*calculated
**value obtained in a separate experiment

The free drug concentration with no micelles present was calculated as follows. The slope of the Drug 2 concentration versus NaTC/POPC concentration line was determined for both the solutions formed from the dispersions of Examples 30 and 31 and the solution formed from crystalline Drug 2. The ratio of these slopes was 8.5, showing the concentration enhancement provided by the formation of polymer/drug assemblies. The Drug 2 solubility in aqueous solution without micelles was determined to be approximately 0.010 μg/mL in separate experiments. The enhancement of 8.5 was used to calculate a concentration of 0.085 μg/mL for free drug in aqueous solution formed from the dispersions of Examples 30 and 31 without micelles.

### Example 36

This example demonstrates formation of a solid aggregated polymer/drug assembly. Polymer/drug assemblies were first formed in aqueous solution by adding a solid dispersion (similar to Example 31) to a buffer solution, as described in Example 6. The solid dispersion was made using a process similar to that described for Example 31, with the following exceptions: the sprayed solution comprised 1.7 wt % Drug 2, 5.1 wt % HPMCAS-MF, and 93.2 wt % acetone; the feed rate was 190 g/min, and the temperature of the inlet drying gas was 135° C. and the drying gas and evaporated solvent exited at 50° C. After drying, the solid dispersion contained 25 wt % Drug 2. Following addition of the solid dispersion to the buffer, centrifuged supernatant was frozen in liquid nitrogen and then lyophilized overnight to isolate the solid aggregated polymer/drug assemblies in powdered form. The solid aggregated polymer/drug assemblies contained 17.8 wt % Drug 2.

Control C8 consisted of the original 25% wt % Drug 2/HPMCAS-MF dispersion.

### Example 37

The concentration-enhancement provided by the solid aggregated polymer/drug assemblies of Example 36 was demonstrated in a dissolution test. For this test, 10.1 mg of the solid aggregated polymer/drug assemblies of Example 36, or 7.2 mg of C8, was added to a microcentrifuge tube, and the test was performed as described in Example 7. A sufficient amount of each material was added so that the concentration of drug would have been 1000 μg/mL, if all of the drug had dissolved. The results are shown in Table 18.

TABLE 18

| | | Drug 1 | |
|---|---|---|---|
| | Time | Concentration | AUC |
| Example | (min) | (μg/mL) | (min*μg/mL) |
| 36 | 0 | 0 | 0 |
| | 4 | 832 | 1,700 |
| | 10 | 840 | 6,700 |
| | 20 | 833 | 15,000 |
| | 40 | 816 | 31,500 |
| | 90 | 810 | 2,200 |
| | 1200 | 365 | 724,300 |
| C8 | 0 | 0 | 0 |
| | 4 | 99 | 200 |
| | 10 | 298 | 1,400 |
| | 20 | 502 | 5,400 |
| | 40 | 659 | 17,000 |
| | 90 | 686 | 50,600 |
| | 1200 | 306 | 601,200 |

The concentrations of drug obtained in these samples were used to determine the values of Cₘₐₓ₉₀, the ratio of (Cₘₐₓ₉₀/C_{dose})*100 and AUC₉₀. The results are summarized in Table 19.

TABLE 19

| | | | AUC₉₀ |
|---|---|---|---|
| Example | Cₘₐₓ₉₀(μg/mL) | (Cₘₐₓ₉₀/C_{dose})*100 | (min*μg/mL) |
| 36 | 840 | 84 | 72,200 |
| C8 | 686 | 69 | 50,600 |

As can be seen from the data, the polymer/drug assemblies of Examples 36 provided greater concentration-enhancement than the original dispersion, with a Cₘₐₓ₉₀ for the test composition 1.2-fold that of the control and the AUC₉₀ being 1.4-fold that of the control. The higher ratio of Cₘₐₓ₉₀/C_{dose} obtained for the composition of the invention shows the higher fraction of drug in solubilized form.

### Example 38

This example demonstrates polymer/drug assemblies formed from the ionizable cellulosic polymer cellulose acetate phthalate (CAP). To form the solid dispersion, a spray solution containing 0.8 wt % Drug 2, 7.2 wt % CAP, and 92 wt % acetone was spray-dried using a Niro spray-dryer. The solution was spray-dried by directing an atomizing spray using a two-fluid external-mix spray nozzle at 2.8 bar at a feed rate of 200 g/min into the stainless-steel chamber of the spray-dryer. Nitrogen drying gas was introduced to the dryer at a temperature of 180° C. and a flow rate of about 1900 gm/min. Drying gas and evaporated solvent exited the dryer at a temperature of 67° C. The resulting particles were collected via a cyclone and then dried in a Gruenberg solvent tray-dryer at 40° C. for at least 8 hours.

Control C9 consisted of the polymer CAP alone in solution.

### Example 39

A solution formed from the solid dispersions of Example 38 (or Control C9) was analyzed using light-scattering to demonstrate the formation of polymer/drug assemblies in solution. For DLS analysis, 200 mg of the dispersion of Example 38 was added to 50 mL PBS, stirred, and then equilibrated to 37° C. for two hours. A sufficient amount of dispersion was added so that the total Drug 2 concentration would have been 400 μg/mL, if all of the drug were to dissolve. Light-scattering was measured using a PSS-NICOMP 380 Submicron Particle Sizer as described in Example 3. The mean particle sizes (hydrodynamic radius) for the bulk of particles in solution are shown in Table 20.

TABLE 20

| | DLS Mean |
|---|---|
| | Particle Size |
| Example No. | (nm) |
| 38 | 246 |
| C9 | 13 |

In solutions containing Drug 2, particles were present with a mean size larger than polymer alone, C9. This demonstrates the formation of polymer/drug assemblies in solution.

### Example 40

This example demonstrates polymer/drug assemblies formed from a blend of polymers. A 25 wt % Drug 2 solid dispersion was formed with a polymer blend of HPMCAS-MF and HPMC and then added to aqueous solution to form polymer/drug assemblies. To form the solid dispersion, a spray solution containing 0.6 wt % Drug 2, 1.2 wt % HPMC E3 Prem LV, 0.6 wt % HPMCAS-MF, 88.1 wt % methanol, and 9.5 wt % water was prepared. The solution was spray-dried using a Niro spray-dryer by directing an atomizing spray using a two-fluid external-mix spray nozzle at 2.7 bar at a feed rate of 100 g/min into the stainless-steel chamber of the spray-dryer. Drying gas was introduced at a flow rate of about 1800 g/min and at a temperature of 168° C. and the drying gas and evaporated solvent exited the chamber 44° C. The dispersion was collected via a cyclone and then dried in a Gruenberg solvent tray-dryer at 40° C. for at least 8 hours.

### Example 41

Aqueous solutions formed from the solid dispersion of Example 40 were analyzed using DLS and NMR, as described in Examples 3 and 4. For NMR analysis of Drug 2 in micelles, the solid dispersion (1000 μg/mL total Drug 2 concentration, if all of the drug dissolved) was added to PBS at 37° C. containing 2% NaTC/POPC. The free drug concentration was analyzed as in Example 33. Results are shown in Table 21.

TABLE 21

| | DLS Mean | NMR Free Drug 2 | |
|---|---|---|---|
| | Particle | Conc. in 2% | |
| Example | Size | NaTC/POPC-PBS | Solubility |
| No. | (nm) | (μg/mL) | Enhancement |
| 40 | 265 | 71 | 12 |

The polymer/drug assemblies provided a free drug concentration that was 12-fold the solubility of the crystalline Drug 2 in 2% NaTC/POPC in PBS (6 μg/mL).

### Example 42

This example demonstrates formation of solid aggregated polymer/drug assemblies by spray-drying. A solid dispersion formed in the manner described in Example 30 was added to a buffer to first form an aqueous solution containing polymer/drug assemblies. The buffer contained 1 wt % ammonium carbonate in HPLC-grade water, adjusted to pH 6.5 using glacial acetic acid. A sufficient amount of solid dispersion was added to the buffer solution to contain 1 wt % dispersion (that is, 1 gm of dispersion was added to 99 gm buffer solution). The buffer was stirred, then centrifuged 1 minute at 13,000 G. The supernatant was spray-dried to isolate the solid aggregated polymer/drug assemblies in powdered form. To spray-dry the buffer solution, the solution was atomized using a two-fluid external-mix spray nozzle at a feed rate of 76 g/min into the stainless-steel chamber of a Niro spray-dryer. Drying gas was introduced at a flow rate of about 1900 g/min and a temperature of 280° C.; and gas and evaporated solvent exited the dryer at 92° C. The aggregated polymer/drug assemblies contained 8 wt % Drug 2. value for C_{max,90} is less than 0.1 μg/mL, the detection limit of the experiment.

The concentrations of drug obtained in these samples were used to determine the values of Cₘₐₓ₉₀ and AUC₉₀. The results are shown in Table 23. As can be seen from the data, the solid aggregated polymer/drug assemblies of Example 42 provided concentration-enhancement over that of crystalline drug alone, with a Cₘₐₓ₉₀ for the test composition of more than 9000-fold that of the control and the AUC₉₀ of more than 76,000-fold that of the control.

TABLE 23

| | | AUC₉₀ |
|---|---|---|
| Example | Cₘₐₓ₉₀(μg/mL) | (min*μg/mL) |
| 42 | 925 | 76,100 |
| C10 | <0.10 | <1 |

### Example 45

Differential scanning calorimetry (DSC) was used to examine the solid aggregated polymer/drug assemblies of Example 36, a 25% Drug 2/HPMCAS-MF dispersion, and a physical mixture of HPMCAS-MF and Drug 2 (containing both amorphous and crystalline drug forms). The samples were equilibrated for a minimum of 18 hours at 0% relative humidity. Sample pans were crimped and sealed in a dry environmental chamber, then loaded into the furnace of a Perkin-Elmer Pyris 1 DSC with a robotic arm. The samples were heated at 10° C./min up to 150° C. The DSC scans of these three samples are shown in FIG. 4.

As shown in FIG. 4, the DSC scan of the physical mixture (upper curve) shows the glass transition temperature (T_{g}) of the drug (38° C.), the T_{g} of the polymer (122° C.) , and the sharp melting peak (97° C.) of crystalline Drug 2. The solid dispersion (middle curve) shows a T_{g} (96° C.) that is intermediate to the drug and polymer T_{g}s, indicating that the drug and polymer are homogeneously combined. In the DSC scan of the solid aggregated polymer/drug assemblies of Example 36, a T_{g} is not observed, indicating a physical state distinct from that of crystalline drug, a physical mixture of drug and polymer, or a homogeneous dispersion.

### Examples 46 and 47

This example demonstrates polymer/drug assemblies with another drug. Amorphous solid dispersions of 2-phenanthrenecarboxamide, 4b,5,6,7,8,8a,9,10-octahydro-7-hydroxy-N-[(2-methyl-3-pyridinyl)methyl]-4b-(phenylmethyl)-7-(3,3,3-trifluoropropyl)-, (4bS,7S,8aR)- (Drug 3) and HPMCAS-MF were made by mixing Drug 3 in a solvent together with HPMCAS-MF to form a spray solution. For Example 46, the solution comprised 0.042 wt % Drug 3, 0.373 wt % HPMCAS, and 99.585 wt % 1/1 ethyl acetate/methanol. For Example 47, the solution comprised 0.2 wt % Drug 3, 0.6 wt % HPMCAS, and 99.2 wt % acetone. The dispersions were spray-dried by pumping the solution into a “mini” spray-dryer apparatus at a rate of 1.3 mL/min. as described for Examples 16 and 17. After drying, Example 46 SDD contained 10 wt % Drug 3, and the dispersion of Example 47 contained 25 wt % Drug 3.

### Example 48

Solutions formed using the dispersions of Examples 46 and 47 were evaluated using light scattering to demonstrate the formation of polymer/drug assemblies in solution. For DLS analysis, 100 mg of the dispersion of Example 46 or 40 mg of the dispersion of Example 47 were added to respective tubes containing 50 mL PBS and equilibrated to 37° C. for two hours. Sufficient dispersion was added so that the total Drug 3 concentration for these solutions would have been 2 mg/mL, if all of the drug had dissolved. Light-scattering was measured using a PSS-NICOMP 380 Submicron Particle Sizer as described in Example 3. The mean particle sizes (hydrodynamic radius) for the bulk of particles in solution are shown in Table 24.

TABLE 24

| | DLS Mean |
|---|---|
| | Particle Size |
| Example No. | (nm) |
| 46 | 832 |
| 47 | 731 |

In solutions containing Drug 3, particles are present with a mean size larger than polymer alone (C1, Table 1). This demonstrates the formation of polymer/drug assemblies in solution.

### Example 49

To determine the composition of the polymer/drug assemblies, solutions formed using dispersions of Example 46 and Example 47 in PBS were analyzed using HPLC and NMR, as described in Example 4. Because of the low solubility of Drug 3 (0.004 μg/ml), it was not possible to directly observe free drug by NMR measurements. To measure the free drug concentration using NMR, 2 wt % NaTC/POPC was added to the PBS solution, and the free drug concentration in PBS without NaTC/POPC was calculated as described in Example 33. The results are shown in Table 25 below.

TABLE 25

| | | | NMR | NMR | | | | | |
|---|---|---|---|---|---|---|---|---|---|
| | Added | Added | Free | Free | HPLC | NMR | NMR | Calculated | |
| | Total | HPMCAS- | Drug 3 | Polymer | Total | Drug 3 | Polymer in | Drug 3 in | Calculated |
| | Drug 3 | MF | Conc. in | Conc. In | Dissolved | in Pre- | Precip- | Assem- | Polymer in |
| Ex. | Conc. | Conc. | Solution | Solution | Drug 3 | cipitate | itate | blies | Assemblies |
| No. | (μg/mL) | (μg/mL) | (μg/mL) | (μg/mL) | (μg/mL) | (μg/mL) | (μg/mL) | (μg/mL) | (μg/mL) |
| 46 | 200 | 1800 | 2.0* | 1443 | 119 | 81 | 278 | 117 | 79 |
| 47 | 200 | 600 | 2.1* | 370 | 37 | — | 479 | — | — |

The dispersion of Example 46 (10 wt % Drug 3) formed polymer/drug assemblies which contained 60 wt % Drug 3.

The data in Table 25 show that approximately 98 wt % of the total dissolved drug is contained in polymer/drug assemblies. In addition, the free drug concentration for both Examples 46 and 47 was about 500-fold the solubility of the crystalline Drug 3.

### Examples 50-52

These examples demonstrate the formation of polymer/drug assemblies of Drug 3. and CAP. Solid dispersions of 10, 25, and 50 wt % Drug 3. were formed with CAP by spray-drying and the dispersions added to aqueous buffer solutions to form polymer/drug assemblies. To form the solid dispersion of Example 50, a solution containing 0.1 wt % Drug 3, 0.9 wt % CAP, and 99 wt % acetone was spray-dried using a Niro spray drier. The solution feed rate was 100 gm/min. Drying gas was introduced into the dryer at a flow rate of about 1900 gm/min and a temperature of 90° C.; and drying gas and evaporated solvent exited at a temperature of 50° C. To form the solid dispersion of Example 51, a solution containing 0.1 wt % Drug 3, 0.3 wt % CAP, and 99.6 wt % acetone was spray-dried using a Niro spray drier. The solution feed rate was 90 gm/min. Drying gas was introduced into the dryer at a flow rate of about 1900 gm/min and a temperature of 90° C.; and drying gas and evaporated solvent exited at a temperature of 50° C. To form the solid dispersion of Example 52, a solution containing 0.1 wt % Drug 3, 0.1 wt % CAP, and 99.8 wt % acetone was spray-dried using a mini spray drier. The solution feed rate was 1.3 mLs/min with a drying gas temperature of 100° C. and a flow rate of 1.0 SCFM. The solid dispersions of Examples 50 and 51 were collected via a cyclone and then dried in a Gruenberg solvent tray-dryer; the solid dispersion of Example 52 were dried in a vacuum dessicator.

### Example 53

To determine the composition of the polymer/drug assemblies, solutions were formed using the dispersions of Examples 50, 51, and 52 and were analyzed using NMR. Solid dispersions were added to PBS containing 2 wt % NaTC/POPC. A sufficient amount of Drug 3. dispersion was added to solution such that if all of the drug had dissolved, the total Drug 3. concentration would have been 1600 μg/mL. The concentration of drug in micelles was measured by NMR spectroscopy. The results are shown in Table 26 below.

TABLE 26

| | | | NMR Free |
|---|---|---|---|
| | Added Total | | Drug 3 |
| | Drug 3 | Added CAP | Concentration |
| Example | Concentration | Concentration | in Solution |
| No. | (μg/mL | (μg/mL) | (μg/mL) |
| 50 | 1600 | 14,400 | 2.4 |
| 51 | 1600 | 4,800 | 2.1 |
| 52 | 1600 | 1,600 | 1.6 |

The data in Table 26 show that the free drug concentration for Examples 50, 51, and 52 is about 400-fold, 500-fold, and 600-fold, respectively, the solubility of the crystalline Drug 3. (about 0.004 μg/mL).

### Examples 54-63

These examples demonstrate polymer/drug assemblies formed from a fourth drug. Solid aggregated polymer/drug assemblies of the low-solubility drug 5-(2-(4-(3-benzisothiazolyl)-piperazinyl)ethyl-6-chlorooxindole (Ziprasidone) (Drug 4) and an amphiphilic polymer were prepared. Ziprasidone (mesylate salt) and a “high granular” (AQUOT-HG) grade of HPMCAS (HPMCAS-HG, manufactured by Shin Etsu), or PVP, 29,000 Dalton molecular weight (Aldrich), were dissolved in a warm (50° C.) organic solvent to form a clear solution. Other additives, if present, were included after the initial solution was prepared. Solution compositions for Examples 54-63 are listed in Table 27.

The solid aggregated polymer/drug assemblies were prepared by mixing the above organic solution with an aqueous receptor solution comprised of 50 mM sodium 3-(4-morpholinyl)propanesulfonate, 150 mM NaCl, pH 7.4 (MOPS buffer.) After 10 minutes, the mixture was centrifuged at 13,000 G for 1 minute, and the aqueous layer was decanted from the precipitated polymer/drug assembly.

TABLE 27

| | Zipra- | | | | | | | |
|---|---|---|---|---|---|---|---|---|
| | sidone | | Polymer | | Solvent | | | Additive |
| Ex. | Mesylate | | Amount | | Amount | | | Amount |
| No. | (mg) | Polymer | (g) | Solvent | (g) | Treatment | Additive | (g) |
| 54 | 800 | HPMCAS-HG | 1.67 | NMP | 10.33 | moist | none | — |
| | | | | | | precipitate | | |
| 55 | 800 | HPMCAS-HG | 1.67 | NMP | 10.33 | lyophilized | none | — |
| | | | | | | precipitate | | |
| 56 | 800 | HPMCAS-HG | 1.67 | NMP | 10.33 | lyophilized | none | — |
| | | | | | | precipitate | | |
| 57 | 800 | HPMCAS-HG | 1.67 | DMSO | 10.33 | moist | none | — |
| | | | | | | precipitate | | |
| 58 | 800 | HPMCAS-HG | 1.67 | DMSO | 10.33 | lyophilized | none | — |
| | | | | | | precipitate | | |
| 59 | 800 | PVP | 1.67 | NMP | 10.33 | moist | none | — |
| | | | | | | precipitate | | |
| 60 | 800 | HPMCAS-HG | 1.67 | NMP | 10.33 | moist | H₂O | 2.31 |
| | | | | | | precipitate | Tween 80 | 0.15 |
| 61 | 800 | HPMCAS-HG | 1.67 | NMP | 10.33 | moist | H₂O | 2.31 |
| | | | | | | precipitate | SLS* | 0.015 |
| 62 | 800 | HPMCAS-HG | 1.67 | NMP | 10.33 | moist | HPMC | 0.065 |
| | | | | | | precipitate | K100M | |
| 63 | 240 | HPMCAS-HG | 0.501 | NMP | 3.099 | moist | none | — |
| | | sodium | | | | precipitate | | |
| | | salt | | | | | | |

### Examples 64 through 67

Solid polymer/drug assemblies of ziprasidone and polymer (either HPMCAS, PVP, or polyvinyl alcohol [PVA]) were prepared by dissolving ziprasidone free-base in a warm (45° C.) 90:10 (volume) mixture of NMP and water. For Examples 64 and 15 65, HPMCAS-HG and a neutralizing amount of aqueous KOH was dissolved in the ziprasidone solution. For Example 66, PVA was dissolved in the ziprasidone solution. For Example 67, PVP was dissolved in the ziprasidone solution. The polymer/drug assemblies were precipitated by slowly adding a solution of 20 20:80 NMP:water (vol:vol) followed by water (containing a surfactant for Examples 65-67). The solid polymer/drug assemblies were isolated by centrifuging at 21,000 G for 10 minutes, pouring off the supernatant, and air-drying the solid product. The resultant polymer/drug assemblies contained 80-95 wt % ziprasidone. The solution compositions for Examples 64-67 are summarized in Table 28.

TABLE 28

| | Zipra- | | | | | | | |
|---|---|---|---|---|---|---|---|---|
| | sidone | | Polymer | | Solvent | | | Additive |
| Ex. | Mesylate | | Amount | | Amount | | | Amount |
| No. | (mg) | Polymer | (g) | Solvent | (g) | Treatment | Additive | (g) |
| 64 | 1.00 | HPMCAS-HG | 2.5 | NMP | 93 | air-dried | H₂O | 100 |
| | | potassium | | H₂O | 10 | precipitate | | |
| | | salt | | | | | | |
| 65 | 0.50 | HPMCAS-HG | 1.25 | NMP | 46.5 | air-dried | SLS | 0.008 |
| | | potassium | | H₂O | 5 | precipitate | H₂O | 100 |
| | | salt | | | | | | |
| 66 | 0.50 | PVA | 0.102 | NMP | 46.5 | air-dried | SLS | 0.003 |
| | | | | H₂O | 5 | precipitate | H₂O | 100 |
| 67 | 0.50 | PVP | 0.102 | NMP | 46.5 | air-dried | SLS | 0.003 |
| | | | | H₂O | 5 | precipitate | H₂O | 100 |

### Control C11

Control C11 consisted of a solid dispersion of ziprasidone and HPMCAS. Ziprasidone free-base was dissolved in warm (50° C.) tetrahydrofuran. This solution was combined with a solution of HPMCAS-HG dissolved in methanol with a neutralizing amount of aqueous NaOH. The combined solution comprised 0.65 wt % ziprasidone, 1.9 wt % HPMCAS (sodium salt), 39.0 wt % methanol, 15.0 wt % water, and 43.9 wt % tetrahydrofuran. The solution was then spray-dried by directing an atomizing spray using a pressure nozzle at 11.7 bar. The feed rate was 140 g/min into the stainless steel chamber of a Niro spray-dryer, and the temperature of the drying gas was 200° C. at a flow rate of about 1900 g/min, while the drying gas and evaporated solvent exited at a temperature of 74° C . The resultant amorphous dispersion was collected via a cyclone and then dried in a vacuum desiccator overnight at room temperature. After drying, the solid dispersion contained 25 wt % ziprasidone.

### Controls C12 and C13

Controls C12 and C13 consisted of solid dispersions of ziprasidone and HPMCAS that were prepared in a manner similar to C11, except that the drug/polymer ratios were different, and the neutralizing amount of aqueous NaOH was not added. After drying, the solid dispersions contained 30 wt % (Control C12) and 10 wt % (Control C13) ziprasidone.

### Example 68

This example shows that solid aggregated polymer/drug assemblies provide concentration enhancement. Approximately 30 mg of a moist sample of the solid polymer/drug assembly of Example 54, or 56.08 mg of C11, was added to a microcentrifuge tube. The tubes were placed in a 37° C. temperature-controlled chamber, and 450 μL of MOPS buffer was added to each of the tubes. The samples were mixed using a vortex mixer for 60 seconds. The samples were centrifuged at 13,000 G for 1 minute, and 10 μL of the resultant supernatant solution was removed, diluted with 1.5 mL methanol, and analyzed using HPLC. The contents of each respective tube were again mixed by vortexing and allowed to stand undisturbed at 37° C. until the next sample was taken. Samples were collected at 10, 60, and 1200 minutes. The results are shown in Table 29.

TABLE 29

| | | Ziprasidone |
|---|---|---|
| | Time | Concentration |
| Example | (min) | (μg/mL) |
| 54 | 0 | 0 |
| | 10 | 400 |
| | 60 | — |
| | 1200 | 1087 |
| C11 | 0 | 0 |
| | 10 | 245 |
| | 60 | 392 |
| | 1200 | 144 |

As can be seen from the data, the solid polymer/drug assemblies provided greater concentration enhancement than the solid dispersion. The Cₘₐₓ₁₀ for the solid polymer/drug assemblies of Example 54 was 1.6-fold that of C11, and the concentration at 1200 minutes (C₁₂₀₀) was 7.54-fold that of C11.

### Example 69

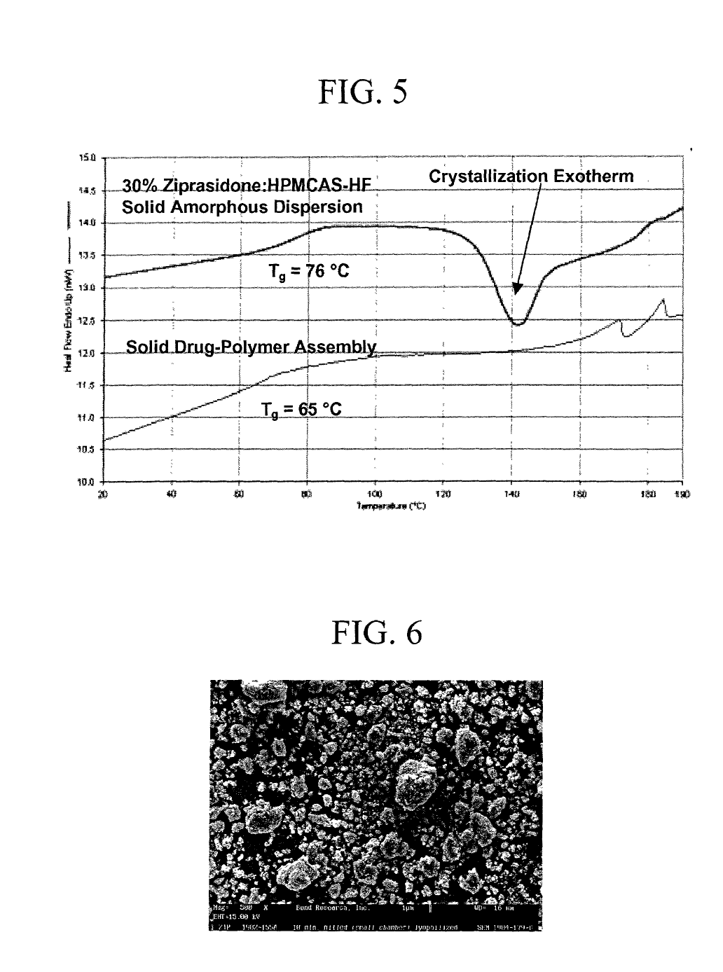

This example shows the difference in thermal properties between the polymer/drug assemblies of Example 55 and the solid drug dispersion C12. DSC scans were performed as described in Example 45. As shown in FIG. 5, the materials have different T_{g}s, and the polymer/drug assembly shows no exotherm corresponding to crystallization of the drug. This demonstrates that drug crystallization is inhibited in the solid aggregated polymer/drug assemblies relative to the corresponding polymer/drug dispersion. Thus, the drug crystallization rate in the solid aggregated polymer/drug assemblies is less than the drug crystallization rate in the corresponding dispersion.

### Example 70

This example shows that the addition of sodium lauryl sulfate (SLS) to the polymer and drug solution prior to forming the solid aggregated polymer/drug assemblies modifies the solid polymer/drug assemblies such that they provide further concentration enhancement. Approximately 30 mg of a moist sample of the solid polymer/drug assembly of Example 61 was added to a microcentrifuge tube. Control 14 consisted of a sample prepared as described in Example 61 except that SLS was not included; approximately 25 mg of C14 was also added to a microcentrifuge tube. The tubes were placed in a 37° C. temperature-controlled chamber, and 250 μL of MOPS buffer were added to the tubes. The samples were mixed using a vortex mixer for 60 seconds. The samples were centrifuged at 13,000 G for 1 minute, and 10 μL of the resulting supernatant solution was removed, diluted with 1.5 mL methanol, and analyzed using HPLC. The contents of each respective tube were again mixed by vortexing and allowed to stand undisturbed at 37° C. until the next sample was taken. Samples were collected at 10, 60, and 1200 minutes. The 1200-minute samples were then filtered through 0.65 μm centrifuging filter tubes and the filtrates were sampled. The results are shown in Table 30.

TABLE 30

| | | Ziprasidone |
|---|---|---|
| | Time | Concentration |
| Example | (min) | (μg/mL) |
| 61 | 0 | 0 |
| | 10 | 511 |
| | 60 | 774 |
| | 1200 | 657 |
| | 1200- | 444 |
| | filtered | |
| C14 | 0 | 0 |
| | 10 | 301 |
| | 60 | 398 |
| | 1200 | 508 |
| | 1200- | 266 |
| | filtered | |

As can be seen from the data, the SLS-modified solid polymer/drug assemblies provided greater concentration enhancement than the unmodified assemblies. The Cₘₐₓ₆₀ of the SLS-modified solid polymer/drug assemblies was 1.9-fold that of C14, C₁₂₀₀ was 1.3-fold that of C14, and C_{1200filtered} was 1.7-fold that of C14.

### Example 71

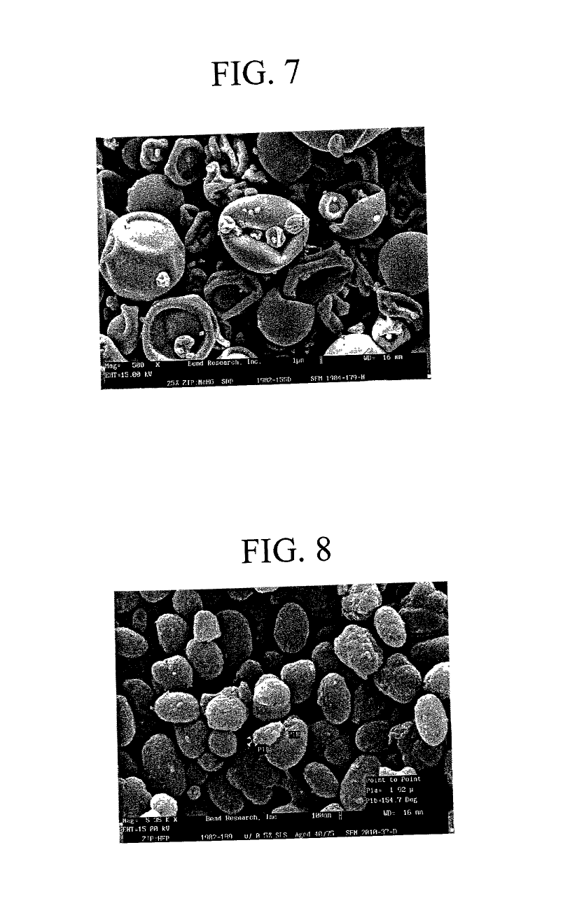

This example shows the difference in particle size between the processed (milled) solid aggregated polymer/drug assembly of Example 56 and the solid dispersion of C11. FIGS. 6 and 7 show the scanning electron micrographs (SEM) of Example 56 and Control C11, respectively. As can be seen from these SEMs, the average particle size for the solid aggregated polymer/drug assembly is much smaller than for the analogous dispersion. This solid aggregated polymer/drug assembly more readily disperses in aqueous solution relative to the corresponding dispersion.

### Example 72

This example demonstrates that solid aggregated polymer/drug assemblies prepared by low-concentration precipitation provide concentration enhancement. A 16.11-mg sample of the solid aggregated polymer/drug assemblies of Example 64 (prepared from a 3.4 wt % solution) was added to a microcentrifuge tube. For comparison, approximately the same amount of the solid polymer/drug assemblies of Example 54 (prepared from a 23.9 wt % solution) was added to a microcentrifuge tube. The tubes were placed in a 37° C. temperature-controlled chamber, and 250 μL of MOPS buffer were added to the tubes. The samples were mixed using a vortex mixer for 60 seconds. The samples were centrifuged at 13,000 G for 1 minute, and 10 μL of the resulting supernatant solution was removed, diluted with 1.5 mL methanol, and analyzed using HPLC. The contents of each respective tube were again mixed by vortexing and allowed to stand undisturbed at 37° C. until the next sample was taken. Samples were collected at 10, 60, and 1200 minutes. The results are shown in Table 31.

TABLE 31

| | | Ziprasidone |
|---|---|---|
| | Time | Concentration |
| Example | (min) | (μg/mL) |
| 64 | 0 | 0 |
| | 10 | 1233 |
| | 60 | 4540 |
| | 1200 | 3173 |
| 54 | 0 | 0 |
| | 10 | 318 |
| | 60 | 499 |
| | 1200 | 520 |

As can be seen from the data, the solid aggregated polymer/drug assemblies prepared by low-concentration precipitation provided greater concentration enhancement than the solid aggregated polymer/drug assemblies precipitated from a higher- concentration solution. The Cₘₐₓ₆₀ for Example 64 was 9.1-fold that of Example 54, and C₁₂₀₀ was 6.1-fold that of Example 54.

### Example 73

This example shows that modification of solid aggregated polymer/drug assemblies prepared by low-concentration precipitation provides concentration enhancement. A 16.9-mg sample of the solid aggregated polymer/drug assemblies of Example 65 (prepared with SLS) was placed in a microcentrifuge tube. For comparison, approximately the same amount of the solid aggregated polymer/drug assemblies of Example 64 (prepared without SLS) was added to a microcentrifuge tube. The tubes were placed in a 37° C. temperature-controlled chamber, and 250 μL of MOPS buffer were added to the tubes. The samples were mixed using a vortex mixer for 60 seconds. The samples were centrifuged at 13,000 G for 1 minute, and 10 μL of the resulting supernatant solution was removed, diluted with 1.5 mL methanol, and analyzed using HPLC. The supernatants were removed and fresh receptor was added to each tube. The contents of each respective tube were again mixed by vortexing and allowed to stand undisturbed at 37° C. until the next sample was taken. Samples were collected at 10, 10 minutes post-redissolution (20), 60, and 1200 minutes. The results are shown in Table 32.

TABLE 32

| | | Ziprasidone |
|---|---|---|
| | Time | Concentration |
| Example | (min) | (μg/mL) |
| 65 | 0 | 0 |
| | 10 | 1908 |
| | 20 | 6238 |
| | 60 | 2888 |
| | 1200 | 3118 |
| 64 | 0 | 0 |
| | 10 | 3089 |
| | 20 | 1764 |
| | 60 | 2274 |
| | 1200 | 826 |

As can be seen from the data, the solid polymer/drug assemblies modified with SLS (Example 65) provided greater concentration enhancement than the solid polymer/drug assemblies without SLS. The Cₘₐₓ₆₀ for Example 65 was 2.0-fold that of Example 64, and the C₁₂₀₀ was 3.8-fold that of Example 64.

### Example 74

This example demonstrates that solid aggregated polymer/drug assemblies prepared by low-concentration precipitation provide concentration enhancement in PBS. Sample of the solid aggregated polymer/drug assemblies of Example 65 or solid dispersion of C13 were added to microcentrifuge tubes. It all of the ziprasidone completely dissolved, the concentrations of the solutions would have been 400 μg/mL. The tubes were placed in a 37° C. temperature-controlled chamber, and 1.25 mL of phosphate buffered saline (PBS) at pH 6.5 was added to the tubes. The samples were mixed using a vortex mixer for 60 seconds. The samples were centrifuged at 13,000 G for 1 minute, and 50 μL of the resulting supernatant solution was removed, diluted with 1.5 mL methanol, and analyzed using HPLC. The contents of each respective tube were again mixed by vortexing and allowed to stand undisturbed at 37° C. until the next sample was taken. Sample were collected at 4, 10, 30, 60, 90, and 1200 minutes. The results are shown in Table 33.

TABLE 33

| | | Ziprasidone |
|---|---|---|
| | Time | Concentration |
| Example | (min) | (μg/mL) |
| 65 | 0 | 0 |
| | 4 | 16 |
| | 10 | 33 |
| | 30 | 87 |
| | 60 | 66 |
| | 90 | 57 |
| | 1200 | 198 |
| C13 | 0 | 0 |
| | 4 | 47 |
| | 10 | 53 |
| | 20 | 46 |
| | 60 | 31 |
| | 90 | 25 |
| | 1200 | 18 |

As can be seen from the data, the solid aggregated polymer/drug assemblies provided greater concentration enhancement upon dissolution in PBS relative to that provided by the solid dispersion. The Cₘₐₓ₁₀ of the solid polymer/drug assemblies was 2.1-fold that of C13, and C₁₂₀₀ was 11-fold that of C13.

### Example 75

This example shows that different polymers may be used to form polymer/drug assemblies with ziprasidone. A 16.9-mg sample of the solid aggregated polymer/drug assemblies of Example 66 (prepared with PVA), or approximately the same amount of the solid aggregated polymer/drug assemblies of Example 67 (prepared with PVP) was placed in a microcentrifuge tube. The tubes were placed in a 37° C. temperature-controlled chamber, and 250 μL of MOPS buffer were added to the tubes. The samples were mixed using a vortex mixer for 60 seconds. The samples were centrifuged at 13,000 G for 1 minute, and 10 μL of the resulting supernatant solution was removed, diluted with 1.5 mL methanol, and analyzed using HPLC. The supernatants were removed and fresh receptor was added to each tube. The contents of each respective tube were again mixed by vortexing and allowed to stand undisturbed at 37° C. until the next sample was taken. Samples were collected at 10, 20, 60, and 1200 minutes. The results are shown in Table 34.

TABLE 34

| | | Ziprasidone |
|---|---|---|
| | Time | Concentration |
| Example | (min) | (μg/mL) |
| 66 | 0 | 0 |
| | 10 | 290 |
| | 20 | 300 |
| | 60 | 944 |
| | 1200 | 370 |
| 67 | 0 | 0 |
| | 10 | 174 |
| | 20 | 507 |
| | 60 | 335 |
| | 1200 | 448 |

As can be seen from the data, the solid polymer/drug assemblies containing PVA or PVP provided concentration-enhancement of ziprasidone.

### Example 76

This example shows the solid-state morphology of the polymer/drug assemblies prepared by low-concentration precipitation. FIG. 8 is a SEM of Example 65 showing that the solid particles have a fairly narrow particle size and shape, with an average particle diameter of about 2 to 3 μm.

### Example 77

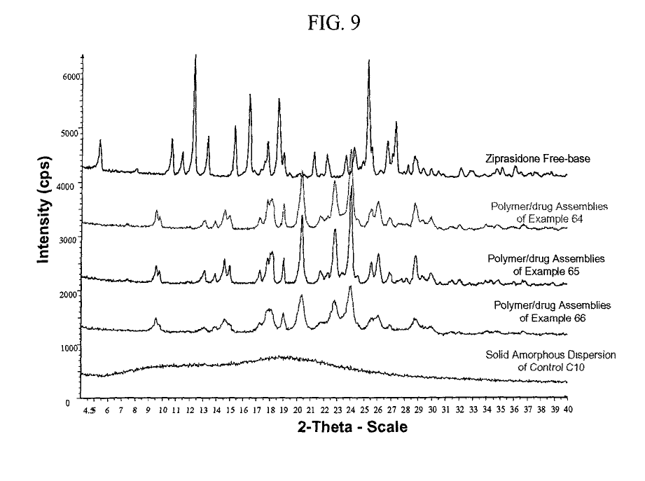

This example shows the semi-ordered nature of the low-concentration precipitated polymer/drug assemblies (Examples 64-66) as demonstrated by their powder X-ray diffraction patterns. FIG. 9 shows the powder X-ray diffraction patterns for (1) crystalline ziprasidone free base, (2) the polymer/drug assemblies of Example 64, (3) the polymer/drug assemblies of Example 65, (4) the polymer/drug assemblies of Example 66, and (5) a solid amorphous dispersion of 10 wt % ziprasidone in HPMCAS. These data indicate that the polymer/drug assemblies are “semi-ordered,” that is, they are more ordered than the solid amorphous dispersion of ziprasidone in HPMCAS but less ordered than crystalline ziprasidone free-base. This is apparent from the width of the scattering lines which are broader than crystalline drug but narrower than amorphous drug.

### Example 78

Solid aggregated polymer/drug assemblies of Drug 2 were prepared by dissolving 282.5 mg Drug 2 and 1.1224 g HPMCAS-MF in 28.0 mL methanol. The solution was heated to 37° C. and stirred for 45 minutes to dissolve the drug and polymer. The solution was cooled to 22° C., and 11 mL water was added slowly (solution remained clear). The polymer/drug solution was added dropwise to 172 mL 50 mM ammonium acetate. Next, the solution was centrifuged at 13,000 G for 3 minutes. The solid aggregated polymer/drug assemblies were isolated by lyophilizing the supernatant overnight. The resultant solid aggregated polymer/drug assemblies contained 16.7 wt % Drug 2.

### Example 79

The concentration-enhancement provided by the solid aggregated polymer/drug assemblies of Example 78 was demonstrated in a dissolution test. For this test, 10.8 mg of the solid aggregated polymer/drug assemblies of Example 78 was added to a microcentrifuge tube. A sufficient amount of material was added so that the concentration of Drug 2 would have been 1000 μg/mL, if all of the drug had dissolved. The test was performed as described in Example 7. The results are shown in Table 35. Control 8 (25 wt % Drug 2/HPMCAS dispersion) is included in Table 35 for comparison.

TABLE 35

| | | Drug 2 | |
|---|---|---|---|
| | Time | Concentration | AUC |
| Example | (min) | (μg/mL) | (min*μg/mL) |
| Ex 78 | 0 | 0 | 0 |
| | 4 | 768 | 1,500 |
| | 10 | 740 | 6,100 |
| | 20 | 701 | 13,300 |
| | 40 | 762 | 27,900 |
| | 90 | 665 | 63,600 |
| | 1200 | 372 | 639,100 |
| C8 | 0 | 0 | 0 |
| | 4 | 99 | 200 |
| | 10 | 298 | 1,400 |
| | 20 | 502 | 5,400 |
| | 40 | 659 | 17,000 |
| | 90 | 686 | 50,600 |
| | 1200 | 306 | 601,200 |

The concentrations of drug obtained in these samples were used to determine the values of Cₘₐₓ₉₀ and AUC₉₀. The results are summarized in Table 36.

TABLE 36

| | | AUC₉₀ |
|---|---|---|
| Example | Cₘₐₓ₉₀(μg/mL) | (min*μg/mL) |
| Ex 78 | 768 | 63,600 |
| C8 | 686 | 50,600 |

As can be seen from the data, the solid aggregated polymer/drug assemblies of Example 78 provided greater concentration enhancement than the control dispersion, with the AUC₉₀ for the test composition being 1.26-fold that of the control.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. An aqueous solution comprising:
(a) a low-solubility drug;
(b) an amphiphilic polymer that is at least partially dissolved in said solution;
(c) a portion of said drug and a portion of said polymer being present in said solution as amorphous polymer/drug assemblies, said polymer/drug assemblies having a diameter of from 20 nm to 5000 nm;
(d) said solution having a total dissolved drug concentration of at least 2-fold that of an equilibrium concentration of said drug provided by a control composition consisting of an equivalent amount of said drug in crystalline form alone;
(e) said solution having a free drug concentration of at least 1.5-fold that of said equilibrium concentration provided by said control composition; and
(f) said amphiphilic polymer is non-cellulosic.

2. An aqueous solution comprising:
(a) a low-solubility drug;
(b) an amphiphilic polymer that is at least partially dissolved in said solution;
(c) a portion of said drug and a portion of said polymer being present in said solution as amorphous polymer/drug assemblies, said polymer/drug assemblies having a diameter of from 20 nm to 5000 nm;
(d) said solution having a total dissolved drug concentration of at least 2-fold that of an equilibrium concentration of said drug provided by a control composition consisting of an equivalent amount of said drug in crystalline form alone;
(e) said solution having a free drug concentration of at least 1.5-fold that of said equilibrium concentration provided by said control composition; and
(f) said amphiphilic polymer comprises a neutral cellulosic polymer provided said amphiphilic polymer is not solely hydroxypropyl methyl cellulose.

3. An aqueous solution comprising:
(a) a low-solubility drug;
(b) an amphiphilic polymer that is at least partially dissolved in said solution;
(c) a portion of said drug and a portion of said polymer being present in said solution as amorphous polymer/drug assemblies, said polymer/drug assemblies having a diameter of from 20 nm to 5000 nm;
(d) said solution having a total dissolved drug concentration of at least 2-fold that of an equilibrium concentration of said drug provided by a control composition consisting of an equivalent amount of said drug in crystalline form alone;
(e) said solution having a free drug concentration of at least 1.5-fold that of said equilibrium concentration provided by said control; and
(f) said amphiphilic polymer is an ionizable cellulosic polymer, provided said cellulosic polymer is not solely hydroxypropyl methyl cellulose acetate succinate.

4. The solution of any one of claims 1-3 wherein said free drug concentration is at least 2-fold said equilibrium concentration.

5. The solution of claim 4 wherein said free drug concentration is at least 4-fold said equilibrium concentration.

6. The solution of any one of claims 1-3 wherein said total dissolved drug concentration is at least 4-fold said equilibrium concentration.

7. The solution of any one of claims 1-3 wherein said total dissolved drug concentration is at least 10-fold said equilibrium concentration.

8. The solution of any one of claims 1-3 wherein said polymer/drug assemblies have a half-life for disassociation of said drug from said polymer/drug assemblies of less than 1000 seconds.

9. The solution of any one of claims 1-3 wherein said polymer drug/assemblies comprise from 1 wt % to 98 wt % drug.

10. The solution of any one of claims 1-3 wherein upon standing at 37° C. but without stirring, at least 25 wt % of said polymer/drug assemblies remain suspended in said solution at least 90 minutes following formation of said solution.

11. The solution of claim 10 wherein at least 50 wt % of said polymer/drug assemblies remain suspended in said solution at least 90 minutes following formation of said solution.

12. The solution of any one of claims 1-3 wherein said amphiphilic polymer has an aqueous solubility of at least about 0.1 mg/ml.

13. The solution of claim 12 wherein said aqueous solubility of said amphiphilic polymer is less than about 100 mg/ml.

14. The solution of claim 1 wherein said amphiphilic polymer is ionizable.

15. The solution of claim 14 wherein said amphiphilic polymer is selected from the group consisting of carboxylic acid functionalized polymethacrylates, carboxylic acid functionalized polyacrylates, amine-functionalized polyacrylates and polymethacrylates, high molecular weight proteins and carboxylic acid functionalized starches.

16. The solution of claim 14 wherein said amphiphilic polymer is a copolymer comprising a hydrophobic repeat unit and a hydrophilic repeat unit.

17. The solution of claim 1 wherein said amphiphilic polymer is neutral.

18. The solution of claim 17 wherein said amphiphilic polymer is selected from the group consisting of vinyl polymers and copolymers having at least one substituent selected from the group comprising hydroxyl, alkylacyloxy, and cyclicamido; vinyl copolymers of at least one hydrophilic, hydroxyl-containing repeat unit and at least one hydrophobic, alkyl-, or aryl-containing repeat unit; polyvinyl alcohols that have at least a portion of their repeat units in the unhydrolyzed form; polyvinyl alcohol polyvinyl acetate copolymers; polyethylene glycol polypropylene glycol copolymers; polyvinyl pyrrolidone; polyethylene polyvinyl alcohol copolymers; and polyoxyethylene-polyoxypropylene block copolymers.

19. The composition of claim 3 wherein said concentration-enhancing polymer is selected from the group consisting of hydroxypropyl cellulose acetate succinate, hydroxypropyl methyl cellulose acetate, hydroxypropyl methyl cellulose succinate, hydroxypropyl methyl cellulose phthalate, hydroxypropyl methyl cellulose acetate phthalate, hydroxyethyl methyl cellulose, hydroxyethyl methyl cellulose succinate, hydroxyethyl cellulose acetate succinate, hydroxyethyl methyl cellulose acetate succinate, hydroxyethyl methyl cellulose acetate phthalate, hydroxyethyl cellulose acetate, hydroxyethyl ethyl cellulose, carboxymethyl ethyl cellulose, cellulose acetate phthalate, hydroxypropyl cellulose acetate phthalate, methyl cellulose acetate phthalate, ethyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate, carboxyethyl cellulose, and carboxymethyl cellulose.

20. The solution of any one of claims 1-3 wherein said aqueous solution is a use environment.

21. The solution of claim 20 wherein said use environment is in vitro.

22. The solution of claim 20 wherein said use environment is in vivo.

23. The solution of claim 20 wherein said use environment is the gastrointestinal tract of an animal.

24. A method for forming polymer/drug assemblies comprising:
(a) forming a solid amorphous dispersion comprising a low-solubility drug and a non-cellulosic amphiphilic polymer;
(b) administering said dispersion to an aqueous solution in a sufficient amount so as to provide a free drug concentration that exceeds an equilibrium concentration of said drug in said solution obtained by administering said drug in crystalline form alone so as to form said polymer/drug assemblies having a diameter of from 20 nm to 5000 nm.

25. A method for forming polymer/drug assemblies comprising:
(a) forming a solid amorphous dispersion comprising a low-solubility drug and a non-ionizable cellulosic polymer, provided said amphiphilic polymer is not solely hydroxypropyl methyl cellulose;
(b) administering said dispersion to an aqueous solution in a sufficient amount so as to provide a free drug concentration that exceeds an equilibrium concentration of said drug in said solution obtained by administering said drug in crystalline form alone so as to form said polymer/drug assemblies having a diameter of from 20 nm to 5000 nm.

26. A method for forming polymer/drug assemblies comprising:
(a) forming a solid amorphous dispersion comprising a low-solubility drug and an ionizable cellulosic polymer, provided said polymer is not solely hydroxypropyl methyl cellulose acetate succinate;
(b) administering said dispersion to an aqueous solution in a sufficient amount so as to provide a free drug concentration that exceeds an equilibrium concentration of said drug in said solution obtained by administering said drug in crystalline form alone so as to form said polymer/drug assemblies having a diameter of from 20 nm to 5000 nm.

27. A method for forming polymer/drug assemblies comprising:
(a) forming a solid amorphous dispersion comprising a low-solubility drug and a matrix;
(b) administering said dispersion to an aqueous solution in a sufficient amount so as to provide a dissolved drug concentration that at least temporarily exceeds an equilibrium concentration of said drug in said solution obtained by administering said drug in crystalline form alone;
(c) administering an amphiphilic polymer to said solution in a sufficient amount so as to form said polymer/drug assemblies having a diameter of from 20 nm to 5000 nm.

28. A method for forming polymer/drug assemblies comprising:
(a) forming a solid solubility-improved form of a low-solubility drug;
(b) administering said solid solubility improved form of said drug to an aqueous solution in a sufficient amount so as to provide at least temporarily a dissolved drug concentration that exceeds an equilibrium concentration of said drug in said solution obtained by administering said drug in crystalline form alone;
(c) administering an amphiphilic polymer to said solution in a sufficient amount so as to form said polymer/drug assemblies having a diameter of from 20 nm to 5000 nm.

29. A method for forming polymer/drug assemblies comprising:
(a) forming a first solution comprising a low-solubility drug dissolved in a solvent;
(b) administering said first solution to an aqueous solution in a sufficient amount so as to provide at least temporarily a dissolved drug concentration in said aqueous solution that exceeds an equilibrium concentration provided by said drug in crystalline form alone in said solution;
(c) administering an amphiphilic polymer to said solution in a sufficient amount so as to form said polymer/drug assemblies having a diameter of from 20 nm to 5000 nm.

30. The method of any one of claims 24-29 wherein said aqueous solution has a resulting free drug concentration of at least 1.5-fold said equilibrium concentration of said drug in crystalline form alone.

31. The method of claim 30 wherein said resulting free drug concentration is at least 2-fold said equilibrium concentration of said drug in crystalline form alone.

32. The method of any one of claims 24-29 wherein said aqueous solution has a resulting total dissolved drug concentration is at least 2-fold said equilibrium concentration of said drug in crystalline form alone.

33. The method of claim 32 wherein said resulting total dissolved drug concentration is at least 4-fold said equilibrium concentration of said drug in crystalline form alone.

34. The method of any one of claims 24-29 wherein said polymer/drug assemblies have a disassociation time constant of less than 1000 seconds.

35. The method of any one of claims 24-29 wherein said polymer drug/assemblies comprise from 5 wt % to 98 wt % drug.

36. The method of any one of claims 24-29 wherein upon standing at 25° C. but without stirring, at least 25 wt % of said polymer/drug assemblies remain suspended in said solution at least 90 minutes following formation of said solution.

37. The solution of claim 36 wherein at least 50 wt % of said polymer/drug assemblies remain suspended in said solution at least 90 minutes following formation of said solution.

38. The method of any one of claims 24-29 wherein said amphiphilic polymer has an aqueous solubility of from about 0.1 mg/ml.

39. The method of claim 38 wherein said aqueous solubility of said amphiphilic polymer is less than about 100 mg/ml.

40. A pharmaceutical composition comprising solid aggregated polymer/drug assemblies, said solid aggregated polymer/drug assemblies comprising a low-solubility drug and an amphiphilic polymer and, upon administering to an aqueous use environment, said polymer/drug assemblies providing a maximum total dissolved drug concentration in said use environment that is at least 1.1-fold that provided by a control composition consisting of a solid amorphous dispersion of an equivalent amount of said amphiphilic polymer and an equivalent amount of said drug, said solid aggregated polymer/drug assemblies being administered in a sufficient amount so that the ratio of maximum total dissolved drug provided by said control composition to the total amount of drug administered is less than about 0.6.

41. A pharmaceutical composition comprising solid aggregated polymer/drug assemblies, said solid aggregated polymer/drug assemblies comprising a low-solubility drug and an amphiphilic polymer and said drug being present in said solid aggregated polymer/drug assemblies in a semi-ordered, non-crystalline state.

42. The pharmaceutical composition of claim 41 wherein said drug in said semi-ordered state is demonstrated by a physical stability that is improved relative to a control composition comprising a solid amorphous dispersion of said amphiphilic polymer and said drug.

43. The pharmaceutical composition of claim 41 wherein the drug crystallization rate in said composition is less than about 90% the drug crystallization rate in said control composition consisting of amorphous drug alone.

44. A pharmaceutical composition comprising solid aggregated polymer/drug assemblies, said solid aggregated polymer/drug assemblies comprising a low-solubility drug and an amphiphilic polymer, said solid aggregated polymer/drug assemblies being formed by a process comprising forming a solution containing polymer/drug assemblies, wherein a substantial portion of said polymer/drug assemblies in said solution have a diameter of from 20 nm to 5000 nm, and isolating said solid aggregated polymer/drug assemblies from said solution.

45. The composition of claim 44 wherein said amphiphilic polymer has an aqueous solubility of at least about 0.1 mg/ml.

46. The composition of claim 44 wherein said aqueous solubility of said amphiphilic polymer is less than 100 mg/ml.

47. The composition of claim 44 wherein said amphiphilic polymer has a hydrophobic portion and a hydrophilic portion.

48. The composition of claim 44 wherein said polymer is a cellulosic ionizable polymer.

49. The composition of claim 47 wherein said polymer is selected from the group consisting of hydroxypropyl cellulose acetate succinate, hydroxypropyl methyl cellulose acetate, hydroxypropyl methyl cellulose succinate, hydroxypropyl methyl cellulose acetate succinate, hydroxypropyl methyl cellulose phthalate, hydroxypropyl methyl cellulose acetate phthalate, hydroxyethyl methyl cellulose, hydroxyethyl methyl cellulose succinate, hydroxyethyl cellulose acetate succinate, hydroxyethyl methyl cellulose acetate succinate, hydroxyethyl methyl cellulose acetate phthalate, hydroxyethyl cellulose acetate, hydroxyethyl ethyl cellulose, hydroxypropyl methyl cellulose, carboxymethyl ethyl cellulose, cellulose acetate phthalate, hydroxypropyl cellulose acetate phthalate, methyl cellulose acetate phthalate, ethyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate, carboxyethyl cellulose, and carboxymethyl cellulose.

50. The composition of claim 44 wherein said amphiphilic polymer is a non-ionizable cellulosic polymer.

51. The composition of claim 50 wherein said amphiphilic polymer is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

52. The composition of claim 44 wherein said polymer is an ionizable, non-cellulosic polymer.

53. The composition of claim 52 wherein said polymer is selected from the group consisting of carboxylic acid functionalized polymethacrylates, carboxylic acid functionalized polyacrylates, amine-functionalized polyacrylates, amine-fuctionalized polymethacrylates.

54. The composition of claim 44 wherein said amphiphilic polymer is a non-ionizable, non-cellulosic polymer.

55. The composition of claim 54 wherein said amphiphilic polymer is selected from the group consisting of vinyl polymers and copolymers having at least one substituent selected from the group comprising hydroxyl, alkylacyloxy, and cyclicamido; vinyl copolymers of at least one hydrophilic, hydroxyl-containing repeat unit and at least one hydrophobic, alkyl-, or aryl-containing repeat unit; polyvinyl alcohols that have at least a portion of their repeat units in the unhydrolyzed form; polyvinyl alcohol polyvinyl acetate copolymers; polyethylene glycol polypropylene glycol copolymers; polyvinyl pyrrolidone; polyethylene polyvinyl alcohol copolymers; and polyoxyethylene-polyoxypropylene block copolymers.

56. The composition of claim 44 wherein said composition when administered to a use environment provides a dissolution area under the concentration versus time curve of at least 1.25-fold the corresponding area under the curve for a time period of at least 90 minutes provided by a control composition comprising an equivalent amount of undispersed amorphous drug alone.

57. The composition of claim 44 wherein said composition when administered to a use environment provides a maximum concentration of said drug in said use environment that is at least 1.25-fold a maximum concentration of said drug provided by a control composition comprising an equivalent amount of undispersed amorphous drug alone.

58. The composition of claim 44 wherein said composition when administered to a use environment provides a relative bioavailability of at least 1.25 relative to a control composition comprising an equivalent amount of undispersed amorphous drug alone.

59. A method for forming a pharmaceutical composition comprising:
(a) forming a solution comprising a low-solubility drug, an amphiphilic polymer and a solvent, wherein a portion of said drug and a portion of said polymer are each present in said solution in the form of polymer/drug assemblies having a diameter of from 50 nm to 2000 nm; and
(b) isolating solid aggregated polymer drug/assemblies from said solution, said solid aggregated polymer/drug assemblies comprising said low-solubility drug and said amphiphilic polymer.

60. The method of claim 59 comprising the step of removing said solvent from said solution to isolate said first set of solid aggregated polymer/drug assemblies.

61. The method of claim 60 wherein said solvent is removed by spray-drying.

62. The method of claim 60 wherein said solvent is removed by lyophilization.

63. The method of claim 60, further comprising the step of removing precipitate from said solution prior to removing said solvent.

64. The method of claim 59, further comprising the step of precipitating said polymer/drug assemblies from said solution followed by drying to isolate said solid aggregated polymer/drug assemblies.

65. The method of claim 59, further comprising the step of filtering said polymer/drug assemblies from said solution followed by drying to isolate said solid aggregated polymer/drug assemblies.

66. An aqueous solution comprising:
(a) a low-solubility drug;
(b) an amphiphilic polymer that is at least partially dissolved in said solution;
(c) a portion of said drug and a portion of said polymer being present in said solution as amorphous polymer/drug assemblies, said polymer/drug assemblies having a diameter of from 20 nm to 5000 nm;
(d) said solution having a total dissolved drug concentration of at least 2-fold that of an equilibrium concentration of said drug provided by a control composition consisting of an equivalent amount of said drug in crystalline form alone;
(e) said solution having a free drug concentration of at least 1.5-fold that of said equilibrium concentration provided by said control composition; and
(f) wherein said drug is a CETP inhibitor.

67. A composition as defined in claim 66 wherein said CETP inhibitor is an oxy substituted 4-carboxyamino-2-methyl-1,2,3,4-tetrahydroquinoline having the Formula I 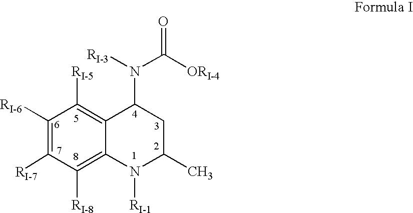
or a pharmaceutically acceptable salt, enantiomer, or stereoisomer of said compound;
wherein R_{I-1} is hydrogen, Y_{I}, W_{I}—X_{I}, W_{I}—Y_{I};
wherein W_{I} is a carbonyl, thiocarbonyl, sulfinyl or sulfonyl;
X_{I} is —O—Y_{I}, —S—Y_{I}, —N (H) —Y_{I} or —N—(Y_{I}) ₂;
wherein Y_{I} for each occurrence is independently Z_{I} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{I};
wherein Z_{I} is a partially saturated, fully saturated or fully unsaturated three to eight membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;
wherein said Z_{I} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino, said (C₁-C6)alkyl substituent is also optionally substituted with from one to nine fluorines; R_{I-3} is hydrogen or Q_{I};
wherein Q_{I} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{I};
wherein V_{I} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;
wherein said V_{I} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carbamoyl, mono-N- or di-N,N- (C₁-C₆) alkylcarbamoyl, carboxyl, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino, said (C₁-C₆)alkyl or (C₂-C₆)alkenyl substituents are also optionally substituted with from one to nine fluorines;
R_{I-4} is Q_{I-1} or V_{I-1},
wherein Q_{I-1} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{I-1};
wherein V_{I-1} is a partially saturated, fully saturated or fully unsaturated three to six membered ring optionally having one to two heteroatoms selected independently from oxygen, sulfur and nitrogen;
wherein said V_{I-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is also optionally substituted with from one to nine fluorines;
wherein either R_{I-3} must contain V_{I} or R_{I-4} must contain V_{I-1}; and
R_{I-5}, R_{I-6}, R_{I-7} and R_{I-8} are each independently hydrogen, hydroxy or oxy wherein said oxy is substituted with T_{I} or a partially saturated, fully saturated or fully unsaturated one to twelve membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with T_{I};
wherein T_{I} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;
wherein said T_{I} substituent is optionally mono-, di- or tri-substituted independently with halo, (C₁-C₆)alkyl, (C₂-C₆)alkenyl, hydroxy, (c₁-C₆)alkoxy, (C₁-₆) alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines. cm 68. A composition according to claim 67 further comprising a compound which is [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester; or a compound which is [2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester; or a compound which is [2R, 4S] 4-[(3,5-Bis-trifluoromethyl-benzyl)-methoxycarbonyl-amino]-2-ethyl-6-trifluoromethyl-3,4-dihydro-2H-quinoline-1-carboxylic acid isopropyl ester.
